(19) United States
(12) Patent Application Publication
(10) Pub. No.: US 2022/0143168 A1
(43) Pub. Date: May 12, 2022

(54) Vaccines targeting H. influenzae

(71) Applicant: Evaxion Biotech A/S, Hørsholm (DK)

(72) Inventors: Andreas Holm Mattsson, Hørsholm (DK); Christian Skjødt Hansen, Hørsholm (DK)

(21) Appl. No.: 17/433,945

(22) PCT Filed: Feb. 27, 2020
(86) PCT No.: PCT/EP2020/055138

§ 371 Date: Aug. 25, 2021

(30) Foreign Application Priority Data
Feb. 27, 2019 (EP) 19159645.1

(51) Int. Cl. A61K 39/102; C07K 14/285; A61K 39/39

(52) U.S. Cl. CPC A61K 39/102; A61K 39/39; C07K 14/285

(57) ABSTRACT

Immunogenic proteins from nontypable Haemophilus influenzae as well as nucleic acids, vectors and transformed cells useful for expression of the proteins. Methods for prophylaxis of infection with nontypable Haemophilus influenzae using the proteins, nucleic acids, vectors or transformed cells.

### FIELD OF THE INVENTION

The present invention relates to the field of antimicrobial prophylaxis and therapy. In particular the present invention relates to proteins and polynucleotides derived from nontypable Haemophilus influenzae and their use in prophylaxis, treatment, and diagnosis of infection with H. influenzae. The invention further relates to vectors comprising the polynucleotides, transformed host organisms expressing the polynucleotides, antibodies (mono- or polyclonal) specific for the polypeptides as well as diagnostic, prophylactic and therapeutic uses and methods. Finally, also methods of preparation are part of the invention.

### BACKGROUND OF THE INVENTION

Haemophilus influenzae is a pathogenic Gram-negative, facultatively anaerobic coccobacillus belonging to the Pasteurellaceae family. The bacterium exists exclusively associated with human epithelia, and is absolutely dependent on exogenous NAD (factor V) and a source of heme (factor X) for aerobic growth.

Isolates of H. influenzae are divided into encapsulated and non-encapsulated strains depending on the presence or non-presence of a polysaccharide capsule. There are six types of encapsulated strains, which express distinct capsular polysaccharides; these may in turn be determined/distinguished immunologically due their specific reactivity with antisera raised against the polysaccharides. Non-encapsulated H. influenzae isolates are termed nontypable H. influenzae (NTHi) because they cannot be distinguished in the same manner and instead requires biotyping, classification of outer membrane protein molecular weight, genetic classification by electrophoresis or PCR, etc. Also whole-genome sequencing may be utilised.

Pathoaenesis of NTHi Infection

NTHi are human epithelial commensals that occasionally cause disease: for instance they may cause otitis media (OM), lower respiratory tract or sinus infections; also the urogenital tract can be a target for infection. In children, OM is typically seen. In adults, lower respiratory tract infections with NTHi are secondary complications to chronic obstructive lung disease.

Key to NTHi pathogenesis is the ability of the bacteria to bind to the human respiratory tract epithelial layer. NTHi exhibits a high degree of specificity towards the epithelia of the nasopharynx, eustachian tubes and middle ear where NTHi is believed to preferentially bind to damaged epithelia. NTHi also appears capabkle of penetrating epithelia and to survive intracellularly within epithelial cells.

NTHi maintains its presence on epithelia despite the host immune response due to a number of immune-evasive factors, such as production of an IgA1 protease. Also, lipooligosaccharide (LOS) may allow the bacterium to modulate the immune response in a way favorable to bacterial survival. Importantly, bacterial proteins are known to exhibit antigenic drift, allowing evasion of host immune responses.

Otitis Media (OM)

OM constitutes a major burden to children and the health system, where it is the major diagnosis in young children and the prevalence appears to be increasing.

The ultimate cause of OM is infectious, comprising bacteria (NTHi, Streptococcus pneumoniae, Moraxella catarrhalis) as well as viruses (respiratory syncytial virus, rhinovirus, influenza virus, parainfluenza virus, metapneumovirus and coronavirus). Bacteria are isolated from >75% of OM cases, and the NTHi incidence appears to be increasing, where it currently is believed to cause 35-50% of acute OM cases.

NTHi is sensitive to antibiotics (ampicillin, amoxicillin) but with the advent of antibiotically resistant strains of NTHi, prophylactic vaccination has become an attractive option. But since effective vaccination against certain other OM causing pathogens has paved the way for NTHi as an infectious agent causing the disease due to so-called pathogen replacement.

Vaccination Against Otitis Media

An effective NTHi vaccine would be of major benefit in preventing NTHi infections and attempts to provide such a vaccine have been made, cf. e.g. U.S. Pat. No. 8,617,574. However, as also noted in this US patent, there is a continuing need for several effective immunogens in order to minimize the risk that pathogen replacement will occur between different strains of NTHi.

### OBJECTS OF THE INVENTION

It is an object of embodiments of the invention to provide Haemophilus influenzae (in particular nontypable Haemophilus influenzae, NTHi) derived antigenic polypeptides that may serve as constituents in vaccines against Haemophilus influenzae infections and in diagnosis of Haemophilus influenzae infections. It is also an object to provide nucleic acids, vectors, transformed cells, vaccine compositions, and other useful means for molecular cloning as well as for therapy and diagnosis with relevance for Haemophilus influenzae, in particular NTHi.

### SUMMARY OF THE INVENTION

It has been found by the present inventor(s) that nontypable Haemophilus influenzae expresses a number of proteins, which are candidates as vaccine targets as well as candidates as immunizing agents for preparation of antibodies that target nontypable Haemophilus influenzae.

So, in a 1^{st} aspect the present invention relates to a polypeptide comprising

a) an amino acid sequence selected from the group consisting of any one of SEQ ID NOs: 1-30, or

b) an amino acid sequence consisting of at least or exactly 5 (normally at least or exactly 9) contiguous amino acid residues from any one of SEQ ID NOs: 1-30, or

c) an amino acid sequence having a sequence identity of at least 60% with the amino acid sequence of a),

d) an amino acid sequence having a sequence identity of at least 60% with the amino acid sequence of b), or

e) an assembly of amino acids derived from any one of SEQ ID NOs: 1-30 which has essentially the same 3D conformation as in the protein from which said assembly is derived so as to constitute a B-cell epitope,

said polypeptide being antigenic in a mammal.

In a 2^{nd} aspect, the invention relates to an isolated nucleic acid fragment, which comprises

i) a nucleotide sequence encoding a polypeptide of the 1^{st} aspect of the invention and of any embodiment of the 1^{st} aspect disclosed herein, or

ii) a nucleotide sequence consisting of the part of any one of SEQ ID NOs: 31-90 that encodes any one of SEQ ID NOs: 1-30,

iii) a nucleotide sequence consisting of a fragment of at least 12 consecutive nucleotides of the nucleotide sequence defined in ii and in same reading frame,

iv) a nucleotide sequence having a sequence identity of at least 60% with the nucleotide sequence in i) or ii),

v) a nucleotide sequence having a sequence identity of at least 60% with the nucleotide sequence in iii),

vi) a nucleotide sequence complementary to the nucleotide sequence in any one of i)-v), or

vii) a nucleotide sequence which hybridizes under highly stringent conditions with the nucleotide sequence in i)-vi).

In a 3^{rd} aspect, the invention relates to a vector comprising the nucleic acid of the 2^{nd} aspect of the invention and of any embodiment of said 2^{nd} aspect, such as a cloning vector or an expression vector.

In a 4^{th} aspect, the invention relates to a transformed cell, which carries the vector of the 3^{rd} aspect of the invention and of any embodiment of the 3^{rd} aspect disclosed herein. Also included in this aspect is a cell line derived from a transformed cell of the invention.

In a 5^{th} aspect, the invention relates to a pharmaceutical composition comprising
a polypeptide of the 1^{st} aspect of the invention and of any embodiment of the 1^{st} aspect disclosed herein,
a nucleic acid fragment of to the 2^{nd} aspect of the invention and of any embodiment of the 2^{nd} aspect disclosed herein,
a vector of the 3^{rd} aspect of the invention and of any embodiment of the 3^{rd} aspect disclosed herein, or
a cell of the 4^{th} aspect of the invention and of any embodiment of the 4^{th} aspect disclosed herein; and

a pharmaceutically acceptable carrier, vehicle or diluent.

In a 6^{th} aspect, the invention relates to a method for inducing immunity in an animal by administering at least once an immunogenically effective amount of
a polypeptide of the 1^{st} aspect of the invention and of any embodiment of the 1^{st} aspect disclosed herein,
a nucleic acid fragment of to the 2^{nd} aspect of the invention and of any embodiment of the 2^{nd} aspect disclosed herein,
a vector of the 3^{rd} aspect of the invention and of any embodiment of the 3^{rd} aspect disclosed herein,
a cell of the 4^{th} aspect of the invention and of any embodiment of the 4^{th} aspect disclosed herein, or
a pharmaceutical composition of the 5^{th} aspect of the invention or of any embodiment of the 5^{th} aspect disclosed herein

so as to induce adaptive immunity against nontypable Haemophilus influenzae in the animal.

In a 7^{th} aspect, the invention relates to a polyclonal antibody in which the antibodies specifically bind to at least one polypeptide of the 1^{st} aspect of the invention and of any embodiment of the 1^{st} aspect disclosed herein, and which is essentially free from antibodies binding specifically to other nontypable Haemophilus influenzae polypeptides; or a an isolated monoclonal antibody or antibody analogue which binds specifically to a polypeptide according to the 1^{st} aspect of the invention and of any embodiment of the 1^{st} aspect disclosed herein.

In an 8^{th} aspect, the invention relates to a pharmaceutical composition comprising an antibody of the 7^{th} aspect of the invention and of any embodiment of the 7^{th} aspect disclosed herein and a pharmaceutically acceptable carrier, vehicle or diluent.

In an 9^{th} aspect, the invention relates to a method for prophylaxis, treatment or amelioration of infection with nontypable Haemophilus influenzae, comprising administering a therapeutically effective amount of 1) an antibody of the 7^{th} aspect of the invention and of any embodiment of the 7^{th} aspect disclosed herein or 2) a pharmaceutical composition of the 8^{th} aspect of the invention and of any embodiment of the 8^{th} aspect disclosed herein, to an individual in need thereof.

In a 10^{th} aspect, the invention relates to a method for determining, quantitatively or qualitatively, the presence of nontypable Haemophilus influenzae, in a sample, the method comprising contacting the sample with an antibody of the 7^{th} aspect of the invention and of any embodiment of the 7^{th} aspect disclosed herein and detecting the presence of antibody bound to material in the sample.

In an 11^{th} aspect, the invention relates to a method for determining, quantitatively or qualitatively, the presence of antibodies specific for nontypable Haemophilus influenzae, in a sample, the method comprising contacting the sample with a polypeptide of the 1^{st} aspect of the invention and of any embodiment of the 1^{st} aspect disclosed herein, and detecting the presence of antibody said polypeptide.

In a 12^{th} aspect, the invention relates to a method for determining, quantitatively or qualitatively, the presence of a nucleic acid characteristic of nontypable Haemophilus influenzae in a sample, the method comprising contacting the sample with a nucleic acid fragment of the 2^{nd} aspect of the invention and of any embodiment of the 2^{nd} aspect disclosed herein, and detecting the presence of nucleic acid in the sample that hybridized to said nucleic acid fragment.

In a 13^{th} aspect, the invention relates to a method for the preparation of the polypeptide of the 1^{st} aspect of the invention and of any embodiment thereof, comprising
culturing a transformed cell of the 4^{th} aspect of the invention and of any embodiment of the 4^{th} aspect disclosed herein, insofar as these relate to a cell capable of expressing the polypeptide of the invention, under conditions that facilitate that the transformed cell expresses the nucleic acid fragment of the 2^{nd} aspect of the invention, option i), and of any embodiment thereof, and subsequently recovering said polypeptide, or
preparing said polypeptide by means of solid or liquid phase peptide synthesis.

In a 14^{th} aspect, the invention relates to a method for determining whether a substance, such as an antibody, is potentially useful for treating infection with nontypable Haemophilus influenzae, the method comprising contacting the polypeptide of the 1^{st} aspect of the invention and of any embodiment thereof with the substance and subsequently establishing whether the substance has at least one of the following characteristics:

1) the ability to bind specifically to said polypeptide,

2) the ability to compered with said polypeptide for specific binding to a ligand/receptor,

3) the ability to specifically inactivate said polypeptide.

In a 15^{th} aspect, the invention relates to a method for determining whether a substance, such as a nucleic acid, is potentially useful for treating infection with nontypable Haemophilus influenzae, the method comprising contacting the substance with the nucleic acid fragment of the 2^{nd} aspect of the invention and of any embodiment thereof, and subsequently establishing whether the substance has either the ability to

1) bind specifically to the nucleic acid fragment, or

2) bind specifically to a nucleic acid that hybridizes specifically with the nucleic acid fragment.

In a 16^{th} aspect, the invention relates to the polypeptide of the 1^{st} aspect of the invention and of any embodiment of the 1^{st} aspect disclosed herein, for use as a pharmaceutical, notably for use as a pharmaceutical in the treatment, prophylaxis or amelioration of infection with Haemophilus influenzae.

In a 17^{th} aspect, the invention relates to a nucleic acid fragment of the 2^{nd} aspect of the invention and of any embodiment of the 1^{st} aspect disclosed herein, or a vector of the 3^{rd} aspect of the invention and of any embodiment of the 2^{nd} aspect disclosed herein, for use as a pharmaceutical, notably for use as a pharmaceutical in the treatment, prophylaxis or amelioration of infection with nontypable Haemophilus influenzae.

In an 18^{th} aspect of the invention, the invention relates to a cell of the 4^{th} aspect of the invention and of any embodiment of the 4^{th} aspect disclosed herein for use as a pharmaceutical, notably for use as a pharmaceutical in the treatment, prophylaxis or amelioration of infection with nontypable Haemophilus influenzae.

Finally, in a 19^{th} aspect, the invention relates to an antibody, antibody fragment or antibody analogue of the 7^{th} aspect of the invention and of any embodiment of the 7^{th} aspect disclosed herein, use as a pharmaceutical, notably use as a pharmaceutical in the treatment, prophylaxis or amelioration of infection with nontypable Haemophilus influenzae.

### LEGENDS TO THE FIGURE

FIG. 1: CFUs in lungs of BALB/c female mice after IN infection with non-typeable H. influenzae.

*p<0.05, **p<0.01, *** p<0.001, **** p<0.0001 vs PBS group; 1Way ANOVA, Bonferroni's multiple comparison test.

FIG. 2: Antibody titres vs. vaccine antigens from vaccinated mice.

A: Titers against antigens from mice vaccinated with vaccine 1 in Example 1.

B: Titers against antigens from mice vaccinated with vaccine 2 in Example 1.

C: Titers against antigens from mice vaccinated with vaccine 4 in Example 1.

FIG. 3: CFUs in lungs of BALB/c female mice after IN infection with non-typeable H. influenzae.

*p<0.05 vs. group 5, #p<0.05 vs. group 4; Kruskal-Wallis test, Dunn's multiple comparison test.

FIG. 4: Antibody titres vs. vaccine antigens from vaccinated mice.

A: Titers against antigens from mice vaccinated with vaccine 1 in Example 2.

B: Titers against antigens from mice vaccinated with vaccine 2 in Example 2.

C: Titers against antigens from mice vaccinated with vaccine 3 in Example 2.

D: Titers against antigens from mice vaccinated with vaccine 4 in Example 2

### DETAILED DISCLOSURE OF THE INVENTION

### Definitions

For the purposes of the present invention, the names “Haemophilus influenzae” and “H. influenzea” generally refer to the nontypable isolates and strains. So, unless otherwise indicated, these to terms refer to the nonytypable H. influenzea, NTHi.

The term “polypeptide” is in the present context intended to mean both short peptides of from 2 to 10 amino acid residues, oligopeptides of from 11 to 100 amino acid residues, and polypeptides of more than 100 amino acid residues. Further-more, the term is also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively The term “amino acid sequence” s the order in which amino acid residues, connected by peptide bonds, lie in the chain in peptides and proteins.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid) according to the following formula: (N_{ref}−N_{dif})·100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAAC-3′ and 5′-ATACGGGAC-3′ will provide the sequence identity 77.8% (N_{ref}=9 and N_{dif}=2). It will be understood that such a sequence identity determination requires that the two aligned sequences are aligned so that there are no overhangs between the two sequences: each amino acid in each sequence will have to be matched with a counterpart in the other sequence.

An “assembly of amino acids” means two or more amino acids bound together by physical or chemical means.

The “3D conformation” is the 3 dimensional structure of a biomolecule such as a protein. In monomeric polypeptides/proteins, the 3D conformation is also termed “the tertiary structure” and denotes the relative locations in 3 dimensional space of the amino acid residues forming the polypeptide.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Hybridization under “stringent conditions” is herein defined as hybridization performed under conditions by which a probe will hybridize to its target sequence, to a detectably greater degree than to other sequences. Stringent conditions are target-sequence-dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Specificity is typically the function of post-hybridization washes, the critical factors being the ionic strength and temperature of the final wash solution. Generally, stringent wash temperature conditions are selected to be about 5° C. to about 2° C. lower than the melting point (Tm) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tm, which is also called the melting temperature. Formulas are available in the art for the determination of melting temperatures.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterologous nucleic acid sequence

The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

### Specific Embodiments of the Invention

The Polypeptides of the Invention

In some embodiments the at least 5 contiguous amino acids referred to in option b) in the definition of the 1^{st} aspect of the invention constitute at least or exactly or at most 6, such as at least or exactly or at most 7, at least or exactly or at most 8, at least or exactly or at most 9, at least or exactly or at most 10, at least or exactly or at most 11, at least or exactly or at most 12, at least or exactly or at most 13, at least or exactly or at most 14, at least or exactly or at most 15, at least or exactly or at most 16, at least or exactly or at most 17, at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27 at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94 contiguous amino acid residues.

The number of contiguous amino acids in option b) can be higher, for all of SEQ ID NOs. 2-30. Another way to phrase this is that for each of SEQ ID NOs: 1-30, the number of the contiguous amino acid residues is at least or exactly or at most N-n, where N is the length of the sequence ID in question and n is any integer between 1 and N-5; that is, the at least or exactly 5 contiguous amino acids can be at least any number between 5 and the length of the reference sequence minus one, in increments of one.

Insofar as embodiment b relates to SEQ ID NOs: 2-30, the at least 5 contiguous amino acids referred to in option b) in the definition of the 1^{st} aspect of the invention may also constitute at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139, at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, or at least or exactly or at most 153 contiguous amino acid residues.

Insofar as embodiment b relates to SEQ ID NOs: 3-30, the at least 5 contiguous amino acids referred to in option b) in the definition of the 1^{st} aspect of the invention may also constitute at least or exactly or at most 154, at least or exactly or at most 155, at least or exactly or at most 154, at least or exactly or at most 156, at least or exactly or at most 157, at least or exactly or at most 158, at least or exactly or at most 159, at least or exactly or at most 160, at least or exactly or at most 161, at least or exactly or at most 162, at least or exactly or at most 163, at least or exactly or at most 164, at least or exactly or at most 165, at least or exactly or at most 166, at least or exactly or at most 167, at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186 contiguous amino acid residues.

Insofar as embodiment b relates to SEQ ID NOs: 4-30, the at least 5 contiguous amino acids referred to in option b) in the definition of the 1^{st} aspect of the invention may also constitute at least or exactly or at most 187, at least or exactly or at most 188, at least or exactly or at most 189, at least or exactly or at most 190, at least or exactly or at most 191, or at least or exactly or at most 192 contiguous amino acid residues.

Insofar as embodiment b relates to SEQ ID NOs: 5-30, the at least 5 contiguous amino acids referred to in option b) in the definition of the 1^{st} aspect of the invention may also constitute at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, at least or exactly or at most 208, at least or exactly or at most 209, at least or exactly or at most 210, at least or exactly or at most 211, at least or exactly or at most 212, at least or exactly or at most 213, at least or exactly or at most 214, or at least or exactly or at most 215 contiguous amino acid residues.

Insofar as embodiment b relates to SEQ ID NOs: 6-30, the at least 5 contiguous amino acids referred to in option b) in the definition of the 1^{st} aspect of the invention may also constitute at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, or at least or exactly or at most 224 contiguous amino acid residues.

Insofar as embodiment b relates to SEQ ID NOs: 7-30, the at least 5 contiguous amino acids referred to in option b) in the definition of the 1^{st} aspect of the invention may also constitute at least or exactly or at most 225, at least or exactly or at most 226, at least or exactly or at most 227, at least or exactly or at most 228, at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, at least or exactly or at most 241, at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 261, at least or exactly or at most 262, or at least or exactly or at most 263 contiguous amino acid residues.

Insofar as embodiment b relates to SEQ ID NOs: 8-30, the at least 5 contiguous amino acids referred to in option b) in the definition of the 1^{st} aspect of the invention may also constitute at least or exactly or at most 264, at least or exactly or at most 265, at least or exactly or at most 266, at least or exactly or at most 267, at least or exactly or at most 268, at least or exactly or at most 269, at least or exactly or at most 270, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, at least or exactly or at most 285, at least or exactly or at most 286, at least or exactly or at most 287, at least or exactly or at most 288, at least or exactly or at most 289, at least or exactly or at most 290, at least or exactly or at most 291, at least or exactly or at most 292, at least or exactly or at most 293, at least or exactly or at most 294, at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, at least or exactly or at most 321, at least or exactly or at most 322, at least or exactly or at most 323, at least or exactly or at most 324, at least or exactly or at most 325, at least or exactly or at most 326, at least or exactly or at most 327, at least or exactly or at most 328, at least or exactly or at most 329, at least or exactly or at most 330, at least or exactly or at most 331, at least or exactly or at most 332, at least or exactly or at most 333, at least or exactly or at most 334, at least or exactly or at most 335, at least or exactly or at most 336, at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, at least or exactly or at most 345, at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, at least or exactly or at most 356, at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367, at least or exactly or at most 368, at least or exactly or at most 369, at least or exactly or at most 370, at least or exactly or at most 371, at least or exactly or at most 372, at least or exactly or at most 373, at least or exactly or at most 374, at least or exactly or at most 375, at least or exactly or at most 376, at least or exactly or at most 377, or at least or exactly or at most 378 contiguous amino acid residues.

Insofar as embodiment b relates to SEQ ID NOs: 9-30, the at least 5 contiguous amino acids referred to in option b) in the definition of the 1^{st} aspect of the invention may also constitute at least or exactly or at most 379, at least or exactly or at most 380, at least or exactly or at most 381, at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, at least or exactly or at most 397, at least or exactly or at most 398, at least or exactly or at most 399, at least or exactly or at most 400, at least or exactly or at most 401, at least or exactly or at most 402, at least or exactly or at most 403, or at least or exactly or at most 404 contiguous amino acid residues.

Insofar as embodiment b relates to SEQ ID NOs: 10-30, the at least 5 contiguous amino acids referred to in option b) in the definition of the 1^{st} aspect of the invention may also constitute at least or exactly or at most 405, at least or exactly or at most 406, or at least or exactly or at most 407 contiguous amino acid residues.

Insofar as embodiment b relates to SEQ ID NOs: 11-30, the at least 5 contiguous amino acids referred to in option b) in the definition of the 1^{st} aspect of the invention may also constitute at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, at least or exactly or at most 420, at least or exactly or at most 421, at least or exactly or at most 422, at least or exactly or at most 423, at least or exactly or at most 424, at least or exactly or at most 425, at least or exactly or at most 426, or at least or exactly or at most 427 contiguous amino acid residues.

Insofar as embodiment b relates to SEQ ID NOs: 12-30, the at least 5 contiguous amino acids referred to in option b) in the definition of the 1^{st} aspect of the invention may also constitute at least or exactly or at most 428, at least or exactly or at most 429, at least or exactly or at most 430, at least or exactly or at most 431, at least or exactly or at most 432, at least or exactly or at most 433, at least or exactly or at most 434, at least or exactly or at most 435, at least or exactly or at most 436, at least or exactly or at most 437, at least or exactly or at most 438, at least or exactly or at most 439, at least or exactly or at most 440, at least or exactly or at most 441, at least or exactly or at most 442, at least or exactly or at most 443, at least or exactly or at most 444, at least or exactly or at most 445, at least or exactly or at most 446, or at least or exactly or at most 447 contiguous amino acid residues.

Insofar as embodiment b relates to SEQ ID NOs: 13-30, the at least 5 contiguous amino acids referred to in option b) in the definition of the 1^{st} aspect of the invention may also constitute at least or exactly or at most 448, at least or exactly or at most 449, at least or exactly or at most 450, at least or exactly or at most 451, at least or exactly or at most 452, at least or exactly or at most 453, at least or exactly or at most 454, at least or exactly or at most 455, at least or exactly or at most 456, at least or exactly or at most 457, at least or exactly or at most 458, at least or exactly or at most 459, at least or exactly or at most 460, at least or exactly or at most 461, at least or exactly or at most 462, or at least or exactly or at most 463 contiguous amino acid residues.

Insofar as embodiment b relates to SEQ ID NOs: 14-30, the at least 5 contiguous amino acids referred to in option b) in the definition of the 1^{st} aspect of the invention may also constitute at least or exactly or at most 464, at least or exactly or at most 465, at least or exactly or at most 466, at least or exactly or at most 467, at least or exactly or at most 468, at least or exactly or at most 469, at least or exactly or at most 470, at least or exactly or at most 471, or at least or exactly or at most 472 contiguous amino acid residues.

Insofar as embodiment b relates to SEQ ID NOs: 15-30, the at least 5 contiguous amino acids referred to in option b) in the definition of the 1^{st} aspect of the invention may also constitute at least or exactly or at most 473, at least or exactly or at most 474, at least or exactly or at most 475, at least or exactly or at most 476, at least or exactly or at most 477, at least or exactly or at most 478, at least or exactly or at most 479, at least or exactly or at most 480, at least or exactly or at most 481, at least or exactly or at most 482, at least or exactly or at most 483, at least or exactly or at most 484, at least or exactly or at most 485, at least or exactly or at most 486, at least or exactly or at most 487, at least or exactly or at most 488, at least or exactly or at most 489, at least or exactly or at most 490, at least or exactly or at most 491, at least or exactly or at most 492, at least or exactly or at most 493, at least or exactly or at most 494, at least or exactly or at most 495, at least or exactly or at most 496, at least or exactly or at most 497, at least or exactly or at most 498, at least or exactly or at most 499, at least or exactly or at most 500, at least or exactly or at most 501, at least or exactly or at most 502, at least or exactly or at most 503, at least or exactly or at most 504, at least or exactly or at most 505, at least or exactly or at most 506, at least or exactly or at most 507 contiguous amino acid residues.

Insofar as embodiment b relates to SEQ ID NOs: 16-30, the at least 5 contiguous amino acids referred to in option b) in the definition of the 1^{st} aspect of the invention may also constitute at least or exactly or at most 508, at least or exactly or at most 509, at least or exactly or at most 510, at least or exactly or at most 511, at least or exactly or at most 512, at least or exactly or at most 513, at least or exactly or at most 514, at least or exactly or at most 515, at least or exactly or at most 516, at least or exactly or at most 517, at least or exactly or at most 518, at least or exactly or at most 519, at least or exactly or at most 520, at least or exactly or at most 521, at least or exactly or at most 522, at least or exactly or at most 523, at least or exactly or at most 524, at least or exactly or at most 525, at least or exactly or at most 526, at least or exactly or at most 527, at least or exactly or at most 528, at least or exactly or at most 529, at least or exactly or at most 530, at least or exactly or at most 531, at least or exactly or at most 532, at least or exactly or at most 533, at least or exactly or at most 534, at least or exactly or at most 535, at least or exactly or at most 536, at least or exactly or at most 537, at least or exactly or at most 538, at least or exactly or at most 539, at least or exactly or at most 540, at least or exactly or at most 541, at least or exactly or at most 542, at least or exactly or at most 543, at least or exactly or at most 544, at least or exactly or at most 545, at least or exactly or at most 546, at least or exactly or at most 547, at least or exactly or at most 548, at least or exactly or at most 549, at least or exactly or at most 550, at least or exactly or at most 551, at least or exactly or at most 552, at least or exactly or at most 553, at least or exactly or at most 554, at least or exactly or at most 555, at least or exactly or at most 556, at least or exactly or at most 557, at least or exactly or at most 558, at least or exactly or at most 559, at least or exactly or at most 560, at least or exactly or at most 561, at least or exactly or at most 562, at least or exactly or at most 563, at least or exactly or at most 564, at least or exactly or at most 565, at least or exactly or at most 566, at least or exactly or at most 567, at least or exactly or at most 568, at least or exactly or at most 569, at least or exactly or at most 570, at least or exactly or at most 571, at least or exactly or at most 572, at least or exactly or at most 573, at least or exactly or at most 574, at least or exactly or at most 575, at least or exactly or at most 576, at least or exactly or at most 577, at least or exactly or at most 578, at least or exactly or at most 579, at least or exactly or at most 580, at least or exactly or at most 581, at least or exactly or at most 582, at least or exactly or at most 583, at least or exactly or at most 584, at least or exactly or at most 585, at least or exactly or at most 586, at least or exactly or at most 587, at least or exactly or at most 588, at least or exactly or at most 589, at least or exactly or at most 590, at least or exactly or at most 591, at least or exactly or at most 592, at least or exactly or at most 593, at least or exactly or at most 594, at least or exactly or at most 595, at least or exactly or at most 596, at least or exactly or at most 597, at least or exactly or at most 598, at least or exactly or at most 599, at least or exactly or at most 600, at least or exactly or at most 601, at least or exactly or at most 602, at least or exactly or at most 603, at least or exactly or at most 604, at least or exactly or at most 605, at least or exactly or at most 606, at least or exactly or at most 607, at least or exactly or at most 608, at least or exactly or at most 609, at least or exactly or at most 610, at least or exactly or at most 611, at least or exactly or at most 612, at least or exactly or at most 613, at least or exactly or at most 614, at least or exactly or at most 615, at least or exactly or at most 616, at least or exactly or at most 617, at least or exactly or at most 618, at least or exactly or at most 619, at least or exactly or at most 620, at least or exactly or at most 621, at least or exactly or at most 622, at least or exactly or at most 623, at least or exactly or at most 624, at least or exactly or at most 625, at least or exactly or at most 626, at least or exactly or at most 627, at least or exactly or at most 628, or at least or exactly or at most 629 contiguous amino acid residues.

Insofar as embodiment b relates to SEQ ID NOs: 17-30, the at least 5 contiguous amino acids referred to in option b) in the definition of the 1^{st} aspect of the invention may also constitute at least or exactly or at most 630, at least or exactly or at most 631, at least or exactly or at most 632, at least or exactly or at most 633, at least or exactly or at most 634, at least or exactly or at most 635, at least or exactly or at most 636, at least or exactly or at most 637, at least or exactly or at most 638, at least or exactly or at most 639, at least or exactly or at most 640, at least or exactly or at most 641, at least or exactly or at most 642, at least or exactly or at most 643, at least or exactly or at most 644, at least or exactly or at most 645, at least or exactly or at most 646, at least or exactly or at most 647, at least or exactly or at most 648, at least or exactly or at most 649, at least or exactly or at most 650, at least or exactly or at most 651, at least or exactly or at most 652, at least or exactly or at most 653, at least or exactly or at most 654, at least or exactly or at most 655, at least or exactly or at most 656, at least or exactly or at most 657, at least or exactly or at most 658, at least or exactly or at most 659, at least or exactly or at most 660, at least or exactly or at most 661, at least or exactly or at most 662, at least or exactly or at most 663, at least or exactly or at most 664, or at least or exactly or at most 665 contiguous amino acid residues.

Insofar as embodiment b relates to SEQ ID NOs: 18-30, the at least 5 contiguous amino acids referred to in option b) in the definition of the 1^{st} aspect of the invention may also constitute at least or exactly or at most 666, at least or exactly or at most 667, at least or exactly or at most 668, at least or exactly or at most 669, at least or exactly or at most 670, at least or exactly or at most 671, at least or exactly or at most 672, at least or exactly or at most 673, at least or exactly or at most 674, at least or exactly or at most 675, at least or exactly or at most 676, at least or exactly or at most 677, at least or exactly or at most 678, at least or exactly or at most 679, at least or exactly or at most 680, at least or exactly or at most 681, at least or exactly or at most 682, at least or exactly or at most 683, at least or exactly or at most 684, at least or exactly or at most 685, at least or exactly or at most 686, at least or exactly or at most 687, at least or exactly or at most 688, at least or exactly or at most 689, at least or exactly or at most 690, at least or exactly or at most 691, or at least or exactly or at most 692 contiguous amino acid residues.

Insofar as embodiment b relates to SEQ ID NOs: 19-30, the at least 5 contiguous amino acids referred to in option b) in the definition of the 1^{st} aspect of the invention may also constitute at least or exactly or at most 693, at least or exactly or at most 694, at least or exactly or at most 695, at least or exactly or at most 696, at least or exactly or at most 697, at least or exactly or at most 698, at least or exactly or at most 699, at least or exactly or at most 700, at least or exactly or at most 701, at least or exactly or at most 702, at least or exactly or at most 703, at least or exactly or at most 704, at least or exactly or at most 705, at least or exactly or at most 706, at least or exactly or at most 707, or at least or exactly or at most 708 contiguous amino acid residues.

Insofar as embodiment b relates to SEQ ID NOs: 20-30, the at least 5 contiguous amino acids referred to in option b) in the definition of the 1^{st} aspect of the invention may also constitute at least or exactly or at most 709, at least or exactly or at most 710, at least or exactly or at most 711, at least or exactly or at most 712, at least or exactly or at most 713, at least or exactly or at most 714, at least or exactly or at most 715, at least or exactly or at most 716, at least or exactly or at most 717, at least or exactly or at most 718, at least or exactly or at most 719, at least or exactly or at most 720, at least or exactly or at most 721, at least or exactly or at most 722, at least or exactly or at most 723, at least or exactly or at most 724, at least or exactly or at most 725, at least or exactly or at most 726, at least or exactly or at most 727, at least or exactly or at most 728, at least or exactly or at most 729, at least or exactly or at most 730, at least or exactly or at most 731, at least or exactly or at most 732, at least or exactly or at most 733, at least or exactly or at most 734, at least or exactly or at most 735, at least or exactly or at most 736, at least or exactly or at most 737, at least or exactly or at most 738, at least or exactly or at most 739, at least or exactly or at most 740, at least or exactly or at most 741, at least or exactly or at most 742, at least or exactly or at most 743, at least or exactly or at most 744, at least or exactly or at most 745, at least or exactly or at most 746, at least or exactly or at most 747, at least or exactly or at most 748, at least or exactly or at most 749, at least or exactly or at most 750, at least or exactly or at most 751, at least or exactly or at most 752, at least or exactly or at most 753, at least or exactly or at most 754, at least or exactly or at most 755, at least or exactly or at most 756, at least or exactly or at most 757, at least or exactly or at most 758, at least or exactly or at most 759, at least or exactly or at most 760, at least or exactly or at most 761, at least or exactly or at most 762, at least or exactly or at most 763, at least or exactly or at most 764, at least or exactly or at most 765, at least or exactly or at most 766, at least or exactly or at most 767, at least or exactly or at most 768, at least or exactly or at most 769, at least or exactly or at most 770, at least or exactly or at most 771, at least or exactly or at most 772, at least or exactly or at most 773, at least or exactly or at most 774, at least or exactly or at most 775, at least or exactly or at most 776, at least or exactly or at most 777, at least or exactly or at most 778, at least or exactly or at most 779, at least or exactly or at most 780, at least or exactly or at most 781, at least or exactly or at most 782, at least or exactly or at most 783, at least or exactly or at most 784, at least or exactly or at most 785, at least or exactly or at most 786, at least or exactly or at most 787, at least or exactly or at most 788, at least or exactly or at most 789, at least or exactly or at most 790, at least or exactly or at most 791, at least or exactly or at most 792, at least or exactly or at most 793, at least or exactly or at most 794, at least or exactly or at most 795, at least or exactly or at most 796, at least or exactly or at most 797, at least or exactly or at most 798, or at least or exactly or at most 799 contiguous amino acid residues.

Insofar as embodiment b relates to SEQ ID NOs: 21-30, the at least 5 contiguous amino acids referred to in option b) in the definition of the 1^{st} aspect of the invention may also constitute at least or exactly or at most 800, at least or exactly or at most 801, at least or exactly or at most 802, at least or exactly or at most 803, at least or exactly or at most 804, at least or exactly or at most 805, at least or exactly or at most 806, at least or exactly or at most 807, at least or exactly or at most 808, at least or exactly or at most 809, at least or exactly or at most 810, at least or exactly or at most 811, at least or exactly or at most 812, at least or exactly or at most 813, at least or exactly or at most 814, at least or exactly or at most 815, at least or exactly or at most 816, at least or exactly or at most 817, at least or exactly or at most 818, at least or exactly or at most 819, at least or exactly or at most 820, at least or exactly or at most 821, at least or exactly or at most 822, at least or exactly or at most 823, at least or exactly or at most 824, at least or exactly or at most 825, at least or exactly or at most 826, at least or exactly or at most 827, at least or exactly or at most 828, at least or exactly or at most 829, at least or exactly or at most 830, at least or exactly or at most 831, at least or exactly or at most 832, at least or exactly or at most 833, at least or exactly or at most 834, at least or exactly or at most 835, at least or exactly or at most 836, at least or exactly or at most 837, at least or exactly or at most 838, at least or exactly or at most 839, at least or exactly or at most 840, at least or exactly or at most 841, at least or exactly or at most 842, or at least or exactly or at most 843 contiguous amino acid residues.

Insofar as embodiment b relates to SEQ ID NOs: 22-30, the at least 5 contiguous amino acids referred to in option b) in the definition of the 1^{st} aspect of the invention may also constitute at least or exactly or at most 844, at least or exactly or at most 845, at least or exactly or at most 846, at least or exactly or at most 847, at least or exactly or at most 848, at least or exactly or at most 849, at least or exactly or at most 850, at least or exactly or at most 851, at least or exactly or at most 852, at least or exactly or at most 853, at least or exactly or at most 854, at least or exactly or at most 855, at least or exactly or at most 856, at least or exactly or at most 857, at least or exactly or at most 858, at least or exactly or at most 859, at least or exactly or at most 860, at least or exactly or at most 861, at least or exactly or at most 862, at least or exactly or at most 863, at least or exactly or at most 864, at least or exactly or at most 865, at least or exactly or at most 866, at least or exactly or at most 867, at least or exactly or at most 868, at least or exactly or at most 869, at least or exactly or at most 870, at least or exactly or at most 871, at least or exactly or at most 872, at least or exactly or at most 873, at least or exactly or at most 874, at least or exactly or at most 875, at least or exactly or at most 876, at least or exactly or at most 877, at least or exactly or at most 878, at least or exactly or at most 879, at least or exactly or at most 880, at least or exactly or at most 881, at least or exactly or at most 882, at least or exactly or at most 883, at least or exactly or at most 884, at least or exactly or at most 885, at least or exactly or at most 886, at least or exactly or at most 887, at least or exactly or at most 888, at least or exactly or at most 889, at least or exactly or at most 890, at least or exactly or at most 891, at least or exactly or at most 892, at least or exactly or at most 893, at least or exactly or at most 894, at least or exactly or at most 895, at least or exactly or at most 896, at least or exactly or at most 897, at least or exactly or at most 898, at least or exactly or at most 899, at least or exactly or at most 900, at least or exactly or at most 901, at least or exactly or at most 902, at least or exactly or at most 903, at least or exactly or at most 904, at least or exactly or at most 905, at least or exactly or at most 906, at least or exactly or at most 907, at least or exactly or at most 908, at least or exactly or at most 909, at least or exactly or at most 910, or at least or exactly or at most 911 contiguous amino acid residues.

Insofar as embodiment b relates to SEQ ID NOs: 23-30, the at least 5 contiguous amino acids referred to in option b) in the definition of the 1^{st} aspect of the invention may also constitute at least or exactly or at most 912, at least or exactly or at most 913, or at least or exactly or at most 914 contiguous amino acid residues.

Insofar as embodiment b relates to SEQ ID NOs: 24-30, the at least 5 contiguous amino acids referred to in option b) in the definition of the 1^{st} aspect of the invention may also constitute at least or exactly or at most 915, at least or exactly or at most 916, at least or exactly or at most 917, at least or exactly or at most 918, at least or exactly or at most 919, or at least or exactly or at most 920 contiguous amino acid residues.

Insofar as embodiment b relates to SEQ ID NOs: 25-30, the at least 5 contiguous amino acids referred to in option b) in the definition of the 1^{st} aspect of the invention may also constitute at least or exactly or at most 921, at least or exactly or at most 922, at least or exactly or at most 923, at least or exactly or at most 924, at least or exactly or at most 925, at least or exactly or at most 926, at least or exactly or at most 927, at least or exactly or at most 928, at least or exactly or at most 929, at least or exactly or at most 930, at least or exactly or at most 931, at least or exactly or at most 932, at least or exactly or at most 933, or at least or exactly or at most 934 contiguous amino acid residues.

Insofar as embodiment b relates to SEQ ID NOs: 26-30, the at least 5 contiguous amino acids referred to in option b) in the definition of the 1^{st} aspect of the invention may also constitute at least or exactly or at most 935, at least or exactly or at most 936, at least or exactly or at most 937, at least or exactly or at most 938, at least or exactly or at most 939, at least or exactly or at most 940, at least or exactly or at most 941, at least or exactly or at most 942, at least or exactly or at most 943, at least or exactly or at most 944, at least or exactly or at most 945, at least or exactly or at most 946, at least or exactly or at most 947, at least or exactly or at most 948, at least or exactly or at most 949, at least or exactly or at most 950, at least or exactly or at most 951, at least or exactly or at most 952, at least or exactly or at most 953, at least or exactly or at most 954, at least or exactly or at most 955, at least or exactly or at most 956, at least or exactly or at most 957, at least or exactly or at most 958, at least or exactly or at most 959, at least or exactly or at most 960, at least or exactly or at most 961, at least or exactly or at most 962, at least or exactly or at most 963, at least or exactly or at most 964, at least or exactly or at most 965, at least or exactly or at most 966, at least or exactly or at most 967, at least or exactly or at most 968, at least or exactly or at most 969, at least or exactly or at most 970, at least or exactly or at most 971, at least or exactly or at most 972, at least or exactly or at most 973, at least or exactly or at most 974, at least or exactly or at most 975, at least or exactly or at most 976, at least or exactly or at most 977, at least or exactly or at most 978, at least or exactly or at most 979, at least or exactly or at most 980, at least or exactly or at most 981, at least or exactly or at most 982, at least or exactly or at most 983, at least or exactly or at most 984, at least or exactly or at most 985, at least or exactly or at most 986, or at least or exactly or at most 987 contiguous amino acid residues.

Insofar as embodiment b relates to SEQ ID NOs: 27-30, the at least 5 contiguous amino acids referred to in option b) in the definition of the 1^{st} aspect of the invention may also constitute at least or exactly or at most 988, at least or exactly or at most 989, at least or exactly or at most 990, at least or exactly or at most 991, at least or exactly or at most 992, at least or exactly or at most 993, at least or exactly or at most 994, at least or exactly or at most 995, at least or exactly or at most 996, at least or exactly or at most 997, at least or exactly or at most 998, at least or exactly or at most 999, at least or exactly or at most 1000, at least or exactly or at most 1001, at least or exactly or at most 1002, at least or exactly or at most 1003, at least or exactly or at most 1004, at least or exactly or at most 1005, at least or exactly or at most 1006, at least or exactly or at most 1007, at least or exactly or at most 1008, at least or exactly or at most 1009, at least or exactly or at most 1010, at least or exactly or at most 1011, at least or exactly or at most 1012, at least or exactly or at most 1013, at least or exactly or at most 1014, at least or exactly or at most 1015, at least or exactly or at most 1016, at least or exactly or at most 1017, at least or exactly or at most 1018, at least or exactly or at most 1019, at least or exactly or at most 1020, at least or exactly or at most 1021, at least or exactly or at most 1022, at least or exactly or at most 1023, at least or exactly or at most 1024, at least or exactly or at most 1025, at least or exactly or at most 1026, at least or exactly or at most 1027, at least or exactly or at most 1028, at least or exactly or at most 1029, at least or exactly or at most 1030, at least or exactly or at most 1031, at least or exactly or at most 1032, at least or exactly or at most 1033, at least or exactly or at most 1034, at least or exactly or at most 1035, at least or exactly or at most 1036, at least or exactly or at most 1037, at least or exactly or at most 1038, at least or exactly or at most 1039, at least or exactly or at most 1040, at least or exactly or at most 1041, at least or exactly or at most 1042, at least or exactly or at most 1043, at least or exactly or at most 1044, at least or exactly or at most 1045, at least or exactly or at most 1046, at least or exactly or at most 1047, at least or exactly or at most 1048, at least or exactly or at most 1049, at least or exactly or at most 1050, at least or exactly or at most 1051, at least or exactly or at most 1052, at least or exactly or at most 1053, at least or exactly or at most 1054, at least or exactly or at most 1055, at least or exactly or at most 1056, at least or exactly or at most 1057, at least or exactly or at most 1058, at least or exactly or at most 1059, at least or exactly or at most 1060, at least or exactly or at most 1061, at least or exactly or at most 1062, at least or exactly or at most 1063, at least or exactly or at most 1064, at least or exactly or at most 1065, at least or exactly or at most 1066, at least or exactly or at most 1067, at least or exactly or at most 1068, at least or exactly or at most 1069, at least or exactly or at most 1070, at least or exactly or at most 1071, at least or exactly or at most 1072, at least or exactly or at most 1073, at least or exactly or at most 1074, at least or exactly or at most 1075, at least or exactly or at most 1076, at least or exactly or at most 1077, at least or exactly or at most 1078, at least or exactly or at most 1079, at least or exactly or at most 1080, at least or exactly or at most 1081, at least or exactly or at most 1082, at least or exactly or at most 1083, at least or exactly or at most 1084, at least or exactly or at most 1085, at least or exactly or at most 1086, at least or exactly or at most 1087, at least or exactly or at most 1088, at least or exactly or at most 1089, at least or exactly or at most 1090, at least or exactly or at most 1091, at least or exactly or at most 1092, at least or exactly or at most 1093, at least or exactly or at most 1094, at least or exactly or at most 1095, at least or exactly or at most 1096, at least or exactly or at most 1097, at least or exactly or at most 1098, at least or exactly or at most 1099, at least or exactly or at most 1100, at least or exactly or at most 1101, at least or exactly or at most 1102, at least or exactly or at most 1103, at least or exactly or at most 1104, at least or exactly or at most 1105, at least or exactly or at most 1106, at least or exactly or at most 1107, at least or exactly or at most 1108, at least or exactly or at most 1109, at least or exactly or at most 1110, at least or exactly or at most 1111, at least or exactly or at most 1112, at least or exactly or at most 1113, at least or exactly or at most 1114, at least or exactly or at most 1115, at least or exactly or at most 1116, at least or exactly or at most 1117, at least or exactly or at most 1118, at least or exactly or at most 1119, at least or exactly or at most 1120, at least or exactly or at most 1121, at least or exactly or at most 1122, at least or exactly or at most 1123, at least or exactly or at most 1124, at least or exactly or at most 1125, at least or exactly or at most 1126, at least or exactly or at most 1127, at least or exactly or at most 1128, at least or exactly or at most 1129, at least or exactly or at most 1130, at least or exactly or at most 1131, at least or exactly or at most 1132, at least or exactly or at most 1133, at least or exactly or at most 1134, at least or exactly or at most 1135, at least or exactly or at most 1136, at least or exactly or at most 1137, at least or exactly or at most 1138, at least or exactly or at most 1139, at least or exactly or at most 1140, at least or exactly or at most 1141, at least or exactly or at most 1142, at least or exactly or at most 1143, at least or exactly or at most 1144, at least or exactly or at most 1145, at least or exactly or at most 1146, at least or exactly or at most 1147, at least or exactly or at most 1148, at least or exactly or at most 1149, at least or exactly or at most 1150, at least or exactly or at most 1151, at least or exactly or at most 1152, at least or exactly or at most 1153, at least or exactly or at most 1154, at least or exactly or at most 1155, at least or exactly or at most 1156, at least or exactly or at most 1157, at least or exactly or at most 1158, at least or exactly or at most 1159, at least or exactly or at most 1160, at least or exactly or at most 1161, at least or exactly or at most 1162, at least or exactly or at most 1163, at least or exactly or at most 1164, at least or exactly or at most 1165, at least or exactly or at most 1166, at least or exactly or at most 1167, at least or exactly or at most 1168, at least or exactly or at most 1169, at least or exactly or at most 1170, at least or exactly or at most 1171, at least or exactly or at most 1172, at least or exactly or at most 1173, at least or exactly or at most 1174, at least or exactly or at most 1175, at least or exactly or at most 1176, at least or exactly or at most 1177, at least or exactly or at most 1178, at least or exactly or at most 1179, at least or exactly or at most 1180, at least or exactly or at most 1181, at least or exactly or at most 1182, at least or exactly or at most 1183, at least or exactly or at most 1184, at least or exactly or at most 1185, at least or exactly or at most 1186, at least or exactly or at most 1187, at least or exactly or at most 1188, at least or exactly or at most 1189, at least or exactly or at most 1190, at least or exactly or at most 1191, at least or exactly or at most 1192, at least or exactly or at most 1193, at least or exactly or at most 1194, at least or exactly or at most 1195, at least or exactly or at most 1196, at least or exactly or at most 1197, at least or exactly or at most 1198, at least or exactly or at most 1199, at least or exactly or at most 1200, at least or exactly or at most 1201, at least or exactly or at most 1202, at least or exactly or at most 1203, at least or exactly or at most 1204, at least or exactly or at most 1205, at least or exactly or at most 1206, at least or exactly or at most 1207, at least or exactly or at most 1208, at least or exactly or at most 1209, at least or exactly or at most 1210, at least or exactly or at most 1211, at least or exactly or at most 1212, at least or exactly or at most 1213, at least or exactly or at most 1214, at least or exactly or at most 1215, at least or exactly or at most 1216, at least or exactly or at most 1217, at least or exactly or at most 1218, at least or exactly or at most 1219, at least or exactly or at most 1220, at least or exactly or at most 1221, at least or exactly or at most 1222, at least or exactly or at most 1223, at least or exactly or at most 1224, at least or exactly or at most 1225, at least or exactly or at most 1226, at least or exactly or at most 1227, at least or exactly or at most 1228, at least or exactly or at most 1229, at least or exactly or at most 1230, at least or exactly or at most 1231, at least or exactly or at most 1232, at least or exactly or at most 1233, at least or exactly or at most 1234, at least or exactly or at most 1235, at least or exactly or at most 1236, at least or exactly or at most 1237, at least or exactly or at most 1238, at least or exactly or at most 1239, at least or exactly or at most 1240, at least or exactly or at most 1241, at least or exactly or at most 1242, at least or exactly or at most 1243, at least or exactly or at most 1244, at least or exactly or at most 1245, at least or exactly or at most 1246, at least or exactly or at most 1247, at least or exactly or at most 1248, at least or exactly or at most 1249, at least or exactly or at most 1250, at least or exactly or at most 1251, at least or exactly or at most 1252, at least or exactly or at most 1253, at least or exactly or at most 1254, at least or exactly or at most 1255, at least or exactly or at most 1256, at least or exactly or at most 1257, at least or exactly or at most 1258, at least or exactly or at most 1259, at least or exactly or at most 1260, at least or exactly or at most 1261, at least or exactly or at most 1262, at least or exactly or at most 1263, at least or exactly or at most 1264, at least or exactly or at most 1265, at least or exactly or at most 1266, at least or exactly or at most 1267, at least or exactly or at most 1268, at least or exactly or at most 1269, at least or exactly or at most 1270, at least or exactly or at most 1271, at least or exactly or at most 1272, at least or exactly or at most 1273, at least or exactly or at most 1274, at least or exactly or at most 1275, at least or exactly or at most 1276, at least or exactly or at most 1277, at least or exactly or at most 1278, at least or exactly or at most 1279, at least or exactly or at most 1280, at least or exactly or at most 1281, at least or exactly or at most 1282, at least or exactly or at most 1283, at least or exactly or at most 1284, at least or exactly or at most 1285, at least or exactly or at most 1286, at least or exactly or at most 1287, at least or exactly or at most 1288, at least or exactly or at most 1289, at least or exactly or at most 1290, at least or exactly or at most 1291, at least or exactly or at most 1292, at least or exactly or at most 1293, at least or exactly or at most 1294, at least or exactly or at most 1295, at least or exactly or at most 1296, at least or exactly or at most 1297, at least or exactly or at most 1298, at least or exactly or at most 1299, at least or exactly or at most 1300, at least or exactly or at most 1301, at least or exactly or at most 1302, at least or exactly or at most 1303, at least or exactly or at most 1304, at least or exactly or at most 1305, at least or exactly or at most 1306, at least or exactly or at most 1307, at least or exactly or at most 1308, at least or exactly or at most 1309, at least or exactly or at most 1310, at least or exactly or at most 1311, at least or exactly or at most 1312, at least or exactly or at most 1313, at least or exactly or at most 1314, at least or exactly or at most 1315, at least or exactly or at most 1316, at least or exactly or at most 1317, at least or exactly or at most 1318, at least or exactly or at most 1319, at least or exactly or at most 1320, at least or exactly or at most 1321, at least or exactly or at most 1322, at least or exactly or at most 1323, at least or exactly or at most 1324, at least or exactly or at most 1325, at least or exactly or at most 1326, at least or exactly or at most 1327, at least or exactly or at most 1328, at least or exactly or at most 1329, at least or exactly or at most 1330, at least or exactly or at most 1331, at least or exactly or at most 1332, at least or exactly or at most 1333, at least or exactly or at most 1334, at least or exactly or at most 1335, at least or exactly or at most 1336, at least or exactly or at most 1337, at least or exactly or at most 1338, at least or exactly or at most 1339, at least or exactly or at most 1340, at least or exactly or at most 1341, at least or exactly or at most 1342, at least or exactly or at most 1343, at least or exactly or at most 1344, at least or exactly or at most 1345, at least or exactly or at most 1346, at least or exactly or at most 1347, at least or exactly or at most 1348, at least or exactly or at most 1349, at least or exactly or at most 1350, at least or exactly or at most 1351, at least or exactly or at most 1352, at least or exactly or at most 1353, at least or exactly or at most 1354, at least or exactly or at most 1355, at least or exactly or at most 1356, at least or exactly or at most 1357, at least or exactly or at most 1358, at least or exactly or at most 1359, at least or exactly or at most 1360, at least or exactly or at most 1361, at least or exactly or at most 1362, at least or exactly or at most 1363, at least or exactly or at most 1364, at least or exactly or at most 1365, at least or exactly or at most 1366, at least or exactly or at most 1367, at least or exactly or at most 1368, at least or exactly or at most 1369, at least or exactly or at most 1370, at least or exactly or at most 1371, at least or exactly or at most 1372, at least or exactly or at most 1373, at least or exactly or at most 1374, at least or exactly or at most 1375, at least or exactly or at most 1376, at least or exactly or at most 1377, at least or exactly or at most 1378, at least or exactly or at most 1379, at least or exactly or at most 1380, at least or exactly or at most 1381, at least or exactly or at most 1382, at least or exactly or at most 1383, at least or exactly or at most 1384, at least or exactly or at most 1385, at least or exactly or at most 1386, at least or exactly or at most 1387, at least or exactly or at most 1388, at least or exactly or at most 1389, at least or exactly or at most 1390, or at least or exactly or at most 1391 contiguous amino acid residues.

Insofar as embodiment b relates to SEQ ID NOs: 28-30, the at least 5 contiguous amino acids referred to in option b) in the definition of the 1^{st} aspect of the invention may also constitute at least or exactly or at most 1392, at least or exactly or at most 1393, at least or exactly or at most 1394, at least or exactly or at most 1395, at least or exactly or at most 1396, at least or exactly or at most 1397, at least or exactly or at most 1398, at least or exactly or at most 1399, at least or exactly or at most 1400, at least or exactly or at most 1401, at least or exactly or at most 1402, at least or exactly or at most 1403, at least or exactly or at most 1404, at least or exactly or at most 1405, at least or exactly or at most 1406, at least or exactly or at most 1407, at least or exactly or at most 1408, at least or exactly or at most 1409, at least or exactly or at most 1410, at least or exactly or at most 1411, at least or exactly or at most 1412, at least or exactly or at most 1413, at least or exactly or at most 1414, at least or exactly or at most 1415, at least or exactly or at most 1416, at least or exactly or at most 1417, at least or exactly or at most 1418, at least or exactly or at most 1419, at least or exactly or at most 1420, at least or exactly or at most 1421, at least or exactly or at most 1422, at least or exactly or at most 1423, at least or exactly or at most 1424, at least or exactly or at most 1425, at least or exactly or at most 1426, at least or exactly or at most 1427, at least or exactly or at most 1428, at least or exactly or at most 1429, at least or exactly or at most 1430, at least or exactly or at most 1431, at least or exactly or at most 1432, at least or exactly or at most 1433, at least or exactly or at most 1434, at least or exactly or at most 1435, at least or exactly or at most 1436, at least or exactly or at most 1437, at least or exactly or at most 1438, at least or exactly or at most 1439, at least or exactly or at most 1440, at least or exactly or at most 1441, at least or exactly or at most 1442, at least or exactly or at most 1443, at least or exactly or at most 1444, at least or exactly or at most 1445, at least or exactly or at most 1446, at least or exactly or at most 1447, at least or exactly or at most 1448, at least or exactly or at most 1449, at least or exactly or at most 1450, at least or exactly or at most 1451, at least or exactly or at most 1452, at least or exactly or at most 1453, at least or exactly or at most 1454, at least or exactly or at most 1455, at least or exactly or at most 1456, at least or exactly or at most 1457, at least or exactly or at most 1458, at least or exactly or at most 1459, at least or exactly or at most 1460, at least or exactly or at most 1461, at least or exactly or at most 1462, at least or exactly or at most 1463, at least or exactly or at most 1464, at least or exactly or at most 1465, at least or exactly or at most 1466, at least or exactly or at most 1467, at least or exactly or at most 1468, at least or exactly or at most 1469, at least or exactly or at most 1470, at least or exactly or at most 1471, at least or exactly or at most 1472, at least or exactly or at most 1473, at least or exactly or at most 1474, at least or exactly or at most 1475, at least or exactly or at most 1476, at least or exactly or at most 1477, at least or exactly or at most 1478, at least or exactly or at most 1479, at least or exactly or at most 1480, at least or exactly or at most 1481, at least or exactly or at most 1482, at least or exactly or at most 1483, at least or exactly or at most 1484, at least or exactly or at most 1485, at least or exactly or at most 1486, at least or exactly or at most 1487, at least or exactly or at most 1488, at least or exactly or at most 1489, at least or exactly or at most 1490, or at least or exactly or at most 1491 contiguous amino acid residues.

Insofar as embodiment b relates to SEQ ID NO: 29 or 30, the at least 5 contiguous amino acids referred to in option b) in the definition of the 1^{st} aspect of the invention may also constitute at least or exactly or at most 1492, at least or exactly or at most 1493, at least or exactly or at most 1494, at least or exactly or at most 1495, at least or exactly or at most 1496, at least or exactly or at most 1497, at least or exactly or at most 1498, at least or exactly or at most 1499, at least or exactly or at most 1500, at least or exactly or at most 1501, at least or exactly or at most 1502, at least or exactly or at most 1503, at least or exactly or at most 1504, at least or exactly or at most 1505, at least or exactly or at most 1506, at least or exactly or at most 1507, at least or exactly or at most 1508, at least or exactly or at most 1509, at least or exactly or at most 1510, at least or exactly or at most 1511, at least or exactly or at most 1512, at least or exactly or at most 1513, at least or exactly or at most 1514, at least or exactly or at most 1515, at least or exactly or at most 1516, at least or exactly or at most 1517, at least or exactly or at most 1518, at least or exactly or at most 1519, at least or exactly or at most 1520, at least or exactly or at most 1521, at least or exactly or at most 1522, at least or exactly or at most 1523, at least or exactly or at most 1524, at least or exactly or at most 1525, at least or exactly or at most 1526, at least or exactly or at most 1527, at least or exactly or at most 1528, at least or exactly or at most 1529, at least or exactly or at most 1530, at least or exactly or at most 1531, at least or exactly or at most 1532, at least or exactly or at most 1533, at least or exactly or at most 1534, at least or exactly or at most 1535, at least or exactly or at most 1536, at least or exactly or at most 1537, at least or exactly or at most 1538, at least or exactly or at most 1539, at least or exactly or at most 1540, or at least or exactly or at most 1541 contiguous amino acid residues.

Insofar as embodiment b relates to SEQ ID NO: 30, the at least 5 contiguous amino acids referred to in option b) in the definition of the 1^{st} aspect of the invention may also constitute at least or exactly or at most 1542, at least or exactly or at most 1543, at least or exactly or at most 1544, at least or exactly or at most 1545, at least or exactly or at most 1546, at least or exactly or at most 1547, at least or exactly or at most 1548, at least or exactly or at most 1549, at least or exactly or at most 1550, at least or exactly or at most 1551, at least or exactly or at most 1552, at least or exactly or at most 1553, at least or exactly or at most 1554, at least or exactly or at most 1555, at least or exactly or at most 1556, at least or exactly or at most 1557, at least or exactly or at most 1558, at least or exactly or at most 1559, at least or exactly or at most 1560, at least or exactly or at most 1561, at least or exactly or at most 1562, at least or exactly or at most 1563, at least or exactly or at most 1564, at least or exactly or at most 1565, at least or exactly or at most 1566, at least or exactly or at most 1567, at least or exactly or at most 1568, at least or exactly or at most 1569, at least or exactly or at most 1570, at least or exactly or at most 1571, at least or exactly or at most 1572, at least or exactly or at most 1573, at least or exactly or at most 1574, at least or exactly or at most 1575, at least or exactly or at most 1576, at least or exactly or at most 1577, at least or exactly or at most 1578, at least or exactly or at most 1579, at least or exactly or at most 1580, at least or exactly or at most 1581, at least or exactly or at most 1582, at least or exactly or at most 1583, at least or exactly or at most 1584, at least or exactly or at most 1585, at least or exactly or at most 1586, at least or exactly or at most 1587, at least or exactly or at most 1588, at least or exactly or at most 1589, at least or exactly or at most 1590, at least or exactly or at most 1591, at least or exactly or at most 1592, at least or exactly or at most 1593, at least or exactly or at most 1594, at least or exactly or at most 1595, at least or exactly or at most 1596, at least or exactly or at most 1597, at least or exactly or at most 1598, at least or exactly or at most 1599, at least or exactly or at most 1600, at least or exactly or at most 1601, at least or exactly or at most 1602, at least or exactly or at most 1603, at least or exactly or at most 1604, at least or exactly or at most 1605, at least or exactly or at most 1606, at least or exactly or at most 1607, at least or exactly or at most 1608, at least or exactly or at most 1609, at least or exactly or at most 1610, at least or exactly or at most 1611, at least or exactly or at most 1612, at least or exactly or at most 1613, at least or exactly or at most 1614, at least or exactly or at most 1615, at least or exactly or at most 1616, at least or exactly or at most 1617, at least or exactly or at most 1618, at least or exactly or at most 1619, at least or exactly or at most 1620, at least or exactly or at most 1621, at least or exactly or at most 1622, at least or exactly or at most 1623, at least or exactly or at most 1624, at least or exactly or at most 1625, at least or exactly or at most 1626, at least or exactly or at most 1627, at least or exactly or at most 1628, at least or exactly or at most 1629, at least or exactly or at most 1630, at least or exactly or at most 1631, at least or exactly or at most 1632, at least or exactly or at most 1633, at least or exactly or at most 1634, at least or exactly or at most 1635, at least or exactly or at most 1636, at least or exactly or at most 1637, at least or exactly or at most 1638, at least or exactly or at most 1639, at least or exactly or at most 1640, at least or exactly or at most 1641, at least or exactly or at most 1642, at least or exactly or at most 1643, at least or exactly or at most 1644, at least or exactly or at most 1645, at least or exactly or at most 1646, at least or exactly or at most 1647, at least or exactly or at most 1648, at least or exactly or at most 1649, at least or exactly or at most 1650, at least or exactly or at most 1651, at least or exactly or at most 1652, at least or exactly or at most 1653, at least or exactly or at most 1654, at least or exactly or at most 1655, at least or exactly or at most 1656, at least or exactly or at most 1657, at least or exactly or at most 1658, at least or exactly or at most 1659, at least or exactly or at most 1660, at least or exactly or at most 1661, at least or exactly or at most 1662, at least or exactly or at most 1663, at least or exactly or at most 1664, at least or exactly or at most 1665, at least or exactly or at most 1666, at least or exactly or at most 1667, at least or exactly or at most 1668, at least or exactly or at most 1669, at least or exactly or at most 1670, at least or exactly or at most 1671, at least or exactly or at most 1672, at least or exactly or at most 1673, at least or exactly or at most 1674, at least or exactly or at most 1675, at least or exactly or at most 1676, at least or exactly or at most 1677, at least or exactly or at most 1678, at least or exactly or at most 1679, at least or exactly or at most 1680, at least or exactly or at most 1681, at least or exactly or at most 1682, at least or exactly or at most 1683, at least or exactly or at most 1684, at least or exactly or at most 1685, at least or exactly or at most 1686, at least or exactly or at most 1687, at least or exactly or at most 1688, at least or exactly or at most 1689, at least or exactly or at most 1690, at least or exactly or at most 1691, at least or exactly or at most 1692, at least or exactly or at most 1693, at least or exactly or at most 1694, at least or exactly or at most 1695, at least or exactly or at most 1696, at least or exactly or at most 1697, at least or exactly or at most 1698, at least or exactly or at most 1699, at least or exactly or at most 1700, at least or exactly or at most 1701, at least or exactly or at most 1702, at least or exactly or at most 1703, at least or exactly or at most 1704, at least or exactly or at most 1705, at least or exactly or at most 1706, at least or exactly or at most 1707, at least or exactly or at most 1708, at least or exactly or at most 1709, at least or exactly or at most 1710, at least or exactly or at most 1711, at least or exactly or at most 1712, at least or exactly or at most 1713, at least or exactly or at most 1714, at least or exactly or at most 1715, at least or exactly or at most 1716, at least or exactly or at most 1717, at least or exactly or at most 1718, at least or exactly or at most 1719, at least or exactly or at most 1720, at least or exactly or at most 1721, at least or exactly or at most 1722, at least or exactly or at most 1723, at least or exactly or at most 1724, at least or exactly or at most 1725, at least or exactly or at most 1726, at least or exactly or at most 1727, at least or exactly or at most 1728, at least or exactly or at most 1729, at least or exactly or at most 1730, at least or exactly or at most 1731, at least or exactly or at most 1732, at least or exactly or at most 1733, at least or exactly or at most 1734, at least or exactly or at most 1735, at least or exactly or at most 1736, at least or exactly or at most 1737, at least or exactly or at most 1738, at least or exactly or at most 1739, at least or exactly or at most 1740, at least or exactly or at most 1741, at least or exactly or at most 1742, at least or exactly or at most 1743, at least or exactly or at most 1744, at least or exactly or at most 1745, at least or exactly or at most 1746, at least or exactly or at most 1747, at least or exactly or at most 1748, at least or exactly or at most 1749, at least or exactly or at most 1750, at least or exactly or at most 1751, at least or exactly or at most 1752, at least or exactly or at most 1753, at least or exactly or at most 1754, at least or exactly or at most 1755, at least or exactly or at most 1756, at least or exactly or at most 1757, at least or exactly or at most 1758, at least or exactly or at most 1759, at least or exactly or at most 1760, at least or exactly or at most 1761, at least or exactly or at most 1762, at least or exactly or at most 1763, at least or exactly or at most 1764, at least or exactly or at most 1765, at least or exactly or at most 1766, at least or exactly or at most 1767, at least or exactly or at most 1768, at least or exactly or at most 1769, at least or exactly or at most 1770, at least or exactly or at most 1771, at least or exactly or at most 1772, at least or exactly or at most 1773, at least or exactly or at most 1774, at least or exactly or at most 1775, at least or exactly or at most 1776, at least or exactly or at most 1777, at least or exactly or at most 1778, at least or exactly or at most 1779, at least or exactly or at most 1780, at least or exactly or at most 1781, at least or exactly or at most 1782, at least or exactly or at most 1783, at least or exactly or at most 1784, at least or exactly or at most 1785, at least or exactly or at most 1786, at least or exactly or at most 1787, at least or exactly or at most 1788, at least or exactly or at most 1789, at least or exactly or at most 1790, at least or exactly or at most 1791, at least or exactly or at most 1792, or at least or exactly or at most 1793 contiguous amino acid residues.

In some embodiments, the polypeptide of the invention also has a sequence identity with the amino acid sequence of a) defined above for all embodiments of at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%. Similarly, the polypeptide of the invention in some embodiments also has a sequence identity with the amino acid sequence of b) defined above for all embodiments of at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%.

In any of the embodiments defined by option b) above, the polypeptide of the invention is also one that has at least 5 contiguous amino acid residues defined for option b) above and also has its N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 in any one of SEQ ID NOs: 1-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 in any one of SEQ ID NOs: 2-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, and 183 in any one of SEQ ID NOs: 3-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 184, 185, 186, 187, 188, and 189 in any one of SEQ ID NOs: 4-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 190, 191, 192, 193, 194, 195, 196, 197, 198, 199, 200, 201, 202, 203, 204, 205, 206, 207, 208, 209, 210, 211, and 212 in any one of SEQ ID NOs: 5-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 213, 214, 215, 216, 217, 218, 219, 220, and 221 in any one of SEQ ID NOs: 6-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, and 260 in any one of SEQ ID NOs: 7-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, and 375 in any one of SEQ ID NOs: 8-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 376, 377, 378, 379, 380, 381, 382, 383, 384, 385, 386, 387, 388, 389, 390, 391, 392, 393, 394, 395, 396, 397, 398, 399, 400, and 401 in any one of SEQ ID NOs: 9-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 402, 403, and 404 in any one of SEQ ID NOs: 10-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 405, 406, 407, 408, 409, 410, 411, 412, 413, 414, 415, 416, 417, 418, 419, 420, 421, 422, 423, and 424 in any one of SEQ ID NOs: 11-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 425, 426, 427, 428, 429, 430, 431, 432, 433, 434, 435, 436, 437, 438, 439, 440, 441, 442, 443, and 444 in any one of SEQ ID NOs: 12-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 445, 446, 447, 448, 449, 450, 451, 452, 453, 454, 455, 456, 457, 458, 459, 460 in any one of SEQ ID NOs: 13-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 461, 462, 463, 464, 465, 466, 467, 468, and 469 in any one of SEQ ID NOs: 14-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, and 504 in any one of SEQ ID NOs: 15-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, and 626 in any one of SEQ ID NOs: 16-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, and 662 in any one of SEQ ID NOs: 17-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 663, 664, 665, 666, 667, 668, 669, 670, 671, 672, 673, 674, 675, 676, 677, 678, 679, 680, 681, 682, 683, 684, 685, 686, 687, 688, and 689 in any one of SEQ ID NOs: 18-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 690, 691, 692, 693, 694, 695, 696, 697, 698, 699, 700, 701, 702, 703, 704, and 705 in any one of SEQ ID NOs: 19-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, and 796 in any one of SEQ ID NOs: 20-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, and 840 in any one of SEQ ID NOs: 21-30, with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, and 908 in any one of SEQ ID NOs: 22-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 909, 910, and 911 in any one of SEQ ID NOs: 23-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 912, 913, 914, 915, 916, and 917 in any one of SEQ ID NOs: 24-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 918, 919, 920, 921, 922, 923, 924, 925, 926, 927, 928, 929, 930, and 931 in any one of SEQ ID NOs: 25-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984 in any one of SEQ ID NOs: 26-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, and 1388 in any one of SEQ ID NOs: 27-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, and 1488 in any one of SEQ ID NOs: 28-30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, and 1538 in SEQ ID NO: 29 or 30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

In any of the embodiments defined by option b) above, the polypeptide of the invention is also one that has at least 5 contiguous amino acid residues defined for option b) above and also has its N-terminal amino acid residue corresponding to any one of amino acid residues 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, and 1790 in SEQ ID NO: 30,

with the proviso that the selected amino acid residue satisfies the formula N≤L−n+1, where N is the number of the selected residue, L is the number of amino acid residues in the sequence from which the residue is selected, and n is the number of consecutive amino acid residues defined for option b, that is, if the length of the at least 5 amino acids is higher than 5, then the N-terminal first residue will not be higher numbered than L−n+1.

Particularly interesting polypeptides of the invention are the following:

| | SEQ |
|---|---|
| | ID |
| Polypeptide | NO: |
| NTHI1164-26-1065 (SEQ ID NO: 30, residues 26-1065) | 91 |
| NTHI1164-1066-1794 (SEQ ID NO: 30, residues 1066- | 92 |
| 1794) | |
| NTHI0503-34-408 (SEQ ID NO: 10, residues 34-408) | 93 |
| NTHI0371-22-921 (SEQ ID NO: 24, residues 22-921) | 94 |
| NTHI1169-21-630 (SEQ ID NO: 16, residues 31-630) | 95 |
| NTHI0354-26-1392 (SEQ ID NO: 27, residues 26-1392) | 96 |
| NTHI0354-26-1000 (SEQ ID NO: 27, residues 26-1000) | 97 |
| NTHI0354-1001-1392 (SEQ ID NO: 27, residues 1001- | 98 |
| 1392) | |
| NTHI1450-63-1542 (SEQ ID NO: 29, residues 63-1542) | 99 |
| NTHI1450-63-735 (SEQ ID NO: 29, residues 63-735) | 100 |
| NTHI1450-736-1542 (SEQ ID NO: 29, residues 736- | 101 |
| 1542) | |
| NTHI1983-63-1492 (SEQ ID NO: 28, residues 63-1492) | 102 |
| NTHI1983-63-735 (SEQ ID NO: 28, residues 63-735) | 103 |
| NTHI1983-736-1492 (SEQ ID NO: 28, residues 736- | 104 |
| 1492) | |
| NTHI0358-30-264 (SEQ ID NO: 7, residues 30-264) | 105 |
| NTHI1667-19-187 (SEQ ID NO: 3, residues 19-187) | 106 |
| NTHI0145-28-666 (SEQ ID NO: 17, residues 28-666) | 107 |
| NTHI1101-20-193 (SEQ ID NO: 4, residues 20-193) | 108 |
| NTHI1229-30-216 (SEQ ID NO: 5, residues 30-216) | 109 |
| NTHI1441-25-95 (SEQ ID NO: 1, residues 25-95) | 110 |
| NTHI0830-20-405 (SEQ ID NO: 9, residues 20-405) | 111 |
| NTHI1168-24-912 (SEQ ID NO: 22, residues 24-912) | 112 |
| NTHI0132-27-464 (SEQ ID NO: 13, residues 27-464) | 113 |
| NTHI1230-26-508 (SEQ ID NO: 15, residues 26-508) | 114 |
| NTHI1489-1-448 (SEQ ID NO: 12, full length) | 115 |
| NTHI1390-28-915 (SEQ ID NO: 23, residues 28-915) | 116 |
| NTHI0538-1-935 (SEQ ID NO: 25, full length) | 117 |
| NTHI0532-38-473 (SEQ ID NO: 14, residues 38-473) | 118 |
| NTHI0482-17-154 (SEQ ID NO: 2, residues 17-154) | 119 |
| NTHI0782-25-988 (SEQ ID NO: 26, residues 25-988) | 120 |
| NTHI0448-155-428 (SEQ ID NO: 11, residues 155-428) | 121 |
| NTHI1844-1-844 (SEQ ID NO: 21, full length) | 122 |
| NTHI0156-1-225 (SEQ ID NO: 6, full length) | 123 |
| NTHI1969-28-693 (SEQ ID NO: 18, residues 28-693) | 124 |
| NTHI1084-20-800 (SEQ ID NO: 20, residues 20-800) | 125 |
| NTHI1851-1-379 (SEQ ID NO: 8, full length) | 126 |
| NTHI0369-22-709 (SEQ ID NO: 19, residues 22-709) | 127 |

as well as fragments and sequence variants of these as defined above.

The polypeptide of the invention is in certain embodiments also fused or conjugated to an immunogenic carrier molecule; or, phrased otherwise, the polypeptide of the invention also includes such an immunogenic carrier molecule in addition to the material derived from SEQ ID NOs: 1-30. The immunogenic carrier molecule is a typically polypeptide that induces T-helper lymphocyte responses in a majority of humans, such as immunogenic carrier proteins selected from the group consisting of keyhole limpet hemocyanino or a fragment thereof, tetanus toxoid or a fragment thereof, diphtheria toxoid or a fragment thereof. Other suitable carrier molecules are discussed infra.

In preferred embodiments, the polypeptide of the invention detailed above is capable of inducing an adaptive immune response against the polypeptide in a mammal, in particular in a human being. Preferably, the adaptive immune response is a protective adaptive immune response against infection with Haemophilus influenzae. The polypeptide may in these cases induce a humoral and/or a cellular immune response.

Epitopes

SEQ ID NOs: 1-30 include antigenic determinants (epitopes) that are as such recognized by antibodies and/or when bound to MHC molecules by T-cell receptors. For the purposes of the present invention, B-cell epitopes (i.e. antibody binding epitopes) are of particular relevance.

It is relatively uncomplicated to identify linear B-cell epitopes—one very simple approach entails that antibodies raised against Haemophilus influenzae or Haemophilus influenzae derived proteins disclosed herein are tested for binding to overlapping oligomeric peptides derived from any one of SEQ ID NO: 1-30. Thereby, the regions of the Haemophilus influenzae polypeptide which are responsible for or contribute to binding to the antibodies can be identified.

Alternatively, or additionally, one can produce mutated versions of the polypeptides of the invention, e.g. version where each single non-alanine residue in SEQ ID NOs.: 1-30 are point mutated to alanine—this method also assists in identifying complex assembled B-cell epitopes; this is the case when binding of the same antibody is modified by exchanging amino acids in different areas of the full-length polypeptide.

Also, in silico methods for B-cell epitope prediction can be employed: useful state-of-the-art systems for β-turn prediction is provided in Petersen B et al. (November 2010), Plos One 5(11): e15079; prediction of linear B-cell epitopes, cf: Larsen J E P et al. (April 2006), Immunome Research, 2:2; prediction of solvent exposed amino acids: Petersen B et al (July 2009), BMC Structural Biology, 9:51.

The Nucleic Acid Fragments of the Invention

The nucleic acid fragment of the invention referred to above is preferably is a DNA fragment (such as SEQ ID NOs: 31-60) or an RNA fragment (such as SEQ ID NOs 61-90).

The nucleic acid fragment of the invention typically

1) consists of at least 13, such as at least 14, at least 15, at least 16, at least 17 at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, least 201, 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264, at least 265, at least 266, at least 267, at least 268, at least 269, at least 270, at least 271, at least 272, at least 273, at least 274, at least 275, at least 276, at least 277, at least 278, at least 279, at least 280, at least 281, at least 282, at least 283, at least 284, or at least 285 consecutive nucleotides of the part of any one of SEQ ID NOs: 36-105 that encodes any one of SEQ ID NOs: 1-30, and

2) is in same reading frame as the part of any one of SEQ ID NOs: 36-105 that encodes any one of SEQ ID NOs: 1-30.

Longer fragments are contemplated, i.e. fragments having at least 300 at least 400, at least 500, at least 600, at least 700, at least 800, at least 900, at least 1000, at least 1500, at least 2000, at least 2500, at least 3000, at least 3500, and at least 4000 nucleotides from those of SEQ ID NOs: 31-90 that encompass fragments of such lengths.

The nucleic acid fragment of the 2^{nd} aspect of the invention is typically one wherein the sequence identity defined in iii) is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

The nucleic acid fragment of the 2^{nd} aspect of the invention is also typically one wherein the sequence identity defined in iv) is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

In embodiments of the 2^{nd} aspect of the invention, the nucleic acid sequences are codon optimized for expression in a host cell.

### The Vectors of the Invention

Vectors of the invention fall into several categories discussed infra. One preferred vector of the invention comprises in operable linkage and in the 5′-3′ direction, an expression control region comprising an enhancer/promoter for driving expression of the nucleic acid fragment defined for option i) above, optionally a signal peptide coding sequence, a nucleotide sequence defined for option i), and optionally a terminator. Hence, such a vector constitutes an expression vector useful for effecting production in cells of the polypeptide of the invention. Since the polypeptides of the invention are bacterial of origin, recombinant production is conveniently effected in bacterial host cells, so here it is preferred that the expression control region drives expression in prokaryotic cell such as a bacterium, e.g. in E coli. However, if the vector is to drive expression in mammalian cell (as would be the case for a DNA or RNA vaccine vector), the expression control region should be adapted to this particular use.

The vector may as indicated further comprises a sequence encoding a signal peptide, which may provide for secretion or membrane integration of the expression product from said vector. For the purposes of nucleic acid vaccination, the signal peptides encoded are typically selected from those described in Williams J. A. Vaccines (Basel). 2013 September; 1(3): 225-249 as well as in the references cited therein.

At any rate, certain vectors of the invention are capable of autonomous replication.

Also, the vector of the invention may be one that is capable of being integrated into the genome of a host cell—this is particularly useful if the vector is use in the production of stably transformed cells, where the progeny will also include the genetic information introduced via the vector. Alternatively, vectors incapable of being integrated into the genome of a mammalian host cell are useful in e.g. nucleic acid vaccination.

Typically, the vector of the invention is selected from the group consisting of a virus, such as a attenuated virus (which may in itself be useful as a vaccine agent), a bacteriophage, a plasmid, a minichromosome, and a cosmid.

A more detailed discussion of vectors of the invention is provided in the following: Polypeptides of the invention may be encoded by a nucleic acid molecule comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found. Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al, 2001; Ausubel et al, 1996, both incorporated herein by reference). In addition to encoding the polypeptides of this invention, a vector of the present invention may encode polypeptide sequences such as a tag or immunogenicity enhancing peptide (e.g. an immunogenic carrier or a fusion partner that stimulates the immune system, such as a cytokine or active fragment thereof). Useful vectors encoding such fusion proteins include pIN vectors, vectors encoding a stretch of histidines, and pGEX vectors, for use in generating glutathione S-transferase (GST) soluble fusion proteins for later purification and separation or cleavage.

Vectors of the invention may be used in a host cell to produce a polypeptide of the invention that may subsequently be purified for administration to a subject or the vector may be purified for direct administration to a subject for expression of the protein in the subject (as is the case when administering a nucleic acid vaccine).

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

Naturally, it may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression (see Sambrook et al, 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to Immunoglobulin Heavy Chain, Immunoglobulin Light Chain, T Cell Receptor, HLA DQα and/or DQβ, β-Interferon, Interleukin-2, Interleukin-2 Receptor, MHC Class II 5, MHC Class II HLA-DRα, β-Actin, Muscle Creatine Kinase (MCK), Prealbumin (Transthyretin), Elastase I, Metallothionein (MTII), Collagenase, Albumin, α-Fetoprotein, γ-Globin, β-Globin, c-fos, c-HA-ras, Insulin, Neural Cell Adhesion Molecule (NCAM), αl-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Polyoma, Retroviruses, Papilloma Virus, Hepatitis B Virus, Human Immunodeficiency Virus, Cytomegalovirus (CMV) IE, and Gibbon Ape Leukemia Virus.

Inducible Elements include MT II—Phorbol Ester (TFA)/Heavy metals; MMTV (mouse mammary tumor virus)—Glucocorticoids; β-Interferon—poly(rl)x/poly(rc); Adenovirus 5 E2—EIA; Collagenase—Phorbol Ester (TPA); Stromelysin—Phorbol Ester (TPA); SV40—Phorbol Ester (TPA); Murine MX Gene—Interferon, Newcastle Disease Virus; GRP78 Gene—A23187; α-2-Macroglobulin—IL-6; Vimentin—Serum; MHC Class I Gene H-2Kb—Interferon; HSP70—E1A/SV40 Large T Antigen; Proliferin—Phorbol Ester/TPA; Tumor Necrosis Factor—PMA; and Thyroid Stimulating Hormonea Gene—Thyroid Hormone.

Also contemplated as useful in the present invention are the dectin-1 and dectin-2 promoters. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of the protein/polypeptide of the current invention in a subject to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

2. Multiple Cloning Sites

Vectors can include a multiple cloning site (MCS), which is a nucleic acid region that contains multiple restriction enzyme sites, any of which can be used in conjunction with standard recombinant technology to digest the vector. Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology.

3. Splicing Sites

Most transcribed eukaryotic RNA molecules will undergo RNA splicing to remove introns from the primary transcripts. If relevant in the context of vectors of the present invention, vectors containing genomic eukaryotic sequences may require donor and/or acceptor splicing sites to ensure proper processing of the transcript for protein expression.

4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

5. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

6. Origins of Replication

In order to propagate a vector in a host cell, it may contain one or more origins of replication sites (often termed “on”), which is a specific nucleic acid sequence at which replication is initiated. Alternatively an autonomously replicating sequence (ARS) can be employed if the host cell is yeast.

7. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

The Transformed Cells of the Invention

Transformed cells of the invention are useful as organisms for producing the polypeptide of the invention, but also as simple “containers” of nucleic acids and vectors of the invention.

Certain transformed cells of the invention are capable of replicating the nucleic acid fragment defined for option i) of the second aspect of the invention. Preferred transformed cells of the invention are capable of expressing the nucleic acid fragment defined for option i).

For recombinant production it is convenient, but not a prerequisite that the transformed cell according is prokaryotic, such as a bacterium, but generally both prokaryotic cells and eukaryotic cells may be used.

Suitable prokaryotic cells are bacterial cells selected from the group consisting of Escherichia (such as E. coli.), Bacillus [e.g. Bacillus subtilis], Salmonella, and Mycobacterium [preferably non-pathogenic, e.g. M. bovis BCG]. Generally, in the event the prokaryotic cell is for use as a live vaccine agent, it is a prerequisite that it is attenuated and/or non-pathogenic.

Eukaryotic cells can be in the form of yeasts (such as Saccharomyces cerevisiae) and protozoans. Alternatively, the transformed eukaryotic cells are derived from a multicellular organism such as a fungus, an insect cell, a plant cell, or a mammalian cell.

For production purposes, it is advantageous that the transformed cell of the invention is is stably transformed by having the nucleic acid defined above for option i) stably integrated into its genome, and in certain embodiments it is also preferred that the transformed cell secretes or carries on its surface the polypeptide of the invention, since this facilitates recovery of the polypeptides produced. A particular version of this embodiment is one where the transformed cell is a bacterium and secretion of the polypeptide of the invention is into the periplasmic space.

An interesting production system is the use of plants. For instance, proteins can be produced at low cost in plants using an Agrobacterium transfection system to genetically modify plants to express genes that encode the protein of interest. One commercially available platform are those provided by iBio CMO LLC (8800 HSC Pkwy, Bryan, Tex. 77807, USA) and iBio, Inc (9 Innovation Way, Suite 100, Newark, Del. 19711, USA) and disclosed in e.g. EP 2 853 599, EP 1 769 068, and EP 2 192 172. Hence, in such systems the vector is an Agrobacterium vector or other vector suitable for transfection of plants.

As noted above, stably transformed cells are preferred—these i.a. allows that cell lines comprised of transformed cells as defined herein may be established—such cell lines are particularly preferred aspects of the invention.

Further details on cells and cell lines are presented in the following:

Suitable cells for recombinant nucleic acid expression of the nucleic acid fragments of the present invention are prokaryotes and eukaryotes. Examples of prokaryotic cells include E. coli; members of the Staphylococcus genus, such as S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens. Examples of eukaryotic cells include mammalian cells; insect cells; yeast cells such as members of the Saccharomyces genus (e.g. S. cerevisiae), members of the Pichia genus (e.g. P. pastoris), members of the Hansenula genus (e.g. H. polymorpha), members of the Kluyveromyces genus (e.g. K. lactis or K. fragilis) and members of the Schizosaccharomyces genus (e.g. S. pombe). As mentioned above, the nucleic acid sequence of the present invention can be appropriately codon optimized to facilitate effective expression from each of the transformed cells of the invention.

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2 nd Edition, Cold Spring Harbor Laboratory Press, 1989.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials or from other depository institutions such as Deutsche Sammlung vor Mikroorganismen und Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins. Bacterial cells used as host cells for vector replication and/or expression include Staphylococcus strains, DH5α, JMI 09, and KC8, as well as a number of commercially available bacterial hosts such as SURE® Competent Cells and SOLOP ACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect el/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Amplification of Nucleic Acids

Nucleic acids used as a template for amplification may be isolated from cells, tissues or other samples according to standard methodologies (Sambrook et al, 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to sequences of genes identified herein are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Methods of Gene Transfer

Suitable methods for nucleic acid delivery to effect expression of compositions of the present invention are believed to include virtually any method by which a nucleic acid (e.g., DNA, including viral and nonviral vectors, as well as RNA) can be introduced into a cell, a tissue or an organism, as described herein or as would be known to one of ordinary skill in the art.

Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859), including microinjection (U.S. Pat. No. 5,789,215); by electroporation (U.S. Pat. No. 5,384,253); by calcium phosphate precipitation; by using DEAE dextran followed by polyethylene glycol; by direct sonic loading; by liposome mediated transfection; by microprojectile bombardment (PCT Application Nos. WO 94/09699 and 95/06128; U.S. Pat. Nos. 5,610,042; 5,322,783 5,563,055, 5,550,318, 5,538,877 and 5,538,880); by agitation with silicon carbide fibers (U.S. Pat. Nos. 5,302,523 and 5,464,765); by Agrobacterium mediated transformation (U.S. Pat. Nos. 5,591,616 and 5,563,055); or by PEG mediated transformation of protoplasts (U.S. Pat. Nos. 4,684,611 and 4,952,500); by desiccation/inhibition mediated DNA uptake. Through the application of techniques such as these, organelle(s), cell(s), tissue(s) or organism(s) may be stably or transiently transformed.

Recently, the development of RNA vaccines has shown great promise. Hence technology for RNA vaccine delivery and expression are within the ambit of the present application. Generally the teachings provided in Deering R. P. et al., Expert Opin Drug Deliv. 2014 June; 11(6):885-99 can be followed in order to effect vaccination with RNA.

The Antibodies of the Invention—and their Production/Isolation

Antibodies directed against the proteins of the invention are useful for affinity chromatography, immunoassays, and for distinguishing/identifying Pseudomonas proteins as well as for passive immunisation and therapy.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 10-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antiserum is obtained by bleeding the immunized animal into a glass or plastic container, incubating the blood at 25 C for one hour, followed by incubating at 4° C. for 2-18 hours. The serum is recovered by centrifugation (eg. 1,000 g for 10 minutes). About 20-50 ml per bleed may be obtained from rabbits.

Monoclonal antibodies are prepared using the standard method of Köhler & Milstein [Nature (1975) 256: 495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (elg. hypoxanthine, aminopterin, thymidine medium, “HAT”). The resulting hybridomas are plated by limiting dilution, and are assayed for production of antibodies, which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly 32p and l25I), electron-dense reagents, enzymes, and ligands having specific binding partners. Enzymes are typically detected by their activity. For example, horseradish peroxidase is usually detected by its ability to convert 3,3′, 5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, 1151 may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with, l251, or with an anti-biotin MAb labeled with HRP. Other permutations and possibilities will be readily apparent to those of ordinary skill in the art, and are considered as equivalents within the scope of the instant invention.

According to the invention, the isolated monoclonal antibody or antibody analogue is preferably a monoclonal antibody selected from a multi-domain antibody such as a murine antibody, a chimeric antibody such as a humanized antibody, a fully human antibody, and single-domain antibody of a llama or a camel, or which is an antibody analogue selected from a fragment of an antibody such as an Fab or an F(ab′)₂, an scFV; cf. also the definition of the term “antibody” presented above.

Compositions of the Invention; Vaccines

Pharmaceutical compositions, in particular vaccines, according to the invention may either be prophylactic (i.e. suited to prevent infection) or therapeutic (i.e. to treat disease after infection).

In some embodiments of the invention, the pharmaceutical compositions such as vaccines include merely one single antigen, immunogen, polypeptide, protein, nucleic acid or vector of the invention, but in other embodiments, the pharmaceutical compositions comprise “cocktails” of the antigens or of the immunogens or of the polypeptides or of the protein or of the nucleic acids or of the vectors of the invention.

In particularly interesting embodiments, the pharmaceutical composition is an MVA vector mentioned herein, which encodes and can effect expression of at least 2 nucleic acid fragments of the invention.

An embodiment of a pharmaceutical composition of the invention comprises exactly Y or at least Y distinct (i.e. having non-identical primary structure) polypeptides of the invention described herein, where each of said Y or at least Y distinct polypeptides comprises an immunogenic amino acid sequence present in or derived from any one of SEQ ID NOs: 1-30 and wherein said Y or at least Y distinct polypeptides together comprise immunogenic amino acid sequences present in or derived from Y or at least Y of SEQ ID NOs. 1-30, wherein Y is an integer selected from 2, 3, 4, 5, 6, 7, 8, 9, 10, 11, 12, 13, 15, 16, 17, 18, 19, 20, 21, 22, 23, 24, 25, 26, 27, 28, 29, and 30.

Another embodiment of a pharmaceutical composition of the invention comprises a peptide/polypeptide comprising or consisting of an immunogenic amino acid sequence present in or derived from SEQ ID NO: 1 in combination with at least one Haemophilus influenzae peptide/polypeptide, in particular with at least one peptide/polypeptide comprising or consisting of an immunogenic amino acid sequence present in or derived from any one of SEQ ID NOs: 2-30. Another embodiment of a pharmaceutical composition of the invention comprises a peptide/polypeptide comprising or consisting of an immunogenic amino acid sequence present in or derived from SEQ ID NO: 2 in combination with at least one Haemophilus influenzae peptide/polypeptide, in particular with at least one peptide/polypeptide comprising or consisting of an immunogenic amino acid sequence present in or derived from any one of SEQ ID NOs: 1, and 3-30. Another embodiment of a pharmaceutical composition of the invention comprises a peptide/polypeptide comprising or consisting of an immunogenic amino acid sequence present in or derived from SEQ ID NO: 3 in combination with at least one Haemophilus influenzae peptide/polypeptide, in particular with at least one peptide/polypeptide comprising or consisting of an immunogenic amino acid sequence present in or derived from any one of SEQ ID NOs: 1, 2, and 4-30. Another embodiment of a pharmaceutical composition of the invention comprises a peptide/polypeptide comprising or consisting of an immunogenic amino acid sequence present in or derived from SEQ ID NO: 4 in combination with at least one Haemophilus influenzae peptide/polypeptide, in particular with at least one peptide/polypeptide comprising or consisting of an immunogenic amino acid sequence present in or derived from any one of SEQ ID NOs: 1-3, and 5-30. Another embodiment of a pharmaceutical composition of the invention comprises a peptide/polypeptide comprising or consisting of an immunogenic amino acid sequence present in or derived from SEQ ID NO: 5 in combination with at least one Haemophilus influenzae peptide/polypeptide, in particular with at least one peptide/polypeptide comprising or consisting of an immunogenic amino acid sequence present in or derived from any one of SEQ ID NOs: 1-4, and 6-30. Another embodiment of a pharmaceutical composition of the invention comprises a peptide/polypeptide comprising or consisting of an immunogenic amino acid sequence present in or derived from SEQ ID NO: 6 in combination with at least one Haemophilus influenzae peptide/polypeptide, in particular with at least one peptide/polypeptide comprising or consisting of an immunogenic amino acid sequence present in or derived from any one of SEQ ID NOs: 1-5, and 7-30. Another embodiment of a pharmaceutical composition of the invention comprises a peptide/polypeptide comprising or consisting of an immunogenic amino acid sequence present in or derived from SEQ ID NO: 7 in combination with at least one Haemophilus influenzae peptide/polypeptide, in particular with at least one peptide/polypeptide comprising or consisting of an immunogenic amino acid sequence present in or derived from any one of SEQ ID NOs: 1-6, and 8-30. Another embodiment of a pharmaceutical composition of the invention comprises a peptide/polypeptide comprising or consisting of an immunogenic amino acid sequence present in or derived from SEQ ID NO: 8 in combination with at least one Haemophilus influenzae peptide/polypeptide, in particular with at least one peptide/polypeptide comprising or consisting of an immunogenic amino acid sequence present in or derived from any one of SEQ ID NOs: 1-7, and 9-30. Another embodiment of a pharmaceutical composition of the invention comprises a peptide/polypeptide comprising or consisting of an immunogenic amino acid sequence present in or derived from SEQ ID NO: 9 in combination with at least one Haemophilus influenzae peptide/polypeptide, in particular with at least one peptide/polypeptide comprising or consisting of an immunogenic amino acid sequence present in or derived from any one of SEQ ID NOs: 1-8, and 10-30. Another embodiment of a pharmaceutical composition of the invention comprises a peptide/polypeptide comprising or consisting of an immunogenic amino acid sequence present in or derived from SEQ ID NO: 10 in combination with at least one Haemophilus influenzae peptide/polypeptide, in particular with at least one peptide/polypeptide comprising or consisting of an immunogenic amino acid sequence present in or derived from any one of SEQ ID NOs: 1-9, and 11-30. Another embodiment of a pharmaceutical composition of the invention comprises a peptide/polypeptide comprising or consisting of an immunogenic amino acid sequence present in or derived from SEQ ID NO: 11 in combination with at least one Haemophilus influenzae peptide/polypeptide, in particular with at least one peptide/polypeptide comprising or consisting of an immunogenic amino acid sequence present in or derived from any one of SEQ ID NOs: 1-10, and 12-30. Another embodiment of a pharmaceutical composition of the invention comprises a peptide/polypeptide comprising or consisting of an immunogenic amino acid sequence present in or derived from SEQ ID NO: 12 in combination with at least one Haemophilus influenzae peptide/polypeptide, in particular with at least one peptide/polypeptide comprising or consisting of an immunogenic amino acid sequence present in or derived from any one of SEQ ID NOs: 1-11, and 13-30. Another embodiment of a pharmaceutical composition of the invention comprises a peptide/polypeptide comprising or consisting of an immunogenic amino acid sequence present in or derived from SEQ ID NO: 13 in combination with at least one Haemophilus influenzae peptide/polypeptide, in particular with at least one peptide/polypeptide comprising or consisting of an immunogenic amino acid sequence present in or derived from any one of SEQ ID NOs: 1-12, and 14-30. Another embodiment of a pharmaceutical composition of the invention comprises a peptide/polypeptide comprising or consisting of an immunogenic amino acid sequence present in or derived from SEQ ID NO: 14 in combination with at least one Haemophilus influenzae peptide/polypeptide, in particular with at least one peptide/polypeptide comprising or consisting of an immunogenic amino acid sequence present in or derived from any one of SEQ ID NOs: 1-13, and 15-30. Another embodiment of a pharmaceutical composition of the invention comprises a peptide/polypeptide comprising or consisting of an immunogenic amino acid sequence present in or derived from SEQ ID NO: 15 in combination with at least one Haemophilus influenzae peptide/polypeptide, in particular with at least one peptide/polypeptide comprising or consisting of an immunogenic amino acid sequence present in or derived from any one of SEQ ID NOs: 1-14, and 16-30. Another embodiment of a pharmaceutical composition of the invention comprises a peptide/polypeptide comprising or consisting of an immunogenic amino acid sequence present in or derived from SEQ ID NO: 16 in combination with at least one Haemophilus influenzae peptide/polypeptide, in particular with at least one peptide/polypeptide comprising or consisting of an immunogenic amino acid sequence present in or derived from any one of SEQ ID NOs: 1-15, and 17-30. Another embodiment of a pharmaceutical composition of the invention comprises a peptide/polypeptide comprising or consisting of an immunogenic amino acid sequence present in or derived from SEQ ID NO: 17 in combination with at least one Haemophilus influenzae peptide/polypeptide, in particular with at least one peptide/polypeptide comprising or consisting of an immunogenic amino acid sequence present in or derived from any one of SEQ ID NOs: 1-16, and 18-30. Another embodiment of a pharmaceutical composition of the invention comprises a peptide/polypeptide comprising or consisting of an immunogenic amino acid sequence present in or derived from SEQ ID NO: 18 in combination with at least one Haemophilus influenzae peptide/polypeptide, in particular with at least one peptide/polypeptide comprising or consisting of an immunogenic amino acid sequence present in or derived from any one of SEQ ID NOs: 1-17, and 19-30. Another embodiment of a pharmaceutical composition of the invention comprises a peptide/polypeptide comprising or consisting of an immunogenic amino acid sequence present in or derived from SEQ ID NO: 19 in combination with at least one Haemophilus influenzae peptide/polypeptide, in particular with at least one peptide/polypeptide comprising or consisting of an immunogenic amino acid sequence present in or derived from any one of SEQ ID NOs: 1-18, and 20-30. Another embodiment of a pharmaceutical composition of the invention comprises a peptide/polypeptide comprising or consisting of an immunogenic amino acid sequence present in or derived from SEQ ID NO: 20 in combination with at least one Haemophilus influenzae peptide/polypeptide, in particular with at least one peptide/polypeptide comprising or consisting of an immunogenic amino acid sequence present in or derived from any one of SEQ ID NOs: 1-19, and 21-30. Another embodiment of a pharmaceutical composition of the invention comprises a peptide/polypeptide comprising or consisting of an immunogenic amino acid sequence present in or derived from SEQ ID NO: 21 in combination with at least one Haemophilus influenzae peptide/polypeptide, in particular with at least one peptide/polypeptide comprising or consisting of an immunogenic amino acid sequence present in or derived from any one of SEQ ID NOs: 1-20, and 22-30. Another embodiment of a pharmaceutical composition of the invention comprises a peptide/polypeptide comprising or consisting of an immunogenic amino acid sequence present in or derived from SEQ ID NO: 22 in combination with at least one Haemophilus influenzae peptide/polypeptide, in particular with at least one peptide/polypeptide comprising or consisting of an immunogenic amino acid sequence present in or derived from any one of SEQ ID NOs: 1-21, and 23-30. Another embodiment of a pharmaceutical composition of the invention comprises a peptide/polypeptide comprising or consisting of an immunogenic amino acid sequence present in or derived from SEQ ID NO: 23 in combination with at least one Haemophilus influenzae peptide/polypeptide, in particular with at least one peptide/polypeptide comprising or consisting of an immunogenic amino acid sequence present in or derived from any one of SEQ ID NOs: 1-22, and 24-30. Another embodiment of a pharmaceutical composition of the invention comprises a peptide/polypeptide comprising or consisting of an immunogenic amino acid sequence present in or derived from SEQ ID NO: 24 in combination with at least one Haemophilus influenzae peptide/polypeptide, in particular with at least one peptide/polypeptide comprising or consisting of an immunogenic amino acid sequence present in or derived from any one of SEQ ID NOs: 1-23, and 25-30. Another embodiment of a pharmaceutical composition of the invention comprises a peptide/polypeptide comprising or consisting of an immunogenic amino acid sequence present in or derived from SEQ ID NO: 25 in combination with at least one Haemophilus influenzae peptide/polypeptide, in particular with at least one peptide/polypeptide comprising or consisting of an immunogenic amino acid sequence present in or derived from any one of SEQ ID NOs: 1-24, and 26-30. Another embodiment of a pharmaceutical composition of the invention comprises a peptide/polypeptide comprising or consisting of an immunogenic amino acid sequence present in or derived from SEQ ID NO: 26 in combination with at least one Haemophilus influenzae peptide/polypeptide, in particular with at least one peptide/polypeptide comprising or consisting of an immunogenic amino acid sequence present in or derived from any one of SEQ ID NOs: 1-25, and 27-30. Another embodiment of a pharmaceutical composition of the invention comprises a peptide/polypeptide comprising or consisting of an immunogenic amino acid sequence present in or derived from SEQ ID NO: 27 in combination with at least one Haemophilus influenzae peptide/polypeptide, in particular with at least one peptide/polypeptide comprising or consisting of an immunogenic amino acid sequence present in or derived from any one of SEQ ID NOs: 1-26, and 28-30. Another embodiment of a pharmaceutical composition of the invention comprises a peptide/polypeptide comprising or consisting of an immunogenic amino acid sequence present in or derived from SEQ ID NO: 28 in combination with at least one Haemophilus influenzae peptide/polypeptide, in particular with at least one peptide/polypeptide comprising or consisting of an immunogenic amino acid sequence present in or derived from any one of SEQ ID NOs: 1-27, 29, and 30. Another embodiment of a pharmaceutical composition of the invention comprises a peptide/polypeptide comprising or consisting of an immunogenic amino acid sequence present in or derived from SEQ ID NO: 29 in combination with at least one Haemophilus influenzae peptide/polypeptide, in particular with at least one peptide/polypeptide comprising or consisting of an immunogenic amino acid sequence present in or derived from any one of SEQ ID NOs: 1-28 and 30. Another embodiment of a pharmaceutical composition of the invention comprises a peptide/polypeptide comprising or consisting of an immunogenic amino acid sequence present in or derived from SEQ ID NO: 30 in combination with at least one Haemophilus influenzae peptide/polypeptide, in particular with at least one peptide/polypeptide comprising or consisting of an immunogenic amino acid sequence present in or derived from any one of SEQ ID NOs: 1-29.

These embodiments entail combinations of peptides/polypeptides which are admixed with each other. Alternatively, the same combinations of peptides/polypeptides can be constructed as fusion polypeptides. Another alternative entails compositions where the immunogens are nucleic acids (DNA or RNA) encoding the peptide combinations or, preferably, encoding such fusion polypeptides.

Another embodiment of the pharmaceutical composition of the invention comprises Z or at least Z distinct nucleic acid molecules each encoding a polypeptide of the invention, where each of said Z or at least Z distinct nucleic acid molecules encodes an immunogenic amino acid sequence present in or derived from any one of SEQ ID NOs: 1-30, and wherein said at Z or least Z distinct nucleic acid molecules together encode immunogenic amino acid sequences present in or derived from at Z or least Z of SEQ ID NOs. 1-30, wherein Z is an integer selected from 1, 2, 3, 4, 5, 6, 7, 8, 9, 10, 11, 12, 13, 15, 16, 17, 18, 19, 20, 21, 22, 23, 24, 25, 26, 27, 28, 29, and 30. Also, such a pharmaceutical composition may include nucleic acids that encode several immunogenic amino acid sequences disclosed herein, either as separate encoded species or as peptides fused to each other. So one variation of this embodiment is one single nucleic acid molecule, which encodes one or more of the polypeptides disclosed above or one or more of the combinations of peptides disclosed above.

Vaccines of the invention typically comprise immunising antigen(s), immunogen(s), polypeptide(s), protein(s) or nucleic acid(s), usually in combination with “pharmaceutically acceptable carriers”, which include any carrier that does not itself induce the production of antibodies harmful to the individual receiving the composition or targeting the protein/pathogen. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogen, cf. the description of immunogenic carriers supra.

The pharmaceutical compositions of the invention thus typically contain an immunological adjuvant, which is commonly an aluminium based adjuvant or one of the other adjuvants described in the following: Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi adjuvant system (RAS), (Ribi Immunochem, Hamilton, Mont.) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc.; and (6) other substances that act as immunostimulating agents to enhance the effectiveness of the composition. Alum and MF59™ adjuvants are preferred.

Muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (eg. the immunising antigen or immunogen or polypeptide or protein or nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (eg. WO 98/20734). Additional formulations suitable for other modes of administration include oral, pulmonary and nasal formulations, suppositories, and transdermal applications. In the case of nucleic acid vaccination and antibody treatment, also the intravenous or intraarterial routes may be applicable.

Dosage treatment may be a single dose schedule or a multiple dose schedule. The vaccine may be administered in conjunction with other immunoregulatory agents.

As an alternative to protein-based vaccines, DNA vaccination (also termed nucleic acid vaccination or gene vaccination) may be used [eg. Robinson & Torres (1997) Seminars in Immunol 9: 271-283; Donnelly et al. (1997) Avnu Rev Immunol 15: 617-648; later herein]. Also vaccination with RNA is an interesting and highly promising technology, cf. the above-mentioned reference by Deering R. P. et al.

Treatment Methods of the Invention

The method of the sixth aspect of the invention generally relates to induction of immunity and as such also entails method that relate to treatment, prophylaxis and amelioration of disease.

When immunization methods entail that a polypeptide of the invention or a composition comprising such a polypeptide is administered the animal (e.g. the human) typically receives between 0.5 and 5,000 μg of the polypeptide of the invention per administration.

In preferred embodiments of this aspect, the immunization scheme includes that the animal (e.g. the human) receives a priming administration and one or more booster administrations.

Preferred embodiments of this aspect of the invention comprise that the administration is for the purpose of inducing protective immunity against Haemophilus influenzae. In turn this means that the administration is a prophylactic or therapeutic treatment of otitis media and/or sinusitis and/or meningitis and/or bacteremia, in particular in children, when these diseases are caused by H. influenzae. It also means that the administration is a prophylactic or therapeutic treatment of otitis media (OM), lower respiratory tract infections and/or inflammation, sinus infections (sinusitis), and urogenital tract infections and/or inflammation. In this embodiment it is particularly preferred that the protective immunity is effective in reducing the risk of attracting infection with Haemophilus influenzae or is effective in treating or ameliorating infection with Haemophilus influenzae.

As mentioned herein, the preferred vaccines of the invention induce humoral immunity, so it is preferred that the administration is for the purpose of inducing antibodies specific for Haemophilus influenzae and wherein said antibodies or B-lymphocytes producing said antibodies are subsequently recovered from the animal.

But, as also mentioned the method of this aspect may also be useful in antibody production, so in other embodiments the administration is for the purpose of inducing antibodies specific for Haemophilus influenzae and wherein B-lymphocytes producing said antibodies are subsequently recovered from the animal and used for preparation of monoclonal antibodies.

Pharmaceutical compositions can as mentioned above comprise polypeptides, antibodies, or nucleic acids of the invention. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. Reference is however made to the ranges for dosages of immunologically effective amounts of polypeptides, cf. above.

However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles.

Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

As is apparent from the claims, the invention also relates to related aspect and embodiments to the treatment and prophylaxis disclosed herein: the invention also includes aspects and embodiments where
the polypeptide of the invention is for use as a pharmaceutical, in particular for use as a pharmaceutical in the treatment, prophylaxis or amelioration of infection with Haemophilus influenzae;
the nucleic acid fragment of the invention or the vector of the invention is for use as a pharmaceutical, in particular for use as a pharmaceutical in the treatment, prophylaxis or amelioration of infection with Haemophilus influenzae;
the transformed cell of the invention is for use as a pharmaceutical, in particular for use as a pharmaceutical in the treatment, prophylaxis or amelioration of infection with Haemophilus influenzae.
the antibody, antibody fragment or antibody analogue of the invention is for use as a pharmaceutical, in particular for use as a pharmaceutical in the treatment, prophylaxis or amelioration of infection with Haemophilus influenzae.

Each of these aspect also entail that the treatment, prophylaxis or amelioration is of conditions selected from otitis media (OM), lower respiratory tract infections and/or inflammation, sinus infections and/or inflammation, and urogenital tract infection and/or inflammation.

### Example 1

Titration Study for Determination of Optimum Challenge Inoculum

The objective of the experiment was to determine the challenge dose of Non-typeable Haemophilus influenzae to be subsequently used for vaccine testing in efficacy studies.

The study was carried out in two rounds: In the initial study four inoculum sizes with 1 log difference between them was evaluated in regards to CFU count in lung tissue 6 and 48 hours post challenge, whereas the second study focused on a more narrow selection of inocula; here the CFU was determined at 6 and 24 hours post challenged.

Microorganism:

Non-typeable Haemophilus influenzae strain 2019

Animal Identification

Species: Mus musculus

Strain: Balb/c

Sex: Female

Origin: Charles River, Germany

Identification: 1-48 in each round

Age: 6 wks upon arrival

Animal Husbandry
Acclimatization period: 5 days
Caging/Hosing: Sealsafe plus mouse IVC green line system (TECNIPLAST S.p A., Italy); in solid bottom cages type GM500SU; three animals were housed per cage on irradiated, corn cob for laboratory animals (Scobis Due—Mucedola, Italy) with a provision of one cotton nestlet for nest making, a Des Res paper shelter (Lillico Serving Biotechnology, UK) and Wood Gnawing Blocks (Certified, Bio-Serv USA).
Room temperature: 22° C.±2° C.
Relative humidity: 55%±10%
Air changes: 15-20 air changes per hour
Lighting cycle: Light cycle of 12 hours light (7:00 to 19:00)/12 hours dark.
Diet: Pelleted food for mice; ad libitum
Brand diet: DS VRF 1 (P), UK
Water: Drinking water from municipal water main (bottles TECNIPLAST S.p.A. Italy); ad libitum.

Allocation of Animals to Groups

Animals arrived in groups of 10 and they are grouped randomly into groups as designated in table 1 upon arrival. At D1 all animals was weighed and each mouse was identified by number, as well as with tail mark within the cage. Starting body weight was be recorded into the prepared table. Each single cage will have a tag, indicating experiment number, progressive cage and animal number (given).

Health Check

All animals were subjected to a detailed physical examination by a veterinarian to ensure that they are in a good state of health prior to start of the study.

Materials

Narkamon 100, Bioveta, Czech Republic;

Xylazine 2%, Alfasan International B.V., Woerden, Netherlands;

1 mL syringes; BD Plastipak; ref 300013; Becton Dickinson GmbH, Spain;

Sterile, single use needles; BD Microlance 3, 25 G 1″ Nr.18; 0.5×25 mm, ref 300400;

PBS, Sigma, cat. no. P4417, Germany;

Precellys lysing kit, ref. no. KT03961-1-302.7, Bertin technologies, France

Inoculum Preparation/Application Dose for Intranasal Challenge

Glycerol stocks of Non-typeable Haemophilus influenzae were thawed, centrifuged and bacteria were re-suspended in 1 mL of PBS for each group of animals.

Preparation of Anaesthesia

The working solution was 1 mL of Narkamon and 0.2 mL of Xylazine added into 9 mL saline. Dose volume was 10 mL/kg b.w.

Study Groups

| | | IN Infection with | |
|---|---|---|---|
| | | Haemophilus influenzae | |
| Group No | | Inoculum size | |
| (first round) | #/group | 50 μL/mouse* | CFU in lungs |
| 1.1 | 12 | ≈1.0 × 10⁹ | 6 hrs PI. |
| 1.2 | | ≈1.0 × 10⁸ | (n = 6/group) |
| 1.3 | | ≈1.0 × 10⁷ | 48 hrs PI |
| 1.4 | | ≈1.0 × 10⁶ | (n = 6/group) |

*The exact inoculum size was determined experimentally, see under results

| | | IN Infection with | |
|---|---|---|---|
| | | Haemophilus influenzae | |
| Group No | | Inoculum size | |
| (second round) | #/group | 50 μL/mouse* | CFU in lungs |
| 2.1 | 12 | ≈5.0 × 10⁷ | 6 hrs PI. |
| 2.2 | | ≈1.0 × 10⁷ | (n = 6/group) |
| 2.3 | | ≈5.0 × 10⁶ | 24 hrs PI |
| 2.4 | | ≈1.0 × 10⁶ | (n = 6/group) |

*The exact inoculum size was determined experimentally, see under results

Procedure

Each mouse was challenged with an intranasal (IN) administration of 50 μL/mouse of a suspension containing bacteria under ketamine/xylazine anaesthesia. Animals were examined clinically twice daily up to 48 hrs p.i. All unexpected events will be recorded.

In a first set of experiments, mice from each group was scarified at 6 and 48 hrs PI, in the second set of experiments, mice were sacrificed at 6 and 24 hrs PI. Lungs were aseptically removed from the thoracic cavity and placed into a pre-weighed sterile Precellys test tube containing 2 mL of sterile PBS. Post-sampling, Precellys test tubes were weighed once again. Lungs were homogenized in sterile PBS by Precellys Evolution homogenizer, BERTIN Technologies (7 mL per test tube; Speed: 6800 RPM; Cycle: 4×15 s; Pause: 5 s). Thereafter, the homogenized samples were serially diluted for CFU determination: 100 μL of homogenates were transferred to 96 well U-bottom plates where serial dilutions in PBS were made by transferring 10 μL of homogenates to 90 μL of PBS. After each dilution, pipetting tips were discarded. 9 serial dilutions were made, out of which 10 μL were used for inoculation of agar plates in 5 replicates. Agar plates were then incubated overnight at 37° C. and CFU counted.

All animals found seriously ill and those who met the humane endpoint criteria as defined in the CARE_ZG_End point table were euthanized with Euthasol. The read-out consisted in CFU counts in lung tissue at 6 hr and 48 hrs post infection (PI).

Data was processed using Microsoft Excel SW. Statistical analysis and graphical presentation was performed using GraphPad Prism software (version 7.04). D'Agostino & Pearson omnibus normality test was used to determine whether to use parametric or non-parametric tests for each readout. 1 way ANOVA—combined with Dunnett's multiple comparison test or Kruskall-Wallis test with Dunn's multiple comparison test was used based on the results from D'Agostino & Pearson omnibus normality test. Differences between groups was considered statistically significant when p<0.05.

All animal related research was conducted in accordance with 2010/63/EU and National legislation regulating the use of laboratory animals in scientific research and for other purposes (Official Gazette 55/13). An Institutional Committee on Animal Research Ethics (CARE-Zg) oversees that animal related procedures are not compromising the animal welfare.

Results

Infection with the inoculum size of 1.0×10⁸ CFU/50 μL resulted in 9.45f0.19 Log 10 CFU in lungs at 6 h PI, while after 48 h, the PI count was 6.63±0.27 Log 10 CFU. Infection with the inoculum size of 1.1×10⁷ CFU/50 μL resulted in 8.75f0.12 Log 10 CFU in lungs at 6 h PI, while after 48 h, the PI count was 6.69±0.14 Log 10 CFU. Infection with the inoculum size of 1.06×10⁶ CFU/50 μL resulted in 7.21±0.12 Log 10 CFU in lungs at 6 h PI, while after 48 h the PI count was 5.22f0.23 Log 10 CFU. Infection with the inoculum size of 1.06×10⁵ CFU/50 μL resulted in 5.25f0.18 Log 10 CFU in lungs at 6 h PI, while after 48 h the PI count was 4.12±0.23 Log 10 CFU.

Lung infection was thus successfully induced after IN challenge with all inoculum sizes of non-typeable Haemophilus influenzae strain 2019 in female BALB/c mice. A decrease of CFU counts between 6 hrs and 48 hrs at all inoculum sizes was however observed.

Based on these findings, it was in round 2 investigated in more detail in an almost identical experiment whether to use the inoculum size 5×10⁷, 1×10⁷, 5×10⁶, or 1×10⁶ CFU/50 μL/mouse. In order to minimize the decrease in CFU counts, the CFU was in contrast to the initial experiment determined at 6 hr and at 24 hr PI. The results here were:

Infection with the inoculum size of 4.23×10⁷ CFU/50 μL resulted in 8.53f0.09 Log 10 CFU in lungs at 6 h PI, while at 24 h the PI count was 7.51±0.15 Log 10 CFU. Infection with the inoculum size of 9.0×10⁶ CFU/50 μL resulted in 7.80±0.16 Log 10 CFU in lungs at 6 h PI, while at 24 h PI the count was 6.52±0.22 Log 10 CFU. Infection with the inoculum size of 4.60×10⁶ CFU/50 μL resulted in 7.20±0.16 Log 10 CFU in lungs at 6 h PI, while at 24 h the PI count was 6.32±0.20 Log 10 CFU. Infection with the inoculum size of 9.38×10⁵ CFU/50 μL resulted in 6.74±0.12 Log 10 CFU in lungs at 6 h PI, while at 24 h the PI count was 6.09±0.27 Log 10 CFU.

Thus, also in the follow up experiment Lung infection was successfully induced after IN challenge with all inoculum sizes and also in this round a decrease of CFU counts between 6 and 24 hrs PI at all inoculum sizes was observed.

On the basis of these results, it was decided to carry out efficacy testing of the experimental vaccines in mice challenged with an inoculum of ≈1×10⁷ CFU the bacteria.

### Example 2

Efficacy Testing of 3 Combination Vaccines and Own Positive Control in a Murine Model of Non-Typeable Haemophilus influenzae Induced Lung Infection

The aim of this Example was to evaluate the protective effects of immunization with 3 combination vaccines and a whole cell vaccine after intranasal (IN) challenge with non-typeable Haemophilus influenzae strain 2019 in BALB/c female mice.

Mice underwent three immunizations with 14-day intervals between each vaccination. At day 36, mice were challenged with non-typeable Haemophilus influenzae strain 2019, using an inoculum size of 7.75×10⁶ CFU/50 μL via the intranasal route under deep ketamine+xylazine anesthesia. Challenged animals were sacrificed 6 hrs PI in order to determine CFUs in lungs.

Experimental Details

### List of Abbreviations (Also Used in Example 3)

BW Body weight

PBS Phosphate-Buffered Saline

CFU Colony forming units

ON Over night

PI Post infection

rpm Revolutions per minute

SC Subcutaneous administration

IN Intra nasal administration

MHB Mueller Hinton Broth

LW Lung weight

Microorganism:

Non-typeable Haemophilus influenzae strain 2019

Animal Identification

Species: Mus musculus

Strain: Balb/c

Sex: Female

Origin: Charles River, Germany

Identification: 1-80

Age: 6 wks upon arrival

Animal Husbandry
Acclimatization period: 5 days
Caging/Hosing: TECNIPLAST S.p.A. Italy, cages, type III, polysulfone cage with a 3-4 cm thick Scobis Duo, Mucedola, Italy with a provision of one cotton nestlet for nest making and a Des Res paper shelter (Lillico Serving Biotechnology, UK), as well as with ASPEN BLOCKS, MEDIUM (20×20×100 mm), LBS (Serving Biotechnology, UK).
Room temperature: 22° C.±2° C.
Relative humidity: 55%±10%
Air changes: 15-20 air changes per hour
Lighting cycle: Light cycle of 12 hours light (7:00 to 19:00)/12 hours dark.
Diet: Pelleted food for mice; ad libitum
Brand diet: SDS VRF 1 (P), UK
Water: Drinking water from municipal water main (bottles TECNIPLAST S.p.A. Italy); ad libitum.

Allocation of Animals to Groups

Animals were grouped randomly into 5 groups of 16 animals as designated in the following table:

| | | Adjuvants | Blood | Infection | Experimental |
|---|---|---|---|---|---|
| Group | Vaccine | regime | sampling | (D36) | procedures |
| 1 | Vaccine | Combination | D2 + D35 | ≈1.0 × 10⁷ | Immunization |
| | #1 | vaccines: | | CFU/50 μL | (SC): |
| 2 | Vaccine | 1^{st} IFA/ | | IN/mouse | D0; D14 |
| | #2 | ALUgel | | | and D28 |
| 3 | PBS | 2^{nd} and 3^{rd} | | | Blood |
| 4 | Vaccine | ALUgel | | | collection: |
| | #3 | Positive | | | D2 and D35 |
| 5 | Whole | control: | | | IN challenge: |
| | cell | ALUgel | | | D36 |
| | | | | | Lung sampling |
| | | | | | for CFU: |
| | | | | | D36: 6 hrs PI |

On D2 all animals were weighed and each mouse was identified by number, as well as with a tail mark within the cage. Initial body weight was recorded into the prepared table. Each single cage had a tag, indicating experiment number, progressive cage and animal number (given).

Health Check

All animals were subjected to a detailed physical examination by a veterinarian to ensure that they are in a good state of health prior to start of the study.

Test Vaccines

Vaccine No. 1 (Group 1):

| | | | |
|---|---|---|---|
| Left: | NTHI1164-1066-1794 | Right: | NTHI0503-34-408 |
| | NTHI1164-26-1065 | | NTHI0371-22-921 |
| | NTHI1169-21-630 | | NTH10358-30-264 |

Vaccine No. 2 (Group 2):

| | | | |
|---|---|---|---|
| Left: | NTHI0354-1001-1392 | Right: | NTHI1667-19-187 |
| | NTHI0354-26-1000 | | NTHI1101-20-193 |
| | NTHI0354-26-1392 | | NTH11229-30-216 |
| | NTHI1450-63-1542 | | |
| | NTHI1450-736-1542 | | |

Vaccine No. 3 (Group 4):

Left: NTHI0370-27-565 Right: PE-PilA
(NTHI0370 is disclosed in US patent application publication no. 2012/0045457 as the protein having SEQ ID NO: 191. The PE-PilA fusion protein is disclosed in US patent application publication no. 2014/0056934 as the protein having SEQ ID NO: 177).

Whole Cell (Group 5):

Batch #01 NTHI, strain 2019

Materials
Narkamon 100, Bioveta, Czech Republic
Xylazine 2%, Alfasan International B.V., Woerden, Netherlands
1 mL syringes; BD Plastipak; ref 300013; Becton Dickinson GmbH, Spain
Sterile, single use needles; BD Microlance 3, 25 G 1″ Nr.18; 0.5×25 mm, ref 300400, Becton Dickinson Drogheda, Ireland
Alhydrogel 2% Brenntag, DK, distributed by InvivoGen, cat. code: vac-alu-250
0.9% NaCL solution, PLIVA
PBS, SIGMA, P4417
IFA, Sigma-Aldrich, cat. no. F5506
BBL™ Mueller Hinton Broth, REF 212322, Becton Dickinson
Precellys lysing kit, ref. no. KT03961-1-302.7, Bertin technologies, France

Experimental Setup

Inocula were prepared from frozen bacterial stocks. The bacterial suspension was centrifuged (8000×RPM, 10 min, +4° C.) and re-suspended in 180 mL of HTM media. To this bacterial solution, 60 mL 50% glycerol solution was added giving all together 240 mL of bacteria in 12.5% glycerol final. Suspension was aliquoted in 240 tubes with 1 mL of suspension per tube and stored at −80° C. Glycerol stocks of Non-typeable Haemophilus influenzae were thawed, centrifuged and bacteria were re-suspended in PBS for each group of animals.

Actual inoculum size was confirmed by plating prepared suspensions on surface of Mueller Hinton Agar plate supplemented with 5% defibrinated sheep blood. Plates were incubated at 37° C., 5% CO₂ and colonies counted.

Procedures

The anesthetic working solution consisted of 1 mL of Narkamon, 0.2 mL of Xylazine, and 9 mL of saline and was does in the animals at 10 mL/kg.

The vaccines were composed according as shown in the following table and as described in the protocols below. Some protein solutions exceed 2200 μl after refolding; here excess buffers can be aspirated off to reach the 2200 μl volume because protein is adsorbed to the AlOH adjuvant:

| | | | 1st immunization | | | | 2nd and 3rd immunization | | |
|---|---|---|---|---|---|---|---|---|---|
| | Injection | Sample | Volume | Proteins | Aspirate to | | Volume | Proteins | |
| Group | site | volume | AlOH | in urea | final volume | IFA | AlOH | in urea | Add NaCl |
| 1 | Right | 2120 μl | 800 μl | Yes | 1100 μl | 1100 μl | 800 μl | Yes | Aspirate to final |
| | | | | | | | | | volume of 2200 μl |
| | Left | 2020 μl | 800 μl | No | 1100 μl | 1100 μl | 800 μl | No | Aspirate to final |
| | | | | | | | | | volume of 2200 μl |
| 2 | Right | 3120 μl | 1050 μl | Yes | 1100 μl | 1100 μl | 1050 μl | Yes | Aspirate to final |
| | | | | | | | | | volume of 2200 μl |
| | Left | 1800 μl | 1050 μl | No | 1100 μl | 1100 μl | 1050 μl | No | Aspirate to final |
| | | | | | | | | | volume of 2200 μl |
| 3 | Right | 1550 μl | 800 μl | Yes | 1100 μl | 1100 μl | 800 μl | Yes | Aspirate to final |
| | | | | | | | | | volume of 2200 μl |
| | Left | 890 μl | 800 μl | No | 1100 μl | 1100 μl | 800 μl | No | Add 510 μl |
| 4 | | 390 μl | 520 μl | Yes | 1100 μl | 1100 μl | 520 μl | Yes | Aspirate to final |
| | | | | | | | | | volume of 2200 μl |
| 5 | | 530 μl 1^{st} and | 520 μl | No | Ready to go | 1100 μl | 520 μl | No | Add 1090 μl |
| | | 1620 μl 2^{nd} | | | | | | | |
| | | and 3^{rd} | | | | | | | |

Protocol for Formulation of Vaccines Using Proteins in Urea Buffer

Urea was included in some buffers as a means to solubilize proteins and prevent precipitation. This protocol describes how formulations for immunizations were prepared using proteins solubilized in urea buffer, with aluminum hydroxide and Incomplete Freund's Adjuvant (IFA). Approximately 50% of the protein amount is expected to have the native folding when absorbed to aluminum hydroxide and therefore an immunization dose of 30 μg protein per animal was aimed at. For proteins not requiring urea buffer to be soluble the recommended immunization dose was 15 μg protein per animal. Following absorption of the protein to aluminum hydroxide, the protein-alum complex was pelleted by centrifugation and the supernatant containing urea discarded.

Detailed formulation protocol (1st immunization—protein with aluminum hydroxide and IFA):
1. Aluminum hydroxide (Alhydrogel 2.0%, Brenntag) was mixed with protein in a ratio of 100 μL aluminum hydroxide to 125 μg protein. For an immunization dose of 30 μg protein, 24 μL aluminum hydroxide is used per mouse.
2. The mixture was incubated with end-over-end rotation for 1 hour.
3. A volume of 0.9% NaCl corresponding to the double protein-aluminum hydroxide solution volume was added, to reduce the urea molarity and slowly refold the protein.
4. The mixture from 3 was incubated with end-over-end rotation for 20 min.
5. Steps 3-4 were repeated until urea concentration was reduced to 1 M.
6. The protein-alum complex was pelleted by centrifugation at 1,000 rpm for 2 min and supernatant was removed.
7. The protein-alum complex was washed once in 0.9% NaCl to remove excess urea.
8. The protein-alum complex was pelleted by centrifugation at 1,000 rpm for 2 min and supernatant was removed.
9. 0.9% NaCl corresponding to half of the final desired volume was added.
10. reund's incomplete adjuvant (Sigma) was added in a ratio of 1:1 (volume) and the the mixture was vortexed thoroughly for 1 hour.
11. The formulation was then ready for immunization.

Detailed formulation protocol (2^{nd} and 3^{rd} immunizations—protein with aluminum hydroxide):

1. Steps 1-8 as described above were carried out.

2. A physiological saline solution was added to the final desired volume.

3. The formulation was then ready for immunization

Protein prepared according to this protocol was—as indicated in the table above—administered to the mice as separate injections in their right thigh in this Example.

Protocol for Formulation of Vaccines Using Soluble Proteins

This protocol describes how formulations with aluminum hydroxide and Incomplete Freund's Adjuvant (IFA) were prepared for protein that was soluble and did no require solubilisation in urea and refolding of protein. Following absorption of the protein to aluminum hydroxide, the protein-alum complex was pelleted by centrifugation and the supernatant containing was removed so the injection volume dif not exceed 100 μl.

Detailed formulation protocol (1^{st} immunization—protein with aluminum hydroxide and IFA):
1. Aluminum hydroxide (Alhydrogel 2.0%, Brenntag) was mixed with protein in a ratio of 100 μL aluminum hydroxide to 125 μg protein. For an immunization dose of 15 μg protein, 12 μL aluminum hydroxide was used per mouse. When immunizing with a combination vaccine, 15 μg of each protein was pooled and mixed with the appropriate amount of aluminum hydroxide
2. 0.9% NaCl was added so the final volume was 50 μL per immunization (if the volume exceeded 50 μL, the optional step 3a was carried out)
3. The mixture is incubated with end-over-end rotation for 1 hour.
   3a Optional: The protein-alum complex was pelleted by centrifugation at 1,000 rpm for 2 min and supernatant removed to reach the final desired volume.
4. 50 μL Freund's incomplete adjuvant Sigma (ratio of 1:1 volume) was added per immunization and the mixture was vortexed thoroughly for 1 hour.
5. The formulation was then ready for immunization.
2^{nd} and 3^{rd} immunization (protein together with aluminum hydroxide)
1. Formulation steps 1-3 as described immediately above was carried out.
2. Add 0.9% NaCl to the final desired volume 100 μL per immunization.
3. The formulation was then ready for immunization.

Protein formulated according this protocol was administered as injections in the left thigh of the mice in this Example.

Volume of administration was 100 μL/mouse/injection site.

Vaccination

Mice were vaccinated with SC injection for all vaccine groups at D0, D14 and D28.

Each mouse was immunized by an SC injection of 100 μL of formulation per injection site. 15 μg of each protein was administered to each mouse per immunization.

Blood Sampling and ELISA Testing

At D2 and D35 blood samples were obtained for serum preparation from all mice by tail vein puncture after warming in warming cabinet for 5 minutes at 38° C. Sample size of whole blood was ≤100 μL. After acquisition, blood was centrifuged at 3500 rpm for 15 minutes. Serum samples were stored frozen at −80° C.

The ELISA protocol for testing titers was as follows:

2 ELISA plates were coated over night at 4° C. with 1p protein in 1×PBS

The plates were washed 3× in PBS-tw and blocked for at least 15 min or stores in PBS-Tw at 4° C. until use.

Serum/plasma samples were diluted 1:50 by taking 20 μl sera and mixing with 980 μl PBS-Tw

150 μl PBS-tw was added in column 1 and 100 μl in each of the wells.

50 μl of the sera samples (mouse nr #) was added to the first well and 2-fold dilutions were made, whereafter the wells were incubated for 2 hours followed by 3 times wash PBS-tw

100 μl secondary antibody (rabbit anti-mouse HRP IgG) diluted 1:10.000 in PBS-tw was added to each well and incubated for ½ hour followed by 3 times wash in PBS-tw.

An OPD (o-phenylenediamine to 2,3-diaminophenazine) solution was prepared by mixing 1.5 ml pre-warmed and vortexed liquid OPD in 150 ml colouring buffer and adding 60 μl H₂O₂ (100× stock). 100 μl were added to the wells and allowed to react for 25 minutes and finally read at 490 nm.

Infection, Mortality Monitoring and Body Weight Recording

At D36 mice were infected with 50 μL of bacterial suspension IN under ketamine/xylazine anaesthesia.

6 hrs post infection mice were weighed and overdosed with ketamine+xylazine. Lungs were aseptically removed and placed into sterile Precellys test tubes containing 2 mL of sterile PBS. Precellys test tubes were weighed and weights were recorded pre- and post-sampling.

Lungs were homogenized in sterile PBS using a BERTIN Technologies homogenizer. After homogenizing, a serial dilution of the homogenates were used for CFU determination.

Read-outs from the experiment were CFUs in lung tissue 6 hrs PI, single pre-immune sera, and single immune sera.

Data were processed using Microsoft Excel SW. Statistical analysis and graphical presentation were performed using GraphPad Prism software (version 7.04). Differences between groups were considered statistically significant when p<0.05.

Results

The Inoculum size was confirmed by plating the −5th dilution of the inoculum in 4 replicates. Average number of CFU per plate was 155 colonies, which is a value of 1.55×10⁸ CFU/mL, as shown in the following table:

Confirmed inoculum sizes appear from the following table:

| Inoculum | CFU | | | | | CFU/mL | | |
|---|---|---|---|---|---|---|---|---|
| dilution | 1 | 2 | 3 | 4 | Average | Dilutions | SD | CV |
| −5 | 145 | 179 | 161 | 135 | 155.0 | 1.55 × 10⁸ | 19 | 0.12 |

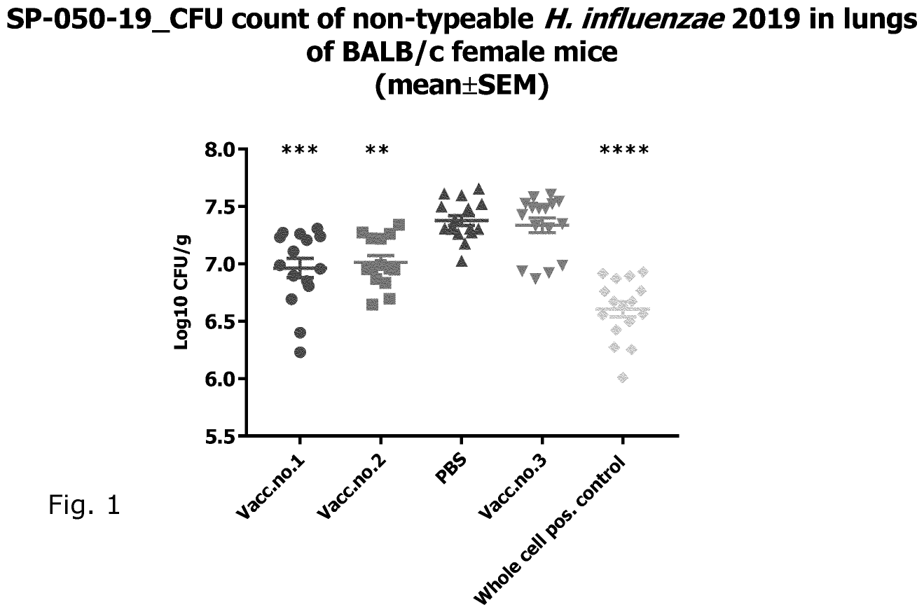

CFUs in lungs are presented in FIG. 1 and the following table.

| | Average Log10 CFU (mean ± SD) | | | | |
|---|---|---|---|---|---|
| Time PI | Vaccine #1 | Vaccine #2 | PBS | Vaccine #3 | Whole cell |
| 6 hours | 6.96 ± 0.32 | 7.04 ± 0.21 | 7. ± 0.25 | 7.33 ± 0.25 | 6.60 ± 0.26 |

As depicted in FIG. 1, a statistically significant decrease in CFU counts was observed in groups vaccinated with vaccine no. 1, vaccine no. 2 and whole cell suspension in comparison to the PBS treated group. P<0.0001 was observed in the group that received the Whole cell suspension, whilst in the group that received Vaccine no. 1 it was 0.0002, and with Vaccine no. 2 it was 0.0019.

Statistically higher CFU numbers in lungs was observed in the group treated with Vaccine no. 3 in comparison to group treated with the Whole cell (p<0.0001), as well as when compared to the both Vaccine no. 1 and no. 2 treated groups (p<0.01). (Animal no. 3 from Group 1 and animals no. 17 and 18 (Group 2) succumbed to the anesthesia during infection at D36).

Titer Determinations

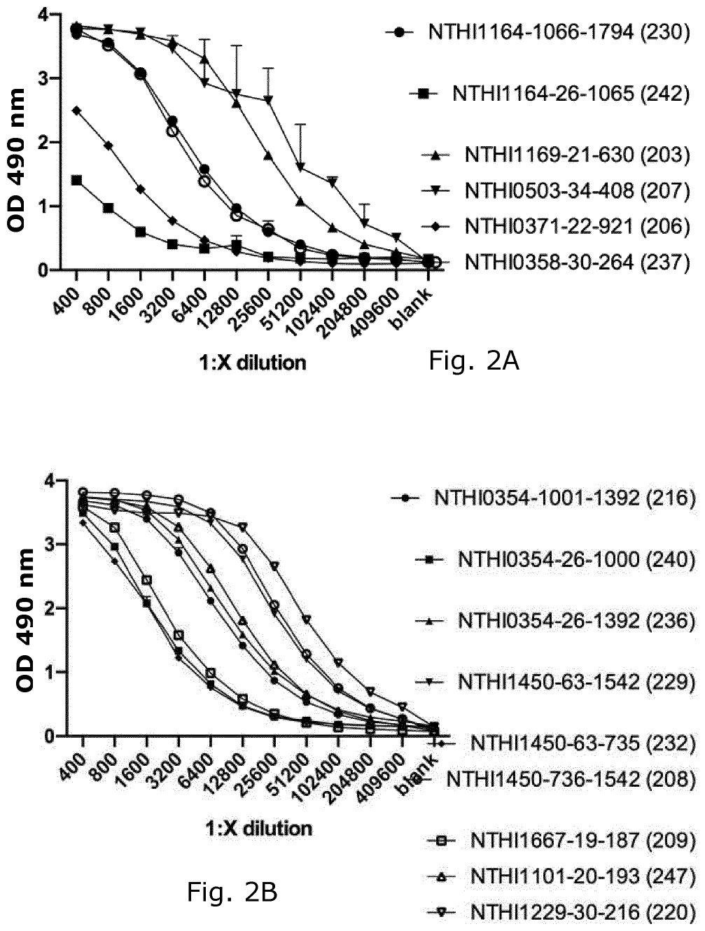

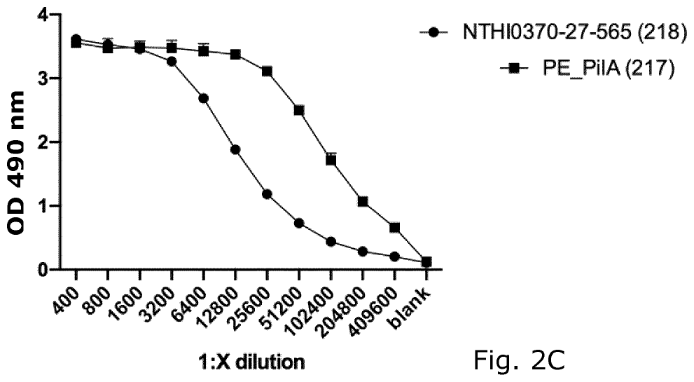

Titers for antibodies against the individual antigens of the combination vaccines were determined in sera from the mice in Groups 1, 2, and 4. The results are provided in FIGS. 2A-2C.

Discussion and Conclusions

The protective effects of immunization with 3 combination vaccines and a whole cell vaccine were tested after intranasal (IN) challenge with non-typeable Haemophilus influenzae strain 2019 in BALB/c female mice. The mice underwent three immunizations with 14-days interval periods between each vaccination. At day 36, mice were challenged with non-typeable Haemophilus influenzae strain 2019, using an inoculum size of 7.75×10⁶ CFU/50 μL via the intranasal route under deep ketamine+xylazine anesthesia. Challenged animals were sacrificed 6 hrs PI in order to determine CFUs in lungs.

Vaccination with vaccines no. 1 and no. 2 resulted in a significant decrease of CFU in lungs when compared to the PBS control group. A significant decrease of CFU in lungs was also observed in the whole cell vaccine group when compared to PBS control group. In the group vaccinated with Vaccine no. 3, the CFU count in lungs was significantly higher than in vaccine no. 1, no. 2 and the Whole cell treated group and it did not differ significantly from the CFU count in the negative control group.

Further, all antigens in vaccines 1 and 2 induced antigen specific antibodies as demonstrated in the ELISA experiments.

In conclusion, subcutaneous vaccination with Vaccine. no. 1, Vaccine no. 2 and the Whole cell vaccine exhibited protective effect against non-typeable Haemophilus influenzae strain 2019 in the model of IN induced pneumonia in BALB/c female mice.

### Example 3

Vaccine Study with Three Evaxion Combo Vaccines and One Positive Control in Non-Typeable Heamophilus Influenzae Induced Lung Infection

The aim of this Example, which was carried out using essentially the same protocols as in Example 2, was to investigate the protective effects of immunization with three combination vaccines and one control vaccine after intranasal (IN) challenge with non-typeable Haemophilus influenzae strain 2019 in BALB/c female mice.

Experimental Procedures

Unless specified in the following, this example was carried out using the same materials and methods as those used in Example 2. One minor difference was the age of the mice tested; in the present example, the mice were 7 weeks at initiation of the test.

Allocation of animals to vaccine test groups were in the present Example made according to the following table:

| | | Adjuvants | Blood | Infection | Experimental |
|---|---|---|---|---|---|
| Group | Vaccine | regime | sampling | (D36) | procedures |
| 1 | Vaccine | Combina- | D3 + D35 | ≈1.0 × 10⁷ | Immunization |
| | #1 | tion | | CFU/50 μL | (SC): |
| 2 | Vaccine | vaccines: | | IN/mouse | D0; D14 |
| | #2 | 1^{st} IFA/ | | | and D28 |
| 3 | Vaccine | ALUgel | | | Blood |
| | #3 | 2^{nd} and 3^{rd} | | | collection: |
| 4 | Vaccine | ALUgel | | | D3 and D35 |
| | #4 | Positive | | | IN challenge: |
| | (positive | control: | | | D36 |
| | control) | ALUgel | | | Lung sampling |
| 5 | Placebo | | | | for CFU: |
| | | | | | D36: 6 hrs PI |

The composition of the vaccines tested in the present Example were as follows:

| | | Immunization | |
|---|---|---|---|
| | | side of the | |
| Group | Vaccine antigens | mouse | Protein in urea |
| Group 1, Vaccine | NTHI1983-63-1492 | Right | Yes |
| 1 | NTHI1983-63-735 | Right | Yes |
| | NTHI1983-736-1492 | Right | Yes |
| | NTHI0145-28-666 | Right | Yes |
| | NTHI0482-17-154 | Left | No |
| | NTHI1441-25-95 | Left | No |
| Group 2, Vaccine | NTHI0132-27-464 | Right | Yes |
| 2 | NTHI1489-1-448 | Right | Yes |
| | NTHI1083-24-197 | Left | No |
| | NTHI1844-1-844 | Left | No |
| | NTHI0448-155-428 | Left | No |
| Group 3, Vaccine | NTHI0538-1-935 | Right | Yes |
| 3 | NTHI0156-1-225 | Right | Yes |
| | NTHI1969-28-693 | Right | Yes |
| | NTHI1084-20-800 | Left | Yes |
| | NTHI0369-22-709 | Left | Yes |
| Group 4, Vaccine | NTHI1390-28-915 | Right | Yes |
| 4, positive control | NTHI0830-20-405 | Left | No |
| Group 5 | Alum + IFA | Left + Right | N/A |
| Placebo control | | | |

Proteins NTHI1390 and NTHI0830 in the positive control have previously been shown to be highly protective in US 2012/0045457.

Preparation of vaccine compositions followed the protocol of Example 2 and as in Example 2, the fraction of the vaccine combination that contained urea-solubilized protein was administered in the right thigh of the mice, whereas the protein that was readily soluble was administered in the left thigh.

The following table summarizes the composition of each vaccine and its administration schedule. To reach the final volume of 2160 μl, excess buffer was if necessary discarded after refolding by aspirating off after AlOH adsorption of antigen.

| | | | 1st immunization | | | | 2nd and 3rd immunization | | | |
|---|---|---|---|---|---|---|---|---|---|---|
| | Injection | Sample | Volume | Proteins | Aspirate to | | Volume | Proteins | Add | |
| Group | site | volume | AlOH | in urea | final volume | IFA | AlOH | in urea | NaCl | |
| 1 | Right | 882 μl | 1040 μl | Yes | Refold | 1080 μl | 1040 μl | Yes | Refold | |
| | Left | 673 μl | 520 μl | | −113 μl | 1080 μl | 520 μl | | 967 | μl |
| 2 | Right | 1093 μl | 520 μl | Yes | Refold | 1080 μl | 520 μl | Yes | Refold | |
| | Left | 1504 μl | 780 μl | | −1204 μl | 1080 μl | 780 μl | | −124 | μl |
| 3 | Right | 1900 μl | 780 μl | Yes | Refold | 1080 μl | 780 μl | Yes | Refold | |
| . | Left | 658 μl | 520 μl | Yes | −98 μl | 1080 μl | 520 μl | Yes | 982 | μl |
| 4 | Right | 658 μl | 260 μl | Yes | Refold | 1080 μl | 260 μl | Yes | Refold, | |
| | Left | 720 μl | 260 μl | | 100 | 1080 μl | 260 μl | | 1182 | μl |

| | | | | | | | | | |
|---|---|---|---|---|---|---|---|---|---|
| 5 | Right | 800 μl | 520 μl | Yes | Refold | 1080 μl | 520 μl | Yes | Refold |
| | Left | 600 μl | 780 μl | | −300 | 1080 μl | 780 μl | | 780 |

Final volume in each tube: 2160 μl.

Results

The Inoculum size was confirmed by plating the −5th dilution of the inoculum in 4 replicates. Average number of CFU per plate was 147.3 colonies, which is a value of 1.47×10⁸ CFU/mL, as shown in the following table:

| Inoculum | CFU | | | | | CFU/mL | | |
|---|---|---|---|---|---|---|---|---|
| dilution | 1 | 2 | 3 | 4 | Average | Dilutions | SD | CV |
| −5 | 124 | 129 | 171 | 155 | 147.3 | 1.47 × 10⁸ | 19 | 0.12 |

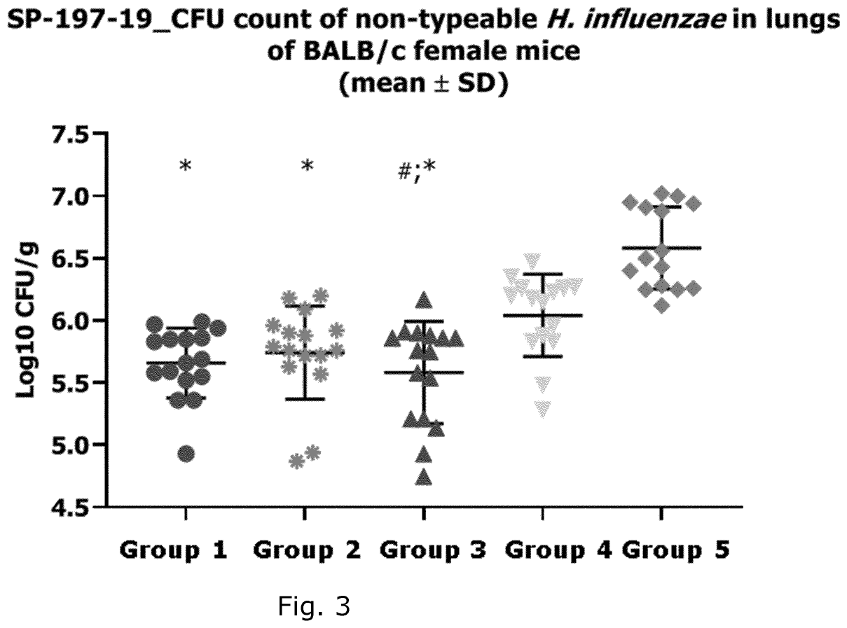

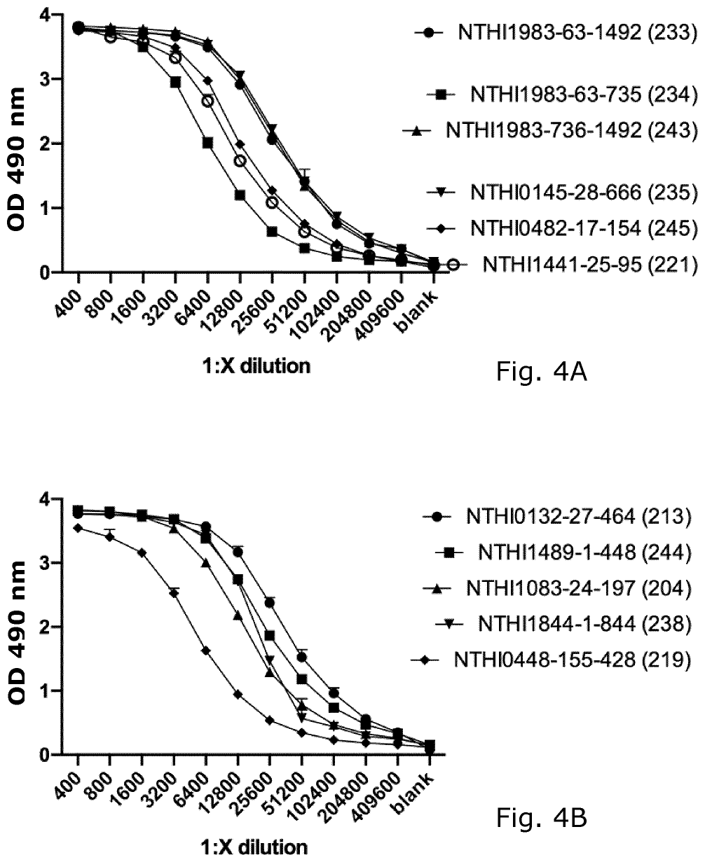

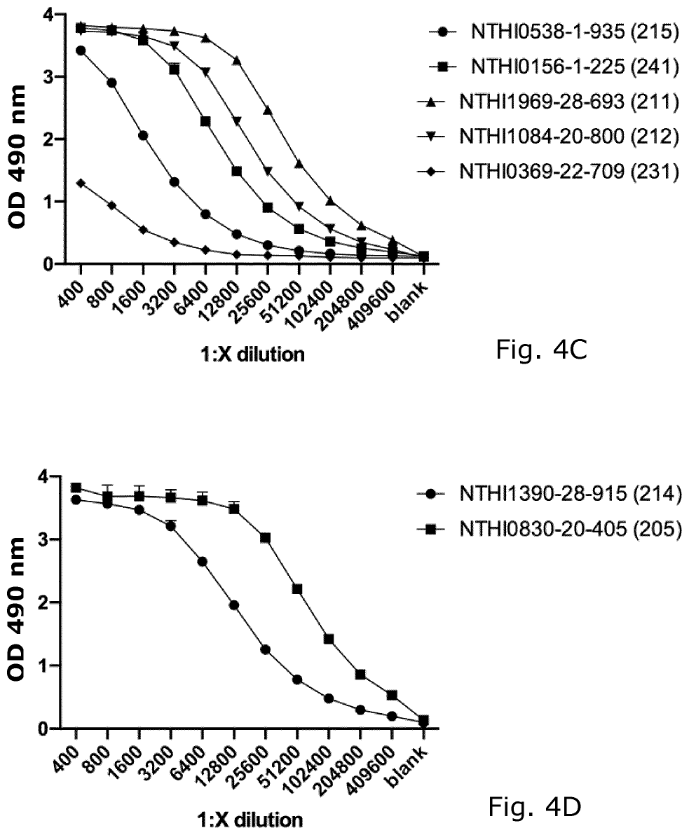

CFUs (Mean Log 10) in lungs 6 hours PI are presented in FIG. 3 and the following table:

| Group | Mean ± SD |
|---|---|
| Vaccine 1 | 5.66 ± 0.49 |
| Vaccine 2 | 5.74 ± 0.52 |
| Vaccine 3 | 5.58 ± 0.54 |
| Vaccine 4, positive control | 6.04 ± 0.42 |
| Placebo control | 6.58 ± 0.32 |

As depicted in FIG. 3, a statistically significant decrease in CFU counts was observed in groups 1, 2 and 3 in comparison to group 5 (p<0.05). Group 3 exhibit statistically significantly difference in CFU count when compared to the positive control in group 4 (P<0.05).

Animal no. 62 from Group 4 and animal no. 65 (Group 5) succumbed to the anesthesia during infection at D36.

An antibody titer determination carried out as in Example 2 revealed that all antigens in the 4 tested combination vaccines elicited specific antibody production in the vaccinated animals.

Result Summary

Vaccination with vaccines no. 1 and no. 2 in Example 1 and vaccines 1-4 in Example 2 resulted in significant decrease of CFU in lungs when compared to placebo. This emphasizes that the antigens used in the 6 vaccines are effective vaccine antigens.

Sequence Information

The proteins having the amino acid sequences numbered 1-30 in the sequence listing are named according to the following table:

| Sequence | Name | Sequence: | Name |
|---|---|---|---|
| SEQ ID NO: 1 | NTHI1441 | SEQ ID NO: 16 | NTHI1169 |
| SEQ ID NO: 2 | NTHI0482 | SEQ ID NO: 17 | NTHI0145 |
| SEQ ID NO: 3 | NTHI1667 | SEQ ID NO: 18 | NTHI1969 |
| SEQ ID NO: 4 | NTHI1101 | SEQ ID NO: 19 | NTHI0369 |
| SEQ ID NO: 5 | NTHI1229 | SEQ ID NO: 20 | NTHI1084 |
| SEQ ID NO: 6 | NTHI0156 | SEQ ID NO: 21 | NTHI1844 |
| SEQ ID NO: 7 | NTHI0358 | SEQ ID NO: 22 | NTHI1168 |
| SEQ ID NO: 8 | NTHI1851 | SEQ ID NO: 23 | NTHI1390 |
| SEQ ID NO: 9 | NTHI0830 | SEQ ID NO: 24 | NTHI0371 |
| SEQ ID NO: 10 | NTHI0503 | SEQ ID NO: 25 | NTHI0538 |
| SEQ ID NO: 11 | NTHI0448 | SEQ ID NO: 26 | NTHI0782 |
| SEQ ID NO: 12 | NTHI1489 | SEQ ID NO: 27 | NTHI0354 |
| SEQ ID NO: 13 | NTHI0132 | SEQ ID NO: 28 | NTHI1983 |
| SEQ ID NO: 14 | NTHI0532 | SEQ ID NO: 29 | NTHI1450 |
| SEQ ID NO: 15 | NTHI1230 | SEQ ID NO: 30 | NTHI1164 |

A number of the polypeptides of the invention are fragments of the full-length, native polypeptides. Such fragments are named as follows: NTHIXXXX_Y-Z, where XXXX is the 4 digit number in the polypeptide designation, Y is the number of the N-terminal amino acid residue in the fragment and Z is the number of the C-terminal amino acid residue in the fragment. For instance, NTHI1168-100-400 would be the polypeptide having the amino acid sequence SEQ ID NO: 22, residues 100-400.

The amino acid sequences of the polypeptides disclosed herein are derived from the following SEQ ID NOs:

SEQ ID NO: 1 (membrane protein):
MKKLATLTAL ASALTMAVAT AVQAESKSSN TDNTATPCVG DKCVKTKAAE GKCGEGKCGA
DKAKSAEGKC GEGKCGASKP KAAEGKCGEG KCGSK
SEQ ID NO: 2 (hypothetical protein):
MKKAFWLVLS SAIVLAACDD KPKMTEQTKT TDQAKMAAEQ PDQKPVLINY KQIALDKSQQ
AIDTAKVAQD KSVQANEAVK VAMQKQQEAE IALSQGGEEA EKLSKQKMDE SNAAAELALK
LSEESKAASA LSIQLSKEAE DAANKANQQA QAEK
SEQ ID NO: 3 (histone):
MKKLTLALVL GSALAVTGCF DKQEAKQKVE DTKQTVASVA SETKDAAANT MTEVKEKAQQ
LSTDVKNKVA EKVEDAKEVI KSATETASEK ATEIKEAVSE KASEMKEAAS EKASEMKEAA
SEKASEMKEA ASEKASEMKE AASEKASEMK EAASEKVGEM KEKATEMKEA VSEKATQAVD
AVKEATK
SEQ ID NO: 4 (hypothetical protein):
MKKKNQILVS LSIVALLGGC SEEQVQRDVY QSLDDCLADW KKIELCEADK NTESTQKTET
TPQQGLGLNI RDNGNAESAV KNPAENNVQA NQSENNAEST AKAESTDPSL GAAIAGGVIG
YMAARAISSF LGPSYHPGNR AVTTPTGQVV QPQTNRSVGK PMLVKGNAGS MNSKPVSRGG
FSSPNNTHRS SGG
SEQ ID NO: 5 (cytochrome c nitrite reductase pentaheme):
MNLTSLINKS AKALALSAAF VALPFLAYAD DAQKPADHVT YEPQLDNQRD PNQYCAKCHK
FDKIDKNQTL DRSGGELHFG KFHGAHLDKK NPNNGKAITC VSCHGNVSEN HRRGAKDVMR
FEGDIFGNKK PMYSVQEQNQ VCFACHQPDK LREKLWAHDV HAMKLPCASC HTLHPKEDAM
KGIQPKQRVK LCVDCHGKQQ KRKAEQDKLT KQKDKQ
SEQ ID NO: 6 (hypothetical protein):
MGIKNKPITR PCPQCGRNYQ YRRASGRTFE LCEYCRNLDC VVCGKKVPPE RGRKNTCCAE
CEKLKIHNIQ NAHYAKRIAE DPELNKRNHA KARENRKADP ERMHEHLEAQ RERHYRRVQD
PNYLATRKVY QAQRWQDKKD EILAQRREFW DSLSDVEKAE RLERNQAIQR KHKAKKRDQL
KLDPQKWAEY QEYQRTKRRE HRQRKALNEL MVGTKELLNV TNKDK
SEQ ID NO: 7 (cell envelope protein TonB):
MQQTKRSLLG LLISLIAHGI IIGFILWNWN EPSDSANSAQ GDISTSISME LLQGMVLEEP
APEPEDVQKE PEPEPENVQK EPEPEKQEIV EDPTIKPEPK KIKEPEKEKP KPKEKPKNKP
KKEVKPQKKP INKELPKGDE NIDSSANVND KASTTSAANS NAQVAGSGTD TSEIAAYRSA
IRREIESHKR YPTRAKIMRK QGKVSVSFNV GADGSLSGAK VTKSSGDESL DKAALDAINV
SRSVGTRPAG FPSSLSVQIS FTLQ
SEQ ID NO: 8 (hypothetical protein):
MEKAGYERAS DSDSSFSGGG GWREDNSSDS YRSTSDRWND HKSRYGKDKV YTDAFNERRN
NSSWSGGHSA ISRTISEKYH SLSNGQMSAA VPEKDQKTLT GGLFGKSYSN APYSERTPSI
FDRNIRGSMT LNNGDVWSSD PQYSSVRERA DINSYDRIKR GEELNLIGRA VGGVFSGVGG
AATTPVGKIA ESAANFGLSH VGDLSRQFKS NQEQAYYDSL TPEGKAYYDT RVDFINKSYK
NAREKYETND KWIDRGITAA QVGLSALGPP GAMLGSGIGL LGKAINKKDT MTKSLRDLTE
TLNSNALNNH IAQQNELAEK ERQAYKEFMA GRDLRSDNTQ PKGILNTMYN RMQNIDPDKQ
VKTSDVPNLR NYWANIIVS
SEQ ID NO: 9 (membrane protein):
MNKSFLLLPL SLVVLSACTS NSPAPISDAD GNLSPSVVQS VNGSNVGGAW QPEIQKNSLP
TMGNMVTPQP NFQPINQQPA MPTKPAQPAF QPSPKTVVSA PTVQTKTVTK TIADCVDGQH
INIPRNPNTN APDYSKISKG SYKGNTYKVN KGDTMFLIAY LAGIDVKELA ALNNLSEPYN
LSLGQVLKIS NCGTKTVTTT VSVKQPAVTA STAMPAKPAV TYTPGANGTQ IGSDGTIIGP
IKSEAGISSS VSVATSSTQV TSSVNNANST PINANVVAPI ASNVAWQWPT SGNIIQGFSS
TDGGNKGIDI SGSRGQAVKA AAAGRIVYAG NALRGYGNLI IIKHNDDFLS AYAHNDKILV
ADQQEVKAGQ DIAKMGSSGT NTVKLHFEIR YKGKSVDPVR YLPKR
SEQ ID NO: 10 (cell envelope integrity protein TolA):
MQNNRQKKGI NAFAISILLH FILFGLLILS SLYHTVEIMG GGEGEGDVIG AVIVDTGTAA
QEWGRIQQQK KGQADKQKRP EPVVEEKPPE PNQEEIKHQQ EVQRQEEIKH QQEVQRQEEL
KRQQEQQRQQ EIKKQQEQAR QEALEKQKQA EEARAKQAAE AAKLKADAEA KRLAAAAKQA
EEEAKAKAAE IAAQKAKQEA ETKAKLEAEA KAKAAAEAKA KAEAEAKAKA AAEAKAKAEA
EAKAKAAAEA KAKADAEAKA KAAAEAKAAA EAKRKADQAS LDDFFNGGDV GGGSASKGGN
TNKGGTQGSG AALGSGDGGK VGDQYAGVIK KEIQRRFLKD PNFAGKVCRI KIQLGRDGTI
LGYQKISGSD DICSAALSAV ARTKKVPAAP SDEIYEKYKS PIIDFDIR
SEQ ID NO: 11 (opacity-associated protein OapA):
MDKNQQSSQN ELDLGLNQEP ITPKKTIQPS SSILGKAKGL FAKKNHVQTN FQQRKEPTFG
DSSTQENDPL IPSENLKKVQ KPVLQTSSTE ENISAVDEEI SAENNADEPV EKAEKPILAQ
PEKWKILQVL PAKHRRLFMA IFVLVILLII FFALKPSSDT VESFTQSNSN EVPVQFQSLD
QSQPLETTIL DNPPAQNQMA VEQANQSEFA PKAEEAANNT TAQNPLVENA PMQQNVVQSP
SQMPNEMAAA SVMPMQPAQA EQPKATVPVQ PMKKAVEPQV AHKDTVKKEV KVAENAQAPS
KATEQNVAKT AGNAPIVEAK PVQVKKEKKV QIVDAKPVSK SAASRLSAKT LTVPKGVSLM
QVFRDNQLNI SDVNAMSKAA GAGNVLSSFK SGDKVTVSVN NQGRVNEMRL SNGARFVRQS
DGSYQYKK
SEQ ID NO: 12 (hypothetical protein):
MAEENKEIIA YKGFKQDWTC RGYQYEVGKT YEHKGNVKAC ESGFHACEYP LDVLSYYSPA
VSKFAVVKMS GETSKDSDDT KIASAKITIE TEINLPEMIK KAVEWIKGKV DWDAAKVSNT
GDWSAATNTG DWSAATNTGD QSAATNTGNW SAATNTGDQS AATNTGDQSA ATNTGYRSVA
TNTGYRSVAT NTGDQSAATN TGGQSAATNT GDQSAATNTG YRSVATNTGD QSAATNTGYR
SVATNTGDQS AATNTGGQSA ATNTGDQSAA TNTGYRSVAT NTGDQSAATN TGNWSAATNT
GDQSAATNTG DQSAATNTGY RSVATNTGDR SAATNTGYRS AATNTGYRSV ATNTGDRSAA
TNTGYRSVAT NTGDQSVAEI SGKQSIAVAL GWQSKAKASI NGAIVCVYRN HDGELIHIKA
SKVGENNIKA DTWYTLDEFG EFVEVKDD
SEQ ID NO: 13 (integrating conjugative element protein):
MKANKAFYVI GGITALVVTL MAGFLFSGSS STNTAETKKE DTLDISFKDL SPEELRAMGI
EGDTSKDTVR TLIGKVKEHN KRFDEVILQN ERLVKENEQL KNQANNTDYQ IQQAVQAETG
ALINEINALK DQVVQAGNQV VGNGNAQANP NAPLPINGAT TGPQVETRGG INWVNPSDMP
TTDRQGNALQ GIGVNGAGIG FPKTFDNNVN DIRKSNDGLQ SSTGNNAIFN TATPFFTIAA
NSTLTNSVAM TALVGRVPID NKVTEPYPFK LLIGRDNLIA NGIELPDIEG AIVSGTASGD
WTLSCVRGDV KSLTFVFSDG RIVSTDNSDD KIGWLSDPHG VPCIPGERRT NAPEYLTTNF
FLTGASAAAQ GLSSSQSTTV VDGGAVIGAV TGNNGKYILG QALGGGLRET ADWFRQRYGQ
MFDAVYVPPG KEVAVHIEKQ IDIDYDRMSR KVKYGQASRQ SNLD
SEQ ID NO: 14 (peptidase M23):
MQHVKLARDR RKKRTYIKVG VFFVAILLIL TGILLTIKDK SEENPIFSTS DSGEYHELNT
SPNKNSTALQ PDEDATSYDD ELQAKDDEVD EVKLSSDDLG TLPQHAQDAL NGLLDAADQA
IRITDQFSYT VTEGDTLKDV LVLSGLDDSS VQPLIKLDPE LAHLKAGQQF YWILNKNDNL
EYLNWLVSEK EERIYERLED GKFKRQVIEK KSIWRKEVLK GEIQNSLNSS LREQGLDTRQ
ISQLSNALQW QVSLRKLKKG TQFAILVSRE YLGDKLTGQG NVEALRISSG GKNYYAVQAA
NGRYYNQQGE TLGKGFARYP LQRQARVSSP FNPNRRHPVT GRVRPHKGVD FSVSQGTPVI
APADGTVEKV AYQAGGAGRY VMLRHGREYQ TVYMHLSKSL VKAGQTVKKG ERIALSGNTG
ISTGPHLHYE FRINGRAVNP LTVKLPGTSS GMTSAERKQF LVRVREAEKM LKP
SEQ ID NO: 15 (ammonia-forming cytochrome c nitrite reductase
(nrfA)):
MNAFKKSLIV AASFASLSLF NSATAELVYK PLEQPVEPAK PDLKIESVNE KFAEKYPNQY
NSWRSTANGD GENIIYADEE NPRLIVLWGG YAFAKEYNAP RGHFYAVTDV RNILRTGAPK
TANDGPQAMA CWTCKGPDVP RLIAEWGEKD YFNAKWAKGG PEIVNSIGCA DCHDTTSKDF
AEGKPALRIA RPHVLRALDA LEKATAEKDK AEGRPHNNLS FNTAARTEKR AEICANCHVE
YYFAGDIKQV TFPWDNGQTA DDIEKYYDDI GFTDWTHSLS KAPMLKAQHP DFEIWSLGMH
GKNGVTCVDC HMPKVQGADG KVYTDHQIQN PFDAFDHTCA NCHDQSKEKL RDIVTSRKKE
VKDVMGRLED QVVKSHFEAK AAWDAGATKE EMEAALMDIR HAQWRWDYTA ASHGGHMHAP
EVVLRVLASG LDKVADARTK LAVILTKHGV KTPVQIPDIS TADKAWKVMG IDIEKERKAK
EEFLKTVVPQ WEQQAREKGL LVDPPAQK
SEQ ID NO: 16 (transferrin-binding protein 2 (Tbp2)):
MKSVPLITGG LSFLLSACSG GGGSFDVDDV SNPSSSKPRY QDDTSSSRTK SNLEKLSIPS
LGGGMKLVAQ NLSGNKEPSF LNENGYISYF SSPSTIEDDV KNVKTENKIH TNPIGLEPNR
ALQDPNLQKY VYSGLYYIEN WKDFSKLATE KKAYSGHYGY AFYYGNKTAT DLPVSGVATY
KGTWDFITAT KYGQNYSLFS NARGQAYFRR SATRGDIDLE NNSKNGDIGL ISEFSADFGT
KKLTGQLSYT KRKTDIQQYE KEKLYDIDAH IYSNRFRGKV TPTKSTSDEH PFTSEGTLEG
GFYGPNAEEL GGKFLARDKR VFGVFSAKET PETEKEKLSK ETLIDGKLIT FSTKTADATT
STTASTTADV KTDEKNFTTK DISSFGEADY LLIDNYPVPL FPEGDTDDFV TSKHHDIGNK
TYKVEACCKN LSYVKFGMYY EDKEKKNTNQ TGQYHQFLLG LRTPSSQIPV TGNVKYLGSW
FGYIGDDKTS YSTTGNKQQD KNAPAEFDVN FDNKTLTGKL KRADSQNTVF NIEATFKNGS
NAFEGKATAN VVIDPKNTQA TSKVNFTTTV NGAFYGPHAT ELGGYFTYNG NNPTATNSES
SSTVPSPPNS PNARAAVVFG AKRQVEKTNK
SEQ ID NO: 17 (integrating conjugative element protein):
MKLNHALKAS YIAISCAILS STMPVSASNS NPFGFNKADS ILSDMVFYQI GGGVGYMAPP
SRGTIPAAEF GIGWKANLMC GNFDIKTSIK NQLNGLTEGF KDLYSNVIES ATGAVASLPA
MVIQRANPQL YDILTNGLYQ GKIDFNSLKT SCEEMSKKLA DATLNGRWSQ SSDMESFKDI
TSTEPDATRA KKRLEESQGK EGKEWVGGKK RGGQGQEPIK IVEDVAKAGY NMQNKRNVLE
SGRISGSSCS GLLCQTWDNP KDMTDWLTNV IGEKQLTTCK TDCGTASSSR AGVGLTPEIE
KENIKTVTQL QKALNMSTPS VEVLAELSST TVPVTRGLIE SLREDPDAQV LGQRLASEIA
VAKTMEKMLL ARRAVLTGMR EPNVANNKDA QEELEKILTL IDREIGQIRM EIDLQKSLTG
NSAVAILQNK ELREYNTGTG NVTRDSVDKR ISDLANGTNS NAAELADADN INIPRNNVTL
EVPQMSNSLS YTGTGGTSGN SRGNTSYSSV PAIKGTALDQ ATGLLKKFEG FSDKAYWDVN
AYRTGYGSDT ITKADGTIVK VIKDIVVSRA DAERDLARRT QEFANRARNN VSSSTWDKLP
PNAQAALTSY AYNYGSLTKD VIAAAQASAQ SGDMNALANA VRNRQNNNNG VNAKRRNQEA
DYILGK
SEQ ID NO: 18 (tail-specific protease):
MKFKMSKNVI CYAWLSVCLS SAIPAFAVQP TLKPSDISIP AISEESQLAT KRATTRLIQS
HYRKIKLDDD FSEKIFDRYI KNLDFSHNTF LQSDIDELRQ KYGTKLDEQL SQGDLSAAFD
IYDVMMKRRY ERYTYALSLL DKEPDLNGQD QIEIDREKAA APQTEADANK LWDARVKNDI
INLKLKDKKW SEIKAKLTKR YNLAIRRLTQ TKADDIVQIY LNAFAREIDP HTSYLSPRTA
KSFNESINLS LEGIGTTLQS EDDEISIKSL VPGAPAERSK KLHPGDKIIG VGQATGDIED
VVGWRLEDLV EKIKGKKGTK VRLEIEPAKG GKSRIITLVR DKVRIEDQAA KLTFEKVSGK
NIAVIKIPSF YIGLTEDVKK LLVKLENQKA EALIVDLREN GGGALTEAVA LSGLFIADGP
VVQVRDAYQR IRVHEDDDAT QQYKGLLFVM INRYSASASE IFAAAMQDYR RGIIIGQNTF
GKGTVQQSRS LNFIYDLDQS PLGVLQYTIQ KFYRVNGGST QLKGVAADIN FPEIIDAKEY
GEDKEDNALA WDKIPSASYT EVGNIDYIDN AVNILNEKHL ARIAKDPEFV ALNEELKVRN
ERRDRKFLSL NYKMRKAEND KDDARRLKDL NERFKREGKK ALKDIDDLPK DYEAPDFFLK
EAEKIAADFV IFNSDQKINQ ANGLSEAKTE SKK
SEQ ID NO: 19 (heme/hemopexin utilization protein C):
MRFSKLSLAI TTTLVTANAL AQSVELDSIN VIATRDPSRF TYTPQKQSKD SLLSKQATSV
AAALEDIPNV DVRGGSRSIA QKPNIRGLSD NRVVQVIDGV RQNFDLAHRG SYFLPMSLIQ
EIEVIKGPSS SLWGSGALGG VVAMRTPNAL DLLKNNDKFG VKIRQGYQTA NNLSEKDVSV
FAANDKFDVL ISGFYNNADN LRIGKGNKLN NTAYKQFGGL AKFGWQINDA NRVELSHRET
RFKQTAPGNN EAKNELTNEQ IIEKINEYHN PLNNFPPKAK PSLEEFYSGV RARLGGVSYL
SDQQIPDQST VFNYYLTPDN PYLNTHIALY NNKTIEKEQR KVSGVKDQTK LITRGINLRN
SSELSHISFV YGVDYMRDKI STERGTNDKD AKFRAEPYNA NSNITGVYLI AHIPLFGEKL
LLSPSVRYDH YDTSSKTVKY KDNHLSPATK LTWKVTNWLD FTAKYNEAFR APSMQERFVS
GAHFGTSTRV GDIINSFVAN PNLRPETAKN KEITANLHFD SLFKQGDKFK IEATYFRNDV
KDLINLKRLD DPNANGALSR TNSQYQNIAN ARLSGIELQA QYQTERLTLF TNYGSTKGRD
KDSGEALSNI AASKIGVGAD YALVKDKFTV GATITHYAAQ HRVPKDHAVT YPSYILTDLR
ATYAPLKGEW KNLRLDFALE NLFDRKYQPA FSLMEGTGRN AKISAVYSF
SEQ ID NO: 20 (outer membrane protein assembly factor BamA):
MKKLLIASLL FGTTTTVFAA PFVAKDIRVD GVQGDLKQQI RASLPVRAGQ RVTDNDVANI
VRSLFVSGRF DDVKAHQEGD VLVVSVVAKS IISDVKIKGN SIIPTEALKQ NLDANGFKVG
DVLIREKLNE FAKSVKEHYA SVGRYNATVE PIVNTLPNNR AEILIQINED DKAKLASLTF
KGNESVSSST LQEQMELQPD SWWKLWGNKF EGAQFEKDLQ SIRDYYLNNG YAKAQIIKTD
VQLNDEKTKV NVTIDVNEGL QYDLRSARII GNLGGMSAEL EPLLSALHLN DTFRRSDIAD
VENAIKAKLG ERGYGSATVN SVPDFDDANK TLAITLVVDA GRRLTVRQLR FEGNTVSADS
TLRQEMRQQE GTWYNSQLVE LGKIRLDRTG FFETVENRID PINGSNDEVD VVYKVKERNT
GSINFGIGYG TESGISYQAS VKQDNFLGTG AAVSIAGTKN DYGTSVNLGY TEPYFTKDGV
SLGGNVFFEN YDNSKSDTSS NYKRTTYGSN VTLGFPVNEN NSYYVGLGHT YNKISNFALE
YNRNLYIQSM KFKGNGIKTN DFDFSFGWNY NSLNRGYFPT KGVKASLGGR VTIPGSDNKY
YKLSADVQGF YPLDRDHLWV VSAKASAGYA NGFGNKRLPF YQTYTAGGIG SLRGFAYGSI
GPNAIYAAQN GNSNSNSNGT FNKISSDVIG GNAITTASAE LIVPTPFVSD KSQNTVRTSL
FVDAASVWNT KWKSDKSGLD NNVLKSLPDY GKSSRIRAST GVGFQWQSPI GPLVFSYAKP
IKKYENDDVE QFQFSIGGSF
SEQ ID NO: 21 (translation initiation factor IF-2):
MTEDVKADAP KKLSIQRRTK TTVSSTTTGG KSKEVQVEVR KKRIVKIDIA QQEEAKLKAQ
QEAEAKKIAE QKAAEEKARL EAEKAATKKE ADEKSKAEKA KAETAKPAKS AVDSKAKFVD
PEKEKRKAEE AELRRKAEEV ARQKAEEQAR RAAEEAKRYA EADDSDNESS SEDYSDYNLS
SRYALEAEDE EDRRNENRGR GKNKVAKAKK GGRDDENSKN SKNERESNRK NQKDAKFGKG
KNGKKGTALQ QAFTKPVQVV KADVVIGETI TVAELANKMS VKATEIIKVM MKMGEMVTIN
QVIDQETAQL VAEELGHKVI LRNENELEEA VLGDRDVNAE KVIRAPVVTI MGHVDHGKTS
LLDYIRKAKV AAGEAGGITQ HIGAYHVEMD DGKMITFLDT PGHAAFTSMR ARGAKATDIV
VLVVAADDGV MPQTIEAIQH AKAAGAPLVV AVNKIDKPEA NPDRVEQELL QHDVISEKFG
GDVQFVPVSA KKGTGVDDLL DAILLQSEVL ELTAVKDGMA SGVVIESYLD KGRGPVATIL
VQSGTLRKGD IVLCGFEYGR ARAMRDENGK EVDEAGPSIP VELLGLSGVP AAGDEATVVR
DEKKAREVAL YRQGKFREVK LARQQKAKLE NMFSNMSEGD VAELNVIVKA DVQGSVEAIV
QALNELSTNE VKVKVVGSGV GGITETDATL ATASNAIIVG FNVRADATAR RVIEAENIDL
RYYSIIYELL NEIKAAMSGM LEPEFKQEII GLAEVRDVFR HPKFGAIAGC MVTEGVVKRN
NPIRVLRDNV VIFEGELESL RRFKDDVSEV RNGMECGIGV KNYNDVKVGD QIEVFEVVEV
KRSI
SEQ ID NO: 22 (ligand-gated channel):
MTKKPYFRLS IISCLLISCY VKAETQSIKD TKEAISSEVD TQSTEDSELE TISVTAEKVR
DRKDNEVTGL GKIIKTSESI SREQVLNIRD LTRYDPGISV VEQGRGASSG YSIRGMDRNR
VALLVDGLPQ TQSYVVQSPL VARSGYSGTG AINEIEYENV KAVEISKGGS SSEYGNGALA
GSVTFQSKSA ADILEGDKSW GIQTKNAYSS KNKGFTHSLA VAGKQGGFEG LAIYTQRNSI
ETQVHKDALK GVQSYNRLIA KEDGSNAYFV MEDECPKDYN SCIPSAKPPA KLSSQRETVS
VSDYTGANRI KPNPMKYESQ SWFLRGGYHF SEQHYIGGIF EFTQQKFDIR DMTFPAYLRS
TEKPDLENSS FYPKQDYGAY QRIEDGRGVK YASGLYFDEH HRKQRVGIEY IYENKNKAGI
IDKAVLSANQ QNIILDSYMQ HTHCSLYPNP SKNCRPTLDK PYSYYHSDRN VYKEKHNMLQ
LNLEKKIQQN WLTHQIVFNL GFDDFTSALQ HKDYLTRRVT ATAKSISEKP GETPRRNGFK
LQPYLYPKPN ASFVGRDHCN YQGSSSNYSD CKVRLIKGKN YYFAARNNMA LGKYVDLGLG
IRYDVSRTKA NESTISVGKF KNFSWNTGIV IKPTEWLDLS YRLSTGFRNP SFAEMYGWRY
GGKNDEVYVG KFKPETSRNQ EFGLALKGDF GNIEISHFSN AYRNLIAFAE ELKNGKGKGN
YGYHNAQNAK LVGVNITAQL DFNGLWKRIP YGWYATFAYN RVKVKDQKIN AGLASVSSYL
FDAIQPSRYI IGLGYDHPSN TWGINTMFTQ SKAKSQNELL GQRSLGNTSR GVKSTRKLTR
AWHILDVSGY YMVNRSILFR LGVYNLLNYR YVTWEAVRQT AQGAVNQHQN VGNYTRYAAS
GRNYTLTLEM KF
SEQ ID NO: 23 (TonB-dependent receptor):
MKKAIKLNLI TLGLINTIGM TITQAQAEET LGQIDVVEKV ISNDKKPFTE AKAKSTRENV
FKETQTIDQV IRSIPGAFTQ QDKGSGVVSV NIRGENGLGR VNTMVDGVTQ TFYSTALDSG
QSGGSSQFGA AIDPNFIAGV DVNKSNFSGA SGINALAGSA NFRTLGVNDV ITDDKPFGII
LKGMTGSNAT KSNFMTMAAG RKWLDNGGYV GVVYGYSQRE VSQDYRIGGG ERLASLGQDI
LAKEKEAYFR NAGYVLNDAG QWTPDLSKNA WSCNEDPPRL AENIENVKCT HYSFDPRKND
RKEILKKLKA GTKPENIDEL QNGKDGIKET DKSFERNKDQ YSVAPIEPGS LQSRSRSHLL
KFEYGDDHQN LGAQIRTLDN KIGSRKIENR NYQVNYNFNN NNYLDLNLMA AHNIGKTIYP
KGGFFAGWRV ADKLITKNVA NIVDINNSHT FLLPKEIDLK TTLGFNYFTN EYSKNRFPEE
LSLFYNDDSH DQGNYSNLGR FRGNRNLLPQ RSVILQPSGK QKFKTVYFDT ALSKGIYHLN
YSVNFTHYAF NGEYVGYENT ANQINEPILH KSGHKKAFNH SATLSAELSD YFMPFFTYSR
THRMPNIQEM FFSQVSNAGV NTALKPEQSD TYQLGFNTYK KGLFTQDDVL GIKLVGYRSF
IKNYIHNVYG VWWRDGVVPT WADSTGFRFT IAHQNYQPIV KKSGAELELN YDMGRFFANV
SYAYQRTNQP TNYADASSRP NNASKEDILK QGYGLSRVSM LPKDYGRLEI GTRWFDQKLT
LGMAARYYGK SKRATIEEEY INGSHFELKT SGKRTYYVVK KTEEIKKQPI ILDLHVSYEP
IKDLIIKAEV QNLLDKRYVD PLDAGNDAAS QRYYSSLNDS ICSKSQDCED GGKDKTVLYN
FARGRTYILS LNYKF
SEQ ID NO: 24 (hypothetical protein):
MYKLNVISLI ILTTYTGATY ASTQGLPQEG LPQEDTVVFG NVTIDKTTAD KMTITQSSPT
TQINWKSFDI GQNKEVEFKQ PSPNAVAYNR VTGGNASQIQ GKLTANGKVY LANPNGVIIT
KGAEINVAGL LATTKDLEQI SENGNGNGNS YQFTRKTKDG QELKEGKVLN EGKIKAKDFV
VLNGDEVINK EEINVTNGKV YLSSGDNFTF TLLPDSGISV ALEDNAVRGI VKNEGIIKAE
DITLNAKGRK EALDSLVMNN GVLEATKVSN KNGKVVLSAD EIKLDDKSNI KGDMVSFVAD
VTSNKELKDN IKITSQTGSK VTSPKIDFKG KSVNINGNFG RDDSKAHYND EHKTLKTEVN
IDVPDDENIR IANIEDEDKD STGSFIQTDA LSSLLANNGK VTLKGNNVNI SGRIHIDSFR
GSDSLLKLTN KGHIKINNAD IHSKGRLFFI TSLQNEEDFK SNITITDSKI NLGNGAMGLG
RSVNESDYDN KYQKTEGSQR KKFNVKMSNV EFNQVDDVIL AGGFEKVNLD KIVATGQINF
YIDGGVSRNR TNGEPWKYEY GVLDLDKRTQ LSELDQGRRR WGYYYDLELD MNRAYLYRFD
LFAAKNTRRS TIKGTEINIS NSNINLKNGF VHLLAEKIKL DNSKIDITFD KDNSQDTLAQ
TNRLGMNGKV SMINSHIKIV GDEKSGISPT GTYATMFLIG ELIGEKSSIF VKSHQGYTFK
TDGDTKIAGK NSKEDLKITA INTGGRAAEE VLINGALGSA DNDANIANMA FTIGDSANTK
TTIENADITA LAPNGGTAYL SSKDVEIEVK PNSDFTFFEL PREKNLNQTK INGASTKLSE
RGFARLYDKI NGVRASNLSA EQLNVTDSSE KIINTNLVSS LDVEKLVSVA VCDAGKGCEE
QQFGDKGNNT KVSVGELEAE Q
SEQ ID NO: 25 (ribonuclease E):
MKRMLINATQ KEELRVALVD GQRLFDLDIE SPGHEQKKAN IYKGKITRVE PSLEAAFVDY
GAERHGFLPL KEIAREYFPD DYVFQGRPNI RDILTEGQEV IVQVNKEERG NKGAALTTFV
SLAGSYLVLM PNNPRAGGIS RRIEGDERTE LKEALSSLDV PEGVGLIVRT AGVGKSPEEL
QWDLKVLLHH WEAIKQASQS RPAPFLIHQE SDVIVRAIRD YLRRDIGEIL IDSPKIFEKA
KEHIKLVRPD FINRVKLYQG EVPLFSHYQI ESQIESAFQR EVRLPSGGSI VIDVTEALTA
IDINSARSTR GGDIEETALN TNLEAADEIA RQLRLRDLGG LVVIDFIDMT PIRHQREVEN
RIRDAVRPDR ARIQISRISR FGLLEMSRQR LSPSLGESSH HICPRCQGTG KVRDNESLSL
SILRLLEEEA LKENTKQVHT IVPVQIASYL LNEKRKAISN IEKRHNVDII VAPNEAMETP
HFSVFRLRDG EEVNELSYNL AKIHCAQDEN TEESLLSRNV ETTAVIEQPA VESAVVALSI
SEAAPTPVER KSNEPSLLAK IIAKIKGLFA TKSEENKPKN NRTSRNPNRN QRRSQDRRSS
RRPRSENNET ERTEEQVRNV RERNQRRPRR NLVEESIAES AVNSTPVFEA KEERTEPVTQ
RRQRRDLRKR VRVEDNETVV ENNFSTTEKM PEVDVITVQN NDEKPVHQNQ RSERQERQRR
TPRHLRAANN QRRRRNQEPK SPMPLFAAVV SPELASGKAW IDYSTVNLPK ENHFLSVDEL
LEQEKTKKGF ITPAMGIVVE EKSPDVKPAL DFITQPANES VQKKVQESLD RLSSYKPQEV
VESIDPAINV DEPETLEKVS KFVRTYEFNG RLGTISSVPH TKAEMTLAKA NDEMPEAFPI
RAWQDSRYYF YGKGAAGHHC AISHVYSEPT RAKSE
SEQ ID NO: 26 (ligand-gated channel protein):
MTNFRLNVLA YSVMLGLTAG VAYAAQPTNQ PTNQPTNQPT NQNGNVSEQL EQINVSGSTE
NSDTKIPPKI AETVKTAKTL EREQANNIKD IVKYETGVTV VEAGRFGQSG FAIRGVDENR
VAINIDGLRQ AETLSSQGFK ELFEGYGNFN NTRNGAEIET LKEVNITKGA NSIKSGSGSL
GGSVIYKTKD ARDYLLNKDY YVSYKKGYAT ENNQSFNTLT LAGRYKKFDA LVVTTSRNGH
ELENYDYKNA NSLTQGKKRE KADPYKIEQD STLLKLSFNP TENHRFTLAA DLYEHRSRGQ
DLSYTLKYQK TDPNLLEVDS RHTNDKTKRR NISFSYENFS QTPFWDTLKI TYSDQRIKTR
ARTDDYCDAG VRYCEGTANP AGLKLTDGKI TRRDGSELQF EKKDKNIDNN IYDFDKFIDT
DDRVIEGKLG LRRSSGTWYD CSIFDCKDKT KMKIFETEHP YGYGTTGTWK KDFELEIKKL
NDKNFARVKD ANNKTYSILP SSPGYLERLW QERDLDTNTQ QLNLDLTKDF KTWRVEHNLQ
YGSSYNTTMK RMVNRAGNDA SDVQWWAEPT LGYSLLYDKP HTCKTAYGGW KANLCPRVDP
KFSFLLPIKT KEKSVYLFDN VVITDYLSFD LGYRYDNIHY QPKYKHGVTP KLPDDIVKGL
FIPLPSGKNN NDDPEVKKNV QQNIDYIAKQ NKKYKAHSYS FVSTIDPTSF LRLQLKYSKG
FRAPTSDEMY FTFKHPDFTI LPNTNLKPEI AKTKEIAFTL HNDDWGFIST SLFKTNYKNF
IDLIFKGEKD FKLVSGGSTL PFSLYQNINR DSAVVKGIEI NSKVFLGKMA KFMDGFNLSY
KYTYQKGRMD GNIPMNAIQP KTMVYGLGYD HPSQKFGFNF YTTHVASKNP EDTYDIYAKD
KNQTNTSIKW RSKSYTILDL IGYVQPIKNL TIRAGVYNLT NRKYITWDSA RSIRSFGTSN
VIDQKTGQGI NRFYAPGRNY KMSVQFEF
SEQ ID NO: 27 (adhesin):
MKKTVFRLNF LTACISLGIV SQAWAGHTYF GIDYQYYRDF AENKGKFAVG AKDIEVYNKK
GELVGKSMTK APMIDFASVA RSGYAALVGD QYIVSVAHNG GYTNADFGAE GQNPDQHRFT
YQIVKRNNYK PDQKHHYNGD YHMPRLHKFV TESSPFDMTG SMKGQTYADK EKYPMRVRIG
SGDQYWRNEQ DKGTQVAEAY GYLIAGNTHM QTGAGNGDVS LSGDVRKGND YGPMPTAGSF
GDSGSPMFIY DATEKKWLIN GVLRTGNPSL GRENTFQLVR KNYFDEIFSA NLKTTIFDFR
TPKNQQNQHY TFHSKNDGSG TITSGAGKIY KVKLANYNLS MKGTEPVYTN GGVNNYSPAL
SHGENLYFID YGKGVLTFSN NIDQGAGGLY FEGDFIVSPT HNETWKGAGI SISDESTVIW
KVNGVENDRL SKIGKGTLHI QAKGKNLGSI SVGDGKVILE QQEDDQHKKQ AFSEIGLVSG
RGTVQLNDDK QFDTDKFYFG FRGGRLDLNG HSLTFKRIQN TDEGAMIVNH NTTEVANVTI
TGNESIVLPN GNNINKLNYS KEIAYNGWFG ETDENKHNGR LNLIYKPTTE DRTLLLSGGT
NLKGDITQEG GTLVFSGRPT PHAYNHLNRP NELGRPQGEV VIDDDWINRT FKAENFQIKG
GSAVVSRNVS SIEGNWTISN NANATFGVVP NQQNTICTRS DWTGLTTCKT ENLTDTKVIN
SIPTTQINGS INLTDNATAN VKGLAKLNGN VTLTNHSQFT LSNNATQIGN IRLSDNSTAT
VDNANLNGNV HLTNSAQFYL KNSHLSHQIQ GDKGTTVTLE NATWTMPSDT TLQNLTLNNS
TVTLNSAYSA SPNNTPRRRR RSLETETTPT SAEHRFNTLT VNGKLSGQGT FQFTSSLFGY
KSDKLKLSND AEGDYILSVR NTGKEPETLE QLTLVESKDN QPLSDKLTFT LENDHVDAGA
LRYVLVKNNG EFRLHNPIKE QELHNDLVRA EQAEQTLEAK QVEPTAETQT SKAKVRLRRA
VLPDTLPAQS LLNALEAKQA EPNAKTQKSK AKTKKARSKR ALRAVFSDPP PDQSQLDVLK
AALEVINAQP QVGKERQAQE KQGKQKDLIS RYSNSALSEL SATVNSMLSV QDELDRLFVD
QAQSAVWTNI AQDKRRYDSD AFRAYQQKTN LRQIGVQKAL DNGRIGAVFS HSRSDNTFDE
QVKNHATLTM MSGFAQYQWG DLQFGVNVGA GISASKMAEE QSRKIHRKAI NYGVNASYQF
RLGQLGIQPY LGVNRYFIER ENYQSEDVKV QTPSLAFNRY NAGIRVDYTF TPTDNISVKP
YFFVNYVDVS NANVQTTVNS TMLQQSFGRY WQKEVGLKAE ILHFQLSAFI SKSQGSQLGK
QQNVGVKLGY RW
SEQ ID NO: 28 (adhesin):
MNKIYRLKFS KRLNALVAVS ELTRGCDHST EKGSEKPVRT KVRHLALKPL SAILLSLGMA
SIPQSVLASG LQGMSVVHGT ATMQVDGNKT TIRNSVNAII NWKQFNIDQN EMVQFLQESS
NSAVFNRVTS DQISQLKGIL DSNGQVFLIN PNGITIGKDA IINTNGFTAS TLDISNENIK
ARNFTLEQTK DKALAEIVNH GLITVGKDGS VNLIGGKVKN EGVISVNGGS ISLLAGQKIT
ISDIINPTIT YSIAAPENEA INLGDIFAKG GNINVRAANI RNQGKLSADS VSKDKSGNIV
LSAKEGEAEI GGVISAQNQQ AKGGKLMITG DKVTLKTGAV IDLSGKEGGE TYLGGDERGE
GKNGIQLAKK TSLEKGSTIN VSGKEKGGRA IVWGDIALID GNINAQGKDI AKTGGFVETS
GHYLSIGNDA AVEAKEWLLD PDNVTISNGN DDQSQLKDDR GDSPNKILAD NKHTVNNKTL
STALAKGIGV NISAKKKVNV TADINVHNGT LTLHSEQGGV EINGDITSEQ NGNLTIKAGS
WVDVHKNITI GMGFLNITAG GSVAFEKAGG DKGRAASDAK IVAQGVITAG SGQDFRFNNV
SLNGTGRGLK FITAKGNKGN FSAKFDGVLN ISGNISINHT ANNQLSYFHR QGYTYWNLTQ
LNVDSDSSFS LTSIKDAIKV GGYDNAKDKK NTGGIGFTRD TIFNVKQGAR VDISYTLPIS
PVKNSRIAAV NFDGNITVKG GGVVNLKFNA LSNNYKTPGV NISSRFINVT EGSQLNITGS
MPSTTLFNVA NDLIINATNS FVSIKEIEGT DTHLDTGLKV NGNVTIKGGN VTLGSNKAKT
KFDKNVTVEK GANLTLASAN FGNHKGALTV AGNINTQGKL VATGDTIDVS GDFTVGNDAT
FNGNTNNNLN ITGNFTNNGT SIIDVKKGAA KLGNITNEGS LNITTHANTN QKTIITGNIT
NKKGDLNIRD NKNNAEIQIG GNISQKEGNL TISSDKVNIT KQITIKAGVN GENSDSGTEN
NANLTIKTKT LELTNNLNIS GFHKAEITAK DNSDLIIGKA SSDSGNAGAQ KVIFDKVKDS
KISAGNHNVT LNSEVETSNG NSNAAGDSNG NNAGLTISAK DVAVNNNITS HKTINISATT
GNVTTKEGTT INATTGGVEV TAKTGDIKGG IESKSGGVTL TATGDTLAVG NISGNTVSVT
ANSGTLTTKA DSTIKGTGSV TTLSQSGDIG GTISGKTVSV TATTDSLTVK GGAKINATEG
TATLTASSGK LTTEASSSIT SAKGQVDLSA RDGNIGGSIN AANVTLNTTG TLTTVKGSSI
NANSGTLVIN AEDAKLDGTA SGDRTVVNAT NASGSGSVTA VTSSSVNITG DLSTINGLNI
ISKNGKNTVV LKGAEIDVKY IQPGVASAEE VIEAKRALEK VKDLSDEERE TLAKLGVSAV
RFVEPNNAIT VNTQNEFTTR PSSQVTISEG KACFSSGDGA AVCTNVADDG QQ
SEQ ID NO: 29 (HMW2A, high molecular weight adhesin 2):
MNKIYRLKFS KRLNALVAVS ELTRGCDHST EKGSEKPVRT KVRHLALKPL SAILLSLGMA
SIPQSVLASG LQGMSVVHGT ATMQVDGNKT TIRNSVNAII NWKQFNIDQN EMVQFLQENN
NSAVFNRVTS DQISQLKGIL DSNGQVFLIN PNGITIGKEA IINTNGFTAS TLDISNENIK
ARNFTLEQTK DKALAEIVNH GLITVGKDGS VNLIGGKVKN EGVISVNGGS ISLLAGQKIT
ISDIINPTIT YSIAAPENEA INLGDIFAKG GNINVRAATI RNKGKLSADS VSKDKSGNII
LSAKEGEAEI SGVISAQNQQ AKGGKLMITG DKVTLKTGAV IDLSGKEGGE TYLGGDERGE
GKNGIQLAKK TSLEKGSTIN VSGKEKGGRA IVWGDIALID GNINAQGSDI AKTGGFVETS
GHYLSIDSNA IVKTKEWLLD PDNVTIEAPS LSRADTDISS EFPIGDGTEN SPKKNADKTI
LTNETISNFL QNAKVMNITA KRKLTVNSSI SIGSRSHLIL HSEGQGDGGV QIDGDITSEG
GNLTINSGGW VDVHKNITLG TGFLNITAGG SVAFEKGGNN ARNATDAQIT AQGTITVNKD
DKQFRFNNVS INGTGEGLKF IANQNNFTHK FDGEINISGI VTINQTTKKD AKYWHASKDS
YWNVSSLTLN DDAKFTFIKF VDSGSNSQDL RSARRRFAGV HFNGTGGKTN FNIGANAKAL
FKLKPNAATD PKKELPITFN ANITATGSSD SSVMFDIHAN LTSRAASINM DSINITGGLD
FSITSHNRNS NAFEIKKDLT INATNSKFSL KQTKDLFENQ YTGDAINSTR NLTILGGNVT
LGGENSSSNI TGNITIAAEA NVTLQAYADN SIKGHKKKTL TLGNVSTSGN LSLTGSKVEV
KGDLAVLNGA TFKGETNDSL NITGTFTNNG TADINIKRGV VNIQGDITNK GGLNITTNAQ
KNQKTIINGN ITNKKGNLNI TNNGNDTEIQ IGGNISQKEG NLTISSDKVN ITKQITIKAG
VDEKDSSSST ASDANLTIKT KELKLVEDLN ISGFNKAEIT AKDGSDLTIG NTNSADGTNA
KKVTFNQVKD SKISANDHNV TLNSKVETSG NTDNTGDGSG NNAGLTIAAK NVEVKNNITS
NKTVNITASE KLTTKADATI NATTGNVEVT AKTGDIKGEV KSTSGNVNIT ANGDTLNVSN
VSGNAVTITA DKGKLTTQES STISGTESVT TSSQSGDIGG AISGNTVSVK ATNDLITKAN
SKIEAKTGEA NVTSATGIIG GTISGNTVNV TANTGSLTIK GGAKVDATNG AATLTAESGK
LTTQAGSTIT SNNGQTTLTA KDGSIAGSID AANVTLNTTG TLTTVVGSSI NANEGTLVIN
AQDATLNGDA SGDRTEVNAV NASGSGSVTA VTSSSVNITG DLSTINGLNI ISKNGKNTVV
LKGAEIDVKY IQPGVASAEE VIEAKRALEK VKDLSDEERE TLAKLGVSAV RFVEPNNAIT
VNTQNEFTTR PSSQVTISEG KACFSSGDGA AVCTNVADDG QQ
SEQ ID NO: 30 (peptidase):
MLNKKFKLNF IALTVAYALT PYTEAALVRN DVDYQIFRDF AENKGKFSVG ATNVEVRDNK
NNNLGSALPK DIPMIDFSAV DVDKRIATLV NPQYVVGVKH VGNGVGELHF GNLNGNWNPK
FGNSIQHRDV SWEENRYYTV EKNNFSSELN GKTQNNEKDK QYTSNKKDVP SELYGQALVK
EQQNQKRRED YYMPRLDKFV TEVAPIEAST TSSDAGTYND QNKYPAFVRL GSGSQFIYKK
GSHYELILEE KNEKKEIIHR WDVGGDNLKL VGNAYTYGIA GTPYKVNHTD DGLIGFGDST
EDHNDPKEIL SRKPLTNYAV LGDSGSPLFV YDKSKEKWLF LGAYDFWGGY KKKSWQEWNI
YKPQFAENIL KKDSAGLLKG NTQYNWTSKG NTSLISGTSE SLSVDLVDNK NLNHGKNVTF
EGSGNLTLNN NIDQGAGGLF FEGDYEVKGT SENTTWKGAG ISVAEGKTVK WKVHNPQFDR
LAKIGKGKLI VEGRGDNKGS LKVGDGTVVL KQQTTTGQHA FASVGIVSGR STVVLNDDNQ
VDPNSIYFGF RGGRLDANGN NLTFEHIRNI DDGARLVNHN MTNASNITIT GAGLITNPSQ
VTIYTPAITA DDDNYYYVPS IPRGKDLYFS NTCYKYYALK QGGSPTAEMP CYSSEKSDAN
WEFMGDNQND AQKKAMVYIN NRRMNGFNGY FGEEATKADQ NGKLNVTFSG KSDQNRFLLT
GGTNLNGELK VEKGTLFLSG RPTPHARDIA NISSTEKDKH FAENNEVVVE DDWINRTFKA
TNINVTNNAT LYSGRNVESI TSNITASNKA KVHIGYKAGD TVCVRSDYTG YVTCHNDTLS
TKALNSFNPT NLRGNVNLTE SANFTLGKAN LFGTINSTEN SQVNLKENSH WYLTGNSDVH
QLDLANGHIH LNNVSDATKE TKYHTLNISN LSGNGSFYYW VDFTKNQGDK VVVTKSAKGT
FTLQVANKTG EPNHNELTLF DASNATERSG LNVSLANGKV DRGAWSYTLK ENSGRYYLHN
PEVERRNQTV DTPSIATANN MQADVPSVSN NHEETARVEA PIPLPAPPAP ATGSAMANEQ
PETRPAETVQ PTMEDTNTTH PSGSEPQADT TQADDPNSES VPSETIEKVA ENSPQESETV
AKNEQKATET TAQNDEVAKE AKPTVEANTQ TNELAQNGSE TEETQEAETA RQSEINSTEE
TVVEDDPTIS EPKSRPRRSI SSSSNNINLA GTEDTAKVET EKTQEAPQVA FQASPKQEEP
EMAKQQEQPK TVQSQAQPET TTQQAEPARE NVSTVNNVKE AQPQAKPTTV AAKETTASNS
EQKETAQPVA NPKTAENKAE NPQSTETTDE NIHQPEAHTA VASTEVVTPE NATTPIKPVE
NKTTEAEQPV TETTTVSTEN PVVKNPENTT PATTQSTVNS EAVQSETATT EAVVSQSKVT
SAEETTVAST QETTVDNSGS TPQPRSRRTR RSAQNSYEPV ELHTENAENP QSGNDVATQL
VLRDLTSTNT NAVISDAMAK AQFVALNVGK AVSQHISQLE MNNEGQYNVW VSNTSMKENY
SSSQYRHFSS KSAQTQLGWD QTISSNVQLG GVFTYVRNSN NFDKASSKNT LAQANLYSKY
YMDNHWYLAV DLGYGNFQSN LQTNHNAKFA RHTAQFGLTA GKAFNLGNFA VKPTVGVRYS
YLSNANFALA KDRIKVNPIS VKTAFAQVDL SYTYHLGEFS ITPILSARYD ANQGSGKINV
DRYDFAYNVE NQQQYNAGLK LKYHNVKLSL IGGLTKAKQA EKQKTAEVKL SFSF

1. A composition comprising a polypeptide, wherein the polypeptide comprises:
a) an amino acid sequence selected from the group consisting of any one of SEQ ID NOs: 10, 1-9, and 11-30, or
b) an amino acid sequence consisting of at least or exactly 9 contiguous amino acid residues from any one of SEQ ID NOs: 10, 1-9, and 11-30, or
c) an amino acid sequence having a sequence identity of at least 60% with the amino acid sequence of a), or
d) an amino acid sequence having a sequence identity of at least 60% with the amino acid sequence of b),
said polypeptide being antigenic in a mammal,
said composition further comprising an immunological adjuvant.

2. The composition according to claim 1, wherein the at least or exactly 9 contiguous amino acids are at least or exactly or at most 10, at least or exactly or at most 11, at least or exactly or at most 12, at least or exactly or at most 13, at least or exactly or at most 14, at least or exactly or at most 15, at least or exactly or at most 16, at least or exactly or at most 17, at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27 at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, or at least or exactly or at most 94, contiguous amino acid residues.

3. The composition according to claim 1, wherein the sequence identity with the amino acid sequence of a) or b), which is defined in c) and d), is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

4. (canceled)

5. The composition according to claim 1, wherein the at least 9 contiguous amino acid residues has an N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, and 87 in any one of SEQ ID NOs: 1-30, or
any one of amino acid residues 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, and 146 in any one of SEQ ID NOs: 2-30, or
any one of amino acid residues 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, and 179 in any one of SEQ ID NOs: 3-30, or
any one of amino acid residues 180, 181, 182, 183, 184, and 185 in any one of SEQ ID NOs: 4-30, or
any one of amino acid residues 186, 187, 188, 189, 190, 191, 192, 193, 194, 195, 1%, 197, 198, 199, 200, 201, 202, 203, 204, 205, 206, 207, and 208 in any one of SEO ID NOs: 5-30, or
any one of amino acid residues 209, 210, 211, 212, 213, 214, 215, 216, and 217 in any one of SEQ ID NOs: 6-30, or
any one of amino acid residues 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, and 256 in any one of SEQ ID NOs: 7-30, or
any one of amino acid residues 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 229, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, and 371 in any one of SEQ ID NOs: 8-30, or
any one of amino acid residues 372, 373, 374, 375, 376, 377, 378, 379, 380, 381, 382, 383, 384, 385, 386, 387, 388, 389, 390, 391, 392, 393, 394, 395, 396, and 397 in any one of SEQ ID NOs: 9-30, or
any one of amino acid residues 398, 399, and 400 in any one of SEQ ID NOs: 10-30, or
any one of amino acid residues 401, 402, 403, 404, 405, 406, 407, 408, 409, 410, 411, 412, 413, 414, 415, 416, 417, 418, 419, and 420 in any one of SEO ID NOs: 11-30, or
any one of amino acid residues 421, 422, 423, 424, 425, 426, 427, 428, 429, 430, 431, 432, 433, 434, 435, 436, 437, 438, 439, and 440 in any one of SEQ ID NOs: 12-30, or
any one of amino acid residues 441, 442, 443, 444, 445, 446, 447, 448, 449, 450, 451, 452, 453, 454, 455, and 456 in any one of SEQ ID NOs: 13-30, or
any one of amino acid residues 457, 458, 459, 460, 461, 462, 463, 464, and 465 in any one of SEQ ID NOs: 14-30, or
any one of amino acid residues 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 in any one of SEO ID NOs: 15-30, or
any one of amino acid residues 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 56, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, and 622 in any one of SEO ID NOs: 16-30, or
any one of amino acid residues 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, and 658 in any one of SEQ ID NOs: 17-30, or
any one of amino acid residues 659, 660, 661, 662, 663, 664, 665, 666, 667, 668, 669, 670, 671, 672, 673, 674, 675, 676, 677, 678, 679, 680, 681, 682, 683, 684, and 685 in any one of SEQ ID NOs: 18-30, or
any one of amino acid residues 686, 687, 688, 689, 690, 691, 692, 693, 694, 695, 696, 697, 698, 699, 700, and 701 in any one of SEQ ID NOs: 19-30, or
any one of amino acid residues 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, and 792 in any one of SEQ ID NOs: 20-30, or
any one of amino acid residues 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, and 836 in any one of SEQ ID NOs: 21-30, or
any one of amino acid residues 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, and 904 in any one of SEO ID NOs: 22-30, or
any one of amino acid residues 905, 906, and 907 in any one of SEQ ID NOs: 23-30, or any one of amino acid residues 908, 909, 910, 911, 912, and 913 in any one of SEO ID NOs: 24-30, or
any one of amino acid residues 914, 915, 916, 917, 918, 919, 920, 921, 922, 923, 924, 925, 926, and 927 in any one of SEQ ID NOs: 25-30, or
any one of amino acid residues 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, and 980 in any one of SEQ ID NOs: 26-30, or
any one of amino acid residues 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, and 1384 in any one of SEO ID NOs: 27-30, or
any one of amino acid residues 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, and 1484 in any one of SEO ID NOs: 28-30, or
any one of amino acid residues 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, and 1534 in SEQ ID NO: 29 or 30, or
any one of amino acid residues 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, and 1786 in SEO ID NO: 30,
with the proviso that the selected amino acid residue satisfies the formula N≤L−n+1, where N is the number of the selected residue, L is the number of amino acid residues in SEQ ID NO: 1-30, and n is the number of consecutive amino acid residues.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The composition according to claim 1, which is fused or conjugated to an immunogenic carrier molecule.

36. The pelypeptide composition according to claim 35, wherein the immunogenic carrier molecule is a polypeptide that induces T-helper lymphocyte responses in a majority of humans, such as immunogenic carrier proteins selected from the group consisting of keyhole limpet hemocyanin or a fragment thereof, tetanus toxoid or a fragment thereof, diphtheria toxoid or a fragment thereof.

37. The composition according to claim 1, which is capable of inducing an adaptive immune response against the polypeptide in a mammal.

38. The composition according to claim 37, which is capable of inducing, in the mammal, a protective adaptive immune response against infection with Haemophilus influenzae.

39. The composition according to claim 37, which induces a humoral and/or a cellular immune response.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. The composition according to claim 1, wherein the immunological adjuvant is an aluminium based adjuvant.

65. A method for inducing immunity in an animal by administering at least once an immunogenically effective amount of a composition according to claim 1 so as to induce adaptive immunity against Haemophilus influenzae in the animal.

66. The method according to claim 65, wherein the animal receives between 0.5 and 5,000 μg of the polypeptide defined in claim 1 per administration.

67. The method according to claim 65, wherein the animal receives a priming administration and one or more booster administrations.

68. The method according to claim 65, wherein the animal is a human being.

69. The method according to claim 65, wherein the administration is for the purpose of inducing protective immunity against Haemophilus influenzae.

70. The method according to claim 69, wherein the protective immunity is effective in reducing the risk of contracting infection with nontypable Haemophilus influenzae or is effective in treating or ameliorating infection with nontypable Haemophilus influenzae.

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. The composition according to claim 37, wherein said mammal is a human being.

94. The composition according to claim 38, which induces a humoral and/or a cellular immune response.


SEQUENCE LISTING (127 sequences)

SEQ ID NO: 1 - protein, 95 aa, Haemophilus influenzae
MKKLATLTALASALTMAVATAVQAESKSSNTDNTATPCVGDKCVKTKAAEGKCGEGKCGADKAKSAEGKCGEGKCGASKPKAAEGKCGEGKCGSK

SEQ ID NO: 2 - protein, 154 aa, Haemophilus influenzae
MKKAFWLVLSSAIVLAACDDKPKMTEQTKTTDQAKMAAEQPDQKPVLINYKQIALDKSQQAIDTAKVAQDKSVQANEAVKVAMQKQQEAEIALSQGGEEAEKLSKQKMDESNAAAELALKLSEESKAASALSIQLSKEAEDAANKANQQAQAEK

SEQ ID NO: 3 - protein, 187 aa, Haemophilus influenzae
MKKLTLALVLGSALAVTGCFDKQEAKQKVEDTKQTVASVASETKDAAANTMTEVKEKAQQLSTDVKNKVAEKVEDAKEVIKSATETASEKATEIKEAVSEKASEMKEAASEKASEMKEAASEKASEMKEAASEKASEMKEAASEKASEMKEAASEKVGEMKEKATEMKEAVSEKATQAVDAVKEATK

SEQ ID NO: 4 - protein, 193 aa, Haemophilus influenzae
MKKKNQILVSLSIVALLGGCSEEQVQRDVYQSLDDCLADWKKIELCEADKNTESTQKTETTPQQGLGLNIRDNGNAESAVKNPAENNVQANQSENNAESTAKAESTDPSLGAAIAGGVIGYMAARAISSFLGPSYHPGNRAVTTPTGQVVQPQTNRSVGKPMLVKGNAGSMNSKPVSRGGFSSPNNTHRSSGG

SEQ ID NO: 5 - protein, 216 aa, Haemophilus influenzae
MNLTSLINKSAKALALSAAFVALPFLAYADDAQKPADHVTYEPQLDNQRDPNQYCAKCHKFDKIDKNQTLDRSGGELHFGKFHGAHLDKKNPNNGKAITCVSCHGNVSENHRRGAKDVMRFEGDIFGNKKPMYSVQEQNQVCFACHQPDKLREKLWAHDVHAMKLPCASCHTLHPKEDAMKGIQPKQRVKLCVDCHGKQQKRKAEQDKLTKQKDKQ

SEQ ID NO: 6 - protein, 225 aa, Haemophilus influenzae
MGIKNKPITRPCPQCGRNYQYRRASGRTFELCEYCRNLDCVVCGKKVPPERGRKNTCCAECEKLKIHNIQNAHYAKRIAEDPELNKRNHAKARENRKADPERMHEHLEAQRERHYRRVQDPNYLATRKVYQAQRWQDKKDEILAQRREFWDSLSDVEKAERLERNQAIQRKHKAKKRDQLKLDPQKWAEYQEYQRTKRREHRQRKALNELMVGTKELLNVTNKDK

SEQ ID NO: 7 - protein, 264 aa, Haemophilus influenzae
MQQTKRSLLGLLISLIAHGIIIGFILWNWNEPSDSANSAQGDISTSISMELLQGMVLEEPAPEPEDVQKEPEPEPENVQKEPEPEKQEIVEDPTIKPEPKKIKEPEKEKPKPKEKPKNKPKKEVKPQKKPINKELPKGDENIDSSANVNDKASTTSAANSNAQVAGSGTDTSEIAAYRSAIRREIESHKRYPTRAKIMRKQGKVSVSFNVGADGSLSGAKVTKSSGDESLDKAALDAINVSRSVGTRPAGFPSSLSVQISFTLQ

SEQ ID NO: 8 - protein, 379 aa, Haemophilus influenzae
MEKAGYERASDSDSSFSGGGGWREDNSSDSYRSTSDRWNDHKSRYGKDKVYTDAFNERRNNSSWSGGHSAISRTISEKYHSLSNGQMSAAVPEKDQKTLTGGLFGKSYSNAPYSERTPSIFDRNIRGSMTLNNGDVWSSDPQYSSVRERADINSYDRIKRGEELNLIGRAVGGVFSGVGGAATTPVGKIAESAANFGLSHVGDLSRQFKSNQEQAYYDSLTPEGKAYYDTRVDFINKSYKNAREKYETNDKWIDRGITAAQVGLSALGPPGAMLGSGIGLLGKAINKKDTMTKSLRDLTETLNSNALNNHIAQQNELAEKERQAYKEFMAGRDLRSDNTQPKGILNTMYNRMQNIDPDKQVKTSDVPNLRNYWANIIVS

SEQ ID NO: 9 - protein, 405 aa, Haemophilus influenzae
MNKSFLLLPLSLVVLSACTSNSPAPISDADGNLSPSVVQSVNGSNVGGAWQPEIQKNSLPTMGNMVTPQPNFQPINQQPAMPTKPAQPAFQPSPKTVVSAPTVQTKTVTKTIADCVDGQHINIPRNPNTNAPDYSKISKGSYKGNTYKVNKGDTMFLIAYLAGIDVKELAALNNLSEPYNLSLGQVLKISNCGTKTVTTTVSVKQPAVTASTAMPAKPAVTYTPGANGTQIGSDGTIIGPIKSEAGISSSVSVATSSTQVTSSVNNANSTPINANVVAPIASNVAWQWPTSGNIIQGFSSTDGGNKGIDISGSRGQAVKAAAAGRIVYAGNALRGYGNLIIIKHNDDFLSAYAHNDKILVADQQEVKAGQDIAKMGSSGTNTVKLHFEIRYKGKSVDPVRYLPKR

SEQ ID NO: 10 - protein, 408 aa, Haemophilus influenzae
MQNNRQKKGINAFAISILLHFILFGLLILSSLYHTVEIMGGGEGEGDVIGAVIVDTGTAAQEWGRIQQQKKGQADKQKRPEPVVEEKPPEPNQEEIKHQQEVQRQEEIKHQQEVQRQEELKRQQEQQRQQEIKKQQEQARQEALEKQKQAEEARAKQAAEAAKLKADAEAKRLAAAAKQAEEEAKAKAAEIAAQKAKQEAETKAKLEAEAKAKAAAEAKAKAEAEAKAKAAAEAKAKAEAEAKAKAAAEAKAKADAEAKAKAAAEAKAAAEAKRKADQASLDDFFNGGDVGGGSASKGGNTNKGGTQGSGAALGSGDGGKVGDQYAGVIKKEIQRRFLKDPNFAGKVCRIKIQLGRDGTILGYQKISGSDDICSAALSAVARTKKVPAAPSDEIYEKYKSPIIDFDIR

SEQ ID NO: 11 - protein, 428 aa, Haemophilus influenzae
MDKNQQSSQNELDLGLNQEPITPKKTIQPSSSILGKAKGLFAKKNHVQTNFQQRKEPTFGDSSTQENDPLIPSENLKKVQKPVLQTSSTEENISAVDEEISAENNADEPVEKAEKPILAQPEKWKILQVLPAKHRRLFMAIFVLVILLIIFFALKPSSDTVESFTQSNSNEVPVQFQSLDQSQPLETTILDNPPAQNQMAVEQANQSEFAPKAEEAANNTTAQNPLVENAPMQQNVVQSPSQMPNEMAAASVMPMQPAQAEQPKATVPVQPMKKAVEPQVAHKDTVKKEVKVAENAQAPSKATEQNVAKTAGNAPIVEAKPVQVKKEKKVQIVDAKPVSKSAASRLSAKTLTVPKGVSLMQVFRDNQLNISDVNAMSKAAGAGNVLSSFKSGDKVTVSVNNQGRVNEMRLSNGARFVRQSDGSYQYKK

SEQ ID NO: 12 - protein, 448 aa, Haemophilus influenzae
MAEENKEIIAYKGFKQDWTCRGYQYEVGKTYEHKGNVKACESGFHACEYPLDVLSYYSPAVSKFAVVKMSGETSKDSDDTKIASAKITIETEINLPEMIKKAVEWIKGKVDWDAAKVSNTGDWSAATNTGDWSAATNTGDQSAATNTGNWSAATNTGDQSAATNTGDQSAATNTGYRSVATNTGYRSVATNTGDQSAATNTGGQSAATNTGDQSAATNTGYRSVATNTGDQSAATNTGYRSVATNTGDQSAATNTGGQSAATNTGDQSAATNTGYRSVATNTGDQSAATNTGNWSAATNTGDQSAATNTGDQSAATNTGYRSVATNTGDRSAATNTGYRSAATNTGYRSVATNTGDRSAATNTGYRSVATNTGDQSVAEISGKQSIAVALGWQSKAKASINGAIVCVYRNHDGELIHIKASKVGENNIKADTWYTLDEFGEFVEVKDD

SEQ ID NO: 13 - protein, 464 aa, Haemophilus influenzae
MKANKAFYVIGGITALVVTLMAGFLFSGSSSTNTAETKKEDTLDISFKDLSPEELRAMGIEGDTSKDTVRTLIGKVKEHNKRFDEVILQNERLVKENEQLKNQANNTDYQIQQAVQAETGALINEINALKDQVVQAGNQVVGNGNAQANPNAPLPINGATTGPQVETRGGINWVNPSDMPTTDRQGNALQGIGVNGAGIGFPKTFDNNVNDIRKSNDGLQSSTGNNAIFNTATPFFTIAANSTLTNSVAMTALVGRVPIDNKVTEPYPFKLLIGRDNLIANGIELPDIEGAIVSGTASGDWTLSCVRGDVKSLTFVFSDGRIVSTDNSDDKIGWLSDPHGVPCIPGERRTNAPEYLTTNFFLTGASAAAQGLSSSQSTTVVDGGAVIGAVTGNNGKYILGQALGGGLRETADWFRQRYGQMFDAVYVPPGKEVAVHIEKQIDIDYDRMSRKVKYGQASRQSNLD

SEQ ID NO: 14 - protein, 473 aa, Haemophilus influenzae
MQHVKLARDRRKKRTYIKVGVFFVAILLILTGILLTIKDKSEENPIFSTSDSGEYHELNTSPNKNSTALQPDEDATSYDDELQAKDDEVDEVKLSSDDLGTLPQHAQDALNGLLDAADQAIRITDQFSYTVTEGDTLKDVLVLSGLDDSSVQPLIKLDPELAHLKAGQQFYWILNKNDNLEYLNWLVSEKEERIYERLEDGKFKRQVIEKKSIWRKEVLKGEIQNSLNSSLREQGLDTRQISQLSNALQWQVSLRKLKKGTQFAILVSREYLGDKLTGQGNVEALRISSGGKNYYAVQAANGRYYNQQGETLGKGFARYPLQRQARVSSPFNPNRRHPVTGRVRPHKGVDFSVSQGTPVIAPADGTVEKVAYQAGGAGRYVMLRHGREYQTVYMHLSKSLVKAGQTVKKGERIALSGNTGISTGPHLHYEFRINGRAVNPLTVKLPGTSSGMTSAERKQFLVRVREAEKMLKP

SEQ ID NO: 15 - protein, 508 aa, Haemophilus influenzae
MNAFKKSLIVAASFASLSLFNSATAELVYKPLEQPVEPAKPDLKIESVNEKFAEKYPNQYNSWRSTANGDGENIIYADEENPRLIVLWGGYAFAKEYNAPRGHFYAVTDVRNILRTGAPKTANDGPQAMACWTCKGPDVPRLIAEWGEKDYFNAKWAKGGPEIVNSIGCADCHDTTSKDFAEGKPALRIARPHVLRALDALEKATAEKDKAEGRPHNNLSFNTAARTEKRAEICANCHVEYYFAGDIKQVTFPWDNGQTADDIEKYYDDIGFTDWTHSLSKAPMLKAQHPDFEIWSLGMHGKNGVTCVDCHMPKVQGADGKVYTDHQIQNPFDAFDHTCANCHDQSKEKLRDIVTSRKKEVKDVMGRLEDQVVKSHFEAKAAWDAGATKEEMEAALMDIRHAQWRWDYTAASHGGHMHAPEVVLRVLASGLDKVADARTKLAVILTKHGVKTPVQIPDISTADKAWKVMGIDIEKERKAKEEFLKTVVPQWEQQAREKGLLVDPPAQK

SEQ ID NO: 16 - protein, 630 aa, Haemophilus influenzae
MKSVPLITGGLSFLLSACSGGGGSFDVDDVSNPSSSKPRYQDDTSSSRTKSNLEKLSIPSLGGGMKLVAQNLSGNKEPSFLNENGYISYFSSPSTIEDDVKNVKTENKIHTNPIGLEPNRALQDPNLQKYVYSGLYYIENWKDFSKLATEKKAYSGHYGYAFYYGNKTATDLPVSGVATYKGTWDFITATKYGQNYSLFSNARGQAYFRRSATRGDIDLENNSKNGDIGLISEFSADFGTKKLTGQLSYTKRKTDIQQYEKEKLYDIDAHIYSNRFRGKVTPTKSTSDEHPFTSEGTLEGGFYGPNAEELGGKFLARDKRVFGVFSAKETPETEKEKLSKETLIDGKLITFSTKTADATTSTTASTTADVKTDEKNFTTKDISSFGEADYLLIDNYPVPLFPEGDTDDFVTSKHHDIGNKTYKVEACCKNLSYVKFGMYYEDKEKKNTNQTGQYHQFLLGLRTPSSQIPVTGNVKYLGSWFGYIGDDKTSYSTTGNKQQDKNAPAEFDVNFDNKTLTGKLKRADSQNTVFNIEATFKNGSNAFEGKATANVVIDPKNTQATSKVNFTTTVNGAFYGPHATELGGYFTYNGNNPTATNSESSSTVPSPPNSPNARAAVVFGAKRQVEKTNK

SEQ ID NO: 17 - protein, 666 aa, Haemophilus influenzae
MKLNHALKASYIAISCAILSSTMPVSASNSNPFGFNKADSILSDMVFYQIGGGVGYMAPPSRGTIPAAEFGIGWKANLMCGNFDIKTSIKNQLNGLTEGFKDLYSNVIESATGAVASLPAMVIQRANPQLYDILTNGLYQGKIDFNSLKTSCEEMSKKLADATLNGRWSQSSDMESFKDITSTEPDATRAKKRLEESQGKEGKEWVGGKKRGGQGQEPIKIVEDVAKAGYNMQNKRNVLESGRISGSSCSGLLCQTWDNPKDMTDWLTNVIGEKQLTTCKTDCGTASSSRAGVGLTPEIEKENIKTVTQLQKALNMSTPSVEVLAELSSTTVPVTRGLIESLREDPDAQVLGQRLASEIAVAKTMEKMLLARRAVLTGMREPNVANNKDAQEELEKILTLIDREIGQIRMEIDLQKSLTGNSAVAILQNKELREYNTGTGNVTRDSVDKRISDLANGTNSNAAELADADNINIPRNNVTLEVPQMSNSLSYTGTGGTSGNSRGNTSYSSVPAIKGTALDQATGLLKKFEGFSDKAYWDVNAYRTGYGSDTITKADGTIVKVTKDTVVSRADAERDLARRTQEFANRARNNVSSSTWDKLPPNAQAALTSYAYNYGSLTKDVIAAAQASAQSGDMNALANAVRNRQNNNNGVNAKRRNQEADYILGK

SEQ ID NO: 18 - protein, 693 aa, Haemophilus influenzae
MKFKMSKNVICYAWLSVCLSSAIPAFAVQPTLKPSDISIPAISEESQLATKRATTRLTQSHYRKIKLDDDFSEKIFDRYIKNLDFSHNTFLQSDIDELRQKYGTKLDEQLSQGDLSAAFDIYDVMMKRRYERYTYALSLLDKEPDLNGQDQIEIDREKAAAPQTEADANKLWDARVKNDIINLKLKDKKWSEIKAKLTKRYNLAIRRLTQTKADDIVQIYLNAFAREIDPHTSYLSPRTAKSFNESINLSLEGIGTTLQSEDDEISIKSLVPGAPAERSKKLHPGDKIIGVGQATGDIEDVVGWRLEDLVEKIKGKKGTKVRLEIEPAKGGKSRIITLVRDKVRIEDQAAKLTFEKVSGKNIAVIKIPSFYIGLTEDVKKLLVKLENQKAEALIVDLRENGGGALTEAVALSGLFIADGPVVQVRDAYQRIRVHEDDDATQQYKGLLFVMINRYSASASEIFAAAMQDYRRGIIIGQNTFGKGTVQQSRSLNFIYDLDQSPLGVLQYTIQKFYRVNGGSTQLKGVAADINFPEIIDAKEYGEDKEDNALAWDKIPSASYTEVGNIDYIDNAVNILNEKHLARIAKDPEFVALNEELKVRNERRDRKFLSLNYKMRKAENDKDDARRLKDLNERFKREGKKALKDIDDLPKDYEAPDFFLKEAEKIAADFVIFNSDQKINQANGLSEAKTESKK

SEQ ID NO: 19 - protein, 709 aa, Haemophilus influenzae
MRFSKLSLAITTTLVTANALAQSVELDSINVIATRDPSRFTYTPQKQSKDSLLSKQATSVAAALEDIPNVDVRGGSRSIAQKPNIRGLSDNRVVQVIDGVRQNFDLAHRGSYFLPMSLIQEIEVIKGPSSSLWGSGALGGVVAMRTPNALDLLKNNDKFGVKIRQGYQTANNLSEKDVSVFAANDKFDVLISGFYNNADNLRIGKGNKLNNTAYKQFGGLAKFGWQINDANRVELSHRETRFKQTAPGNNEAKNELTNEQIIEKINEYHNPLNNFPPKAKPSLEEFYSGVRARLGGVSYLSDQQIPDQSTVFNYYLTPDNPYLNTHIALYNNKTIEKEQRKVSGVKDQTKLTTRGINLRNSSELSHISFVYGVDYMRDKISTERGTNDKDAKFRAEPYNANSNTTGVYLIAHIPLFGEKLLLSPSVRYDHYDTSSKTVKYKDNHLSPATKLTWKVTNWLDFTAKYNEAFRAPSMQERFVSGAHFGTSTRVGDIINSFVANPNLRPETAKNKEITANLHFDSLFKQGDKFKIEATYFRNDVKDLINLKRLDDPNANGALSRTNSQYQNIANARLSGIELQAQYQTERLTLFTNYGSTKGRDKDSGEALSNIAASKIGVGADYALVKDKFTVGATITHYAAQHRVPKDHAVTYPSYILTDLRATYAPLKGEWKNLRLDFALENLFDRKYQPAFSLMEGTGRNAKISAVYSF

SEQ ID NO: 20 - protein, 800 aa, Haemophilus influenzae
MKKLLIASLLFGTTTTVFAAPFVAKDIRVDGVQGDLKQQIRASLPVRAGQRVTDNDVANIVRSLFVSGRFDDVKAHQEGDVLVVSVVAKSIISDVKIKGNSIIPTEALKQNLDANGFKVGDVLIREKLNEFAKSVKEHYASVGRYNATVEPIVNTLPNNRAEILIQINEDDKAKLASLTFKGNESVSSSTLQEQMELQPDSWWKLWGNKFEGAQFEKDLQSIRDYYLNNGYAKAQITKTDVQLNDEKTKVNVTIDVNEGLQYDLRSARIIGNLGGMSAELEPLLSALHLNDTFRRSDIADVENAIKAKLGERGYGSATVNSVPDFDDANKTLAITLVVDAGRRLTVRQLRFEGNTVSADSTLRQEMRQQEGTWYNSQLVELGKIRLDRTGFFETVENRIDPINGSNDEVDVVYKVKERNTGSINFGIGYGTESGISYQASVKQDNFLGTGAAVSIAGTKNDYGTSVNLGYTEPYFTKDGVSLGGNVFFENYDNSKSDTSSNYKRTTYGSNVTLGFPVNENNSYYVGLGHTYNKISNFALEYNRNLYIQSMKFKGNGIKTNDFDFSFGWNYNSLNRGYFPTKGVKASLGGRVTIPGSDNKYYKLSADVQGFYPLDRDHLWVVSAKASAGYANGFGNKRLPFYQTYTAGGIGSLRGFAYGSIGPNAIYAAQNGNSNSNSNGTFNKISSDVIGGNAITTASAELIVPTPFVSDKSQNTVRTSLFVDAASVWNTKWKSDKSGLDNNVLKSLPDYGKSSRIRASTGVGFQWQSPIGPLVFSYAKPIKKYENDDVEQFQFSIGGSF

SEQ ID NO: 21 - protein, 844 aa, Haemophilus influenzae
MTEDVKADAPKKLSIQRRTKTTVSSTTTGGKSKEVQVEVRKKRTVKTDIAQQEEAKLKAQQEAEAKKIAEQKAAEEKARLEAEKAATKKEADEKSKAEKAKAETAKPAKSAVDSKAKFVDPEKEKRKAEEAELRRKAEEVARQKAEEQARRAAEEAKRYAEADDSDNESSSEDYSDYNLSSRYALEAEDEEDRRNENRGRGKNKVAKAKKGGRDDENSKNSKNERESNRKNQKDAKFGKGKNGKKGTALQQAFTKPVQVVKADVVIGETITVAELANKMSVKATEIIKVMMKMGEMVTINQVIDQETAQLVAEELGHKVILRNENELEEAVLGDRDVNAEKVTRAPVVTIMGHVDHGKTSLLDYIRKAKVAAGEAGGITQHIGAYHVEMDDGKMITFLDTPGHAAFTSMRARGAKATDIVVLVVAADDGVMPQTIEAIQHAKAAGAPLVVAVNKIDKPEANPDRVEQELLQHDVISEKFGGDVQFVPVSAKKGTGVDDLLDAILLQSEVLELTAVKDGMASGVVIESYLDKGRGPVATILVQSGTLRKGDIVLCGFEYGRARAMRDENGKEVDEAGPSIPVELLGLSGVPAAGDEATVVRDEKKAREVALYRQGKFREVKLARQQKAKLENMFSNMSEGDVAELNVIVKADVQGSVEAIVQALNELSTNEVKVKVVGSGVGGITETDATLATASNAIIVGFNVRADATARRVIEAENIDLRYYSIIYELLNEIKAAMSGMLEPEFKQEIIGLAEVRDVFRHPKFGAIAGCMVTEGVVKRNNPIRVLRDNVVIFEGELESLRRFKDDVSEVRNGMECGIGVKNYNDVKVGDQIEVFEVVEVKRSI

SEQ ID NO: 22 - protein, 912 aa, Haemophilus influenzae
MTKKPYFRLSIISCLLISCYVKAETQSIKDTKEAISSEVDTQSTEDSELETISVTAEKVRDRKDNEVTGLGKIIKTSESISREQVLNIRDLTRYDPGISVVEQGRGASSGYSIRGMDRNRVALLVDGLPQTQSYVVQSPLVARSGYSGTGAINEIEYENVKAVEISKGGSSSEYGNGALAGSVTFQSKSAADILEGDKSWGIQTKNAYSSKNKGFTHSLAVAGKQGGFEGLAIYTQRNSIETQVHKDALKGVQSYNRLIAKEDGSNAYFVMEDECPKDYNSCIPSAKPPAKLSSQRETVSVSDYTGANRIKPNPMKYESQSWFLRGGYHFSEQHYIGGIFEFTQQKFDIRDMTFPAYLRSTEKPDLENSSFYPKQDYGAYQRIEDGRGVKYASGLYFDEHHRKQRVGIEYIYENKNKAGIIDKAVLSANQQNIILDSYMQHTHCSLYPNPSKNCRPTLDKPYSYYHSDRNVYKEKHNMLQLNLEKKIQQNWLTHQIVFNLGFDDFTSALQHKDYLTRRVTATAKSISEKPGETPRRNGFKLQPYLYPKPNASFVGRDHCNYQGSSSNYSDCKVRLIKGKNYYFAARNNMALGKYVDLGLGIRYDVSRTKANESTISVGKFKNFSWNTGIVIKPTEWLDLSYRLSTGFRNPSFAEMYGWRYGGKNDEVYVGKFKPETSRNQEFGLALKGDFGNIEISHFSNAYRNLIAFAEELKNGKGKGNYGYHNAQNAKLVGVNITAQLDFNGLWKRIPYGWYATFAYNRVKVKDQKINAGLASVSSYLFDAIQPSRYIIGLGYDHPSNTWGINTMFTQSKAKSQNELLGQRSLGNTSRGVKSTRKLTRAWHILDVSGYYMVNRSILFRLGVYNLLNYRYVTWEAVRQTAQGAVNQHQNVGNYTRYAASGRNYTLTLEMKF

SEQ ID NO: 23 - protein, 915 aa, Haemophilus influenzae
MKKAIKLNLITLGLINTIGMTITQAQAEETLGQIDVVEKVISNDKKPFTEAKAKSTRENVFKETQTIDQVIRSIPGAFTQQDKGSGVVSVNIRGENGLGRVNTMVDGVTQTFYSTALDSGQSGGSSQFGAAIDPNFIAGVDVNKSNFSGASGINALAGSANFRTLGVNDVITDDKPFGIILKGMTGSNATKSNFMTMAAGRKWLDNGGYVGVVYGYSQREVSQDYRIGGGERLASLGQDILAKEKEAYFRNAGYVLNDAGQWTPDLSKNAWSCNEDPPRLAENIENVKCTHYSFDPRKNDRKEILKKLKAGTKPENIDELQNGKDGIKETDKSFERNKDQYSVAPIEPGSLQSRSRSHLLKFEYGDDHQNLGAQIRTLDNKIGSRKIENRNYQVNYNFNNNNYLDLNLMAAHNIGKTIYPKGGFFAGWRVADKLITKNVANIVDINNSHTFLLPKEIDLKTTLGFNYFTNEYSKNRFPEELSLFYNDDSHDQGNYSNLGRFRGNRNLLPQRSVILQPSGKQKFKTVYFDTALSKGIYHLNYSVNFTHYAFNGEYVGYENTANQINEPILHKSGHKKAFNHSATLSAELSDYFMPFFTYSRTHRMPNIQEMFFSQVSNAGVNTALKPEQSDTYQLGFNTYKKGLFTQDDVLGIKLVGYRSFIKNYIHNVYGVWWRDGVVPTWADSTGFRFTIAHQNYQPIVKKSGAELELNYDMGRFFANVSYAYQRTNQPTNYADASSRPNNASKEDILKQGYGLSRVSMLPKDYGRLEIGTRWFDQKLTLGMAARYYGKSKRATIEEEYINGSHFELKTSGKRTYYVVKKTEEIKKQPIILDLHVSYEPIKDLIIKAEVQNLLDKRYVDPLDAGNDAASQRYYSSLNDSICSKSQDCEDGGKDKTVLYNFARGRTYILSLNYKF

SEQ ID NO: 24 - protein, 921 aa, Haemophilus influenzae
MYKLNVISLIILTTYTGATYASTQGLPQEGLPQEDTVVFGNVTIDKTTADKMTITQSSPTTQINWKSFDIGQNKEVEFKQPSPNAVAYNRVTGGNASQIQGKLTANGKVYLANPNGVIITKGAEINVAGLLATTKDLEQISENGNGNGNSYQFTRKTKDGQELKEGKVLNEGKIKAKDFVVLNGDEVINKEEINVTNGKVYLSSGDNFTFTLLPDSGISVALEDNAVRGIVKNEGIIKAEDITLNAKGRKEALDSLVMNNGVLEATKVSNKNGKVVLSADEIKLDDKSNIKGDMVSFVADVTSNKELKDNIKITSQTGSKVTSPKIDFKGKSVNINGNFGRDDSKAHYNDEHKTLKTEVNIDVPDDENIRIANIEDEDKDSTGSFIQTDALSSLLANNGKVTLKGNNVNISGRIHIDSFRGSDSLLKLTNKGHIKINNADIHSKGRLFFITSLQNEEDFKSNITITDSKINLGNGAMGLGRSVNESDYDNKYQKTEGSQRKKFNVKMSNVEFNQVDDVILAGGFEKVNLDKIVATGQTNFYIDGGVSRNRTNGEPWKYEYGVLDLDKRTQLSELDQGRRRWGYYYDLELDMNRAYLYRFDLFAAKNTRRSTIKGTEINISNSNINLKNGFVHLLAEKIKLDNSKIDITFDKDNSQDTLAQTNRLGMNGKVSMINSHIKIVGDEKSGISPTGTYATMFLIGELIGEKSSIFVKSHQGYTFKTDGDTKIAGKNSKEDLKITAINTGGRAAEEVLINGALGSADNDANIANMAFTIGDSANTKTTIENADITALAPNGGTAYLSSKDVEIEVKPNSDFTFFELPREKNLNQTKINGASTKLSERGFARLYDKINGVRASNLSAEQLNVTDSSEKIINTNLVSSLDVEKLVSVAVCDAGKGCEEQQFGDKGNNTKVSVGELEAEQ

SEQ ID NO: 25 - protein, 935 aa, Haemophilus influenzae
MKRMLINATQKEELRVALVDGQRLFDLDIESPGHEQKKANIYKGKITRVEPSLEAAFVDYGAERHGFLPLKEIAREYFPDDYVFQGRPNIRDILTEGQEVIVQVNKEERGNKGAALTTFVSLAGSYLVLMPNNPRAGGISRRIEGDERTELKEALSSLDVPEGVGLIVRTAGVGKSPEELQWDLKVLLHHWEAIKQASQSRPAPFLIHQESDVIVRAIRDYLRRDIGEILIDSPKIFEKAKEHIKLVRPDFINRVKLYQGEVPLFSHYQIESQIESAFQREVRLPSGGSIVIDVTEALTAIDINSARSTRGGDIEETALNTNLEAADEIARQLRLRDLGGLVVIDFIDMTPIRHQREVENRIRDAVRPDRARIQISRISRFGLLEMSRQRLSPSLGESSHHICPRCQGTGKVRDNESLSLSILRLLEEEALKENTKQVHTIVPVQIASYLLNEKRKAISNIEKRHNVDIIVAPNEAMETPHFSVFRLRDGEEVNELSYNLAKIHCAQDENTEESLLSRNVETTAVIEQPAVESAVVALSISEAAPTPVERKSNEPSLLAKIIAKIKGLFATKSEENKPKNNRTSRNPNRNQRRSQDRRSSRRPRSENNETERTEEQVRNVRERNQRRPRRNLVEESIAESAVNSTPVFEAKEERTEPVTQRRQRRDLRKRVRVEDNETVVENNFSTTEKMPEVDVITVQNNDEKPVHQNQRSERQERQRRTPRHLRAANNQRRRRNQEPKSPMPLFAAVVSPELASGKAWIDYSTVNLPKENHFLSVDELLEQEKTKKGFITPAMGIVVEEKSPDVKPALDFITQPANESVQKKVQESLDRLSSYKPQEVVESIDPAINVDEPETLEKVSKFVRTYEFNGRLGTISSVPHTKAEMTLAKANDEMPEAFPIRAWQDSRYYFYGKGAAGHHCAISHVYSEPTRAKSE

SEQ ID NO: 26 - protein, 988 aa, Haemophilus influenzae
MTNFRLNVLAYSVMLGLTAGVAYAAQPTNQPTNQPTNQPTNQNGNVSEQLEQINVSGSTENSDTKTPPKIAETVKTAKTLEREQANNIKDIVKYETGVTVVEAGRFGQSGFAIRGVDENRVAINIDGLRQAETLSSQGFKELFEGYGNFNNTRNGAEIETLKEVNITKGANSIKSGSGSLGGSVIYKTKDARDYLLNKDYYVSYKKGYATENNQSFNTLTLAGRYKKFDALVVTTSRNGHELENYDYKNANSLTQGKKREKADPYKIEQDSTLLKLSFNPTENHRFTLAADLYEHRSRGQDLSYTLKYQKTDPNLLEVDSRHTNDKTKRRNISFSYENFSQTPFWDTLKITYSDQRIKTRARTDDYCDAGVRYCEGTANPAGLKLTDGKITRRDGSELQFEKKDKNIDNNIYDFDKFIDTDDRVIEGKLGLRRSSGTWYDCSIFDCKDKTKMKIFETEHPYGYGTTGTWKKDFELEIKKLNDKNFARVKDANNKTYSILPSSPGYLERLWQERDLDTNTQQLNLDLTKDFKTWRVEHNLQYGSSYNTTMKRMVNRAGNDASDVQWWAEPTLGYSLLYDKPHTCKTAYGGWKANLCPRVDPKFSFLLPIKTKEKSVYLFDNVVITDYLSFDLGYRYDNIHYQPKYKHGVTPKLPDDIVKGLFIPLPSGKNNNDDPEVKKNVQQNIDYIAKQNKKYKAHSYSFVSTIDPTSFLRLQLKYSKGFRAPTSDEMYFTFKHPDFTILPNTNLKPEIAKTKEIAFTLHNDDWGFISTSLFKTNYKNFIDLIFKGEKDFKLVSGGSTLPFSLYQNINRDSAVVKGIEINSKVFLGKMAKFMDGFNLSYKYTYQKGRMDGNIPMNAIQPKTMVYGLGYDHPSQKFGFNFYTTHVASKNPEDTYDIYAKDKNQTNTSIKWRSKSYTILDLIGYVQPIKNLTIRAGVYNLTNRKYITWDSARSIRSFGTSNVIDQKTGQGINRFYAPGRNYKMSVQFEF

SEQ ID NO: 27 - protein, 1392 aa, Haemophilus influenzae
MKKTVFRLNFLTACISLGIVSQAWAGHTYFGIDYQYYRDFAENKGKFAVGAKDIEVYNKKGELVGKSMTKAPMIDFASVARSGYAALVGDQYIVSVAHNGGYTNADFGAEGQNPDQHRFTYQIVKRNNYKPDQKHHYNGDYHMPRLHKFVTESSPFDMTGSMKGQTYADKEKYPMRVRIGSGDQYWRNEQDKGTQVAEAYGYLIAGNTHMQTGAGNGDVSLSGDVRKGNDYGPMPTAGSFGDSGSPMFIYDATEKKWLINGVLRTGNPSLGRENTFQLVRKNYFDEIFSANLKTTIFDFRTPKNQQNQHYTFHSKNDGSGTITSGAGKIYKVKLANYNLSMKGTEPVYTNGGVNNYSPALSHGENLYFIDYGKGVLTFSNNIDQGAGGLYFEGDFIVSPTHNETWKGAGISISDESTVIWKVNGVENDRLSKIGKGTLHIQAKGKNLGSISVGDGKVILEQQEDDQHKKQAFSEIGLVSGRGTVQLNDDKQFDTDKFYFGFRGGRLDLNGHSLTFKRIQNTDEGAMIVNHNTTEVANVTITGNESIVLPNGNNINKLNYSKEIAYNGWFGETDENKHNGRLNLIYKPTTEDRTLLLSGGTNLKGDITQEGGTLVFSGRPTPHAYNHLNRPNELGRPQGEVVIDDDWINRTFKAENFQIKGGSAVVSRNVSSIEGNWTISNNANATFGVVPNQQNTICTRSDWTGLTTCKTENLTDTKVINSIPTTQINGSINLTDNATANVKGLAKLNGNVTLTNHSQFTLSNNATQIGNIRLSDNSTATVDNANLNGNVHLTNSAQFYLKNSHLSHQIQGDKGTTVTLENATWTMPSDTTLQNLTLNNSTVTLNSAYSASPNNTPRRRRRSLETETTPTSAEHRFNTLTVNGKLSGQGTFQFTSSLFGYKSDKLKLSNDAEGDYILSVRNTGKEPETLEQLTLVESKDNQPLSDKLTFTLENDHVDAGALRYVLVKNNGEFRLHNPIKEQELHNDLVRAEQAEQTLEAKQVEPTAETQTSKAKVRLRRAVLPDTLPAQSLLNALEAKQAEPNAKTQKSKAKTKKARSKRALRAVFSDPPPDQSQLDVLKAALEVINAQPQVGKERQAQEKQGKQKDLISRYSNSALSELSATVNSMLSVQDELDRLFVDQAQSAVWTNIAQDKRRYDSDAFRAYQQKTNLRQIGVQKALDNGRIGAVFSHSRSDNTFDEQVKNHATLTMMSGFAQYQWGDLQFGVNVGAGISASKMAEEQSRKIHRKAINYGVNASYQFRLGQLGIQPYLGVNRYFIERENYQSEDVKVQTPSLAFNRYNAGIRVDYTFTPTDNISVKPYFFVNYVDVSNANVQTTVNSTMLQQSFGRYWQKEVGLKAEILHFQLSAFISKSQGSQLGKQQNVGVKLGYRW

SEQ ID NO: 28 - protein, 1492 aa, Haemophilus influenzae
MNKIYRLKFSKRLNALVAVSELTRGCDHSTEKGSEKPVRTKVRHLALKPLSAILLSLGMASIPQSVLASGLQGMSVVHGTATMQVDGNKTTIRNSVNAIINWKQFNIDQNEMVQFLQESSNSAVFNRVTSDQISQLKGILDSNGQVFLINPNGITIGKDAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAANIRNQGKLSADSVSKDKSGNIVLSAKEGEAEIGGVISAQNQQAKGGKLMITGDKVTLKTGAVIDLSGKEGGETYLGGDERGEGKNGIQLAKKTSLEKGSTINVSGKEKGGRAIVWGDIALIDGNINAQGKDIAKTGGFVETSGHYLSIGNDAAVEAKEWLLDPDNVTISNGNDDQSQLKDDRGDSPNKILADNKHTVNNKTLSTALAKGIGVNISAKKKVNVTADINVHNGTLTLHSEQGGVEINGDITSEQNGNLTIKAGSWVDVHKNITIGMGFLNITAGGSVAFEKAGGDKGRAASDAKIVAQGVITAGSGQDFRFNNVSLNGTGRGLKFITAKGNKGNFSAKFDGVLNISGNISINHTANNQLSYFHRQGYTYWNLTQLNVDSDSSFSLTSIKDAIKVGGYDNAKDKKNTGGIGFTRDTIFNVKQGARVDISYTLPISPVKNSRIAAVNFDGNITVKGGGVVNLKFNALSNNYKTPGVNISSRFINVTEGSQLNITGSMPSTTLFNVANDLIINATNSFVSIKEIEGTDTHLDTGLKVNGNVTIKGGNVTLGSNKAKTKFDKNVTVEKGANLTLASANFGNHKGALTVAGNINTQGKLVATGDTIDVSGDFTVGNDATFNGNTNNNLNITGNFTNNGTSIIDVKKGAAKLGNITNEGSLNITTHANTNQKTIITGNITNKKGDLNIRDNKNNAEIQIGGNISQKEGNLTISSDKVNITKQITIKAGVNGENSDSGTENNANLTIKTKTLELTNNLNISGFHKAEITAKDNSDLIIGKASSDSGNAGAQKVIFDKVKDSKISAGNHNVTLNSEVETSNGNSNAAGDSNGNNAGLTISAKDVAVNNNITSHKTINISATTGNVTTKEGTTINATTGGVEVTAKTGDIKGGIESKSGGVTLTATGDTLAVGNISGNTVSVTANSGTLTTKADSTIKGTGSVTTLSQSGDIGGTISGKTVSVTATTDSLTVKGGAKINATEGTATLTASSGKLTTEASSSITSAKGQVDLSARDGNIGGSINAANVTLNTTGTLTTVKGSSINANSGTLVINAEDAKLDGTASGDRTVVNATNASGSGSVTAVTSSSVNITGDLSTINGLNIISKNGKNTVVLKGAEIDVKYIQPGVASAEEVIEAKRALEKVKDLSDEERETLAKLGVSAVRFVEPNNAITVNTQNEFTTRPSSQVTISEGKACFSSGDGAAVCTNVADDGQQ

SEQ ID NO: 29 - protein, 1542 aa, Haemophilus influenzae
MNKIYRLKFSKRLNALVAVSELTRGCDHSTEKGSEKPVRTKVRHLALKPLSAILLSLGMASIPQSVLASGLQGMSVVHGTATMQVDGNKTTIRNSVNAIINWKQFNIDQNEMVQFLQENNNSAVFNRVTSDQISQLKGILDSNGQVFLINPNGITIGKEAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAATIRNKGKLSADSVSKDKSGNIILSAKEGEAEISGVISAQNQQAKGGKLMITGDKVTLKTGAVIDLSGKEGGETYLGGDERGEGKNGIQLAKKTSLEKGSTINVSGKEKGGRAIVWGDIALIDGNINAQGSDIAKTGGFVETSGHYLSIDSNAIVKTKEWLLDPDNVTIEAPSLSRADTDISSEFPIGDGTENSPKKNADKTILTNETISNFLQNAKVMNITAKRKLTVNSSISIGSRSHLILHSEGQGDGGVQIDGDITSEGGNLTINSGGWVDVHKNITLGTGFLNITAGGSVAFEKGGNNARNATDAQITAQGTITVNKDDKQFRFNNVSINGTGEGLKFIANQNNFTHKFDGEINISGIVTINQTTKKDAKYWHASKDSYWNVSSLTLNDDAKFTFIKFVDSGSNSQDLRSARRRFAGVHFNGTGGKTNFNIGANAKALFKLKPNAATDPKKELPITFNANITATGSSDSSVMFDIHANLTSRAASINMDSINITGGLDFSITSHNRNSNAFEIKKDLTINATNSKFSLKQTKDLFENQYTGDAINSTRNLTILGGNVTLGGENSSSNITGNITIAAEANVTLQAYADNSIKGHKKKTLTLGNVSTSGNLSLTGSKVEVKGDLAVLNGATFKGETNDSLNITGTFTNNGTADINIKRGVVNIQGDITNKGGLNITTNAQKNQKTIINGNITNKKGNLNITNNGNDTEIQIGGNISQKEGNLTISSDKVNITKQITIKAGVDEKDSSSSTASDANLTIKTKELKLVEDLNISGFNKAEITAKDGSDLTIGNTNSADGTNAKKVTFNQVKDSKISANDHNVTLNSKVETSGNTDNTGDGSGNNAGLTIAAKNVEVKNNITSNKTVNITASEKLTTKADATINATTGNVEVTAKTGDIKGEVKSTSGNVNITANGDTLNVSNVSGNAVTITADKGKLTTQESSTISGTESVTTSSQSGDIGGAISGNTVSVKATNDLITKANSKIEAKTGEANVTSATGIIGGTISGNTVNVTANTGSLTIKGGAKVDATNGAATLTAESGKLTTQAGSTITSNNGQTTLTAKDGSIAGSIDAANVTLNTTGTLTTVVGSSINANEGTLVINAQDATLNGDASGDRTEVNAVNASGSGSVTAVTSSSVNITGDLSTINGLNIISKNGKNTVVLKGAEIDVKYIQPGVASAEEVIEAKRALEKVKDLSDEERETLAKLGVSAVRFVEPNNAITVNTQNEFTTRPSSQVTISEGKACFSSGDGAAVCTNVADDGQQ

SEQ ID NO: 30 - protein, 1794 aa, Haemophilus influenzae
MLNKKFKLNFIALTVAYALTPYTEAALVRNDVDYQIFRDFAENKGKFSVGATNVEVRDNKNNNLGSALPKDIPMIDFSAVDVDKRIATLVNPQYVVGVKHVGNGVGELHFGNLNGNWNPKFGNSIQHRDVSWEENRYYTVEKNNFSSELNGKTQNNEKDKQYTSNKKDVPSELYGQALVKEQQNQKRREDYYMPRLDKFVTEVAPIEASTTSSDAGTYNDQNKYPAFVRLGSGSQFIYKKGSHYELILEEKNEKKEIIHRWDVGGDNLKLVGNAYTYGIAGTPYKVNHTDDGLIGFGDSTEDHNDPKEILSRKPLTNYAVLGDSGSPLFVYDKSKEKWLFLGAYDFWGGYKKKSWQEWNIYKPQFAENILKKDSAGLLKGNTQYNWTSKGNTSLISGTSESLSVDLVDNKNLNHGKNVTFEGSGNLTLNNNIDQGAGGLFFEGDYEVKGTSENTTWKGAGISVAEGKTVKWKVHNPQFDRLAKIGKGKLIVEGRGDNKGSLKVGDGTVVLKQQTTTGQHAFASVGIVSGRSTVVLNDDNQVDPNSIYFGFRGGRLDANGNNLTFEHIRNIDDGARLVNHNMTNASNITITGAGLITNPSQVTIYTPAITADDDNYYYVPSIPRGKDLYFSNTCYKYYALKQGGSPTAEMPCYSSEKSDANWEFMGDNQNDAQKKAMVYINNRRMNGFNGYFGEEATKADQNGKLNVTFSGKSDQNRFLLTGGTNLNGELKVEKGTLFLSGRPTPHARDIANISSTEKDKHFAENNEVVVEDDWINRTFKATNINVTNNATLYSGRNVESITSNITASNKAKVHIGYKAGDTVCVRSDYTGYVTCHNDTLSTKALNSFNPTNLRGNVNLTESANFTLGKANLFGTINSTENSQVNLKENSHWYLTGNSDVHQLDLANGHIHLNNVSDATKETKYHTLNISNLSGNGSFYYWVDFTKNQGDKVVVTKSAKGTFTLQVANKTGEPNHNELTLFDASNATERSGLNVSLANGKVDRGAWSYTLKENSGRYYLHNPEVERRNQTVDTPSIATANNMQADVPSVSNNHEETARVEAPIPLPAPPAPATGSAMANEQPETRPAETVQPTMEDTNTTHPSGSEPQADTTQADDPNSESVPSETIEKVAENSPQESETVAKNEQKATETTAQNDEVAKEAKPTVEANTQTNELAQNGSETEETQEAETARQSEINSTEETVVEDDPTISEPKSRPRRSISSSSNNINLAGTEDTAKVETEKTQEAPQVAFQASPKQEEPEMAKQQEQPKTVQSQAQPETTTQQAEPARENVSTVNNVKEAQPQAKPTTVAAKETTASNSEQKETAQPVANPKTAENKAENPQSTETTDENIHQPEAHTAVASTEVVTPENATTPIKPVENKTTEAEQPVTETTTVSTENPVVKNPENTTPATTQSTVNSEAVQSETATTEAVVSQSKVTSAEETTVASTQETTVDNSGSTPQPRSRRTRRSAQNSYEPVELHTENAENPQSGNDVATQLVLRDLTSTNTNAVISDAMAKAQFVALNVGKAVSQHISQLEMNNEGQYNVWVSNTSMKENYSSSQYRHFSSKSAQTQLGWDQTISSNVQLGGVFTYVRNSNNFDKASSKNTLAQANLYSKYYMDNHWYLAVDLGYGNFQSNLQTNHNAKFARHTAQFGLTAGKAFNLGNFAVKPTVGVRYSYLSNANFALAKDRIKVNPISVKTAFAQVDLSYTYHLGEFSITPILSARYDANQGSGKINVDRYDFAYNVENQQQYNAGLKLKYHNVKLSLIGGLTKAKQAEKQKTAEVKLSFSF

SEQ ID NO: 31 - DNA, 288 bp, Haemophilus influenzae
atgaaaaaattagcaacattaacagcattagcaagtgcattaacgatggcagtagcgacagcagtacaggctgaatcaaagtcaagcaatactgataatacagcaacaccttgtgtaggcgataaatgtgtcaaaacgaaagccgcagaaggtaagtgtggtgaaggcaaatgcggtgcagataaagctaaatctgctgaaggtaaatgtggcgagggtaaatgtggtgcgagcaaaccgaaagctgctgaaggcaaatgtggcgaaggtaaatgcggttctaaataa

SEQ ID NO: 32 - DNA, 465 bp, Haemophilus influenzae
atgaaaaaagcattttggttagtattaagttcagcaatagtcttggctgcttgtgatgataaacctaaaatgacggagcaaactaaaacaactgaccaagcgaaaatggctgcagaacaacctgatcaaaagcccgtattgatcaattataaacaaattgccttagacaaatcccagcaagcgattgatacagcgaaagtcgctcaagacaaatcagttcaagctaatgaagccgtgaaagtagcaatgcaaaaacaacaagaggctgaaattgcgctaagtcaaggtggcgaagaggctgaaaaattgtctaaacaaaaaatggacgaatctaatgcagctgcagaattagctttaaaattgtcggaagaatccaaagctgcttctgcactttcaatccaactttccaaagaagcggaagatgcggctaataaggcgaatcaacaagctcaagctgagaaatag

SEQ ID NO: 33 - DNA, 564 bp, Haemophilus influenzae
atgaaaaaattaacattagcattggttttaggttcagctttagccgtgacaggttgttttgataagcaagaagcaaaacaaaaagttgaggatacgaaacaaactgtagcatcagtcgcatcagaaacaaaagatgctgcagctaatacaatgactgaagtaaaggaaaaagcacagcagctttctacagacgtgaaaaataaagtagcagagaaagtggaagatgcgaaagaagtaattaaatcagcaacagaaacagcatcagaaaaagcaactgaaataaaagaggcagtttcagaaaaagcaagtgagatgaaagaagctgcatcagaaaaagcaagtgagatgaaagaagctgcatcagaaaaagcaagtgagatgaaagaagctgcatcagaaaaagcaagtgaaatgaaagaagctgcatcagaaaaagcaagtgagatgaaagaagctgcatcagaaaaagtaggcgaaatgaaagaaaaagcaactgaaatgaaagaggcggtttcagaaaaagcaactcaagcagtagatgcagtgaaagaagcgacaaaataa

SEQ ID NO: 34 - DNA, 582 bp, Haemophilus influenzae
atgaaaaagaaaaatcagattttagttagtttatctattgtggccttattaggaggctgttcagaagagcaagtacaacgtgatgtgtatcagtctttagacgattgtttagcggattggaaaaaaattgagctttgtgaggcagataaaaatactgaaagtactcagaaaactgaaactacaccacagcaggggcttggtttaaatattcgtgataatggtaatgcagaaagtgcggtaaaaaatccagctgaaaataatgtacaagctaatcaaagcgagaacaatgctgaatcaactgcaaaagctgaaagtaccgatccttctttaggagccgcaattgctggtggcgttatagggtatatggcggctcgtgcaattagtagtttccttggcccaagctatcatcctggaaatcgagctgtcaccacaccaacagggcaagttgtgcaacctcaaacgaatcgttctgttggaaaaccaatgctcgtgaaaggaaatgcaggaagcatgaatagcaaacctgtatctcgtggtggttttagtagcccaaataatactcatcgtagcagtgggggctaa

SEQ ID NO: 35 - DNA, 651 bp, Haemophilus influenzae
atgaatttaacttctttaatcaacaaatctgcaaaagcgttagcgttaagtgcggcatttgtagcattgccatttttggcttatgcggatgatgcacaaaaacctgcggatcatgttacttatgaaccgcaattagataaccaacgcgatcctaatcaatattgtgctaaatgccataaattcgacaaaatagataaaaaccaaacgctagatcggtctggtggcgaactccactttggtaaattccatggtgcacatttagataagaaaaatccaaataacggtaaagctattacttgtgtaagctgtcacggcaatgtttccgaaaatcatcgtcgcggtgctaaagatgttatgcgttttgaaggagatattttcggtaataaaaaaccgatgtattcagttcaagaacaaaaccaagtttgttttgcttgccatcaacctgacaaacttcgtgaaaaattatgggctcatgatgtacacgcaatgaaattgccttgcgcaagttgccatacgcttcaccctaaagaagacgcaatgaaaggtattcaaccaaaacaacgcgttaaactttgcgtagattgccatggtaaacaacaaaaacgtaaagctgaacaagataaactaaccaaacaaaaggataaacaatga

SEQ ID NO: 36 - DNA, 678 bp, Haemophilus influenzae
atgggaattaaaaacaagccgataacaagaccatgcccgcagtgcggcagaaattatcaatatcggcgggcaagtgggcgtacttttgagctttgcgaatattgtcgcaatcttgattgtgtagtttgtggtaaaaaagtaccgccggaacgtgggcggaaaaatacttgctgtgcggagtgcgaaaaattgaagatacacaatatccaaaatgcgcactacgccaaacggatagcggaagaccctgagttaaataaacgcaaccacgccaaagcccgcgaaaaccgaaaagcagacccggaacgaatgcacgaacatttggaagcacaaagggaaaggcattatagacgtgtgcaagatccgaattatctagccactcggaaagtttatcaggctcaacgatggcaggataaaaaagatgagatactggctcaacggcgcgaattttgggatagtttaagcgatgttgagaaagcggagcgacttgagcgcaatcaagcaatacaacgtaaacataaagccaaaaaacgtgaccagttgaaattagatccgcaaaagtgggcggaatatcaagagtatcaacgcacaaagcggcgcgaacatagacagagaaaggcgcttaatgaattaatggtcggcacaaaggagttattaaatgttaccaacaaagacaaatag

SEQ ID NO: 37 - DNA, 795 bp, Haemophilus influenzae
atgcagcaaacaaaacgttcgctattgggtttgcttatttctttgatcgcacacggtattattataggatttatcttatggaattggaatgagccaagtgatagtgcaaatagcgcacaaggcgatatatctacaagtatttctatggaactattacagggcatggtgttggaagaacctgctccagagccagaagatgtacaaaaagagccagagcctgagccagaaaatgtacaaaaagagccagaaccagaaaaacaagaaattgtagaagatccaacaataaaacctgaaccgaaaaaaatcaaagaaccagaaaaggaaaagccaaaaccaaaagaaaagccgaagaataaacctaaaaaagaggttaaaccacaaaagaaaccaatcaataaagagctaccaaaaggcgatgaaaatattgattcaagtgccaacgtaaatgataaagcaagtacaacaagtgcagctaatagcaatgcacaggtagcaggaagtggaacggatacgagtgaaatagcggcttaccgttctgcaatccgccgtgaaattgaaagccataaacgttatccaactcgagcgaagataatgcgcaaacaaggaaaagtgagtgtatcttttaatgtaggagctgatggttcattaagtggtgctaaggttacaaaatcctcaggcgatgaaagtttagataaggcggcattagacgctattaacgtatctcgctctgttggaacaagacccgcaggattcccttcgagtttaagtgtgcaaattagttttactcttcaataa

SEQ ID NO: 38 - DNA, 1140 bp, Haemophilus influenzae
atggaaaaagctggctatgagcgagctagtgattctgattcatccttttccggtggaggtggttggcgagaagataacagtagtgatagttatcgtagtacgtcagatagatggaatgaccacaaatctagatacggaaaagacaaagtctatactgatgcatttaatgagcgaagaaataactctagttggagcggtggtcatagcgcaattagccgaacaattagtgaaaaatatcattcactttctaatgggcaaatgagcgccgccgttcctgaaaaagatcagaaaacactcactggcggtttgtttggaaaaagttactccaatgcgccttattctgaacgcactccttctatatttgatagaaacatacgtggttcaatgacattaaataacggcgatgtatggtcaagcgatccccaatattcatccgttcgagaacgggcggacatcaatagttacgaccgtattaaacggggcgaagaattgaacttaattggtcgtgctgtaggaggcgtttttagtggggtgggcggggcagcaacaacgccagttggcaaaattgctgaaagtgcggcaaattttgggctttcccacgttggggatttatctcgacaattcaaaagcaaccaagagcaagcgtattatgatagcctcactccagaggggaaagcgtattacgatacaagagtagatttcatcaataagtcctataagaatgctcgggaaaaatatgaaacgaacgataaatggattgatagaggtattacagctgcacaagtcggtttatctgctttagggcctcctggtgcaatgctagggtctgggattggtttattaggtaaagcgatcaacaaaaaagacacgatgacaaaatcattacgtgatttaacagagacgcttaactctaacgcattaaataaccacatcgcacaacaaaatgaattagctgaaaaagaacgtcaagcctataaggaatttatggctgggcgtgatttacgcagtgacaatacacaaccaaaaggcatactgaacactatgtataatcgtatgcaaaatatagatcctgataaacaggtcaaaacgagtgacgttcctaacctaagaaattattgggcaaatatcatcgtatcatag

SEQ ID NO: 39 - DNA, 1218 bp, Haemophilus influenzae
atgaacaaatcgtttctcttactccctttaagtcttgttgtattatcggcttgtacctctaattctcctgcacctatttctgatgcagatggaaatctttcccctagtgtagtgcaatctgtaaatggcagtaatgtaggtggtgcttggcaacctgaaattcaaaaaaattcattacctacaatgggaaatatggtcacaccacaaccaaattttcaaccgattaatcaacaaccagcaatgccaactaaacctgcacaacctgcatttcaaccctctccgaaaacggtagttagcgcaccgacagtacaaacaaaaacagttaccaaaacaattgctgattgtgtagatggacagcatattaatatcccacgtaatccaaataccaatgcgccagattacagcaaaatttcaaaaggctcttacaaaggcaatacctataaagtaaacaaaggcgatacaatgttcttgattgcttacttagcaggaattgatgtaaaagaattggcagcgttgaataacctatccgaaccttataatttaagtttagggcaagttttaaagatttctaattgtggcacaaaaacggttactacaacggtttctgtaaaacaacctgcagtcacggcatcaacagcaatgccagctaaaccagccgtcacttatactccgggcgcaaacggtactcaaataggatctgacggtactattattggccccattaaatcagaggctggcatatcgtctagtgtatctgtggcaacttcatcaacacaggttacatcatctgtaaataatgcgaatagcacaccgattaatgcaaatgtcgttgcaccaatcgcctcaaatgttgcgtggcaatggccgacttcaggtaatattattcaaggtttctcaagcacagatggcggtaacaaagggattgatattagcggttcacgaggacaagctgtaaaagcggctgcagcagggcgaatagtgtatgctggcaatgctttacgtggttacggtaatttaatcatcatcaaacataatgatgatttcttaagtgcttatgcgcataacgacaaaattcttgttgctgatcaacaagaagtcaaagctggtcaagacatcgccaaaatgggtagctctggtacaaataccgtgaaactccactttgaaattcgctacaaaggcaaatcagtagatccagtgcgttatctaccaaaacgttaa

SEQ ID NO: 40 - DNA, 1227 bp, Haemophilus influenzae
atgcaaaataatcgacaaaagaaaggaatcaatgcttttgctatttctatccttttgcactttatcttgtttggcttattgattttaagttcgctttatcacaccgttgaaattatgggtggtggagaaggtgaaggagatgtaataggggcagtgattgttgatactggtacggctgctcaggaatgggggcgtattcaacaacaaaaaaaagggcaagcggataaacaaaaacgcccagaacctgttgtggaagaaaaaccacctgagcctaatcaagaagagattaagcatcaacaagaagttcaacgacaagaagagattaagcatcaacaagaagttcaacgacaagaagagttaaaacgtcagcaagaacagcaacgtcagcaagaaataaaaaaacaacaagagcaagctcgtcaagaagcgttggaaaaacagaagcaagctgaagaggctagggctaaacaagcggctgaagctgcaaaattaaaagcagatgcagaggctaaacgtttagctgctgcggcaaaacaagctgaagaagaggctaaagcgaaagcagcagaaattgctgctcaaaaagcaaaacaagaggcagaaactaaggcgaaactagaagcggaagctaaggcaaaagccgctgctgaagctaaagcaaaggctgaagcggaggcaaaagcgaaagcagcagctgaggcaaaagcaaaggctgaagcggaggcaaaagcgaaagcagcagctgaggcaaaagcaaaagctgatgcagaagctaaagcgaaagccgctgctgaggcaaaagctgctgcggaagcaaaacgtaaagcagatcaagcaagcctagatgatttctttaacggcggagatgttggtggcggtagtgcatctaaagggggaaacacaaataaaggtggaactcaaggtagcggtgctgcacttggctctggcgatggtggtaaggttggggatcaatacgcaggtgtaattaagaaagagattcaacgtcgtttcttaaaagatccaaattttgcaggaaaggtttgtcgtattaaaattcaattaggtcgagatggcacaatcttggggtatcaaaaaatttcaggctctgatgatatttgttcagctgcattaagtgcggtggctagaacgaaaaaagttccagctgcgccatcagatgaaatttatgaaaaatataaatcaccaattattgactttgatattcgataa

SEQ ID NO: 41 - DNA, 1287 bp, Haemophilus influenzae
atggataaaaatcaacaatcatctcaaaatgaattggatttagggcttaatcaagagccaattacaccaaagaaaacaattcagccaagttcgtcaattttaggtaaagcaaaagggttatttgccaaaaaaaatcacgtgcaaactaactttcagcaacgtaaagaacctacttttggcgattcatcaacgcaagaaaatgatcctttaattccgagtgaaaatttgaaaaaagtgcaaaagcctgttcttcaaacttcttccacagaagaaaatatttctgcggttgatgaagaaatcagtgctgaaaataacgcggatgaacccgtcgaaaaggctgaaaaacctattttagctcaaccagaaaaatggaaaatattacaagtattgccagcaaaacatcgccgtttatttatggctatttttgtgttggttattttattgattattttcttcgcattaaaaccaagttctgacaccgttgagtcttttactcaatctaacagcaatgaagttcctgtgcagttccaatcgttagatcaaagtcagccactggaaaccacgattttagataatcctcctgcacaaaatcaaatggctgtagaacaagctaaccaatctgaatttgcaccaaaagcagaggaagcggcgaataatacgacggctcaaaacccattagtagaaaatgcgccaatgcaacaaaatgttgttcaatctccaagtcaaatgccaaatgaaatggctgcggcatctgttatgcctatgcaaccagctcaagcggaacagccaaaagcaactgtgccagttcagccgatgaaaaaagcggtagagccacaagttgcgcataaagatactgtgaaaaaagaagtgaaagtggcggagaatgcacaggctccatcaaaagcaacggaacaaaacgtcgctaaaacagcaggaaacgcaccgattgttgaagccaaacctgttcaagtaaaaaaagaaaagaaagttcaaatcgttgatgcaaaacctgtgagtaaatctgcagcctctcgtctttctgcgaaaacattaactgtgccgaaaggtgtttcgctgatgcaagtcttccgtgataatcaacttaatatttccgatgtaaacgcaatgagcaaagcagcaggggcgggcaatgttttaagtagctttaaatctggcgataaagtaacggtatctgtgaataatcaagggcgagtaaatgaaatgcgtttatccaatggtgcgcgttttgtgcgtcagtctgatggttcttatcaatataaaaaataa

SEQ ID NO: 42 - DNA, 1347 bp, Haemophilus influenzae
atggctgaagaaaacaaagaaattatagcttataaagggtttaagcaagactggacttgtcgaggttatcagtatgaggtaggcaaaacgtatgagcataaaggtaatgttaaggcttgtgagagtggattccacgcctgcgaatacccgcttgatgtgcttagctattacagtccagcggtaagtaaatttgctgtagttaaaatgagcggcgaaacatcaaaagatagtgatgatacaaaaattgcatctgcaaaaatcacgatcgaaaccgaaattaacttaccggaaatgataaaaaaagccgttgaatggataaaaggtaaagttgattgggatgcagccaaggtatccaatacaggcgattggtcggcagcaaccaatacaggcgattggtcggcagcaaccaatacaggcgatcagtcggcagcaaccaatacaggcaattggtcggcagcaaccaatacaggcgatcagtcggcagcgactaatacaggcgatcagtcggcagcgactaatacaggctatcggtcggtagcgactaatacaggctatcggtcggtagcgactaatacaggcgatcagtcggcagcgactaatacaggcggtcagtcggcagcgactaatacaggcgatcagtcggcagcgactaatacaggctatcggtcggtagcgactaatacaggcgatcagtcggcagcgactaatacaggctatcggtcggtagcgactaatacaggcgatcagtcggcagcgactaatacaggcggtcagtcggcagcgactaatacaggcgatcagtcggcagcgactaatacaggctatcggtcggtagcgactaatacaggcgatcagtcggcagcaaccaatacaggcaattggtcggcagcaaccaatacaggcgatcagtcggcagcgactaatacaggcgatcagtcggcagcgactaatacaggctatcggtcggtagcgactaatacaggcgatcggtcggcagcgactaatacaggctatcggtcggcagcgactaatacaggctatcggtcggtagcgactaatacaggcgatcggtcggcagcgactaatacaggctatcggtcggtagcgactaatacaggcgatcagtcggtagcggaaatatctggcaagcaatctatagctgttgcgcttggttggcaatctaaagctaaggcgagtattaatggtgctattgtttgtgtatatcgcaatcatgatggcgagctaatccatatcaaagcatcaaaagtcggtgaaaataacatcaaagctgatacttggtacacgttagatgaatttggtgagtttgttgaggttaaagatgactaa

SEQ ID NO: 43 - DNA, 1395 bp, Haemophilus influenzae
atgaaagcaaataaagcgttctatgttattggtgggattaccgctcttgtcgtgacgctgatggctggatttcttttctccggtagtagttcaacaaatacagcagaaaccaagaaagaagatactttagatatctcttttaaagatttatcaccggaagaattacgagcaatggggattgagggtgacacatctaaagatacggtaagaacacttataggtaaagtgaaagagcataataagcgatttgatgaagtgattttacaaaatgaacgcttggtaaaagagaatgagcagttaaaaaatcaagcaaacaatacggattatcaaattcaacaggcggtacaagcagaaacaggggcgttaattaatgagattaatgcgttaaaagatcaggttgtacaagctggtaatcaagttgtagggaatggaaatgcccaggcaaatcccaatgctcctctaccaataaacggagcaacaactggccctcaggttgaaactcgagggggaattaattgggtgaaccctagtgatatgccaactacagacagacaagggaatgctctacaaggtattggtgtaaatggtgcaggaatcggttttcccaaaaccttcgataacaatgtgaatgacatccgaaaatctaatgatgggttgcaatctagtacaggtaataatgcaatatttaataccgctacaccatttttcacaattgcagctaactctactttaactaattctgttgctatgactgcgcttgttgggcgtgtgccgattgacaataaggtaaccgagccttatccgtttaaattgcttattggtcgagataacttaattgcaaatggcattgaactgccggatattgaaggcgcaattgtctctggaacagcaagcggtgactggacgctatcttgtgttcgtggtgatgtgaaaagcttaacttttgtgtttagcgatggccgcattgtttcaacagacaattctgacgataaaattggctggttatctgatccacatggtgtgccttgcattcctggagaacgcagaacaaatgctccggaatatctaacgaccaacttcttcttaacgggggcttcagcagctgcacaaggcttatcatcttctcaatcaacaacagtggttgatggcggtgcagtaattggtgcggtaacgggtaataacggtaagtatattcttggtcaagcgttaggtggcggtttgagagaaacagcagactggttccgtcagcgttatggccaaatgtttgatgcggtatatgttccaccgggtaaagaagtggctgtacacatcgaaaaacaaattgatattgattatgaccgcatgtctcgaaaagtgaaatacggacaagcctcaagacaatctaacttggattaa

SEQ ID NO: 44 - DNA, 1422 bp, Haemophilus influenzae
atgcaacacgtaaaattagctcgagatagacgaaaaaaacgcacatatataaaagtaggcgtgttttttgttgctattctgcttattttgacaggcattttacttacaataaaagacaaatctgaagaaaatcccatattttccacctctgacagtggggaatatcacgagttaaatacatcgccaaataaaaattccaccgcacttcaacctgatgaagatgccacctcttatgatgatgaactgcaagcaaaagatgatgaagttgatgaagtaaaactatcttcagatgatctcggtactttacctcaacacgcacaagacgcactcaatggtttattagacgcggccgatcaagccataagaattactgatcaatttagctatacggtaaccgaaggggatacgttaaaagatgttttagttttatctggtttagatgattcatcagtacaaccgttgattaagcttgatcccgaattagcccacttaaaagcaggtcagcaattttactggattttaaataaaaatgataacttggaatatttaaattggctggtttctgaaaaagaagaacgtatttatgaacgtctcgaagatgggaaatttaaacgccaagttattgaaaagaaaagtatttggcgaaaagaagtattaaaaggcgagattcaaaattctttaaatagtagtttaagagaacaaggtcttgatacgcgacaaattagccaacttagtaacgccttacaatggcaagtgagtttacgaaaactaaaaaaaggcactcaatttgccattttagtttctcgtgaatatttaggcgataaacttacaggtcaaggtaatgttgaagcgttacgtatttcatctggtggtaaaaattattatgcagtgcaagcggcaaatggtcgttattataatcagcaaggggaaactttaggcaaaggttttgctcgttatccattgcagcgacaagcgcgagtttcctccccttttaatccaaatcgtcgccatccagtaacaggacgtgttcgtccccataaaggcgtggatttctctgtttctcaaggcacaccagttattgcaccagcagatggtacagttgaaaaggtcgcttatcaggctggcggtgctgggcgttatgttatgttgcgtcacgggcgtgaatatcaaactgtttatatgcacttgagcaaatccttagtaaaagcagggcaaaccgttaaaaaaggcgaacgtattgcgctttctggtaacacgggcatttctacggggccgcatttacattatgaatttcgtattaacggtcgagcagtgaatcctcttactgttaaattacccggtactagcagtggtatgacttcagctgaacggaaacaattccttgttcgtgttcgtgaagctgaaaaaatgttaaaaccttag

SEQ ID NO: 45 - DNA, 1527 bp, Haemophilus influenzae
atgaacgcattcaaaaaaagccttattgtggcagcgtctttcgcgagtttaagcttatttaactcagccactgctgagcttgtatataagccattagaacaacctgttgaacccgcaaaacctgatttgaaaattgaatctgtaaatgaaaaatttgcagaaaaatatccaaatcaatataacagctggcgttctacagcaaacggcgatggcgaaaacattatttatgctgatgaagaaaatccgcgcctaatcgtactttggggtggttatgcatttgcaaaagaatataacgcaccacgcggtcacttttatgcggtaacagatgtgcgtaatattttacgtacaggtgcgccaaaaactgcaaatgatggccctcaagctatggcatgttggacatgtaaaggtcctgatgttccacgtttaatcgcggaatggggtgagaaagattatttcaatgcgaaatgggcaaagggagggcctgaaatcgtgaactcaattggctgtgctgactgtcacgacacaacctctaaagattttgctgaaggcaaacctgcattacgtattgctcgcccacacgtattacgcgcccttgatgctttagaaaaagccactgctgaaaaagataaagcagaaggtcgcccacacaacaatttaagtttcaacacggcggcgcgtactgaaaaacgtgcagaaatttgcgcaaactgccatgttgaatactattttgcaggggacatcaagcaagtaactttcccttgggataatggtcaaaccgcagatgatattgaaaaatattatgatgatattggtttcactgactggactcactcactttctaaagcaccaatgttaaaagcacaacaccctgactttgaaatttggtctttaggtatgcacggtaaaaatggggtaacttgtgtagattgccatatgccgaaagtacaaggcgcagatggtaaagtttatactgaccaccaaatccaaaatccattcgatgcatttgaccacacttgtgcaaattgtcacgatcaaagcaaagaaaaattacgcgatattgttacttcacgtaaaaaagaagtgaaagatgtaatgggtcgtttagaagaccaagttgttaaatctcactttgaagcaaaagcggcttgggacgcaggtgcaactaaagaagaaatggaagcagcattaatggatattcgtcatgctcaatggcgttgggattacactgctgcaagccatggtggtcatatgcatgcgcctgaagtagtacttcgtgtattagcttctggcttagataaagtagcagacgcgcgcactaaacttgctgtaatcttaactaaacacggtgtgaaaactccagttcaaatcccagatatttcaacagctgataaagcgtggaaagtaatgggtattgatattgaaaaagaacgtaaagcaaaagaagaattcttaaaaactgtggtaccgcaatgggagcaacaagctagagaaaaaggcttattagttgatccaccagcacaaaaataa

SEQ ID NO: 46 - DNA, 1893 bp, Haemophilus influenzae
atgaaatctgtacctcttatcactggtggactttcctttttattaagcgcttgtagcgggggaggtggttcttttgatgtagatgacgtctctaatccctcctcttctaaaccacgttatcaagacgatacttcaagttcaagaacaaaatctaatttggaaaagttgtccattccttctttaggaggagggatgaagttagtggctcagaatctgagtggtaataaagaacctagtttcttaaatgaaaatggctatatatcatatttttcctcaccttctacgattgaagatgatgttaaaaatgttaaaacagaaaataaaatacatacaaatccaattgggcttgaacctaatagagcattacaagaccccaatttacaaaaatacgtttattctggtttgtactatattgagaattggaaagacttttccaaattagcaacagaaaaaaaagcctatagtggccattatggttatgcgttttattatggtaataaaactgcaacagacttgccagtaagcggtgtagcaacgtataaaggaacttgggatttcatcactgcaactaaatatggccaaaattattctttgttcagtaatgctagaggtcaagcttattttcgacgtagtgctactcgaggagatattgatttagaaaataattcaaagaatggtgatataggcttaataagtgaatttagtgcagattttgggactaaaaaactgacaggacaactgtcttacaccaaaagaaaaactgatattcaacaatatgaaaaggaaaaactctatgatatagatgcccatatttatagtaatagattcaggggtaaagttactcctacgaaatccacatcggatgaacatccctttaccagcgagggaacattagaaggtggtttttatggacctaatgctgaagaactagggggtaaattcttagctagggataaacgagtttttggggtatttagtgccaaagaaacgccagaaacagaaaaggaaaaattatccaaagaaaccttaattgatggcaagctaattactttctctactaaaacagccgatgcaacaaccagtacaacagccagtacaacagccgatgtaaaaaccgatgaaaaaaactttacgacaaaagatatatcaagttttggtgaagctgattaccttttaattgataattaccctgttcctcttttccctgaaggggatactgatgacttcgtaacgagtaaacatcacgatattggaaataaaacctataaagtagaagcatgttgcaagaatctaagctatgtaaaatttggtatgtattatgaggataaagagaagaaaaacacaaatcaaacaggacaataccaccaatttttgttaggtctccgtactcccagttctcaaattcctgtaacgggaaacgtgaaatatctcggtagttggtttggttatattggtgatgacaagacatcttactccactacaggaaataaacaacaagataaaaatgctcccgccgagtttgatgtcaattttgacaataaaacattaacaggcaaattaaaacgagccgactcacaaaataccgtgtttaacattgaggcaacctttaaaaatggtagtaatgccttcgaaggtaaagcaaccgcaaatgtagtgattgatcccaaaaatacacaagccacatctaaagtcaatttcacgacaacagtaaacggggcattttatggtccgcacgctacagaattaggcggttatttcacctataacggaaacaatcctacagctacaaattctgaaagttcctcaaccgtaccttcaccacccaattcaccaaatgcaagagctgcagttgtctttggagctaaaagacaagtagaaaaaaccaacaagtag

SEQ ID NO: 47 - DNA, 2001 bp, Haemophilus influenzae
atgaaattaaatcatgcactgaaagcatcttatattgctatatcgtgcgcaatcttatcctcaaccatgccagtatcagcttctaatagtaatccgtttggatttaataaagccgattctattttgtctgatatggtgttttatcaaattgggggcggtgtaggttatatggctcccccgagtcgaggtacgattcctgcagctgaatttgggattggttggaaagctaacttgatgtgcggtaattttgacattaagacgagtattaaaaatcagttaaatggtttaactgaaggatttaaagatctttatagtaacgttatcgaatcagccacaggagctgtggcgagtttacctgcaatggttattcaacgagcaaatcctcagttatacgatattcttacgaatggtttatatcaaggtaaaattgattttaatagcttaaaaaccagctgtgaagaaatgtctaagaagcttgcagacgcaactttaaatggtcgttggtctcaaagttcggatatggaatcctttaaagacattacttcaactgaaccagatgctactcgagctaaaaagagacttgaggaatctcaaggtaaggaaggtaaagagtgggtcgggggtaaaaaacgcggtggccaaggacaagaaccaatcaaaattgttgaggatgttgcaaaagctggctacaacatgcaaaacaagcgtaatgtattagaatcaggtcgaatctccggtagtagttgtagcggattactttgtcaaacttgggataatcctaaagatatgacagattggttaacgaatgtgatcggtgagaagcaattaactacttgtaaaacagattgtggtactgctagtagctctcgagctggtgttggtttaacacctgaaattgaaaaagaaaatattaagacggtaactcaattacaaaaagcgcttaacatgagcactccttccgttgaagtacttgcagaattaagttctacaacagttcctgtaactcgtggcttaattgagtctttacgagaagatcccgatgctcaagtattagggcagcgtttagccagtgaaattgctgtagctaaaacaatggagaaaatgcttcttgcacgtcgagctgttttaacgggtatgcgtgagccaaatgtggccaacaataaagatgctcaagaagagttagaaaagatcctcactttaattgaccgagaaataggtcaaattagaatggagattgatctacaaaaatcacttaccgggaattccgcagttgcaatcctacaaaataaagagctacgtgaatataacactggaaccggaaatgttacacgtgacagtgtagataaacggattagcgatttagctaatggtacgaatagcaatgctgctgaattggcagatgctgacaatatcaatattcctcgtaacaatgtcactttagaagtgcctcagatgtcaaactccctgtcttacaccgggactggcggtacatccggaaatagtagaggtaacacttcttattcatcagttccagcaattaaagggactgcacttgatcaagctacagggttgttgaaaaaatttgaagggtttagtgataaagcatattgggatgtcaatgcttatcgcactggatatggttcagatactatcacaaaagcggatggtacaattgtcaaagtaacgaaagatactgtagttagtcgcgctgacgcagagcgtgatttagcacgtcgaacacaagaatttgcaaatagagcgagaaataacgtatcatcttcaacatgggataagttaccgccaaatgcccaagccgcattaacatcttatgcctataactatggctcattaacaaaagatgtgattgctgcagcccaggcatctgcgcaatcaggcgatatgaacgcactagcaaatgcagtaagaaatcgtcaaaacaataataatggtgtaaatgctaaacgtcgtaaccaagaagcagattacattttaggaaaataa

SEQ ID NO: 48 - DNA, 2082 bp, Haemophilus influenzae
atgaaatttaaaatgtcaaaaaatgtgatttgttatgcgtggttatcggtgtgtttgagcagtgcaattcctgcatttgctgtacaacctacattaaaaccaagtgacattagtattccagcgataagtgaagaaagccagttggcaactaagcgtgcaacgacaagattaactcaatcccattatcgtaagattaaattagatgatgatttttccgagaaaatttttgatcgttatattaaaaatttagattttagccacaatacttttttgcagtctgatatagacgaacttcgtcaaaaatatggtactaagttagatgaacagttgagtcaaggcgatctttctgcggcgtttgacatttatgatgtaatgatgaaacgtcgctatgaacgttatacttatgcgttatctttattagataaagagcctgatttaaatggtcaagatcaaattgaaattgatcgtgaaaaagcggctgcgccacaaactgaagctgatgcgaataaactttgggatgctcgtgtaaaaaatgacattattaatcttaaattaaaagataaaaaatggtcagaaatcaaagcaaagcttacaaaacgctataatttagccattcgtcgtttaacgcaaacaaaagcagacgatattgttcaaatttatttgaatgcttttgcgcgtgaaattgatcctcacaccagttatctttccccaagaacagcaaaaagttttaatgaaagtataaatctttctttggaaggcattgggactactttacaatcagaagatgatgaaattagcatcaaatctcttgtacctggtgcgccagcggaacgtagtaaaaagctgcatcctggggataaaatcattggtgttgggcaagcaacaggcgatattgaagacgttgttggttggcgtttagaagatttggtagaaaaaattaaagggaaaaaaggcactaaagttcgcttagaaattgagcctgctaaagggggaaaatcacgtattattactttggttcgtgataaagtccgtattgaagatcaagctgctaaactaacctttgaaaaagtgtcaggaaaaaacattgcggtaataaaaattccaagtttttatattggattaaccgaagatgtcaaaaaactactggttaaattagaaaatcaaaaagctgaggctcttattgttgatttacgtgaaaatggcggtggtgcattaacggaagcggtggcgttaagtggtttatttattgccgatggccctgttgtgcaggttcgcgatgcttatcagcgtattcgtgttcatgaagatgatgatgctacgcaacaatataaggggttattatttgtgatgattaatcgttacagtgcttccgcatcagaaatttttgctgcagcaatgcaagattatcgtcgaggtattatcatcgggcaaaatacttttggtaaaggtacggtgcaacagagtcgttcgttaaattttatctatgatttagatcagagtccgttgggtgttttgcaatatactattcaaaaattttatcgcgttaatggtggttcaacccaattaaaaggtgttgcagccgatattaatttccctgaaattatagatgcgaaagaatatggggaagataaagaagataatgcgcttgcttgggataaaatcccatcagcgagttatacggaagtaggaaatatcgattatattgataatgcagtaaatattctaaatgaaaaacatcttgctcgtattgctaaagatcctgaatttgttgcattgaatgaagaattgaaagtgcgaaatgaacgccgagatcgaaaatttctatcgttgaattataaaatgcgtaaagcagagaatgataaagatgatgcaagacgtttaaaagatttgaacgaacgttttaaacgtgagggtaagaaagcattgaaagatattgatgatttgccgaaagattacgaagcaccagatttcttcttaaaagaagctgaaaaaatagcagctgatttcgttatttttaattcagatcagaaaattaatcaagcaaacggcctatccgaagcaaaaacggaaagtaaaaaataa

SEQ ID NO: 49 - DNA, 2130 bp, Haemophilus influenzae
atgcgattttctaaactttcccttgcaattacaacaaccttagtgacagcaaatgcactagcgcaatccgttgaattagactctatcaacgttattgcgacacgagatccaagtaggtttacttatacgccacaaaaacaatctaaagatagtcttctttccaaacaagctactagtgttgcagctgcgttggaagacattccaaatgttgatgttagaggcggttcgagaagcattgctcaaaaacctaatatccgagggttaagtgataatcgtgttgtgcaagtcattgatggcgtaagacaaaattttgatttagcacatagaggttcttattttcttccaatgtcactcatccaagaaattgaagtaatcaaagggccaagtagctccttatggggtagtggtgcattgggtggtgttgtggcaatgcgtacgccaaatgctttagacttattgaaaaataatgacaaattcggagttaaaattcgccaaggttatcaaactgctaataatttatcggaaaaggatgtttctgtatttgcggcaaatgacaaattcgatgttcttattagtggtttctataataatgcggataatttacgcattggtaaaggcaacaagctgaataatacagcctataaacagtttgggggcttagcaaaatttggttggcaaatcaatgatgcgaaccgtgtggaattatcccaccgcgaaactcgttttaaacaaacagcaccaggcaataatgaggcgaaaaacgaacttactaatgaacaaattatagaaaaaatcaatgagtaccacaacccactcaacaattttccaccgaaagccaaaccatcactagaagagttttactctggagtaagagcacgtcttggcggtgttagttatttaagtgatcaacaaattcctgatcaaagcacggtatttaactattatttaacgcctgataatccttatctaaatacgcatattgcgctatacaacaataagactattgagaaagaacagcgtaaagtcagtggagtgaaagatcagactaaattgactacccgaggtataaatttacgtaactcttccgaattatctcacatttcctttgtttatggggtggattatatgcgagataaaatcagtactgaacgaggcacaaacgataaagatgcgaagtttcgagcggaaccctataatgcaaattcaaacactacaggcgtttatttaattgcccatattccactatttggggaaaaattgctactttcgccaagtgtacgttatgaccactacgatacctcaagtaaaaccgtaaaatataaggataatcatttatcgcctgcaacaaaattaacttggaaagtgaccaattggttagattttactgccaaatataatgaagccttccgagctccatctatgcaagaacggtttgtaagcggcgctcactttggtacaagtactcgggtaggagatattatcaatagctttgtggcaaacccaaatttacgccctgaaacagcgaaaaataaagaaattaccgcaaatctacattttgatagcctgtttaaacaaggcgataaattcaaaattgaagcgacttatttccgcaatgatgtgaaagatttaattaacttaaagagacttgatgatccaaatgcaaatggagcattgtcacgaacaaattcccagtatcaaaacatagctaatgcccgtttaagcggtattgaattgcaagcccaataccaaacagaacgtttaacgctatttactaactatggcagcaccaaaggtagagataaagatagcggcgaagctttatcaaacattgccgcaagcaaaatcggcgtaggggcagattatgctttagtaaaagacaaattcacggtgggagcgacaataacccattacgctgcccaacatcgagtgcctaaagatcacgctgttacctacccaagttatatactgaccgatcttcgtgctacctatgcgccattaaaaggcgaatggaaaaacctgcgtttagattttgcactggaaaacttgtttgatagaaaatatcaaccagcatttagtttaatggaaggcactggtcgtaatgcgaaaattagtgcggtttatagcttctaa

SEQ ID NO: 50 - DNA, 2403 bp, Haemophilus influenzae
atgaaaaaacttctaatcgcaagtttattattcggtacgacaacgactgtgtttgccgcaccttttgtggcaaaagatattcgtgtggatggtgttcaaggtgacttaaaacaacaaatccgagcaagtttacctgttcgtgccggtcagcgtgtgactgacaatgatgtggctaatattgtccgctctttattcgtaagtggtcgattcgatgatgtgaaagcgcatcaagaaggcgatgtgcttgttgttagcgttgtggctaaatcgatcatttcagatgttaaaatcaaaggtaactctattattcccactgaagcactaaaacaaaacttagatgctaacgggtttaaagttggcgatgttttaattcgagaaaaattaaatgaatttgccaaaagtgtaaaagaacactatgcaagtgtaggtcgctataacgcaaccgttgaacctattgtcaatacgctaccaaataatcgcgctgaaattttaattcaaatcaatgaagatgataaagcaaaattggcatcattaactttcaaggggaacgaatctgttagtagcagtacattacaagaacaaatggaattacaacctgattcttggtggaaattatggggaaataaatttgaaggtgcgcaattcgagaaagatttgcagtcaattcgtgattattatttaaataatggctatgccaaagcacaaattactaaaacggatgttcagctaaatgatgaaaaaacaaaagttaatgtaaccattgatgtaaatgaaggtttacagtatgaccttcgtagtgcacgcattataggtaatctgggaggtatgtctgccgagcttgaacctttactttcagcattacatttaaatgatactttccgccgtagtgatattgcagatgtagaaaatgcaattaaagcaaaacttggagaacgcggttacggtagcgcaacggtaaattcagtacctgattttgatgatgcaaataaaacattagcgataacccttgttgttgatgctggacgacgtttaactgttcgccaacttcgctttgaaggaaataccgtttctgctgatagcactttacgtcaggaaatgcgccaacaagaaggaacttggtataattcacaattagttgagttaggaaaaattcgcttagatcgtacaggtttcttcgaaacagtcgaaaaccgaattgatcctatcaatggtagcaatgatgaagtggatgtcgtatataaagtcaaagaacgtaacacgggtagtatcaactttggtattggttacggtacagagagtggtattagttatcaagcaagtgttaaacaagataatttcttaggaacaggggctgcagtaagtatagctggtacgaaaaatgattatggtacgagtgtcaatttgggttataccgagccctattttactaaagatggtgtaagtcttggtggaaatgttttctttgaaaactacgataactctaaaagtgatacatcctctaactataagcgtacgacttatggaagtaatgttactttaggtttccctgtaaatgaaaataactcctattatgtaggattaggccatacctataataaaattagtaactttgctctagaatataaccgtaatttatatattcaatcaatgaaatttaaaggtaatggcattaaaacaaatgactttgatttttcttttggttggaactataacagccttaatagaggctatttcccaactaaaggggttaaagcaagtcttggtggacgagttactattccaggttctgataacaaatactacaaactaagtgcagatgtacagggtttctacccattagacagagatcacctctgggttgtatctgcaaaagcatctgcaggatatgcaaatggttttggaaacaagcgtttaccgttctatcaaacttatacagcgggtggcatcggttcattacgtggttttgcttatggtagtattggacctaacgcaatttatgccgctcaaaatggtaatagtaatagtaatagtaatggtacttttaataagataagttctgatgtgattggtggtaatgcaatcacaactgcgagtgcagaacttattgtaccaacaccgtttgtgagcgataagagccaaaatacggtccgaacttccctatttgttgatgcggcaagtgtttggaatactaagtggaaatcggataagagtgggttagataacaatgtattaaaaagcttacccgattatggcaaatcaagccgtattcgcgcctctacaggtgtcggattccaatggcaatctcctattgggccattggtattctcttatgccaaaccaattaaaaaatatgaaaatgatgatgtcgaacagttccaatttagtattggaggttctttctaa

SEQ ID NO: 51 - DNA, 2535 bp, Haemophilus influenzae
atgactgaagatgttaaggctgatgcgccaaaaaaattaagcattcaacgcagaacaaaaacaacagtgagcagcaccacaactggcggtaaaagcaaagaagtacaagtagaagtacgtaaaaaacgcacagtaaaaactgatattgctcaacaagaagaagcaaaattaaaagcacagcaagaagcggaagcgaaaaaaattgctgaacaaaaagcagcagaagaaaaagctcgtttagaagctgaaaaagcggcaactaagaaagaagctgatgaaaaatcaaaagctgaaaaagcgaaagcagaaaccgctaagccagcaaaaagtgcggtagattctaaagctaaatttgttgatcctgaaaaagagaaacgtaaagcagaagaagctgaacttcgtcgtaaagctgaagaagtggctcgtcaaaaagcagaagaacaagctcgtcgtgcagcagaagaagctaaacgttatgctgaagcagatgattcagacaatgaatcttcttcagaagactattctgattacaatttaagttcaagatatgcacttgaagcagaagatgaagaagatcgtcgtaatgaaaatcgtggtcgaggtaaaaataaagtagcaaaagcaaaaaaaggcggtcgtgacgatgaaaatagcaaaaactcgaaaaacgagcgcgaatctaatcgtaaaaatcaaaaagatgctaagtttggtaaaggtaaaaatggaaaaaaaggcaccgcacttcaacaagcctttactaaaccagttcaagtagtaaaagcagatgttgtgattggcgaaaccattaccgttgcagaacttgcgaacaaaatgtcggtgaaagcgacagaaatcattaaagtgatgatgaaaatgggtgaaatggtcaccatcaaccaagttatcgatcaagaaaccgctcaattagtagcggaagaacttggtcataaagtaattcttcgcaatgaaaatgagcttgaagaagcggtattaggtgatcgcgatgtaaacgcggaaaaagtaacgcgtgcgccagttgtaactattatgggacacgttgatcacggtaaaacttctttacttgactatattcgtaaagcgaaagttgctgcaggcgaagcgggtggtattactcaacatatcggtgcgtatcacgtagaaatggatgatggcaaaatgatcaccttcttggatactccaggacacgcagcgtttacttctatgcgtgcacgtggcgcaaaagcaacagatatcgttgttcttgttgttgcggccgatgatggtgtgatgcctcaaactatcgaagcgattcaacacgcaaaagcagcgggtgcacctttagtggttgcagtaaataaaattgataaaccagaagcaaatccagatcgtgttgagcaagaattattacaacatgatgtcatttctgagaaattcggtggtgatgtgcaattcgttccagtatcggcgaagaaaggtactggggttgatgatttattagatgcaatcttacttcaatctgaagtgcttgaattgactgctgtaaaagacggcatggcaagtggtgtagtcattgaatcttacttggataaaggtcgtggccctgtggcaactatcttggttcaatcaggtacactacgtaaaggtgacatcgtactttgtggttttgaatatggccgtgcgcgcgcaatgcgtgatgaaaacgggaaagaagtggatgaagcaggtccttcaattccagttgaattattaggtctttcaggtgtgcctgcagcgggtgatgaagcaaccgttgtacgtgatgagaaaaaagcacgtgaagtggcattatatcgtcaaggtaaattccgtgaagtgaaattagctcgtcagcaaaaagctaaacttgaaaatatgtttagcaatatgtctgaaggcgatgtggctgaattgaacgttattgtgaaagcggatgtacaaggctctgtagaggcgattgttcaagcgttaaatgaactttctaccaatgaagtaaaagttaaagttgttggttcaggtgtaggtggtattactgaaactgatgcaactttagctaccgcatctaatgccatcattgttggctttaatgttcgagcggatgcaacagctcgtcgtgtcattgaagctgaaaacattgatttacgttactactcaatcatctatgaattgttgaatgaaattaaagcagcaatgagcggtatgttagagcctgaatttaaacaagaaattattggtttagctgaagttcgtgatgtattccgtcatccgaaatttggtgcaatcgcaggctgtatggtaactgagggtgtagtgaaacgtaacaacccaatccgtgtattacgtgacaacgtcgttatctttgaaggggaattagaatctcttcgccgtttcaaagatgatgtatctgaagttcgtaacggtatggaatgtggtatcggcgtgaaaaactacaacgatgtaaaagttggcgaccaaatcgaggtgtttgaagtggttgaggttaagcgttcaatctaa

SEQ ID NO: 52 - DNA, 2739 bp, Haemophilus influenzae
atgactaaaaaaccctattttcgcctaagtattatttcttgtcttttaatttcatgctatgtaaaagcagaaactcaaagtataaaagatacaaaagaagctatatcatctgaagtggacactcaaagtacagaagattcagaattagaaactatctcagtcactgcagaaaaagtaagagatcgtaaagataatgaagtaactggacttggcaaaattatcaaaactagtgaaagtatcagccgagaacaagtattaaatattcgtgatctaacacgctatgatccagggatttcagttgtagaacaaggtcgcggtgcaagttctggatattctattcgtggtatggacagaaatagagttgctttattagtagatggtttacctcaaacgcaatcttatgtagtgcaaagccctttagttgctcgttcaggatattctggcactggtgcaattaatgaaattgaatatgaaaatgtaaaggccgtcgaaataagcaagggggggagttcttctgagtatggtaatggagcactagctggttctgtaacatttcaaagcaaatccgcagccgatatcttagaaggagacaaatcatggggaattcaaactaaaaatgcttattcaagcaaaaataaaggctttacccattctttagctgtagcaggaaaacaaggtggatttgaaggacttgctatttacactcaacgaaattcaattgaaacccaagtccataaagatgcattaaaaggcgtgcaaagttataatcgattaatcgccaaagaagatggatctaatgcatactttgtgatggaagatgagtgtccaaaggattataacagttgtataccttcagccaaaccacctgcgaagttatcctcccaaagagaaaccgtaagcgtttcagattatacgggggctaaccgtatcaaacctaatccaatgaaatatgaaagccagtcttggtttttaagaggaggctatcatttttctgaacaacattatattggtggtatttttgaattcacacaacaaaaatttgatatccgtgatatgacatttcccgcttatttaagatcaacagaaaaaccggatttagaaaatagttctttttatccaaagcaagattatggtgcatatcaacgtattgaggatggccgaggcgttaaatatgcaagtgggctttatttcgatgaacaccatagaaaacagcgtgtaggtattgaatatatttacgaaaataagaacaaagcgggaatcattgacaaagcagtgttaagtgctaatcaacaaaacattatacttgacagttatatgcaacatacacattgcagtctttatcctaatccaagtaagaattgccgcccaacacttgataaaccttattcatactatcattctgatagaaatgtttataaagaaaaacataatatgttgcaattgaatttagagaaaaaaattcaacaaaattggcttactcatcaaattgtcttcaatcttggttttgatgactttacttcagcgcttcagcataaagattatttaactcgacgtgttaccgctacggcaaagagtatttcagagaaacctggtgaaacaccaagaagaaatggtttcaaattacaaccttacttatacccaaaaccaaatgcatcttttgtaggacgagatcattgtaattatcaaggtagctcctctaattatagtgactgtaaagtgcggttaattaaagggaaaaattattatttcgcagcacgcaataatatggcattagggaaatacgttgatttaggtttaggtattcggtatgacgtatctcgtacaaaagctaatgaatcaactattagtgttggtaaatttaaaaatttctcttggaatactggtattgtcataaaaccaacggaatggcttgatctttcttatcgcctttctactggatttagaaatcctagttttgctgaaatgtatggatggcggtatggtggcaagaatgacgaggtttatgtaggtaaatttaagcctgaaacatctcgtaaccaagagtttggtctcgctctaaaaggggattttggtaatattgagatcagtcattttagtaatgcttatcgaaatcttatcgcctttgctgaagaacttaaaaatggaaagggcaagggcaattatggatatcataatgcacaaaatgcaaaattagttggcgtaaatataactgcgcaattagattttaatggtttatggaaacgtattccctacggttggtatgcaacatttgcttataaccgagtaaaagttaaagatcaaaaaatcaatgctggtttagcctccgtaagcagttatttatttgatgccattcagcccagccgttatatcattggtttaggctatgatcatccaagtaatacttggggaattaatacaatgtttactcaatcaaaagcaaaatctcaaaatgaattgctaggacaacgttcattaggtaacacttcaaggggtgtaaaatcaacaagaaaacttactcgggcatggcatatcttagatgtatcgggttattacatggtgaatagaagtattttgttccgattaggagtatataatttattaaactatcgctatgtcacttgggaagcggtgcgtcaaacagcacaaggagcggtcaatcaacatcaaaatgttggtaattatactcgctacgcagcatcaggacgaaactataccttaacattagaaatgaaattctaa

SEQ ID NO: 53 - DNA, 2748 bp, Haemophilus influenzae
atgaagaaagctataaaattaaatttaattacacttggcctaattaatacaatcggtatgacgattacacaagctcaagccgaagaaacattaggacaaattgatgtagtggaaaaagttatatcaaacgataaaaaacctttcactgaagccaaagccaaaagtacacgtgaaaatgtctttaaggaaacacaaaccattgaccaagtgattcggagcattcctggggcatttactcaacaagataaaggctcgggtgtcgtttctgtaaatattcgtggcgaaaatggattaggtcgtgtcaatacgatggttgatggtgtaacccaaaccttctattctacagccttagactctggtcaatcaggcggaagttctcaatttggtgcggcaatcgatcctaattttattgcaggtgtagatgttaataaaagtaacttttcgggagcaagcggtataaatgcgttagcaggcagtgctaattttagaacattaggcgttaatgatgttattaccgatgacaaaccatttggcattattctgaaaggaatgacaggaagcaatgccactaaatccaattttatgacgatggctgctggcagaaaatggcttgataatggtggctatgtaggcgtggtgtatggttatagccaacgtgaagtttcacaagattaccgtattggaggcggagaacgattagcatcattagggcaagatattctcgcgaaagaaaaagaagcttattttcgtaatgcgggttatgttttaaatgatgctggacagtggactcctgatctaagtaagaacgcttggtcttgtaacgaagaccctcctcgtttagcagagaatatagagaatgttaaatgtacacattattcttttgatcctagaaaaaacgatagaaaagaaattctcaaaaaattaaaagctggaacaaaacctgagaatattgacgagctccaaaacggtaaggatggaattaaagaaactgacaaatcatttgaacgtaataaagatcaatatagtgttgcaccaattgagcctggtagtttgcaatcacgctcgcgtagccatttattaaaatttgaatatggcgatgatcaccaaaatttaggagcacaaatacgtacattggataataaaattggttctcgcaaaattgaaaaccgtaattaccaagtcaattataacttcaataataacaactatctcgatcttaatttaatggctgcacataacattggtaaaaccatttatcctaagggtggtttttttgctggctggcgagtggcagacaaacttattaccaaaaatgtggcaaatattgttgatataaacaacagccatactttcttgttgccaaaagaaatcgatttaaaaaccacattaggttttaactattttaccaatgaatacagtaaaaaccgttttccagaagaattaagtttgttttataacgatgattcacatgatcaaggtaactatagtaatttaggacgatttagaggtaatcgtaatttactgccacaacgttcagtgattttacaaccttctggcaagcaaaaatttaaaacggtgtattttgataccgcactttctaaaggtatttaccatttaaattacagcgtgaattttacccattatgcgtttaatggtgagtatgttggatatgaaaatacagcgaatcaaattaatgaaccgattttgcataaatcagggcataaaaaggcattcaatcattccgctacattaagtgcagagctaagcgattattttatgccatttttcacttattcacgcacacacagaatgccgaatattcaagagatgtttttctctcaagtgtctaatgcaggggtaaacacggcattaaaacctgaacaatctgatacctatcaactaggctttaatacttataaaaaaggtctcttcactcaagacgatgtgctaggcatcaaattagtgggttatcgtagctttattaaaaactatatccataatgtttacggtgtttggtggcgagatggcgttgtacctacgtgggcagacagtactggttttcgctttactatcgcacatcaaaattatcaacctatcgtgaaaaaaagcggggcggagttagagcttaattatgatatgggacgtttttttgcgaatgtctcttacgcttatcaacgtactaatcagccaaccaattatgccgatgccagctcacgtccgaataatgcttcaaaagaagacattttgaaacaaggttatggcttatctcgtgtttcaatgctaccaaaagactacggcagattagagattggtacacgttggtttgatcaaaaattaaccttaggtatggcagctcgttattatggaaaaagtaaacgtgcgacaattgaagaagaatatatcaatggatctcactttgaattaaagacctcaggtaaaagaacttactacgttgtgaaaaaaacagaagagattaaaaaacaaccgattattttagatttacacgtcagctatgaaccaatcaaagatttgattattaaagcggaagtacaaaatctattagataaacgttatgttgatccgttagatgctggaaatgatgcggcttcgcaacgttattattcaagtttaaatgattcaatttgtagtaaaagtcaggattgtgaagatggaggaaaagataaaaccgtgctttataactttgcacgtggaagaacttatattctgagtttaaactataaattctaa

SEQ ID NO: 54 - DNA, 2766 bp, Haemophilus influenzae
atgtataaattaaatgtaatttctcttatcattttgaccacttacactggtgcgacatatgcctctacacagggtttgccacaagagggtttgccacaagaggatacagtcgtttttgggaatgtcactattgataaaactactgcagataaaatgacgattactcaaagtagccctacaactcaaataaattggaagagttttgacatcggtcaaaataaagaagtggaatttaaacagcctagcccaaacgctgtggcatataaccgtgtaactggcggcaatgctagtcaaattcaagggaaattgacggcaaacggtaaagtttatttagcaaaccctaatggggttattattaccaaaggggcagaaattaatgtagctggtttacttgcaacaactaaagatttagagcaaatcagtgaaaatggcaatggcaatggcaatagctatcaatttacccgtaaaacaaaagatggtcaggagctaaaagaaggtaaagttctcaatgaagggaaaattaaggcgaaagatttcgtggtacttaatggcgatgaagttatcaataaagaggaaattaacgttacaaatggcaaagtatatttatcctcaggcgataattttacatttaccttattaccagatagcggtataagtgtggcattagaagataatgcagtacggggtattgttaaaaatgaagggattattaaagcagaggacattacgctaaatgcaaaagggcgtaaagaagcattagatagcttggttatgaataatggtgtgttagaagcaactaaagttagcaataaaaacggcaaggttgtgttatctgctgatgaaattaagttagatgataaaagtaatattaaaggggatatggttagttttgtggctgatgttacctcaaataaggaacttaaagacaatataaaaatcacttctcaaactggctcaaaagttacctcgcctaaaatcgattttaaaggcaaaagtgttaatatcaatggtaactttgggcgtgacgatagtaaagcacactataacgatgagcataaaacattaaaaacagaagttaatattgacgtgccagacgatgaaaatattcgtattgcgaatattgaagatgaagataaagatagtacgggttcatttatacaaacagatgcgttaagttcattactagctaataatggaaaagtaactcttaaaggaaataacgttaatatttcaggacgtattcatatagattcctttagaggctcagattcgctattaaagctaactaacaaaggtcatattaagatcaataatgcggatattcattcaaaaggtcgtttattttttataaccagtttgcagaatgaggaagattttaaatcaaatattaccattactgacagcaaaataaatcttggtaacggagctatgggattggggcgtagtgtcaatgaaagcgactacgataacaaatatcagaaaacggaaggatcacaacgcaaaaagtttaatgttaagatgagtaacgtagaatttaatcaggttgatgatgtgattttagcaggtggatttgaaaaagttaatttagataaaatcgttgccacaggacaaacgaatttttacattgatgggggggtatcccgtaatagaacaaatggtgagccttggaaatatgaatacggtgtgttagatttagacaaacgtacgcagttatcggagttagatcaaggcagacgccgttggggatattactatgatttagagttagatatgaatagagcctatttgtatcgttttgatctgtttgccgctaaaaatacaaggcgatcaactattaaaggcacagaaattaatatttctaacagtaatattaatctgaaaaatggatttgtacatttactggcagaaaaaattaaattagataatagcaagattgatattacttttgataaagataattcacaagatactttagctcaaacaaaccgtttaggaatgaatggtaaagtgtcaatgataaactcacatattaaaatcgtaggcgatgagaaaagtggtatatctccaactgggacttatgctacgatgtttttaatcggggaattaataggggaaaaaagttctatttttgtgaaatctcatcagggttacacctttaaaactgatggcgatacaaaaattgcaggtaaaaacagcaaagaggacttgaaaattaccgcaattaacactggtggaagagcagcggaagaagttctaattaatggggcgttgggttcagctgataatgatgctaatatcgccaatatggcatttactattggtgattcagcgaataccaaaacaaccattgaaaatgccgacatcactgccttagcaccaaacggtgggacagcttatttaagctccaaagatgtagaaattgaggtaaaaccaaattcagatttcacattctttgaacttcctcgtgagaaaaaccttaatcaaacaaaaattaacggcgcttctaccaaactcagtgaacgtggttttgctagactttatgacaaaattaacggtgtgcgagccagcaacttgtctgctgagcaattgaatgttacggactcaagcgaaaaaatcattaatacaaatttagtgagtagcttagatgttgaaaaacttgtttctgttgcagtttgtgatgcaggtaaaggttgcgaagaacaacaattcggcgataaaggtaataatactaaagtgagtgttggagagttagaagctgaacaataa

SEQ ID NO: 55 - DNA, 2808 bp, Haemophilus influenzae
atgaaaagaatgttaatcaatgcgactcaaaaagaagagttgcgtgtcgcgcttgtcgatggacaacgcctttttgatttggacattgaaagtccaggacacgaacaaaaaaaagcgaatatctacaaaggaaaaattactcgcgttgagccaagcttagaagcggcttttgtggattatggtgcagaacgtcacggttttttacctttaaaagaaattgcccgtgaatattttcctgatgattatgtttttcaaggtcgtccgaatattcgcgatattttgaccgaaggacaggaagttatcgttcaagttaataaagaagaaagaggaaacaaaggtgcagccttaactacttttgtttctcttgctggcagttatttggtgcttatgccgaataatccacgggcaggaggaatttctcgccgtattgagggcgatgaacgtactgaactaaaagaagcattaagttcattagatgtgcctgaaggtgtaggcttaatcgtccgtactgctggtgtgggaaaatcgccagaagaattacaatgggatttaaaagtattattacatcattgggaagcgatcaaacaagcttcacaaagtcgtccagctccatttttaattcatcaagaaagcgatgtgattgtacgcgctattcgcgattatttgcgtcgtgatattggtgagatcctgattgatagcccaaaaatttttgagaaagcgaaagagcatatcaaacttgtgcgtcctgatttcatcaatcgcgtgaaactttatcaaggcgaagtgccacttttcagtcactatcaaattgaatctcaaattgaatccgcattccaacgtgaagtgcgtttaccttctggcggttcaatcgttattgatgtaactgaagcattaaccgccattgatattaactccgcacgttctactcgtggtggcgatattgaagaaaccgcattaaataccaaccttgaagcggcagatgaaatcgcgcgtcaattacgcttacgtgacttaggtggattagttgtcattgatttcattgatatgacaccaattcgccatcaacgtgaagtagaaaatcgtattcgtgatgcggttcgtccagatcgtgcccgtattcaaattagccgaatttctcgttttggtttgttagaaatgtcacgtcagcgtttaagcccatcattgggcgaatcttcgcaccatatttgccctcgttgtcaaggaacaggcaaagtgcgtgataatgaatctctttctttatctattttacgtttgctggaagaagaagcattaaaagagaacaccaagcaagtacataccattgtgccagtacaaattgcctcttatcttcttaacgaaaaacgcaaagcaatcagtaatattgaaaagcgtcataatgtcgatattattgttgccccaaatgaagcaatggaaacaccgcactttagtgttttccgtttacgtgatggcgaagaagtaaatgaattaagctataacttagcgaaaattcactgcgctcaagatgaaaatactgaagaatcactgctttctcgtaatgtggaaacaacagctgttattgaacaaccagcggttgaaagtgcagtagttgcactttctatttcagaagcagccccaacgccagttgagcgtaaatcaaatgaaccctctttgttagctaaaattattgcgaaaattaaaggtttatttgcgactaaatctgaagaaaacaaaccgaaaaataaccgcacttcgcgcaatcctaatcgtaatcaacgccgttcacaggatcgtcgttcttctcgtcgtccacgttctgaaaataacgaaacagaaagaaccgaagaacaggttcgtaatgtccgtgagcgtaatcaacgccgtccgcgccgtaacttagtggaagaaagtatcgctgaaagtgcggtaaattctacacctgtttttgaggctaaagaagaaagaactgagcctgtaactcaacgccgccaacgccgtgatttacgtaaacgcgtgcgcgttgaagataatgaaacagttgttgaaaataacttcagcacgactgaaaaaatgccagaagtggatgtaattacggttcaaaataatgatgaaaaacccgttcatcagaaccaacgttctgaacgtcaagagcgtcagcgtcgtactccacgtcatttacgtgcagcgaataatcagcgtcgtcgccgtaatcaagagccaaaatcgccaatgccattatttgctgcggtggtttcgcctgaattggcaagtggcaaggcttggattgattattcaacggtaaatctgccaaaagaaaatcatttcttatctgtggatgaattgcttgagcaagagaaaactaaaaaagggtttatcacgccagcaatgggaattgtcgttgaagaaaaatcccctgatgtaaaacctgcattagattttattactcaaccagctaatgaatcggtacagaaaaaagtacaagaatctttagaccgtttaagttcatataaacctcaagaagttgttgaatctattgatccagcaattaatgtagatgaacctgaaacgctcgagaaagtatcaaaatttgtccgcacttatgaatttaatggacgtttaggtacaatttcttcagtaccccatacaaaagcggagatgactttagcgaaagcaaatgatgaaatgccagaggctttcccaattagggcttggcaggattctcgttattatttctatggaaaaggtgcggcaggacatcattgtgcaattagccacgtttattctgaaccaacccgcgcaaaatcagaataa

SEQ ID NO: 56 - DNA, 2967 bp, Haemophilus influenzae
atgaccaattttagattaaacgtgcttgcctattccgttatgcttgggctaacggcaggtgttgcttatgcagctcaaccaaccaaccaaccaaccaaccaaccaaccaaccaaccaaccaaccaaaatggtaatgtttctgaacaactagagcaaattaatgtatctggttctaccgaaaatagtgatacaaaaacaccaccaaaaattgctgaaacggtaaaaacggctaaaacgctggaaagagaacaagcaaacaatattaaagacatcgttaaatacgagacgggcgttactgttgttgaagctgggcgttttgggcaaagcggttttgccattcgtggtgtagatgaaaaccgtgtagcgattaatattgatggattacgtcaagctgaaaccctatcttctcaaggctttaaagagctttttgagggttatggtaacttcaataatacgcgtaatggtgcagaaattgaaactttaaaagaagtaaatattacaaaaggggcaaattcaatcaaaagtggtagtggttccttaggtggatctgtaatttataaaacaaaagatgcgagagattatctccttaacaaggattactatgtaagctacaaaaagggatacgctacagaaaataatcaatcattcaatacccttactcttgcaggacgttataaaaagtttgatgccttagtggttacaacaagcagaaatggacacgaacttgaaaactatgattacaaaaatgcaaatagccttactcaaggtaaaaaaagagaaaaagcagacccatacaaaattgaacaagatagtacattattaaaattatcttttaaccctacagaaaatcatcgttttacccttgcagcagatttatatgaacatcgttctcgtgggcaagatttatcctatacactaaaatatcaaaaaacagatcctaatttactcgaggttgattctagacacaccaatgataaaacaaagagacgtaatatttcctttagctatgaaaatttctctcaaacgccattttgggatacattaaaaatcacttattcagatcaacgtattaaaactcgcgcacgtacagatgactattgtgatgcaggtgtaagatattgtgagggaactgcaaatcctgcgggactaaaattaacagatgggaaaataacacgtcgagatggttcagaacttcaatttgaaaaaaaagataaaaatattgataacaacatctatgacttcgataaatttattgatactgatgatcgagtaatagaaggtaaactaggcctaaggagatcctctggaacttggtatgattgttcaatatttgattgtaaagataaaacaaaaatgaaaatttttgaaactgaacatccttatggctatggcactactggaacgtggaaaaaagactttgaacttgaaataaaaaaattaaatgacaaaaatttcgctagagtaaaggatgcgaataataaaacatactctattcttccctcatctccaggttatttagaacgcctctggcaagagcgagatttagacaccaacacccaacaattaaatttagatttaaccaaagacttcaaaacttggcgtgttgaacataatctacaatatggtagctcatataatacaacgatgaagcgaatggttaatcgtgctggtaatgatgcttctgatgtgcaatggtgggcagagcctacacttggttacagtttgttgtatgataaacctcacacttgtaaaaccgcttatggggggtggaaagctaacctttgccctcgagttgatcctaaattttcattcttattacccattaaaacaaaagaaaaatcagtctatctctttgataatgttgttataactgattatttatcttttgatttgggttatcgttatgacaatatccattatcaaccaaaatataaacacggcgttacaccgaaattacctgatgatattgtgaaaggattgtttattccattaccaagtggtaaaaataataatgatgatcctgaagttaagaaaaacgtacaacaaaatattgactatatcgctaaacaaaacaaaaaatataaagcacattcttacagttttgtttcaacgattgatccaacgagttttcttcgtttacaactaaaatattcaaaaggttttagagcaccaacttcagatgaaatgtatttcacctttaaacaccctgatttcactattttgccaaatactaatctaaaaccagagatagcaaaaacaaaagaaattgcttttacattacataatgatgattggggttttatctccacaagtctatttaaaaccaactataaaaactttatcgacctaatatttaaaggagaaaaagattttaaattagttagcggaggtagcacattaccattttctctttatcaaaatattaatagagatagtgcggtagtaaaaggaatagaaattaattcaaaagtattccttggtaaaatggcaaaatttatggatggatttaacctaagctataaatatacctatcaaaaaggaagaatggatggcaatattcctatgaatgcaattcagcctaaaacgatggtgtatggcttaggctatgaccacccaagccaaaaatttggatttaatttctacactacccacgtagcaagtaaaaatccagaagatacttatgatatttatgcgaaagataaaaaccaaaccaacactagcataaaatggcgcagtaaatcttatactattctagatttaattggatatgtgcaaccaattaaaaatttaaccataagagccggcgtatataatcttacaaaccgtaaatacatcacttgggattctgcgcgttcaattcgttcatttggtacaagtaatgttatagatcaaaagacaggtcaaggcattaaccgcttctacgcaccaggtagaaattataagatgtcagttcaatttgagttttaa

SEQ ID NO: 57 - DNA, 4179 bp, Haemophilus influenzae
atgaaaaaaactgtatttcgtctgaattttttaaccgcttgcatttcattagggatagtatcgcaagcgtgggcaggtcacacttattttgggattgactaccaatattatcgtgattttgctgagaataaagggaagtttgcagtcggtgcgaaagatattgaggtttacaacaaaaaaggggagttggtcggcaaatcaatgacaaaagctccgatgattgattttgcatctgttgctcgtagtggttatgctgcattagtaggcgatcaatatattgtaagtgttgctcacaatgggggatacactaatgctgattttggtgcggaaggacaaaatcccgatcaacatcgttttacttatcaaatagttaaacgtaataattataaacctgatcagaagcatcattataatggggattaccatatgcctcgtttacataaatttgtaactgaaagttctccttttgatatgacaggaagtatgaagggacagacctatgcagacaaagaaaaatacccgatgcgtgtgcggattggttctggtgatcaatattggagaaatgaacaagataaaggaactcaagtagctgaagcttatggatatcttattgcggggaatacccatatgcaaactggtgcaggaaatggagatgttagtttaagtggcgatgtacgtaaaggaaatgattatggacctatgccgacagcaggatcatttggagacagtgggtcacctatgtttatttatgatgctaccgagaaaaaatggcttataaatggtgtattgcgtactggaaacccatccttaggtagagagaatacttttcagctagtccgtaaaaattattttgatgaaattttctctgctaatcttaaaacgaccatatttgatttcagaactcctaaaaatcaacaaaatcagcactacacatttcattcaaaaaatgatgggtcaggcactattacatctggagctggtaaaatttataaagtcaaacttgctaattacaatttatctatgaaaggcacggagcctgtctatactaatggtggtgtaaacaattattctcccgcattaagtcacggcgaaaacctttattttattgattacggaaaaggggtgctaacttttagtaataatatcgatcaaggtgcaggcggtttgtattttgagggagattttatagtttcgcctacccataatgaaacgtggaaaggggcggggatatctatcagtgatgaaagcaccgtaatttggaaagtcaatggcgtagaaaatgatcgcctttctaaaattggtaaaggaacgttacatattcaagcaaaaggtaaaaacttaggctcaattagcgttggcgatggtaaagtaatcttagaacaacaagaggacgatcaacataaaaaacaagcctttagtgaaatcggattggttagcggcagagggactgtccaattaaacgatgataaacaatttgataccgataaattttatttcggctttcgtggtggtcgcttagatcttaatgggcattcattaacctttaaacgtatccaaaatacggacgagggggcgatgattgtgaaccataatacaactgaagtcgctaatgtcactattactgggaacgaaagcattgttctacctaatggaaataatattaataaacttaattacagcaaagaaattgcctacaacggttggtttggcgaaacagatgaaaataaacacaatggacgattaaaccttatttataaaccaaccacagaagatcgtactttactactttcaggtggcacaaacttaaaaggcgatattactcaagaaggcggcactttagtgtttagtggtcgcccaacgccacacgcttacaatcatttaaatcgcccaaacgagcttgggcgacctcaaggcgaagtggttattgatgacgattggatcaaccgcacatttaaagctgaaaacttccaaattaaaggcggaagtgcggtggtttctcgcaatgtttcttcaattgagggaaattggacaatcagcaataacgccaacgccacatttggtgttgtgccaaatcaacaaaataccatttgcacgcgttcagattggacaggcttaacgacttgtaaaacagagaatttaaccgacacaaaagttattaattctataccaacaacacaaatcaatggctctattaatttaactgataatgcaacggcgaatgttaaaggtttagcaaaacttaatggcaatgtcactttaacaaatcacagccaatttacattaagcaacaatgctacccaaataggcaatatccgactttccgacaattcaactgcaacggtggataatgcaaacttgaacggtaatgtgcatttaacgaattctgctcaattttatttaaaaaacagccatctttcgcaccaaattcagggcgacaaaggcacaacagtgacgttggaaaatgcgacttggacaatgcctagcgataccacattgcagaatttaacactaaataacagtactgttacgttaaattcagcttattcagctagtccaaacaatacgccacgccgtcgtcgccgttcattagagacggaaacgacgccaacatcggcagaacatcgtttcaacacattgacagtaaatggtaaattgagcgggcaaggcacattccaatttacttcatctttatttggctataaaagcgataaattaaaattatccaatgacgctgagggcgattacatattatctgttcgcaacacaggcaaagaacccgaaacccttgagcaattaactttggttgaaagcaaagataatcaaccgttatcagacaaactcacattcacgttagaaaatgaccacgttgatgcgggtgcattacgttatgtattagtgaagaataatggcgaattccgcttgcataatccaataaaagagcaggaattgcacaatgatttagtaagagcagagcaagcagaacaaacattagaagccaaacaagttgaaccgactgctgaaacacaaacaagtaaggcaaaagtgcggttaagaagagcggtgttgcctgataccctgcctgctcaaagcctgttaaacgcattagaagccaaacaagctgaaccgaatgctaaaacacaaaaaagtaaggcaaaaacaaaaaaagcgcggtcaaaaagagcattgagagcggtgttttctgatcccccgcctgatcaaagccagttagacgtattaaaagccgcacttgaggttattaatgcccaaccgcaagtgggaaaagaacgtcaagctcaagagaaacaaggcaaacaaaaagacttgatcagccgttactcaaatagtgcgttatcggagttgtctgcaacagtaaatagtatgctttccgttcaagatgaattggatcgtctttttgtagatcaagcacaatctgctgtgtggacaaatatcgcacaggataaaagacgctatgattctgatgcgttccgtgcttatcagcagaaaacgaacttgcgtcaaattggggtgcaaaaagccttagataatggacgaattggggcggttttctcgcatagccgttcagataatacctttgacgaacaggttaaaaatcacgcgacattaacgatgatgtcgggtttcgcccaatatcaatggggcgatttacaatttggtgtaaacgtgggcgcgggaattagtgcgagtaaaatggctgaagaacaaagccgaaaaattcatcgaaaagcgataaattatggtgtgaatgcaagttatcagttccgtttagggcaattgggtattcagccttatttgggtgttaatcgatattttattgaacgtgaaaattatcaatctgaagacgtgaaagtgcaaacaccgagccttgcatttaatcgctataatgctggcattcgagttgattatacgtttaccccgacagataatatcagcgttaagccttatttcttcgttaattatgttgatgtttcaaacgctaacgtacaaaccactgtaaatagcacgatgttgcaacaatcatttgggcgttattggcaaaaagaagtgggattaaaggcagaaattttacatttccaactttccgcttttatctcaaaatctcaaggttcacaactcggcaaacagcaaaatgtgggcgtgaaattggggtatcgttggtaa

SEQ ID NO: 58 - DNA, 4479 bp, Haemophilus influenzae
atgaacaagatatatcgtctcaaattcagcaaacgcctgaatgctttggttgctgtgtctgaattgacacggggttgtgaccattccacagaaaaaggcagtgaaaaacctgttcgtacgaaagtacgccacttggcgttaaagccactttccgctatattgctatctttgggcatggcatccattccgcaatctgttttagcgagcggtttacagggaatgagcgtcgtacacggtacagcaaccatgcaagtagacggcaataaaaccactatccgtaatagcgtcaatgctatcatcaattggaaacaatttaacatcgaccaaaatgaaatggtgcagtttttacaagaaagcagcaactctgccgttttcaaccgtgttacatctgaccaaatctcccaattaaaagggattttagattctaacggacaagtctttttaatcaacccaaatggtatcacaataggtaaagacgcaattattaacactaatggctttacggcttctacgctagatatttctaacgaaaacatcaaagcgcgtaatttcacccttgagcaaaccaaggataaagcgctcgctgaaatcgtgaatcacggtttaattaccgttggtaaagacggtagcgtaaaccttattggcggcaaagtaaaaaatgagggtgtaattagcgtaaatggcggtagcatttctttacttgcagggcaaaaaatcaccatcagcgatataataaacccaaccattacttacagcattgccgcacctgaaaatgaagcaatcaatctaggtgatatctttgctaaaggtggtaatatcaatgtccgcgctgcaaacattcgaaaccaaggtaaactttctgccgattctgtaagcaaagataaaagcggcaacattgttctttccgctaaagagggcgaagcggaaattggcggcgtgatttccgctcaaaatcaacaagccaaaggcggtaagttgatgattacaggtgataaagtcacattaaaaacaggcgcagttatcgacctttcaggtaaagaagggggagagacctaccttggcggcgacgagcgcggcgaaggtaaaaacggcattcaattagcaaagaaaacctctttagaaaaaggctcaacaattaatgtatcaggcaaagaaaaaggcgggcgcgctattgtatggggcgatattgcgttaattgacggcaatattaatgctcaaggtaaagatatcgctaaaactggtggttttgtggaaacgtcggggcattatttatctattggtaatgatgcggctgttgaggcgaaagaatggttgttggatccggataatgttaccattagcaatggtaacgatgatcaatctcaattaaaggacgatagaggcgattcacctaataaaattctagcagataataaacatactgttaataacaaaacattatctacagctttagctaaaggtattggagttaatatttctgctaaaaagaaagtaaatgttactgcagatattaatgttcataatggaacattaacgttacactcagaacaaggtggagttgaaattaacggtgatattacctcagaacaaaatggtaatttaaccattaaagcaggtagctgggttgatgttcacaaaaatatcacgattggtatggggtttttaaatattaccgctgggggttctgtagcttttgaaaaggcagggggtgataaaggccgtgctgcaagtgatgctaaaattgttgctcagggtgtaattactgcaggcagtggtcaagatttccgttttaataatgtatcactaaatggaactggtagagggttgaaattcattacagctaaaggaaataaaggtaatttctcagccaaatttgatggtgtgttaaatatttcaggtaatatttcaattaatcacactgctaataatcaattatcttattttcatcgccagggttatacatattggaatttgacgcagcttaatgttgattctgattctagtttttctttaacatcaataaaagacgcgattaaagttgggggatatgacaatgctaaagacaaaaaaaacacaggagggattggttttacacgagataccattttcaatgtaaaacaaggtgctagagttgatatttcctacaccctaccgatatctccagttaaaaattcaagaattgctgctgtcaattttgacggaaatattactgttaaaggaggaggagttgttaatttaaaatttaatgcgcttagcaataactacaaaactccaggggtgaacatttcctctagatttattaatgttaccgaaggttcgcagctaaacattactggtagtatgccatctacaaccttatttaatgtagcaaatgatttaatcataaacgcaaccaattcatttgtatccattaaagaaattgaaggtacagatactcatcttgatacaggtttaaaggtaaacggtaatgttaccattaaaggagggaatgtcaccctaggttcaaataaagctaaaaccaaatttgataaaaatgtaactgttgaaaagggtgcaaatcttactcttgcgtcagcaaactttggaaatcataagggtgctttaactgtagctggaaatatcaatactcaaggtaagcttgttgcaactggcgatactattgatgtatctggtgattttactgttggaaacgatgctacttttaatggtaatactaataacaacctaaacattaccggcaattttaccaacaacggcacctccataattgatgtaaaaaaaggggcggcaaaactaggtaatattaccaatgaaggtagtttaaatattactactcacgctaacactaatcaaaaaaccattattaccggaaatataaccaataaaaaaggcgacttaaacatcagggataataaaaataatgccgaaatccaaattggcggcaatatctcgcaaaaagaaggcaatctcacaatttcttctgataaagtcaatattaccaagcagataacaatcaaagcaggcgttaatggggagaattctgattcaggcacagaaaacaatgccaatctaaccattaaaaccaaaacgctagaattaacaaacaacctaaacatttcaggctttcataaagcagaaattacagctaaagataacagtgatttaattattggcaaggctagcagtgatagcggtaatgctggtgctcaaaaagtaatctttgacaaggttaaagattcaaaaatctcagctggcaatcacaatgtaacactaaatagcgaagtggaaacgtctaatggtaatagtaatgctgctggcgatagtaatggtaacaacgctggtttaactatttccgcaaaagatgtggcagtaaataataacatcacttctcacaagacaataaatatctctgccacaacaggaaatgtaacaaccaaagaaggcacaaccattaatgcgaccacaggtggcgtggaagtaaccgctaaaacaggtgacattaaaggtggaattgagtccaagtctggcggagtaacacttactgcaaccggagatactcttgctgtaggtaatatttcgggtaacactgttagtgttactgcaaatagcggtacattaaccaccaaagcagactctacaatcaaaggaaccgggagcgtaaccaccttaagtcaatcaggcgatatcggcggtacgatttctggtaagacagtaagtgttacagcaaccaccgacagtttaactgttaaaggtggcgcaaaaattaatgcgacagaaggaactgcaaccttaactgcatcatcgggcaaattaaccaccgaggctagctcaagcatcacttcagccaagggtcaggtagacctttcagctcgggatggcaatattggaggaagtattaatgctgctaatgtgacattaaataccacaggcactttaactaccgtgaagggttcaagcattaacgcaaacagcggcaccttggttattaacgcagaagacgctaagctagatggtacggcatcaggtgaccgcacagtagtaaatgcaactaacgcaagcggctctggtagcgtaactgcggtaacctcaagcagcgtaaacatcactggagatttaagcacaataaacggattaaacatcatttcgaaaaatggtaaaaacactgtagtgttaaaaggtgctgaaattgatgtgaaatacattcagccaggtgtagcaagcgcagaagaagtaattgaagcgaagcgtgcccttgagaaggtaaaagatttatctgatgaagaaagagaaacgctagccaaacttggtgtaagtgccgtacgttttgttgagccaaataatgccattacggttaatacgcagaacgagtttacaaccagaccatcaagtcaagtgacaatttctgaaggtaaggcgtgtttctcaagtggtgatggcgcagcagtatgtaccaatgttgctgacgatggacagcagtag

SEQ ID NO: 59 - DNA, 4629 bp, Haemophilus influenzae
atgaacaagatatatcgtctcaaattcagcaaacgcctgaatgctttggttgctgtgtctgaattgacacggggttgtgaccattccacagaaaaaggcagtgaaaaacctgttcgtacaaaagtacgccacttagcgttaaagccactttccgctatattgctatctttgggcatggcatccattccgcaatctgttttagcgagcggtttacagggaatgagcgtcgtacacggtacagcaaccatgcaagtagacggcaataaaaccaccatccgcaatagtgtcaatgctatcattaattggaaacaatttaacattgaccaaaatgaaatggtgcagtttttacaagaaaacaacaactccgccgtattcaaccgtgttacatctgaccagatttcccaattaaaagggattttagattctaacggacaggtctttttaatcaacccaaatggtatcacaataggtaaagaagcaattattaacactaatggctttactgcttctacgctagacatttctaacgaaaacatcaaggcgcgtaatttcacccttgaacaaaccaaggataaagcactcgctgaaatcgtgaatcacggtttaattaccgttggtaaagacggtagcgtaaaccttattggcggcaaagtaaaaaatgagggtgtaattagcgtaaatggcggtagcatttctttacttgcagggcaaaaaataaccatcagcgacataataaacccaaccattacttacagcattgctgcccctgaaaatgaagcaatcaatctgggcgatatttttgccaaaggtggtaacatcaatgtccgcgccgccactattcgcaataaaggtaaactttctgccgattctgtaagcaaagataaaagcggcaacattattctttccgccaaagagggtgaagcagaaattagcggtgtgatttccgctcaaaatcagcaagccaaaggtggtaagttgatgattacaggcgataaagttacattaaaaacgggtgcagttatcgacctttcaggtaaagaagggggagagacctatcttggcggcgacgagcgcggcgaaggtaaaaacggcattcaattagcaaagaaaacctctttagaaaaaggctcaacaattaatgtatcaggtaaagaaaaaggcgggcgcgctattgtatggggcgatattgcgttaattgacggcaatattaatgctcaaggtagcgatatcgctaaaactggtggctttgtggagacgtcggggcattacttatccattgacagcaatgcaattgttaaaacaaaagagtggctactggaccctgataatgtaacaattgaagctcctagcctttctcgcgctgacactgacattagttcagaatttccaatcggtgacggtactgaaaacagccctaaaaaaaacgccgacaaaacaatactgaccaatgaaaccatttcaaattttctgcaaaatgccaaagtgatgaacataacggcaaagagaaaacttaccgttaatagctcaatcagtatcggaagtagatcccacttaattctccatagtgaaggtcagggggacggaggcgttcagattgatggagatattacttctgaaggcggaaatttaaccattaactccggcggatgggttgacgttcataaaaatattacgcttggtacgggttttttaaatattaccgctgggggttctgtagcttttgagaaagggggcaataacgcacgtaacgcaacagatgctcaaattaccgcacaagggacgataaccgtcaataaagatgataaacaatttagattcaataatgtatctattaacgggacgggcgagggtttaaagtttattgcaaatcaaaataatttcactcataaatttgatggcgaaattaacatatctggaatagtaacaattaaccaaaccacgaaaaaagatgctaaatactggcatgcatcaaaagactcttactggaatgtttcttctcttactttgaatgacgatgcaaaatttacctttataaaattcgttgatagcggctcaaattcccaagatttgaggtcagcacgtagacgttttgcaggcgtacattttaacggcaccggaggcaaaacaaacttcaacatcggagctaacgcaaaagccttatttaaattaaaaccaaacgccgctacagacccaaaaaaagaattacctattacttttaacgccaacattacagctaccggtagcagtgatagctctgtgatgtttgacatacacgccaatcttacctctagagctgccagcataaacatggattcaattaacattaccggcgggcttgacttttccataacatcccataatcgcaatagtaatgcttttgaaatcaaaaaagacttaactataaatgcaactaattcaaaatttagtcttaaacaaacgaaagatttatttgaaaatcaatacacaggagacgccattaattcaactcgtaatctaactattcttggcggcaatgtcactctgggcggggagaattcaagcagtaacattacaggaaatatcaccatcgctgccgaagcaaatgtcacattacaagcttacgccgacaatagcatcaaaggtcataagaaaaaaactctaactcttggtaatgtgtccacctctggaaacttaagcttaactggctcaaaggtggaggtaaaaggcgatcttgccgttttaaacggtgccacttttaaaggagaaaccaatgacagcctaaacattaccggcacctttaccaacaacggtaccgccgacattaatataaaacgaggggtggtaaacatccaaggtgatattaccaataaaggtggtttaaacattaccactaatgcccaaaaaaatcaaaaaaccattattaacggaaatataaccaacaaaaaaggcaacttaaatattacgaataatggtaatgatactgaaatccaaattggcggcaatatctcacaaaaagaaggcaatctcacaatttcttctgacaaagtaaatatcaccaagcagataacaattaaagcaggcgttgatgagaaggattctagttcaagcacagcaagtgatgccaatctaaccattaaaaccaaagagttaaaattggtagaagacctaaacatttcaggctttaataaagcagaaattacagctaaagatggtagtgatttaactattggtaacaccaatagtgctgatggtactaatgccaaaaaagtaacctttaaccaggttaaagattcaaaaatctctgctaacgatcacaatgtgacactaaatagcaaagtggaaacatctggtaatactgacaacactggagacggcagtggcaataatgccggcttaactattgccgcgaaaaatgtagaagtaaaaaacaacattacttctaacaaaacagtaaatatcaccgcgtcagaaaaacttaccaccaaagcggatgcaaccattaatgcaaccactggtaacgtagaagtgacagccaaaacaggtgatattaaaggtgaagtcaaatccacttccggtaatgtaaatattacagcaaacggcgacacgcttaatgtaagtaatgtttcaggcaatgctgttaccatcactgcagataagggcaaattaaccacccaagaaagctctacgattagcggaactgagagtgtaaccacctcaagccaatcaggcgatattggcggtgcaatttctggtaacacggtaagcgttaaagcaaccaatgatttaattaccaaagcaaattcaaaaattgaagcaaaaacaggcgaggctaatgtaacaagtgcaacaggtataattggcggcacaatctctggcaatacagtaaatgttacagcaaatactggtagtttaactattaaaggcggtgcaaaagttgatgcgacaaatggagctgcaaccttaactgcagaatcaggcaaattaaccactcaagcaggctctactattacctcaaacaatggccagacaactcttacagccaaggatggcagtatcgcaggaagtattgatgccgctaatgtgacattaaataccacaggcactttaactaccgtagtgggttcaagcattaacgcaaacgaaggcaccttagttattaacgcacaagacgccacactaaatggtgatgcatcaggcgaccgtacagaagtgaatgcagtcaacgcaagcggctctggtagcgtaactgcggtaacctcaagcagcgtaaacatcactggagatttaagcacaataaacggattaaacatcatttcgaaaaatggtaaaaacactgtagtgttaaaaggtgctgaaattgatgtgaaatacattcagccaggtgtagcaagcgcagaagaagtaattgaagcgaagcgtgcccttgagaaggtaaaagatttatctgatgaagaaagagaaacgctagccaaacttggtgtaagtgccgtacgttttgttgagccaaataatgccattacggttaatacgcagaacgagtttacaaccagaccatcaagtcaagtgacaatttctgaaggtaaggcgtgtttctcaagtggtgatggcgcagcagtatgtaccaatgttgctgacgatggacagcagtag

SEQ ID NO: 60 - DNA, 5385 bp, Haemophilus influenzae
atgctaaataaaaaattcaaactcaattttattgcgcttactgtcgcctacgcattaaccccttatacagaagccgcgttagtgagaaacgatgtggattatcaaatatttcgtgattttgcagagaataaagggaagttttctgttggtgcaacaaatgtggaagtgagagataacaaaaataataatttgggcagtgcgctacctaaagatattccgatgattgattttagtgctgtggatgtagataagagaattgccacgttggtaaatccacaatatgttgtgggggtaaaacacgttggtaacggtgtgggtgaactacattttgggaatttaaatgggaattggaaccctaaatttggaaattctattcagcatcgagatgtatcttgggaggaaaacagatattacactgttgaaaaaaataatttttcttctgaattaaacggaaaaacacaaaataacgaaaaagataaacagtatacatctaataaaaaagatgttccttccgaattatatggacaagccttagttaaagaacagcaaaaccaaaaacgccgtgaagattattatatgccacgccttgataaatttgttaccgaagttgcaccgatagaggcttcaactacaagtagtgatgctggaacatataatgatcagaataaatatcctgcttttgtgagattaggaagtggctctcaatttatctataaaaaaggaagccattatgaattaattttagaagagaaaaacgagaaaaaggaaataatacacagatgggatgtaggtggagataatcttaaattagttggtaacgcttatacctatggtattgcaggcacgccttataaagtgaatcatacagacgatggactcattgggtttggtgattcaaccgaagatcataacgatccaaaagagatattatctcgtaaaccgcttactaattatgctgtcttgggagatagtggctcaccgttatttgtttatgataaatctaaagagaaatggcttttccttggtgcttatgatttttggggagggtataagaaaaaatcttggcaagaatggaatatttataaaccacaatttgctgaaaatattctcaaaaaagacagtgctggtttactgaaaggtaatacgcaatataactggacaagcaaaggtaatacaagcttgatatctggaacatctgaatcattaagtgttgatttagtggataataaaaatctcaatcatgggaaaaatgttacgtttgaaggaagtggaaaccttaccttaaataataatatcgatcaaggtgcaggcggattgttctttgaaggtgattatgaagttaaaggcacttcagaaaatacgacttggaaaggggctggtatatctgttgctgaaggaaaaacagtaaagtggaaagtgcataaccctcaatttgatcgtttagctaaaattggtaaaggaaagctaattgttgaaggaagaggagataataaaggttcgctaaaagtgggcgatggcacagttgttttaaaacaacaaacaactacaggacaacacgcttttgcttctgtaggaattgtaagtggtcgctcaactgttgtacttaatgatgataaccaagtagatccaaattcaatttactttggttttagaggcggtcgattagatgccaatggcaataatctcacttttgaacatatccgtaatattgatgatggtgcaagactagttaatcataatatgactaatgcctcaaatataacgattactggagcaggattaattacaaatccaagtcaggtcactatatatacaccagcaataaccgccgatgatgataattattactatgtgccatctattccaagaggaaaagatctttactttagtaatacctgttataaatactatgcgttaaagcaaggtggaagccctactgcagaaatgccttgctatagctctgaaaaatcagatgctaattgggaattcatgggcgacaatcaaaatgatgcacaaaagaaagccatggtgtacatcaataacagacgtatgaatggctttaatggttattttggcgaggaagcaactaaggcagatcaaaacggtaaattaaatgttacctttagtggcaaaagtgatcaaaatcgctttttattgactggcggaacaaaccttaacggtgaattaaaagttgaaaaaggtacattattcctttctggcagaccaacaccacacgcaagagatattgcaaatatttcttcaacagaaaaagataaacactttgctgaaaataatgaagtggtggtagaagacgactggattaaccgcacttttaaagcaacaaatattaatgtaaccaataacgcaaccctttattcaggtcgcaatgttgaaagtattacttcaaatatcacagcctctaataaagcaaaagtgcatatcggctataaagcaggcgataccgtttgtgtacgttctgactatacgggctatgtgacttgccataatgacactttatctacaaaagcactcaatagttttaatccaactaatttgcgcggtaatgtaaatttaaccgaaagtgcaaactttaccttaggcaaagctaacttatttggcacaattaacagcacggaaaatagccaagttaatttaaaagaaaacagccattggtatttaacaggaaatagtgatgttcatcaattagatctagcaaatgggcatattcatttaaataatgtctcagatgcaacaaaagaaacaaaataccacacgctcaatattagcaatttatcaggtaatggttctttctattattgggttgattttaccaaaaatcagggcgacaaagttgttgtaaccaaatcagccaaaggtacctttacattacaagtggcaaataaaacaggcgagccaaatcataatgaacttacgctttttgatgcgtcaaatgcaactgaaagaagtggcttaaatgtttcactggctaatggcaaagttgatcgaggtgcttggagctatactttgaaagaaaatagtggacgttattacttgcataacccagaagtagaaagaagaaatcaaactgtcgatacgccaagtattgcaacagctaataatatgcaagctgatgtacctagcgtatcaaataaccatgaagaaaccgctcgtgttgaagcaccaattccactgccagcaccacctgcacctgctactggatcagcaatggcaaacgagcaaccagaaactcgtcctgcagaaaccgttcaacctacaatggaagatacaaatactactcacccatcgggatctgaaccacaagctgatacaacacaggctgatgatccaaattcagaaagtgttccatcagagacaattgaaaaagtggctgaaaatagtccgcaagaaagtgaaacagtagcgaaaaacgagcaaaaagcaaccgagacaacagctcaaaatgatgaagttgcaaaagaagctaaaccaactgtggaagctaacactcaaacaaatgaactcgcccaaaatggaagcgaaaccgaggaaactcaagaggctgaaactgctcgtcagtctgaaataaattctactgaagaaacagtagtagaagatgatcctacaatcagtgaaccaaagagcagacctagaagaagtattagctcgtcttctaataatataaatctagcaggaacagaagatactgctaaagtagaaacagagaaaactcaagaagcccctcaagtggcttttcaagcgtctccaaaacaagaagaaccagaaatggctaaacagcaagaacagcctaaaactgttcaatcgcaagcacagccagaaactaccacacagcaagcagagcctgctcgtgaaaatgtttcgactgttaataatgtaaaagaagctcaaccgcaagcaaaacctacaactgttgctgcgaaagagacaactgcatcaaacagtgaacagaaagaaactgctcaacctgtagctaatccaaaaacagctgaaaataaagcagaaaatcctcaatcaacagaaacgacagatgaaaatattcatcagcctgaagcccacactgctgttgcttcaacagaagttgttacacctgaaaatgctacaacacctattaaacctgttgagaataaaactacagaagctgaacaacctgtgacagaaaccacaacagtaagcactgaaaaccctgtagtgaaaaatccagagaatacaacacctgctacaactcaatctacggttaattcagaggcagttcaatcagaaacagcgacaacagaagctgttgttagccagtctaaagtgacttctgctgaagaaacaacagtagcttctactcaagaaacaacagtggataattcaggatctactccacaaccacgaagcagaagaactcgtagatcagctcagaatagttacgagccagtggaactacatactgaaaatgcggagaatccgcaatcgggaaatgatgtggctacacaactagtattgcgcgatctcacaagtacaaacaccaatgcggtaatttctgatgccatggcaaaagcacaatttgttgcattaaatgtagggaaagccgtttctcaacatattagccagttagaaatgaataacgaggggcaatataacgtttgggtatctaatacttcaatgaaagaaaattattcctcaagtcaatatcgtcattttagttctaaaagtgcgcaaactcaacttggttgggatcaaacaatctcaagcaatgttcagttaggtggtgtgtttacttatgttcgcaatagtaacaactttgataaggcaagcagtaaaaataccttagcacaagcaaatctctattctaaatattatatggataatcattggtatttagcggtggatttaggctatggtaatttccaaagcaacttacaaactaatcataatgcgaaatttgctcgccatactgcacaatttggtttaaccgcaggcaaagcatttaatcttggcaattttgctgttaaaccaactgtaggggttcgttatagttacttatcaaacgctaattttgcattagctaaagatcgcattaaagtaaatccaatatctgtcaaaacagcctttgctcaagttgatttaagttatacttatcacttaggcgagttttccattacgccaattttgtctgctcgatatgatgcaaatcaaggcagcggcaaaattaatgtagatagatatgattttgcttacaatgtggaaaaccaacagcaatataacgcaggacttaaattgaaatatcataatgtgaaattaagtctaattggcggattaacaaaagcaaaacaagcggaaaaacaaaaaactgcagaagtgaaactaagttttagtttttaa

SEQ ID NO: 61 - RNA, 288 nt, Haemophilus influenzae
augaaaaaauuagcaacauuaacagcauuagcaagugcauuaacgauggcaguagcgacagcaguacaggcugaaucaaagucaagcaauacugauaauacagcaacaccuuguguaggcgauaaaugugucaaaacgaaagccgcagaagguaaguguggugaaggcaaaugcggugcagauaaagcuaaaucugcugaagguaaauguggcgaggguaaauguggugcgagcaaaccgaaagcugcugaaggcaaauguggcgaagguaaaugcgguucuaaauaa

SEQ ID NO: 62 - RNA, 465 nt, Haemophilus influenzae
augaaaaaagcauuuugguuaguauuaaguucagcaauagucuuggcugcuugugaugauaaaccuaaaaugacggagcaaacuaaaacaacugaccaagcgaaaauggcugcagaacaaccugaucaaaagcccguauugaucaauuauaaacaaauugccuuagacaaaucccagcaagcgauugauacagcgaaagucgcucaagacaaaucaguucaagcuaaugaagccgugaaaguagcaaugcaaaaacaacaagaggcugaaauugcgcuaagucaagguggcgaagaggcugaaaaauugucuaaacaaaaaauggacgaaucuaaugcagcugcagaauuagcuuuaaaauugucggaagaauccaaagcugcuucugcacuuucaauccaacuuuccaaagaagcggaagaugcggcuaauaaggcgaaucaacaagcucaagcugagaaauag

SEQ ID NO: 63 - RNA, 564 nt, Haemophilus influenzae
augaaaaaauuaacauuagcauugguuuuagguucagcuuuagccgugacagguuguuuugauaagcaagaagcaaaacaaaaaguugaggauacgaaacaaacuguagcaucagucgcaucagaaacaaaagaugcugcagcuaauacaaugacugaaguaaaggaaaaagcacagcagcuuucuacagacgugaaaaauaaaguagcagagaaaguggaagaugcgaaagaaguaauuaaaucagcaacagaaacagcaucagaaaaagcaacugaaauaaaagaggcaguuucagaaaaagcaagugagaugaaagaagcugcaucagaaaaagcaagugagaugaaagaagcugcaucagaaaaagcaagugagaugaaagaagcugcaucagaaaaagcaagugaaaugaaagaagcugcaucagaaaaagcaagugagaugaaagaagcugcaucagaaaaaguaggcgaaaugaaagaaaaagcaacugaaaugaaagaggcgguuucagaaaaagcaacucaagcaguagaugcagugaaagaagcgacaaaauaa

SEQ ID NO: 64 - RNA, 582 nt, Haemophilus influenzae
augaaaaagaaaaaucagauuuuaguuaguuuaucuauuguggccuuauuaggaggcuguucagaagagcaaguacaacgugauguguaucagucuuuagacgauuguuuagcggauuggaaaaaaauugagcuuugugaggcagauaaaaauacugaaaguacucagaaaacugaaacuacaccacagcaggggcuugguuuaaauauucgugauaaugguaaugcagaaagugcgguaaaaaauccagcugaaaauaauguacaagcuaaucaaagcgagaacaaugcugaaucaacugcaaaagcugaaaguaccgauccuucuuuaggagccgcaauugcugguggcguuauaggguauauggcggcucgugcaauuaguaguuuccuuggcccaagcuaucauccuggaaaucgagcugucaccacaccaacagggcaaguugugcaaccucaaacgaaucguucuguuggaaaaccaaugcucgugaaaggaaaugcaggaagcaugaauagcaaaccuguaucucguggugguuuuaguagcccaaauaauacucaucguagcagugggggcuaa

SEQ ID NO: 65 - RNA, 651 nt, Haemophilus influenzae
augaauuuaacuucuuuaaucaacaaaucugcaaaagcguuagcguuaagugcggcauuuguagcauugccauuuuuggcuuaugcggaugaugcacaaaaaccugcggaucauguuacuuaugaaccgcaauuagauaaccaacgcgauccuaaucaauauugugcuaaaugccauaaauucgacaaaauagauaaaaaccaaacgcuagaucggucugguggcgaacuccacuuugguaaauuccauggugcacauuuagauaagaaaaauccaaauaacgguaaagcuauuacuuguguaagcugucacggcaauguuuccgaaaaucaucgucgcggugcuaaagauguuaugcguuuugaaggagauauuuucgguaauaaaaaaccgauguauucaguucaagaacaaaaccaaguuuguuuugcuugccaucaaccugacaaacuucgugaaaaauuaugggcucaugauguacacgcaaugaaauugccuugcgcaaguugccauacgcuucacccuaaagaagacgcaaugaaagguauucaaccaaaacaacgcguuaaacuuugcguagauugccaugguaaacaacaaaaacguaaagcugaacaagauaaacuaaccaaacaaaaggauaaacaauga

SEQ ID NO: 66 - RNA, 678 nt, Haemophilus influenzae
augggaauuaaaaacaagccgauaacaagaccaugcccgcagugcggcagaaauuaucaauaucggcgggcaagugggcguacuuuugagcuuugcgaauauugucgcaaucuugauuguguaguuugugguaaaaaaguaccgccggaacgugggcggaaaaauacuugcugugcggagugcgaaaaauugaagauacacaauauccaaaaugcgcacuacgccaaacggauagcggaagacccugaguuaaauaaacgcaaccacgccaaagcccgcgaaaaccgaaaagcagacccggaacgaaugcacgaacauuuggaagcacaaagggaaaggcauuauagacgugugcaagauccgaauuaucuagccacucggaaaguuuaucaggcucaacgauggcaggauaaaaaagaugagauacuggcucaacggcgcgaauuuugggauaguuuaagcgauguugagaaagcggagcgacuugagcgcaaucaagcaauacaacguaaacauaaagccaaaaaacgugaccaguugaaauuagauccgcaaaagugggcggaauaucaagaguaucaacgcacaaagcggcgcgaacauagacagagaaaggcgcuuaaugaauuaauggucggcacaaaggaguuauuaaauguuaccaacaaagacaaauag

SEQ ID NO: 67 - RNA, 795 nt, Haemophilus influenzae
augcagcaaacaaaacguucgcuauuggguuugcuuauuucuuugaucgcacacgguauuauuauaggauuuaucuuauggaauuggaaugagccaagugauagugcaaauagcgcacaaggcgauauaucuacaaguauuucuauggaacuauuacagggcaugguguuggaagaaccugcuccagagccagaagauguacaaaaagagccagagccugagccagaaaauguacaaaaagagccagaaccagaaaaacaagaaauuguagaagauccaacaauaaaaccugaaccgaaaaaaaucaaagaaccagaaaaggaaaagccaaaaccaaaagaaaagccgaagaauaaaccuaaaaaagagguuaaaccacaaaagaaaccaaucaauaaagagcuaccaaaaggcgaugaaaauauugauucaagugccaacguaaaugauaaagcaaguacaacaagugcagcuaauagcaaugcacagguagcaggaaguggaacggauacgagugaaauagcggcuuaccguucugcaauccgccgugaaauugaaagccauaaacguuauccaacucgagcgaagauaaugcgcaaacaaggaaaagugaguguaucuuuuaauguaggagcugaugguucauuaaguggugcuaagguuacaaaauccucaggcgaugaaaguuuagauaaggcggcauuagacgcuauuaacguaucucgcucuguuggaacaagacccgcaggauucccuucgaguuuaagugugcaaauuaguuuuacucuucaauaa

SEQ ID NO: 68 - RNA, 1140 nt, Haemophilus influenzae
auggaaaaagcuggcuaugagcgagcuagugauucugauucauccuuuuccgguggaggugguuggcgagaagauaacaguagugauaguuaucguaguacgucagauagauggaaugaccacaaaucuagauacggaaaagacaaagucuauacugaugcauuuaaugagcgaagaaauaacucuaguuggagcgguggucauagcgcaauuagccgaacaauuagugaaaaauaucauucacuuucuaaugggcaaaugagcgccgccguuccugaaaaagaucagaaaacacucacuggcgguuuguuuggaaaaaguuacuccaaugcgccuuauucugaacgcacuccuucuauauuugauagaaacauacgugguucaaugacauuaaauaacggcgauguauggucaagcgauccccaauauucauccguucgagaacgggcggacaucaauaguuacgaccguauuaaacggggcgaagaauugaacuuaauuggucgugcuguaggaggcguuuuuaguggggugggcggggcagcaacaacgccaguuggcaaaauugcugaaagugcggcaaauuuugggcuuucccacguuggggauuuaucucgacaauucaaaagcaaccaagagcaagcguauuaugauagccucacuccagaggggaaagcguauuacgauacaagaguagauuucaucaauaaguccuauaagaaugcucgggaaaaauaugaaacgaacgauaaauggauugauagagguauuacagcugcacaagucgguuuaucugcuuuagggccuccuggugcaaugcuagggucugggauugguuuauuagguaaagcgaucaacaaaaaagacacgaugacaaaaucauuacgugauuuaacagagacgcuuaacucuaacgcauuaaauaaccacaucgcacaacaaaaugaauuagcugaaaaagaacgucaagccuauaaggaauuuauggcugggcgugauuuacgcagugacaauacacaaccaaaaggcauacugaacacuauguauaaucguaugcaaaauauagauccugauaaacaggucaaaacgagugacguuccuaaccuaagaaauuauugggcaaauaucaucguaucauag

SEQ ID NO: 69 - RNA, 1218 nt, Haemophilus influenzae
augaacaaaucguuucucuuacucccuuuaagucuuguuguauuaucggcuuguaccucuaauucuccugcaccuauuucugaugcagauggaaaucuuuccccuaguguagugcaaucuguaaauggcaguaauguagguggugcuuggcaaccugaaauucaaaaaaauucauuaccuacaaugggaaauauggucacaccacaaccaaauuuucaaccgauuaaucaacaaccagcaaugccaacuaaaccugcacaaccugcauuucaacccucuccgaaaacgguaguuagcgcaccgacaguacaaacaaaaacaguuaccaaaacaauugcugauuguguagauggacagcauauuaauaucccacguaauccaaauaccaaugcgccagauuacagcaaaauuucaaaaggcucuuacaaaggcaauaccuauaaaguaaacaaaggcgauacaauguucuugauugcuuacuuagcaggaauugauguaaaagaauuggcagcguugaauaaccuauccgaaccuuauaauuuaaguuuagggcaaguuuuaaagauuucuaauuguggcacaaaaacgguuacuacaacgguuucuguaaaacaaccugcagucacggcaucaacagcaaugccagcuaaaccagccgucacuuauacuccgggcgcaaacgguacucaaauaggaucugacgguacuauuauuggccccauuaaaucagaggcuggcauaucgucuaguguaucuguggcaacuucaucaacacagguuacaucaucuguaaauaaugcgaauagcacaccgauuaaugcaaaugucguugcaccaaucgccucaaauguugcguggcaauggccgacuucagguaauauuauucaagguuucucaagcacagauggcgguaacaaagggauugauauuagcgguucacgaggacaagcuguaaaagcggcugcagcagggcgaauaguguaugcuggcaaugcuuuacgugguuacgguaauuuaaucaucaucaaacauaaugaugauuucuuaagugcuuaugcgcauaacgacaaaauucuuguugcugaucaacaagaagucaaagcuggucaagacaucgccaaaauggguagcucugguacaaauaccgugaaacuccacuuugaaauucgcuacaaaggcaaaucaguagauccagugcguuaucuaccaaaacguuaa

SEQ ID NO: 70 - RNA, 1227 nt, Haemophilus influenzae
augcaaaauaaucgacaaaagaaaggaaucaaugcuuuugcuauuucuauccuuuugcacuuuaucuuguuuggcuuauugauuuuaaguucgcuuuaucacaccguugaaauuaugggugguggagaaggugaaggagauguaauaggggcagugauuguugauacugguacggcugcucaggaaugggggcguauucaacaacaaaaaaaagggcaagcggauaaacaaaaacgcccagaaccuguuguggaagaaaaaccaccugagccuaaucaagaagagauuaagcaucaacaagaaguucaacgacaagaagagauuaagcaucaacaagaaguucaacgacaagaagaguuaaaacgucagcaagaacagcaacgucagcaagaaauaaaaaaacaacaagagcaagcucgucaagaagcguuggaaaaacagaagcaagcugaagaggcuagggcuaaacaagcggcugaagcugcaaaauuaaaagcagaugcagaggcuaaacguuuagcugcugcggcaaaacaagcugaagaagaggcuaaagcgaaagcagcagaaauugcugcucaaaaagcaaaacaagaggcagaaacuaaggcgaaacuagaagcggaagcuaaggcaaaagccgcugcugaagcuaaagcaaaggcugaagcggaggcaaaagcgaaagcagcagcugaggcaaaagcaaaggcugaagcggaggcaaaagcgaaagcagcagcugaggcaaaagcaaaagcugaugcagaagcuaaagcgaaagccgcugcugaggcaaaagcugcugcggaagcaaaacguaaagcagaucaagcaagccuagaugauuucuuuaacggcggagauguugguggcgguagugcaucuaaagggggaaacacaaauaaagguggaacucaagguagcggugcugcacuuggcucuggcgauggugguaagguuggggaucaauacgcagguguaauuaagaaagagauucaacgucguuucuuaaaagauccaaauuuugcaggaaagguuugucguauuaaaauucaauuaggucgagauggcacaaucuugggguaucaaaaaauuucaggcucugaugauauuuguucagcugcauuaagugcgguggcuagaacgaaaaaaguuccagcugcgccaucagaugaaauuuaugaaaaauauaaaucaccaauuauugacuuugauauucgauaa

SEQ ID NO: 71 - RNA, 1287 nt, Haemophilus influenzae
auggauaaaaaucaacaaucaucucaaaaugaauuggauuuagggcuuaaucaagagccaauuacaccaaagaaaacaauucagccaaguucgucaauuuuagguaaagcaaaaggguuauuugccaaaaaaaaucacgugcaaacuaacuuucagcaacguaaagaaccuacuuuuggcgauucaucaacgcaagaaaaugauccuuuaauuccgagugaaaauuugaaaaaagugcaaaagccuguucuucaaacuucuuccacagaagaaaauauuucugcgguugaugaagaaaucagugcugaaaauaacgcggaugaacccgucgaaaaggcugaaaaaccuauuuuagcucaaccagaaaaauggaaaauauuacaaguauugccagcaaaacaucgccguuuauuuauggcuauuuuuguguugguuauuuuauugauuauuuucuucgcauuaaaaccaaguucugacaccguugagucuuuuacucaaucuaacagcaaugaaguuccugugcaguuccaaucguuagaucaaagucagccacuggaaaccacgauuuuagauaauccuccugcacaaaaucaaauggcuguagaacaagcuaaccaaucugaauuugcaccaaaagcagaggaagcggcgaauaauacgacggcucaaaacccauuaguagaaaaugcgccaaugcaacaaaauguuguucaaucuccaagucaaaugccaaaugaaauggcugcggcaucuguuaugccuaugcaaccagcucaagcggaacagccaaaagcaacugugccaguucagccgaugaaaaaagcgguagagccacaaguugcgcauaaagauacugugaaaaaagaagugaaaguggcggagaaugcacaggcuccaucaaaagcaacggaacaaaacgucgcuaaaacagcaggaaacgcaccgauuguugaagccaaaccuguucaaguaaaaaaagaaaagaaaguucaaaucguugaugcaaaaccugugaguaaaucugcagccucucgucuuucugcgaaaacauuaacugugccgaaagguguuucgcugaugcaagucuuccgugauaaucaacuuaauauuuccgauguaaacgcaaugagcaaagcagcaggggcgggcaauguuuuaaguagcuuuaaaucuggcgauaaaguaacgguaucugugaauaaucaagggcgaguaaaugaaaugcguuuauccaauggugcgcguuuugugcgucagucugaugguucuuaucaauauaaaaaauaa

SEQ ID NO: 72 - RNA, 1347 nt, Haemophilus influenzae
auggcugaagaaaacaaagaaauuauagcuuauaaaggguuuaagcaagacuggacuugucgagguuaucaguaugagguaggcaaaacguaugagcauaaagguaauguuaaggcuugugagaguggauuccacgccugcgaauacccgcuugaugugcuuagcuauuacaguccagcgguaaguaaauuugcuguaguuaaaaugagcggcgaaacaucaaaagauagugaugauacaaaaauugcaucugcaaaaaucacgaucgaaaccgaaauuaacuuaccggaaaugauaaaaaaagccguugaauggauaaaagguaaaguugauugggaugcagccaagguauccaauacaggcgauuggucggcagcaaccaauacaggcgauuggucggcagcaaccaauacaggcgaucagucggcagcaaccaauacaggcaauuggucggcagcaaccaauacaggcgaucagucggcagcgacuaauacaggcgaucagucggcagcgacuaauacaggcuaucggucgguagcgacuaauacaggcuaucggucgguagcgacuaauacaggcgaucagucggcagcgacuaauacaggcggucagucggcagcgacuaauacaggcgaucagucggcagcgacuaauacaggcuaucggucgguagcgacuaauacaggcgaucagucggcagcgacuaauacaggcuaucggucgguagcgacuaauacaggcgaucagucggcagcgacuaauacaggcggucagucggcagcgacuaauacaggcgaucagucggcagcgacuaauacaggcuaucggucgguagcgacuaauacaggcgaucagucggcagcaaccaauacaggcaauuggucggcagcaaccaauacaggcgaucagucggcagcgacuaauacaggcgaucagucggcagcgacuaauacaggcuaucggucgguagcgacuaauacaggcgaucggucggcagcgacuaauacaggcuaucggucggcagcgacuaauacaggcuaucggucgguagcgacuaauacaggcgaucggucggcagcgacuaauacaggcuaucggucgguagcgacuaauacaggcgaucagucgguagcggaaauaucuggcaagcaaucuauagcuguugcgcuugguuggcaaucuaaagcuaaggcgaguauuaauggugcuauuguuuguguauaucgcaaucaugauggcgagcuaauccauaucaaagcaucaaaagucggugaaaauaacaucaaagcugauacuugguacacguuagaugaauuuggugaguuuguugagguuaaagaugacuaa

SEQ ID NO: 73 - RNA, 1395 nt, Haemophilus influenzae
augaaagcaaauaaagcguucuauguuauuggugggauuaccgcucuugucgugacgcugauggcuggauuucuuuucuccgguaguaguucaacaaauacagcagaaaccaagaaagaagauacuuuagauaucucuuuuaaagauuuaucaccggaagaauuacgagcaauggggauugagggugacacaucuaaagauacgguaagaacacuuauagguaaagugaaagagcauaauaagcgauuugaugaagugauuuuacaaaaugaacgcuugguaaaagagaaugagcaguuaaaaaaucaagcaaacaauacggauuaucaaauucaacaggcgguacaagcagaaacaggggcguuaauuaaugagauuaaugcguuaaaagaucagguuguacaagcugguaaucaaguuguagggaauggaaaugcccaggcaaaucccaaugcuccucuaccaauaaacggagcaacaacuggcccucagguugaaacucgagggggaauuaauugggugaacccuagugauaugccaacuacagacagacaagggaaugcucuacaagguauugguguaaauggugcaggaaucgguuuucccaaaaccuucgauaacaaugugaaugacauccgaaaaucuaaugauggguugcaaucuaguacagguaauaaugcaauauuuaauaccgcuacaccauuuuucacaauugcagcuaacucuacuuuaacuaauucuguugcuaugacugcgcuuguugggcgugugccgauugacaauaagguaaccgagccuuauccguuuaaauugcuuauuggucgagauaacuuaauugcaaauggcauugaacugccggauauugaaggcgcaauugucucuggaacagcaagcggugacuggacgcuaucuuguguucguggugaugugaaaagcuuaacuuuuguguuuagcgauggccgcauuguuucaacagacaauucugacgauaaaauuggcugguuaucugauccacauggugugccuugcauuccuggagaacgcagaacaaaugcuccggaauaucuaacgaccaacuucuucuuaacgggggcuucagcagcugcacaaggcuuaucaucuucucaaucaacaacagugguugauggcggugcaguaauuggugcgguaacggguaauaacgguaaguauauucuuggucaagcguuagguggcgguuugagagaaacagcagacugguuccgucagcguuauggccaaauguuugaugcgguauauguuccaccggguaaagaaguggcuguacacaucgaaaaacaaauugauauugauuaugaccgcaugucucgaaaagugaaauacggacaagccucaagacaaucuaacuuggauuaa

SEQ ID NO: 74 - RNA, 1422 nt, Haemophilus influenzae
augcaacacguaaaauuagcucgagauagacgaaaaaaacgcacauauauaaaaguaggcguguuuuuuguugcuauucugcuuauuuugacaggcauuuuacuuacaauaaaagacaaaucugaagaaaaucccauauuuuccaccucugacaguggggaauaucacgaguuaaauacaucgccaaauaaaaauuccaccgcacuucaaccugaugaagaugccaccucuuaugaugaugaacugcaagcaaaagaugaugaaguugaugaaguaaaacuaucuucagaugaucucgguacuuuaccucaacacgcacaagacgcacucaaugguuuauuagacgcggccgaucaagccauaagaauuacugaucaauuuagcuauacgguaaccgaaggggauacguuaaaagauguuuuaguuuuaucugguuuagaugauucaucaguacaaccguugauuaagcuugaucccgaauuagcccacuuaaaagcaggucagcaauuuuacuggauuuuaaauaaaaaugauaacuuggaauauuuaaauuggcugguuucugaaaaagaagaacguauuuaugaacgucucgaagaugggaaauuuaaacgccaaguuauugaaaagaaaaguauuuggcgaaaagaaguauuaaaaggcgagauucaaaauucuuuaaauaguaguuuaagagaacaaggucuugauacgcgacaaauuagccaacuuaguaacgccuuacaauggcaagugaguuuacgaaaacuaaaaaaaggcacucaauuugccauuuuaguuucucgugaauauuuaggcgauaaacuuacaggucaagguaauguugaagcguuacguauuucaucuggugguaaaaauuauuaugcagugcaagcggcaaauggucguuauuauaaucagcaaggggaaacuuuaggcaaagguuuugcucguuauccauugcagcgacaagcgcgaguuuccuccccuuuuaauccaaaucgucgccauccaguaacaggacguguucguccccauaaaggcguggauuucucuguuucucaaggcacaccaguuauugcaccagcagaugguacaguugaaaaggucgcuuaucaggcuggcggugcugggcguuauguuauguugcgucacgggcgugaauaucaaacuguuuauaugcacuugagcaaauccuuaguaaaagcagggcaaaccguuaaaaaaggcgaacguauugcgcuuucugguaacacgggcauuucuacggggccgcauuuacauuaugaauuucguauuaacggucgagcagugaauccucuuacuguuaaauuacccgguacuagcagugguaugacuucagcugaacggaaacaauuccuuguucguguucgugaagcugaaaaaauguuaaaaccuuag

SEQ ID NO: 75 - RNA, 1527 nt, Haemophilus influenzae
augaacgcauucaaaaaaagccuuauuguggcagcgucuuucgcgaguuuaagcuuauuuaacucagccacugcugagcuuguauauaagccauuagaacaaccuguugaacccgcaaaaccugauuugaaaauugaaucuguaaaugaaaaauuugcagaaaaauauccaaaucaauauaacagcuggcguucuacagcaaacggcgauggcgaaaacauuauuuaugcugaugaagaaaauccgcgccuaaucguacuuuggggugguuaugcauuugcaaaagaauauaacgcaccacgcggucacuuuuaugcgguaacagaugugcguaauauuuuacguacaggugcgccaaaaacugcaaaugauggcccucaagcuauggcauguuggacauguaaagguccugauguuccacguuuaaucgcggaauggggugagaaagauuauuucaaugcgaaaugggcaaagggagggccugaaaucgugaacucaauuggcugugcugacugucacgacacaaccucuaaagauuuugcugaaggcaaaccugcauuacguauugcucgcccacacguauuacgcgcccuugaugcuuuagaaaaagccacugcugaaaaagauaaagcagaaggucgcccacacaacaauuuaaguuucaacacggcggcgcguacugaaaaacgugcagaaauuugcgcaaacugccauguugaauacuauuuugcaggggacaucaagcaaguaacuuucccuugggauaauggucaaaccgcagaugauauugaaaaauauuaugaugauauugguuucacugacuggacucacucacuuucuaaagcaccaauguuaaaagcacaacacccugacuuugaaauuuggucuuuagguaugcacgguaaaaaugggguaacuuguguagauugccauaugccgaaaguacaaggcgcagaugguaaaguuuauacugaccaccaaauccaaaauccauucgaugcauuugaccacacuugugcaaauugucacgaucaaagcaaagaaaaauuacgcgauauuguuacuucacguaaaaaagaagugaaagauguaaugggucguuuagaagaccaaguuguuaaaucucacuuugaagcaaaagcggcuugggacgcaggugcaacuaaagaagaaauggaagcagcauuaauggauauucgucaugcucaauggcguugggauuacacugcugcaagccaugguggucauaugcaugcgccugaaguaguacuucguguauuagcuucuggcuuagauaaaguagcagacgcgcgcacuaaacuugcuguaaucuuaacuaaacacggugugaaaacuccaguucaaaucccagauauuucaacagcugauaaagcguggaaaguaauggguauugauauugaaaaagaacguaaagcaaaagaagaauucuuaaaaacugugguaccgcaaugggagcaacaagcuagagaaaaaggcuuauuaguugauccaccagcacaaaaauaa

SEQ ID NO: 76 - RNA, 1893 nt, Haemophilus influenzae
augaaaucuguaccucuuaucacugguggacuuuccuuuuuauuaagcgcuuguagcgggggaggugguucuuuugauguagaugacgucucuaaucccuccucuucuaaaccacguuaucaagacgauacuucaaguucaagaacaaaaucuaauuuggaaaaguuguccauuccuucuuuaggaggagggaugaaguuaguggcucagaaucugagugguaauaaagaaccuaguuucuuaaaugaaaauggcuauauaucauauuuuuccucaccuucuacgauugaagaugauguuaaaaauguuaaaacagaaaauaaaauacauacaaauccaauugggcuugaaccuaauagagcauuacaagaccccaauuuacaaaaauacguuuauucugguuuguacuauauugagaauuggaaagacuuuuccaaauuagcaacagaaaaaaaagccuauaguggccauuaugguuaugcguuuuauuaugguaauaaaacugcaacagacuugccaguaagcgguguagcaacguauaaaggaacuugggauuucaucacugcaacuaaauauggccaaaauuauucuuuguucaguaaugcuagaggucaagcuuauuuucgacguagugcuacucgaggagauauugauuuagaaaauaauucaaagaauggugauauaggcuuaauaagugaauuuagugcagauuuugggacuaaaaaacugacaggacaacugucuuacaccaaaagaaaaacugauauucaacaauaugaaaaggaaaaacucuaugauauagaugcccauauuuauaguaauagauucagggguaaaguuacuccuacgaaauccacaucggaugaacaucccuuuaccagcgagggaacauuagaaggugguuuuuauggaccuaaugcugaagaacuaggggguaaauucuuagcuagggauaaacgaguuuuugggguauuuagugccaaagaaacgccagaaacagaaaaggaaaaauuauccaaagaaaccuuaauugauggcaagcuaauuacuuucucuacuaaaacagccgaugcaacaaccaguacaacagccaguacaacagccgauguaaaaaccgaugaaaaaaacuuuacgacaaaagauauaucaaguuuuggugaagcugauuaccuuuuaauugauaauuacccuguuccucuuuucccugaaggggauacugaugacuucguaacgaguaaacaucacgauauuggaaauaaaaccuauaaaguagaagcauguugcaagaaucuaagcuauguaaaauuugguauguauuaugaggauaaagagaagaaaaacacaaaucaaacaggacaauaccaccaauuuuuguuaggucuccguacucccaguucucaaauuccuguaacgggaaacgugaaauaucucgguaguugguuugguuauauuggugaugacaagacaucuuacuccacuacaggaaauaaacaacaagauaaaaaugcucccgccgaguuugaugucaauuuugacaauaaaacauuaacaggcaaauuaaaacgagccgacucacaaaauaccguguuuaacauugaggcaaccuuuaaaaaugguaguaaugccuucgaagguaaagcaaccgcaaauguagugauugaucccaaaaauacacaagccacaucuaaagucaauuucacgacaacaguaaacggggcauuuuaugguccgcacgcuacagaauuaggcgguuauuucaccuauaacggaaacaauccuacagcuacaaauucugaaaguuccucaaccguaccuucaccacccaauucaccaaaugcaagagcugcaguugucuuuggagcuaaaagacaaguagaaaaaaccaacaaguag

SEQ ID NO: 77 - RNA, 2001 nt, Haemophilus influenzae
augaaauuaaaucaugcacugaaagcaucuuauauugcuauaucgugcgcaaucuuauccucaaccaugccaguaucagcuucuaauaguaauccguuuggauuuaauaaagccgauucuauuuugucugauaugguguuuuaucaaauugggggcgguguagguuauauggcucccccgagucgagguacgauuccugcagcugaauuugggauugguuggaaagcuaacuugaugugcgguaauuuugacauuaagacgaguauuaaaaaucaguuaaaugguuuaacugaaggauuuaaagaucuuuauaguaacguuaucgaaucagccacaggagcuguggcgaguuuaccugcaaugguuauucaacgagcaaauccucaguuauacgauauucuuacgaaugguuuauaucaagguaaaauugauuuuaauagcuuaaaaaccagcugugaagaaaugucuaagaagcuugcagacgcaacuuuaaauggucguuggucucaaaguucggauauggaauccuuuaaagacauuacuucaacugaaccagaugcuacucgagcuaaaaagagacuugaggaaucucaagguaaggaagguaaagagugggucggggguaaaaaacgcgguggccaaggacaagaaccaaucaaaauuguugaggauguugcaaaagcuggcuacaacaugcaaaacaagcguaauguauuagaaucaggucgaaucuccgguaguaguuguagcggauuacuuugucaaacuugggauaauccuaaagauaugacagauugguuaacgaaugugaucggugagaagcaauuaacuacuuguaaaacagauugugguacugcuaguagcucucgagcugguguugguuuaacaccugaaauugaaaaagaaaauauuaagacgguaacucaauuacaaaaagcgcuuaacaugagcacuccuuccguugaaguacuugcagaauuaaguucuacaacaguuccuguaacucguggcuuaauugagucuuuacgagaagaucccgaugcucaaguauuagggcagcguuuagccagugaaauugcuguagcuaaaacaauggagaaaaugcuucuugcacgucgagcuguuuuaacggguaugcgugagccaaauguggccaacaauaaagaugcucaagaagaguuagaaaagauccucacuuuaauugaccgagaaauaggucaaauuagaauggagauugaucuacaaaaaucacuuaccgggaauuccgcaguugcaauccuacaaaauaaagagcuacgugaauauaacacuggaaccggaaauguuacacgugacaguguagauaaacggauuagcgauuuagcuaaugguacgaauagcaaugcugcugaauuggcagaugcugacaauaucaauauuccucguaacaaugucacuuuagaagugccucagaugucaaacucccugucuuacaccgggacuggcgguacauccggaaauaguagagguaacacuucuuauucaucaguuccagcaauuaaagggacugcacuugaucaagcuacaggguuguugaaaaaauuugaaggguuuagugauaaagcauauugggaugucaaugcuuaucgcacuggauaugguucagauacuaucacaaaagcggaugguacaauugucaaaguaacgaaagauacuguaguuagucgcgcugacgcagagcgugauuuagcacgucgaacacaagaauuugcaaauagagcgagaaauaacguaucaucuucaacaugggauaaguuaccgccaaaugcccaagccgcauuaacaucuuaugccuauaacuauggcucauuaacaaaagaugugauugcugcagcccaggcaucugcgcaaucaggcgauaugaacgcacuagcaaaugcaguaagaaaucgucaaaacaauaauaaugguguaaaugcuaaacgucguaaccaagaagcagauuacauuuuaggaaaauaa

SEQ ID NO: 78 - RNA, 2082 nt, Haemophilus influenzae
augaaauuuaaaaugucaaaaaaugugauuuguuaugcgugguuaucgguguguuugagcagugcaauuccugcauuugcuguacaaccuacauuaaaaccaagugacauuaguauuccagcgauaagugaagaaagccaguuggcaacuaagcgugcaacgacaagauuaacucaaucccauuaucguaagauuaaauuagaugaugauuuuuccgagaaaauuuuugaucguuauauuaaaaauuuagauuuuagccacaauacuuuuuugcagucugauauagacgaacuucgucaaaaauaugguacuaaguuagaugaacaguugagucaaggcgaucuuucugcggcguuugacauuuaugauguaaugaugaaacgucgcuaugaacguuauacuuaugcguuaucuuuauuagauaaagagccugauuuaaauggucaagaucaaauugaaauugaucgugaaaaagcggcugcgccacaaacugaagcugaugcgaauaaacuuugggaugcucguguaaaaaaugacauuauuaaucuuaaauuaaaagauaaaaaauggucagaaaucaaagcaaagcuuacaaaacgcuauaauuuagccauucgucguuuaacgcaaacaaaagcagacgauauuguucaaauuuauuugaaugcuuuugcgcgugaaauugauccucacaccaguuaucuuuccccaagaacagcaaaaaguuuuaaugaaaguauaaaucuuucuuuggaaggcauugggacuacuuuacaaucagaagaugaugaaauuagcaucaaaucucuuguaccuggugcgccagcggaacguaguaaaaagcugcauccuggggauaaaaucauugguguugggcaagcaacaggcgauauugaagacguuguugguuggcguuuagaagauuugguagaaaaaauuaaagggaaaaaaggcacuaaaguucgcuuagaaauugagccugcuaaagggggaaaaucacguauuauuacuuugguucgugauaaaguccguauugaagaucaagcugcuaaacuaaccuuugaaaaagugucaggaaaaaacauugcgguaauaaaaauuccaaguuuuuauauuggauuaaccgaagaugucaaaaaacuacugguuaaauuagaaaaucaaaaagcugaggcucuuauuguugauuuacgugaaaauggcgguggugcauuaacggaagcgguggcguuaagugguuuauuuauugccgauggcccuguugugcagguucgcgaugcuuaucagcguauucguguucaugaagaugaugaugcuacgcaacaauauaagggguuauuauuugugaugauuaaucguuacagugcuuccgcaucagaaauuuuugcugcagcaaugcaagauuaucgucgagguauuaucaucgggcaaaauacuuuugguaaagguacggugcaacagagucguucguuaaauuuuaucuaugauuuagaucagaguccguuggguguuuugcaauauacuauucaaaaauuuuaucgcguuaauggugguucaacccaauuaaaagguguugcagccgauauuaauuucccugaaauuauagaugcgaaagaauauggggaagauaaagaagauaaugcgcuugcuugggauaaaaucccaucagcgaguuauacggaaguaggaaauaucgauuauauugauaaugcaguaaauauucuaaaugaaaaacaucuugcucguauugcuaaagauccugaauuuguugcauugaaugaagaauugaaagugcgaaaugaacgccgagaucgaaaauuucuaucguugaauuauaaaaugcguaaagcagagaaugauaaagaugaugcaagacguuuaaaagauuugaacgaacguuuuaaacgugaggguaagaaagcauugaaagauauugaugauuugccgaaagauuacgaagcaccagauuucuucuuaaaagaagcugaaaaaauagcagcugauuucguuauuuuuaauucagaucagaaaauuaaucaagcaaacggccuauccgaagcaaaaacggaaaguaaaaaauaa

SEQ ID NO: 79 - RNA, 2130 nt, Haemophilus influenzae
augcgauuuucuaaacuuucccuugcaauuacaacaaccuuagugacagcaaaugcacuagcgcaauccguugaauuagacucuaucaacguuauugcgacacgagauccaaguagguuuacuuauacgccacaaaaacaaucuaaagauagucuucuuuccaaacaagcuacuaguguugcagcugcguuggaagacauuccaaauguugauguuagaggcgguucgagaagcauugcucaaaaaccuaauauccgaggguuaagugauaaucguguugugcaagucauugauggcguaagacaaaauuuugauuuagcacauagagguucuuauuuucuuccaaugucacucauccaagaaauugaaguaaucaaagggccaaguagcuccuuaugggguaguggugcauugggugguguuguggcaaugcguacgccaaaugcuuuagacuuauugaaaaauaaugacaaauucggaguuaaaauucgccaagguuaucaaacugcuaauaauuuaucggaaaaggauguuucuguauuugcggcaaaugacaaauucgauguucuuauuagugguuucuauaauaaugcggauaauuuacgcauugguaaaggcaacaagcugaauaauacagccuauaaacaguuugggggcuuagcaaaauuugguuggcaaaucaaugaugcgaaccguguggaauuaucccaccgcgaaacucguuuuaaacaaacagcaccaggcaauaaugaggcgaaaaacgaacuuacuaaugaacaaauuauagaaaaaaucaaugaguaccacaacccacucaacaauuuuccaccgaaagccaaaccaucacuagaagaguuuuacucuggaguaagagcacgucuuggcgguguuaguuauuuaagugaucaacaaauuccugaucaaagcacgguauuuaacuauuauuuaacgccugauaauccuuaucuaaauacgcauauugcgcuauacaacaauaagacuauugagaaagaacagcguaaagucaguggagugaaagaucagacuaaauugacuacccgagguauaaauuuacguaacucuuccgaauuaucucacauuuccuuuguuuaugggguggauuauaugcgagauaaaaucaguacugaacgaggcacaaacgauaaagaugcgaaguuucgagcggaacccuauaaugcaaauucaaacacuacaggcguuuauuuaauugcccauauuccacuauuuggggaaaaauugcuacuuucgccaaguguacguuaugaccacuacgauaccucaaguaaaaccguaaaauauaaggauaaucauuuaucgccugcaacaaaauuaacuuggaaagugaccaauugguuagauuuuacugccaaauauaaugaagccuuccgagcuccaucuaugcaagaacgguuuguaagcggcgcucacuuugguacaaguacucggguaggagauauuaucaauagcuuuguggcaaacccaaauuuacgcccugaaacagcgaaaaauaaagaaauuaccgcaaaucuacauuuugauagccuguuuaaacaaggcgauaaauucaaaauugaagcgacuuauuuccgcaaugaugugaaagauuuaauuaacuuaaagagacuugaugauccaaaugcaaauggagcauugucacgaacaaauucccaguaucaaaacauagcuaaugcccguuuaagcgguauugaauugcaagcccaauaccaaacagaacguuuaacgcuauuuacuaacuauggcagcaccaaagguagagauaaagauagcggcgaagcuuuaucaaacauugccgcaagcaaaaucggcguaggggcagauuaugcuuuaguaaaagacaaauucacggugggagcgacaauaacccauuacgcugcccaacaucgagugccuaaagaucacgcuguuaccuacccaaguuauauacugaccgaucuucgugcuaccuaugcgccauuaaaaggcgaauggaaaaaccugcguuuagauuuugcacuggaaaacuuguuugauagaaaauaucaaccagcauuuaguuuaauggaaggcacuggucguaaugcgaaaauuagugcgguuuauagcuucuaa

SEQ ID NO: 80 - RNA, 2403 nt, Haemophilus influenzae
augaaaaaacuucuaaucgcaaguuuauuauucgguacgacaacgacuguguuugccgcaccuuuuguggcaaaagauauucguguggaugguguucaaggugacuuaaaacaacaaauccgagcaaguuuaccuguucgugccggucagcgugugacugacaaugauguggcuaauauuguccgcucuuuauucguaaguggucgauucgaugaugugaaagcgcaucaagaaggcgaugugcuuguuguuagcguuguggcuaaaucgaucauuucagauguuaaaaucaaagguaacucuauuauucccacugaagcacuaaaacaaaacuuagaugcuaacggguuuaaaguuggcgauguuuuaauucgagaaaaauuaaaugaauuugccaaaaguguaaaagaacacuaugcaaguguaggucgcuauaacgcaaccguugaaccuauugucaauacgcuaccaaauaaucgcgcugaaauuuuaauucaaaucaaugaagaugauaaagcaaaauuggcaucauuaacuuucaaggggaacgaaucuguuaguagcaguacauuacaagaacaaauggaauuacaaccugauucuugguggaaauuauggggaaauaaauuugaaggugcgcaauucgagaaagauuugcagucaauucgugauuauuauuuaaauaauggcuaugccaaagcacaaauuacuaaaacggauguucagcuaaaugaugaaaaaacaaaaguuaauguaaccauugauguaaaugaagguuuacaguaugaccuucguagugcacgcauuauagguaaucugggagguaugucugccgagcuugaaccuuuacuuucagcauuacauuuaaaugauacuuuccgccguagugauauugcagauguagaaaaugcaauuaaagcaaaacuuggagaacgcgguuacgguagcgcaacgguaaauucaguaccugauuuugaugaugcaaauaaaacauuagcgauaacccuuguuguugaugcuggacgacguuuaacuguucgccaacuucgcuuugaaggaaauaccguuucugcugauagcacuuuacgucaggaaaugcgccaacaagaaggaacuugguauaauucacaauuaguugaguuaggaaaaauucgcuuagaucguacagguuucuucgaaacagucgaaaaccgaauugauccuaucaaugguagcaaugaugaaguggaugucguauauaaagucaaagaacguaacacggguaguaucaacuuugguauugguuacgguacagagagugguauuaguuaucaagcaaguguuaaacaagauaauuucuuaggaacaggggcugcaguaaguauagcugguacgaaaaaugauuaugguacgagugucaauuuggguuauaccgagcccuauuuuacuaaagaugguguaagucuugguggaaauguuuucuuugaaaacuacgauaacucuaaaagugauacauccucuaacuauaagcguacgacuuauggaaguaauguuacuuuagguuucccuguaaaugaaaauaacuccuauuauguaggauuaggccauaccuauaauaaaauuaguaacuuugcucuagaauauaaccguaauuuauauauucaaucaaugaaauuuaaagguaauggcauuaaaacaaaugacuuugauuuuucuuuugguuggaacuauaacagccuuaauagaggcuauuucccaacuaaagggguuaaagcaagucuugguggacgaguuacuauuccagguucugauaacaaauacuacaaacuaagugcagauguacaggguuucuacccauuagacagagaucaccucuggguuguaucugcaaaagcaucugcaggauaugcaaaugguuuuggaaacaagcguuuaccguucuaucaaacuuauacagcggguggcaucgguucauuacgugguuuugcuuaugguaguauuggaccuaacgcaauuuaugccgcucaaaaugguaauaguaauaguaauaguaaugguacuuuuaauaagauaaguucugaugugauuggugguaaugcaaucacaacugcgagugcagaacuuauuguaccaacaccguuugugagcgauaagagccaaaauacgguccgaacuucccuauuuguugaugcggcaaguguuuggaauacuaaguggaaaucggauaagaguggguuagauaacaauguauuaaaaagcuuacccgauuauggcaaaucaagccguauucgcgccucuacaggugucggauuccaauggcaaucuccuauugggccauugguauucucuuaugccaaaccaauuaaaaaauaugaaaaugaugaugucgaacaguuccaauuuaguauuggagguucuuucuaa

SEQ ID NO: 81 - RNA, 2535 nt, Haemophilus influenzae
augacugaagauguuaaggcugaugcgccaaaaaaauuaagcauucaacgcagaacaaaaacaacagugagcagcaccacaacuggcgguaaaagcaaagaaguacaaguagaaguacguaaaaaacgcacaguaaaaacugauauugcucaacaagaagaagcaaaauuaaaagcacagcaagaagcggaagcgaaaaaaauugcugaacaaaaagcagcagaagaaaaagcucguuuagaagcugaaaaagcggcaacuaagaaagaagcugaugaaaaaucaaaagcugaaaaagcgaaagcagaaaccgcuaagccagcaaaaagugcgguagauucuaaagcuaaauuuguugauccugaaaaagagaaacguaaagcagaagaagcugaacuucgucguaaagcugaagaaguggcucgucaaaaagcagaagaacaagcucgucgugcagcagaagaagcuaaacguuaugcugaagcagaugauucagacaaugaaucuucuucagaagacuauucugauuacaauuuaaguucaagauaugcacuugaagcagaagaugaagaagaucgucguaaugaaaaucguggucgagguaaaaauaaaguagcaaaagcaaaaaaaggcggucgugacgaugaaaauagcaaaaacucgaaaaacgagcgcgaaucuaaucguaaaaaucaaaaagaugcuaaguuugguaaagguaaaaauggaaaaaaaggcaccgcacuucaacaagccuuuacuaaaccaguucaaguaguaaaagcagauguugugauuggcgaaaccauuaccguugcagaacuugcgaacaaaaugucggugaaagcgacagaaaucauuaaagugaugaugaaaaugggugaaauggucaccaucaaccaaguuaucgaucaagaaaccgcucaauuaguagcggaagaacuuggucauaaaguaauucuucgcaaugaaaaugagcuugaagaagcgguauuaggugaucgcgauguaaacgcggaaaaaguaacgcgugcgccaguuguaacuauuaugggacacguugaucacgguaaaacuucuuuacuugacuauauucguaaagcgaaaguugcugcaggcgaagcgggugguauuacucaacauaucggugcguaucacguagaaauggaugauggcaaaaugaucaccuucuuggauacuccaggacacgcagcguuuacuucuaugcgugcacguggcgcaaaagcaacagauaucguuguucuuguuguugcggccgaugauggugugaugccucaaacuaucgaagcgauucaacacgcaaaagcagcgggugcaccuuuagugguugcaguaaauaaaauugauaaaccagaagcaaauccagaucguguugagcaagaauuauuacaacaugaugucauuucugagaaauucgguggugaugugcaauucguuccaguaucggcgaagaaagguacugggguugaugauuuauuagaugcaaucuuacuucaaucugaagugcuugaauugacugcuguaaaagacggcauggcaagugguguagucauugaaucuuacuuggauaaaggucguggcccuguggcaacuaucuugguucaaucagguacacuacguaaaggugacaucguacuuugugguuuugaauauggccgugcgcgcgcaaugcgugaugaaaacgggaaagaaguggaugaagcagguccuucaauuccaguugaauuauuaggucuuucaggugugccugcagcgggugaugaagcaaccguuguacgugaugagaaaaaagcacgugaaguggcauuauaucgucaagguaaauuccgugaagugaaauuagcucgucagcaaaaagcuaaacuugaaaauauguuuagcaauaugucugaaggcgauguggcugaauugaacguuauugugaaagcggauguacaaggcucuguagaggcgauuguucaagcguuaaaugaacuuucuaccaaugaaguaaaaguuaaaguuguugguucagguguaggugguauuacugaaacugaugcaacuuuagcuaccgcaucuaaugccaucauuguuggcuuuaauguucgagcggaugcaacagcucgucgugucauugaagcugaaaacauugauuuacguuacuacucaaucaucuaugaauuguugaaugaaauuaaagcagcaaugagcgguauguuagagccugaauuuaaacaagaaauuauugguuuagcugaaguucgugauguauuccgucauccgaaauuuggugcaaucgcaggcuguaugguaacugaggguguagugaaacguaacaacccaauccguguauuacgugacaacgucguuaucuuugaaggggaauuagaaucucuucgccguuucaaagaugauguaucugaaguucguaacgguauggaaugugguaucggcgugaaaaacuacaacgauguaaaaguuggcgaccaaaucgagguguuugaagugguugagguuaagcguucaaucuaa

SEQ ID NO: 82 - RNA, 2739 nt, Haemophilus influenzae
augacuaaaaaacccuauuuucgccuaaguauuauuucuugucuuuuaauuucaugcuauguaaaagcagaaacucaaaguauaaaagauacaaaagaagcuauaucaucugaaguggacacucaaaguacagaagauucagaauuagaaacuaucucagucacugcagaaaaaguaagagaucguaaagauaaugaaguaacuggacuuggcaaaauuaucaaaacuagugaaaguaucagccgagaacaaguauuaaauauucgugaucuaacacgcuaugauccagggauuucaguuguagaacaaggucgcggugcaaguucuggauauucuauucgugguauggacagaaauagaguugcuuuauuaguagaugguuuaccucaaacgcaaucuuauguagugcaaagcccuuuaguugcucguucaggauauucuggcacuggugcaauuaaugaaauugaauaugaaaauguaaaggccgucgaaauaagcaagggggggaguucuucugaguaugguaauggagcacuagcugguucuguaacauuucaaagcaaauccgcagccgauaucuuagaaggagacaaaucauggggaauucaaacuaaaaaugcuuauucaagcaaaaauaaaggcuuuacccauucuuuagcuguagcaggaaaacaagguggauuugaaggacuugcuauuuacacucaacgaaauucaauugaaacccaaguccauaaagaugcauuaaaaggcgugcaaaguuauaaucgauuaaucgccaaagaagauggaucuaaugcauacuuugugauggaagaugaguguccaaaggauuauaacaguuguauaccuucagccaaaccaccugcgaaguuauccucccaaagagaaaccguaagcguuucagauuauacgggggcuaaccguaucaaaccuaauccaaugaaauaugaaagccagucuugguuuuuaagaggaggcuaucauuuuucugaacaacauuauauuggugguauuuuugaauucacacaacaaaaauuugauauccgugauaugacauuucccgcuuauuuaagaucaacagaaaaaccggauuuagaaaauaguucuuuuuauccaaagcaagauuauggugcauaucaacguauugaggauggccgaggcguuaaauaugcaagugggcuuuauuucgaugaacaccauagaaaacagcguguagguauugaauauauuuacgaaaauaagaacaaagcgggaaucauugacaaagcaguguuaagugcuaaucaacaaaacauuauacuugacaguuauaugcaacauacacauugcagucuuuauccuaauccaaguaagaauugccgcccaacacuugauaaaccuuauucauacuaucauucugauagaaauguuuauaaagaaaaacauaauauguugcaauugaauuuagagaaaaaaauucaacaaaauuggcuuacucaucaaauugucuucaaucuugguuuugaugacuuuacuucagcgcuucagcauaaagauuauuuaacucgacguguuaccgcuacggcaaagaguauuucagagaaaccuggugaaacaccaagaagaaaugguuucaaauuacaaccuuacuuauacccaaaaccaaaugcaucuuuuguaggacgagaucauuguaauuaucaagguagcuccucuaauuauagugacuguaaagugcgguuaauuaaagggaaaaauuauuauuucgcagcacgcaauaauauggcauuagggaaauacguugauuuagguuuagguauucgguaugacguaucucguacaaaagcuaaugaaucaacuauuaguguugguaaauuuaaaaauuucucuuggaauacugguauugucauaaaaccaacggaauggcuugaucuuucuuaucgccuuucuacuggauuuagaaauccuaguuuugcugaaauguauggauggcgguaugguggcaagaaugacgagguuuauguagguaaauuuaagccugaaacaucucguaaccaagaguuuggucucgcucuaaaaggggauuuugguaauauugagaucagucauuuuaguaaugcuuaucgaaaucuuaucgccuuugcugaagaacuuaaaaauggaaagggcaagggcaauuauggauaucauaaugcacaaaaugcaaaauuaguuggcguaaauauaacugcgcaauuagauuuuaaugguuuauggaaacguauucccuacgguugguaugcaacauuugcuuauaaccgaguaaaaguuaaagaucaaaaaaucaaugcugguuuagccuccguaagcaguuauuuauuugaugccauucagcccagccguuauaucauugguuuaggcuaugaucauccaaguaauacuuggggaauuaauacaauguuuacucaaucaaaagcaaaaucucaaaaugaauugcuaggacaacguucauuagguaacacuucaagggguguaaaaucaacaagaaaacuuacucgggcauggcauaucuuagauguaucggguuauuacauggugaauagaaguauuuuguuccgauuaggaguauauaauuuauuaaacuaucgcuaugucacuugggaagcggugcgucaaacagcacaaggagcggucaaucaacaucaaaauguugguaauuauacucgcuacgcagcaucaggacgaaacuauaccuuaacauuagaaaugaaauucuaa

SEQ ID NO: 83 - RNA, 2748 nt, Haemophilus influenzae
augaagaaagcuauaaaauuaaauuuaauuacacuuggccuaauuaauacaaucgguaugacgauuacacaagcucaagccgaagaaacauuaggacaaauugauguaguggaaaaaguuauaucaaacgauaaaaaaccuuucacugaagccaaagccaaaaguacacgugaaaaugucuuuaaggaaacacaaaccauugaccaagugauucggagcauuccuggggcauuuacucaacaagauaaaggcucgggugucguuucuguaaauauucguggcgaaaauggauuaggucgugucaauacgaugguugaugguguaacccaaaccuucuauucuacagccuuagacucuggucaaucaggcggaaguucucaauuuggugcggcaaucgauccuaauuuuauugcagguguagauguuaauaaaaguaacuuuucgggagcaagcgguauaaaugcguuagcaggcagugcuaauuuuagaacauuaggcguuaaugauguuauuaccgaugacaaaccauuuggcauuauucugaaaggaaugacaggaagcaaugccacuaaauccaauuuuaugacgauggcugcuggcagaaaauggcuugauaaugguggcuauguaggcgugguguaugguuauagccaacgugaaguuucacaagauuaccguauuggaggcggagaacgauuagcaucauuagggcaagauauucucgcgaaagaaaaagaagcuuauuuucguaaugcggguuauguuuuaaaugaugcuggacaguggacuccugaucuaaguaagaacgcuuggucuuguaacgaagacccuccucguuuagcagagaauauagagaauguuaaauguacacauuauucuuuugauccuagaaaaaacgauagaaaagaaauucucaaaaaauuaaaagcuggaacaaaaccugagaauauugacgagcuccaaaacgguaaggauggaauuaaagaaacugacaaaucauuugaacguaauaaagaucaauauaguguugcaccaauugagccugguaguuugcaaucacgcucgcguagccauuuauuaaaauuugaauauggcgaugaucaccaaaauuuaggagcacaaauacguacauuggauaauaaaauugguucucgcaaaauugaaaaccguaauuaccaagucaauuauaacuucaauaauaacaacuaucucgaucuuaauuuaauggcugcacauaacauugguaaaaccauuuauccuaagggugguuuuuuugcuggcuggcgaguggcagacaaacuuauuaccaaaaauguggcaaauauuguugauauaaacaacagccauacuuucuuguugccaaaagaaaucgauuuaaaaaccacauuagguuuuaacuauuuuaccaaugaauacaguaaaaaccguuuuccagaagaauuaaguuuguuuuauaacgaugauucacaugaucaagguaacuauaguaauuuaggacgauuuagagguaaucguaauuuacugccacaacguucagugauuuuacaaccuucuggcaagcaaaaauuuaaaacgguguauuuugauaccgcacuuucuaaagguauuuaccauuuaaauuacagcgugaauuuuacccauuaugcguuuaauggugaguauguuggauaugaaaauacagcgaaucaaauuaaugaaccgauuuugcauaaaucagggcauaaaaaggcauucaaucauuccgcuacauuaagugcagagcuaagcgauuauuuuaugccauuuuucacuuauucacgcacacacagaaugccgaauauucaagagauguuuuucucucaagugucuaaugcagggguaaacacggcauuaaaaccugaacaaucugauaccuaucaacuaggcuuuaauacuuauaaaaaaggucucuucacucaagacgaugugcuaggcaucaaauuaguggguuaucguagcuuuauuaaaaacuauauccauaauguuuacgguguuugguggcgagauggcguuguaccuacgugggcagacaguacugguuuucgcuuuacuaucgcacaucaaaauuaucaaccuaucgugaaaaaaagcggggcggaguuagagcuuaauuaugauaugggacguuuuuuugcgaaugucucuuacgcuuaucaacguacuaaucagccaaccaauuaugccgaugccagcucacguccgaauaaugcuucaaaagaagacauuuugaaacaagguuauggcuuaucucguguuucaaugcuaccaaaagacuacggcagauuagagauugguacacguugguuugaucaaaaauuaaccuuagguauggcagcucguuauuauggaaaaaguaaacgugcgacaauugaagaagaauauaucaauggaucucacuuugaauuaaagaccucagguaaaagaacuuacuacguugugaaaaaaacagaagagauuaaaaaacaaccgauuauuuuagauuuacacgucagcuaugaaccaaucaaagauuugauuauuaaagcggaaguacaaaaucuauuagauaaacguuauguugauccguuagaugcuggaaaugaugcggcuucgcaacguuauuauucaaguuuaaaugauucaauuuguaguaaaagucaggauugugaagauggaggaaaagauaaaaccgugcuuuauaacuuugcacguggaagaacuuauauucugaguuuaaacuauaaauucuaa

SEQ ID NO: 84 - RNA, 2766 nt, Haemophilus influenzae
auguauaaauuaaauguaauuucucuuaucauuuugaccacuuacacuggugcgacauaugccucuacacaggguuugccacaagaggguuugccacaagaggauacagucguuuuugggaaugucacuauugauaaaacuacugcagauaaaaugacgauuacucaaaguagcccuacaacucaaauaaauuggaagaguuuugacaucggucaaaauaaagaaguggaauuuaaacagccuagcccaaacgcuguggcauauaaccguguaacuggcggcaaugcuagucaaauucaagggaaauugacggcaaacgguaaaguuuauuuagcaaacccuaaugggguuauuauuaccaaaggggcagaaauuaauguagcugguuuacuugcaacaacuaaagauuuagagcaaaucagugaaaauggcaauggcaauggcaauagcuaucaauuuacccguaaaacaaaagauggucaggagcuaaaagaagguaaaguucucaaugaagggaaaauuaaggcgaaagauuucgugguacuuaauggcgaugaaguuaucaauaaagaggaaauuaacguuacaaauggcaaaguauauuuauccucaggcgauaauuuuacauuuaccuuauuaccagauagcgguauaaguguggcauuagaagauaaugcaguacgggguauuguuaaaaaugaagggauuauuaaagcagaggacauuacgcuaaaugcaaaagggcguaaagaagcauuagauagcuugguuaugaauaaugguguguuagaagcaacuaaaguuagcaauaaaaacggcaagguuguguuaucugcugaugaaauuaaguuagaugauaaaaguaauauuaaaggggauaugguuaguuuuguggcugauguuaccucaaauaaggaacuuaaagacaauauaaaaaucacuucucaaacuggcucaaaaguuaccucgccuaaaaucgauuuuaaaggcaaaaguguuaauaucaaugguaacuuugggcgugacgauaguaaagcacacuauaacgaugagcauaaaacauuaaaaacagaaguuaauauugacgugccagacgaugaaaauauucguauugcgaauauugaagaugaagauaaagauaguacggguucauuuauacaaacagaugcguuaaguucauuacuagcuaauaauggaaaaguaacucuuaaaggaaauaacguuaauauuucaggacguauucauauagauuccuuuagaggcucagauucgcuauuaaagcuaacuaacaaaggucauauuaagaucaauaaugcggauauucauucaaaaggucguuuauuuuuuauaaccaguuugcagaaugaggaagauuuuaaaucaaauauuaccauuacugacagcaaaauaaaucuugguaacggagcuaugggauuggggcguagugucaaugaaagcgacuacgauaacaaauaucagaaaacggaaggaucacaacgcaaaaaguuuaauguuaagaugaguaacguagaauuuaaucagguugaugaugugauuuuagcagguggauuugaaaaaguuaauuuagauaaaaucguugccacaggacaaacgaauuuuuacauugauggggggguaucccguaauagaacaaauggugagccuuggaaauaugaauacgguguguuagauuuagacaaacguacgcaguuaucggaguuagaucaaggcagacgccguuggggauauuacuaugauuuagaguuagauaugaauagagccuauuuguaucguuuugaucuguuugccgcuaaaaauacaaggcgaucaacuauuaaaggcacagaaauuaauauuucuaacaguaauauuaaucugaaaaauggauuuguacauuuacuggcagaaaaaauuaaauuagauaauagcaagauugauauuacuuuugauaaagauaauucacaagauacuuuagcucaaacaaaccguuuaggaaugaaugguaaagugucaaugauaaacucacauauuaaaaucguaggcgaugagaaaagugguauaucuccaacugggacuuaugcuacgauguuuuuaaucggggaauuaauaggggaaaaaaguucuauuuuugugaaaucucaucaggguuacaccuuuaaaacugauggcgauacaaaaauugcagguaaaaacagcaaagaggacuugaaaauuaccgcaauuaacacugguggaagagcagcggaagaaguucuaauuaauggggcguuggguucagcugauaaugaugcuaauaucgccaauauggcauuuacuauuggugauucagcgaauaccaaaacaaccauugaaaaugccgacaucacugccuuagcaccaaacggugggacagcuuauuuaagcuccaaagauguagaaauugagguaaaaccaaauucagauuucacauucuuugaacuuccucgugagaaaaaccuuaaucaaacaaaaauuaacggcgcuucuaccaaacucagugaacgugguuuugcuagacuuuaugacaaaauuaacggugugcgagccagcaacuugucugcugagcaauugaauguuacggacucaagcgaaaaaaucauuaauacaaauuuagugaguagcuuagauguugaaaaacuuguuucuguugcaguuugugaugcagguaaagguugcgaagaacaacaauucggcgauaaagguaauaauacuaaagugaguguuggagaguuagaagcugaacaauaa

SEQ ID NO: 85 - RNA, 2808 nt, Haemophilus influenzae
augaaaagaauguuaaucaaugcgacucaaaaagaagaguugcgugucgcgcuugucgauggacaacgccuuuuugauuuggacauugaaaguccaggacacgaacaaaaaaaagcgaauaucuacaaaggaaaaauuacucgcguugagccaagcuuagaagcggcuuuuguggauuauggugcagaacgucacgguuuuuuaccuuuaaaagaaauugcccgugaauauuuuccugaugauuauguuuuucaaggucguccgaauauucgcgauauuuugaccgaaggacaggaaguuaucguucaaguuaauaaagaagaaagaggaaacaaaggugcagccuuaacuacuuuuguuucucuugcuggcaguuauuuggugcuuaugccgaauaauccacgggcaggaggaauuucucgccguauugagggcgaugaacguacugaacuaaaagaagcauuaaguucauuagaugugccugaagguguaggcuuaaucguccguacugcuggugugggaaaaucgccagaagaauuacaaugggauuuaaaaguauuauuacaucauugggaagcgaucaaacaagcuucacaaagucguccagcuccauuuuuaauucaucaagaaagcgaugugauuguacgcgcuauucgcgauuauuugcgucgugauauuggugagauccugauugauagcccaaaaauuuuugagaaagcgaaagagcauaucaaacuugugcguccugauuucaucaaucgcgugaaacuuuaucaaggcgaagugccacuuuucagucacuaucaaauugaaucucaaauugaauccgcauuccaacgugaagugcguuuaccuucuggcgguucaaucguuauugauguaacugaagcauuaaccgccauugauauuaacuccgcacguucuacucgugguggcgauauugaagaaaccgcauuaaauaccaaccuugaagcggcagaugaaaucgcgcgucaauuacgcuuacgugacuuagguggauuaguugucauugauuucauugauaugacaccaauucgccaucaacgugaaguagaaaaucguauucgugaugcgguucguccagaucgugcccguauucaaauuagccgaauuucucguuuugguuuguuagaaaugucacgucagcguuuaagcccaucauugggcgaaucuucgcaccauauuugcccucguugucaaggaacaggcaaagugcgugauaaugaaucucuuucuuuaucuauuuuacguuugcuggaagaagaagcauuaaaagagaacaccaagcaaguacauaccauugugccaguacaaauugccucuuaucuucuuaacgaaaaacgcaaagcaaucaguaauauugaaaagcgucauaaugucgauauuauuguugccccaaaugaagcaauggaaacaccgcacuuuaguguuuuccguuuacgugauggcgaagaaguaaaugaauuaagcuauaacuuagcgaaaauucacugcgcucaagaugaaaauacugaagaaucacugcuuucucguaauguggaaacaacagcuguuauugaacaaccagcgguugaaagugcaguaguugcacuuucuauuucagaagcagccccaacgccaguugagcguaaaucaaaugaacccucuuuguuagcuaaaauuauugcgaaaauuaaagguuuauuugcgacuaaaucugaagaaaacaaaccgaaaaauaaccgcacuucgcgcaauccuaaucguaaucaacgccguucacaggaucgucguucuucucgucguccacguucugaaaauaacgaaacagaaagaaccgaagaacagguucguaauguccgugagcguaaucaacgccguccgcgccguaacuuaguggaagaaaguaucgcugaaagugcgguaaauucuacaccuguuuuugaggcuaaagaagaaagaacugagccuguaacucaacgccgccaacgccgugauuuacguaaacgcgugcgcguugaagauaaugaaacaguuguugaaaauaacuucagcacgacugaaaaaaugccagaaguggauguaauuacgguucaaaauaaugaugaaaaacccguucaucagaaccaacguucugaacgucaagagcgucagcgucguacuccacgucauuuacgugcagcgaauaaucagcgucgucgccguaaucaagagccaaaaucgccaaugccauuauuugcugcggugguuucgccugaauuggcaaguggcaaggcuuggauugauuauucaacgguaaaucugccaaaagaaaaucauuucuuaucuguggaugaauugcuugagcaagagaaaacuaaaaaaggguuuaucacgccagcaaugggaauugucguugaagaaaaauccccugauguaaaaccugcauuagauuuuauuacucaaccagcuaaugaaucgguacagaaaaaaguacaagaaucuuuagaccguuuaaguucauauaaaccucaagaaguuguugaaucuauugauccagcaauuaauguagaugaaccugaaacgcucgagaaaguaucaaaauuuguccgcacuuaugaauuuaauggacguuuagguacaauuucuucaguaccccauacaaaagcggagaugacuuuagcgaaagcaaaugaugaaaugccagaggcuuucccaauuagggcuuggcaggauucucguuauuauuucuauggaaaaggugcggcaggacaucauugugcaauuagccacguuuauucugaaccaacccgcgcaaaaucagaauaa

SEQ ID NO: 86 - RNA, 2967 nt, Haemophilus influenzae
augaccaauuuuagauuaaacgugcuugccuauuccguuaugcuugggcuaacggcagguguugcuuaugcagcucaaccaaccaaccaaccaaccaaccaaccaaccaaccaaccaaccaaccaaaaugguaauguuucugaacaacuagagcaaauuaauguaucugguucuaccgaaaauagugauacaaaaacaccaccaaaaauugcugaaacgguaaaaacggcuaaaacgcuggaaagagaacaagcaaacaauauuaaagacaucguuaaauacgagacgggcguuacuguuguugaagcugggcguuuugggcaaagcgguuuugccauucgugguguagaugaaaaccguguagcgauuaauauugauggauuacgucaagcugaaacccuaucuucucaaggcuuuaaagagcuuuuugaggguuaugguaacuucaauaauacgcguaauggugcagaaauugaaacuuuaaaagaaguaaauauuacaaaaggggcaaauucaaucaaaagugguagugguuccuuagguggaucuguaauuuauaaaacaaaagaugcgagagauuaucuccuuaacaaggauuacuauguaagcuacaaaaagggauacgcuacagaaaauaaucaaucauucaauacccuuacucuugcaggacguuauaaaaaguuugaugccuuagugguuacaacaagcagaaauggacacgaacuugaaaacuaugauuacaaaaaugcaaauagccuuacucaagguaaaaaaagagaaaaagcagacccauacaaaauugaacaagauaguacauuauuaaaauuaucuuuuaacccuacagaaaaucaucguuuuacccuugcagcagauuuauaugaacaucguucucgugggcaagauuuauccuauacacuaaaauaucaaaaaacagauccuaauuuacucgagguugauucuagacacaccaaugauaaaacaaagagacguaauauuuccuuuagcuaugaaaauuucucucaaacgccauuuugggauacauuaaaaaucacuuauucagaucaacguauuaaaacucgcgcacguacagaugacuauugugaugcagguguaagauauugugagggaacugcaaauccugcgggacuaaaauuaacagaugggaaaauaacacgucgagaugguucagaacuucaauuugaaaaaaaagauaaaaauauugauaacaacaucuaugacuucgauaaauuuauugauacugaugaucgaguaauagaagguaaacuaggccuaaggagauccucuggaacuugguaugauuguucaauauuugauuguaaagauaaaacaaaaaugaaaauuuuugaaacugaacauccuuauggcuauggcacuacuggaacguggaaaaaagacuuugaacuugaaauaaaaaaauuaaaugacaaaaauuucgcuagaguaaaggaugcgaauaauaaaacauacucuauucuucccucaucuccagguuauuuagaacgccucuggcaagagcgagauuuagacaccaacacccaacaauuaaauuuagauuuaaccaaagacuucaaaacuuggcguguugaacauaaucuacaauaugguagcucauauaauacaacgaugaagcgaaugguuaaucgugcugguaaugaugcuucugaugugcaauggugggcagagccuacacuugguuacaguuuguuguaugauaaaccucacacuuguaaaaccgcuuaugggggguggaaagcuaaccuuugcccucgaguugauccuaaauuuucauucuuauuacccauuaaaacaaaagaaaaaucagucuaucucuuugauaauguuguuauaacugauuauuuaucuuuugauuuggguuaucguuaugacaauauccauuaucaaccaaaauauaaacacggcguuacaccgaaauuaccugaugauauugugaaaggauuguuuauuccauuaccaagugguaaaaauaauaaugaugauccugaaguuaagaaaaacguacaacaaaauauugacuauaucgcuaaacaaaacaaaaaauauaaagcacauucuuacaguuuuguuucaacgauugauccaacgaguuuucuucguuuacaacuaaaauauucaaaagguuuuagagcaccaacuucagaugaaauguauuucaccuuuaaacacccugauuucacuauuuugccaaauacuaaucuaaaaccagagauagcaaaaacaaaagaaauugcuuuuacauuacauaaugaugauugggguuuuaucuccacaagucuauuuaaaaccaacuauaaaaacuuuaucgaccuaauauuuaaaggagaaaaagauuuuaaauuaguuagcggagguagcacauuaccauuuucucuuuaucaaaauauuaauagagauagugcgguaguaaaaggaauagaaauuaauucaaaaguauuccuugguaaaauggcaaaauuuauggauggauuuaaccuaagcuauaaauauaccuaucaaaaaggaagaauggauggcaauauuccuaugaaugcaauucagccuaaaacgaugguguauggcuuaggcuaugaccacccaagccaaaaauuuggauuuaauuucuacacuacccacguagcaaguaaaaauccagaagauacuuaugauauuuaugcgaaagauaaaaaccaaaccaacacuagcauaaaauggcgcaguaaaucuuauacuauucuagauuuaauuggauaugugcaaccaauuaaaaauuuaaccauaagagccggcguauauaaucuuacaaaccguaaauacaucacuugggauucugcgcguucaauucguucauuugguacaaguaauguuauagaucaaaagacaggucaaggcauuaaccgcuucuacgcaccagguagaaauuauaagaugucaguucaauuugaguuuuaa

SEQ ID NO: 87 - RNA, 4179 nt, Haemophilus influenzae
augaaaaaaacuguauuucgucugaauuuuuuaaccgcuugcauuucauuagggauaguaucgcaagcgugggcaggucacacuuauuuugggauugacuaccaauauuaucgugauuuugcugagaauaaagggaaguuugcagucggugcgaaagauauugagguuuacaacaaaaaaggggaguuggucggcaaaucaaugacaaaagcuccgaugauugauuuugcaucuguugcucguagugguuaugcugcauuaguaggcgaucaauauauuguaaguguugcucacaaugggggauacacuaaugcugauuuuggugcggaaggacaaaaucccgaucaacaucguuuuacuuaucaaauaguuaaacguaauaauuauaaaccugaucagaagcaucauuauaauggggauuaccauaugccucguuuacauaaauuuguaacugaaaguucuccuuuugauaugacaggaaguaugaagggacagaccuaugcagacaaagaaaaauacccgaugcgugugcggauugguucuggugaucaauauuggagaaaugaacaagauaaaggaacucaaguagcugaagcuuauggauaucuuauugcggggaauacccauaugcaaacuggugcaggaaauggagauguuaguuuaaguggcgauguacguaaaggaaaugauuauggaccuaugccgacagcaggaucauuuggagacagugggucaccuauguuuauuuaugaugcuaccgagaaaaaauggcuuauaaaugguguauugcguacuggaaacccauccuuagguagagagaauacuuuucagcuaguccguaaaaauuauuuugaugaaauuuucucugcuaaucuuaaaacgaccauauuugauuucagaacuccuaaaaaucaacaaaaucagcacuacacauuucauucaaaaaaugaugggucaggcacuauuacaucuggagcugguaaaauuuauaaagucaaacuugcuaauuacaauuuaucuaugaaaggcacggagccugucuauacuaauggugguguaaacaauuauucucccgcauuaagucacggcgaaaaccuuuauuuuauugauuacggaaaaggggugcuaacuuuuaguaauaauaucgaucaaggugcaggcgguuuguauuuugagggagauuuuauaguuucgccuacccauaaugaaacguggaaaggggcggggauaucuaucagugaugaaagcaccguaauuuggaaagucaauggcguagaaaaugaucgccuuucuaaaauugguaaaggaacguuacauauucaagcaaaagguaaaaacuuaggcucaauuagcguuggcgaugguaaaguaaucuuagaacaacaagaggacgaucaacauaaaaaacaagccuuuagugaaaucggauugguuagcggcagagggacuguccaauuaaacgaugauaaacaauuugauaccgauaaauuuuauuucggcuuucgugguggucgcuuagaucuuaaugggcauucauuaaccuuuaaacguauccaaaauacggacgagggggcgaugauugugaaccauaauacaacugaagucgcuaaugucacuauuacugggaacgaaagcauuguucuaccuaauggaaauaauauuaauaaacuuaauuacagcaaagaaauugccuacaacgguugguuuggcgaaacagaugaaaauaaacacaauggacgauuaaaccuuauuuauaaaccaaccacagaagaucguacuuuacuacuuucagguggcacaaacuuaaaaggcgauauuacucaagaaggcggcacuuuaguguuuaguggucgcccaacgccacacgcuuacaaucauuuaaaucgcccaaacgagcuugggcgaccucaaggcgaagugguuauugaugacgauuggaucaaccgcacauuuaaagcugaaaacuuccaaauuaaaggcggaagugcggugguuucucgcaauguuucuucaauugagggaaauuggacaaucagcaauaacgccaacgccacauuugguguugugccaaaucaacaaaauaccauuugcacgcguucagauuggacaggcuuaacgacuuguaaaacagagaauuuaaccgacacaaaaguuauuaauucuauaccaacaacacaaaucaauggcucuauuaauuuaacugauaaugcaacggcgaauguuaaagguuuagcaaaacuuaauggcaaugucacuuuaacaaaucacagccaauuuacauuaagcaacaaugcuacccaaauaggcaauauccgacuuuccgacaauucaacugcaacgguggauaaugcaaacuugaacgguaaugugcauuuaacgaauucugcucaauuuuauuuaaaaaacagccaucuuucgcaccaaauucagggcgacaaaggcacaacagugacguuggaaaaugcgacuuggacaaugccuagcgauaccacauugcagaauuuaacacuaaauaacaguacuguuacguuaaauucagcuuauucagcuaguccaaacaauacgccacgccgucgucgccguucauuagagacggaaacgacgccaacaucggcagaacaucguuucaacacauugacaguaaaugguaaauugagcgggcaaggcacauuccaauuuacuucaucuuuauuuggcuauaaaagcgauaaauuaaaauuauccaaugacgcugagggcgauuacauauuaucuguucgcaacacaggcaaagaacccgaaacccuugagcaauuaacuuugguugaaagcaaagauaaucaaccguuaucagacaaacucacauucacguuagaaaaugaccacguugaugcgggugcauuacguuauguauuagugaagaauaauggcgaauuccgcuugcauaauccaauaaaagagcaggaauugcacaaugauuuaguaagagcagagcaagcagaacaaacauuagaagccaaacaaguugaaccgacugcugaaacacaaacaaguaaggcaaaagugcgguuaagaagagcgguguugccugauacccugccugcucaaagccuguuaaacgcauuagaagccaaacaagcugaaccgaaugcuaaaacacaaaaaaguaaggcaaaaacaaaaaaagcgcggucaaaaagagcauugagagcgguguuuucugaucccccgccugaucaaagccaguuagacguauuaaaagccgcacuugagguuauuaaugcccaaccgcaagugggaaaagaacgucaagcucaagagaaacaaggcaaacaaaaagacuugaucagccguuacucaaauagugcguuaucggaguugucugcaacaguaaauaguaugcuuuccguucaagaugaauuggaucgucuuuuuguagaucaagcacaaucugcuguguggacaaauaucgcacaggauaaaagacgcuaugauucugaugcguuccgugcuuaucagcagaaaacgaacuugcgucaaauuggggugcaaaaagccuuagauaauggacgaauuggggcgguuuucucgcauagccguucagauaauaccuuugacgaacagguuaaaaaucacgcgacauuaacgaugaugucggguuucgcccaauaucaauggggcgauuuacaauuugguguaaacgugggcgcgggaauuagugcgaguaaaauggcugaagaacaaagccgaaaaauucaucgaaaagcgauaaauuauggugugaaugcaaguuaucaguuccguuuagggcaauuggguauucagccuuauuuggguguuaaucgauauuuuauugaacgugaaaauuaucaaucugaagacgugaaagugcaaacaccgagccuugcauuuaaucgcuauaaugcuggcauucgaguugauuauacguuuaccccgacagauaauaucagcguuaagccuuauuucuucguuaauuauguugauguuucaaacgcuaacguacaaaccacuguaaauagcacgauguugcaacaaucauuugggcguuauuggcaaaaagaagugggauuaaaggcagaaauuuuacauuuccaacuuuccgcuuuuaucucaaaaucucaagguucacaacucggcaaacagcaaaaugugggcgugaaauugggguaucguugguaa

SEQ ID NO: 88 - RNA, 4479 nt, Haemophilus influenzae
augaacaagauauaucgucucaaauucagcaaacgccugaaugcuuugguugcugugucugaauugacacgggguugugaccauuccacagaaaaaggcagugaaaaaccuguucguacgaaaguacgccacuuggcguuaaagccacuuuccgcuauauugcuaucuuugggcauggcauccauuccgcaaucuguuuuagcgagcgguuuacagggaaugagcgucguacacgguacagcaaccaugcaaguagacggcaauaaaaccacuauccguaauagcgucaaugcuaucaucaauuggaaacaauuuaacaucgaccaaaaugaaauggugcaguuuuuacaagaaagcagcaacucugccguuuucaaccguguuacaucugaccaaaucucccaauuaaaagggauuuuagauucuaacggacaagucuuuuuaaucaacccaaaugguaucacaauagguaaagacgcaauuauuaacacuaauggcuuuacggcuucuacgcuagauauuucuaacgaaaacaucaaagcgcguaauuucacccuugagcaaaccaaggauaaagcgcucgcugaaaucgugaaucacgguuuaauuaccguugguaaagacgguagcguaaaccuuauuggcggcaaaguaaaaaaugaggguguaauuagcguaaauggcgguagcauuucuuuacuugcagggcaaaaaaucaccaucagcgauauaauaaacccaaccauuacuuacagcauugccgcaccugaaaaugaagcaaucaaucuaggugauaucuuugcuaaaggugguaauaucaauguccgcgcugcaaacauucgaaaccaagguaaacuuucugccgauucuguaagcaaagauaaaagcggcaacauuguucuuuccgcuaaagagggcgaagcggaaauuggcggcgugauuuccgcucaaaaucaacaagccaaaggcgguaaguugaugauuacaggugauaaagucacauuaaaaacaggcgcaguuaucgaccuuucagguaaagaagggggagagaccuaccuuggcggcgacgagcgcggcgaagguaaaaacggcauucaauuagcaaagaaaaccucuuuagaaaaaggcucaacaauuaauguaucaggcaaagaaaaaggcgggcgcgcuauuguauggggcgauauugcguuaauugacggcaauauuaaugcucaagguaaagauaucgcuaaaacuggugguuuuguggaaacgucggggcauuauuuaucuauugguaaugaugcggcuguugaggcgaaagaaugguuguuggauccggauaauguuaccauuagcaaugguaacgaugaucaaucucaauuaaaggacgauagaggcgauucaccuaauaaaauucuagcagauaauaaacauacuguuaauaacaaaacauuaucuacagcuuuagcuaaagguauuggaguuaauauuucugcuaaaaagaaaguaaauguuacugcagauauuaauguucauaauggaacauuaacguuacacucagaacaagguggaguugaaauuaacggugauauuaccucagaacaaaaugguaauuuaaccauuaaagcagguagcuggguugauguucacaaaaauaucacgauugguaugggguuuuuaaauauuaccgcuggggguucuguagcuuuugaaaaggcagggggugauaaaggccgugcugcaagugaugcuaaaauuguugcucaggguguaauuacugcaggcaguggucaagauuuccguuuuaauaauguaucacuaaauggaacugguagaggguugaaauucauuacagcuaaaggaaauaaagguaauuucucagccaaauuugaugguguguuaaauauuucagguaauauuucaauuaaucacacugcuaauaaucaauuaucuuauuuucaucgccaggguuauacauauuggaauuugacgcagcuuaauguugauucugauucuaguuuuucuuuaacaucaauaaaagacgcgauuaaaguugggggauaugacaaugcuaaagacaaaaaaaacacaggagggauugguuuuacacgagauaccauuuucaauguaaaacaaggugcuagaguugauauuuccuacacccuaccgauaucuccaguuaaaaauucaagaauugcugcugucaauuuugacggaaauauuacuguuaaaggaggaggaguuguuaauuuaaaauuuaaugcgcuuagcaauaacuacaaaacuccaggggugaacauuuccucuagauuuauuaauguuaccgaagguucgcagcuaaacauuacugguaguaugccaucuacaaccuuauuuaauguagcaaaugauuuaaucauaaacgcaaccaauucauuuguauccauuaaagaaauugaagguacagauacucaucuugauacagguuuaaagguaaacgguaauguuaccauuaaaggagggaaugucacccuagguucaaauaaagcuaaaaccaaauuugauaaaaauguaacuguugaaaagggugcaaaucuuacucuugcgucagcaaacuuuggaaaucauaagggugcuuuaacuguagcuggaaauaucaauacucaagguaagcuuguugcaacuggcgauacuauugauguaucuggugauuuuacuguuggaaacgaugcuacuuuuaaugguaauacuaauaacaaccuaaacauuaccggcaauuuuaccaacaacggcaccuccauaauugauguaaaaaaaggggcggcaaaacuagguaauauuaccaaugaagguaguuuaaauauuacuacucacgcuaacacuaaucaaaaaaccauuauuaccggaaauauaaccaauaaaaaaggcgacuuaaacaucagggauaauaaaaauaaugccgaaauccaaauuggcggcaauaucucgcaaaaagaaggcaaucucacaauuucuucugauaaagucaauauuaccaagcagauaacaaucaaagcaggcguuaauggggagaauucugauucaggcacagaaaacaaugccaaucuaaccauuaaaaccaaaacgcuagaauuaacaaacaaccuaaacauuucaggcuuucauaaagcagaaauuacagcuaaagauaacagugauuuaauuauuggcaaggcuagcagugauagcgguaaugcuggugcucaaaaaguaaucuuugacaagguuaaagauucaaaaaucucagcuggcaaucacaauguaacacuaaauagcgaaguggaaacgucuaaugguaauaguaaugcugcuggcgauaguaaugguaacaacgcugguuuaacuauuuccgcaaaagauguggcaguaaauaauaacaucacuucucacaagacaauaaauaucucugccacaacaggaaauguaacaaccaaagaaggcacaaccauuaaugcgaccacagguggcguggaaguaaccgcuaaaacaggugacauuaaagguggaauugaguccaagucuggcggaguaacacuuacugcaaccggagauacucuugcuguagguaauauuucggguaacacuguuaguguuacugcaaauagcgguacauuaaccaccaaagcagacucuacaaucaaaggaaccgggagcguaaccaccuuaagucaaucaggcgauaucggcgguacgauuucugguaagacaguaaguguuacagcaaccaccgacaguuuaacuguuaaagguggcgcaaaaauuaaugcgacagaaggaacugcaaccuuaacugcaucaucgggcaaauuaaccaccgaggcuagcucaagcaucacuucagccaagggucagguagaccuuucagcucgggauggcaauauuggaggaaguauuaaugcugcuaaugugacauuaaauaccacaggcacuuuaacuaccgugaaggguucaagcauuaacgcaaacagcggcaccuugguuauuaacgcagaagacgcuaagcuagaugguacggcaucaggugaccgcacaguaguaaaugcaacuaacgcaagcggcucugguagcguaacugcgguaaccucaagcagcguaaacaucacuggagauuuaagcacaauaaacggauuaaacaucauuucgaaaaaugguaaaaacacuguaguguuaaaaggugcugaaauugaugugaaauacauucagccagguguagcaagcgcagaagaaguaauugaagcgaagcgugcccuugagaagguaaaagauuuaucugaugaagaaagagaaacgcuagccaaacuugguguaagugccguacguuuuguugagccaaauaaugccauuacgguuaauacgcagaacgaguuuacaaccagaccaucaagucaagugacaauuucugaagguaaggcguguuucucaaguggugauggcgcagcaguauguaccaauguugcugacgauggacagcaguag

SEQ ID NO: 89 - RNA, 4629 nt, Haemophilus influenzae
augaacaagauauaucgucucaaauucagcaaacgccugaaugcuuugguugcugugucugaauugacacgggguugugaccauuccacagaaaaaggcagugaaaaaccuguucguacaaaaguacgccacuuagcguuaaagccacuuuccgcuauauugcuaucuuugggcauggcauccauuccgcaaucuguuuuagcgagcgguuuacagggaaugagcgucguacacgguacagcaaccaugcaaguagacggcaauaaaaccaccauccgcaauagugucaaugcuaucauuaauuggaaacaauuuaacauugaccaaaaugaaauggugcaguuuuuacaagaaaacaacaacuccgccguauucaaccguguuacaucugaccagauuucccaauuaaaagggauuuuagauucuaacggacaggucuuuuuaaucaacccaaaugguaucacaauagguaaagaagcaauuauuaacacuaauggcuuuacugcuucuacgcuagacauuucuaacgaaaacaucaaggcgcguaauuucacccuugaacaaaccaaggauaaagcacucgcugaaaucgugaaucacgguuuaauuaccguugguaaagacgguagcguaaaccuuauuggcggcaaaguaaaaaaugaggguguaauuagcguaaauggcgguagcauuucuuuacuugcagggcaaaaaauaaccaucagcgacauaauaaacccaaccauuacuuacagcauugcugccccugaaaaugaagcaaucaaucugggcgauauuuuugccaaaggugguaacaucaauguccgcgccgccacuauucgcaauaaagguaaacuuucugccgauucuguaagcaaagauaaaagcggcaacauuauucuuuccgccaaagagggugaagcagaaauuagcggugugauuuccgcucaaaaucagcaagccaaaggugguaaguugaugauuacaggcgauaaaguuacauuaaaaacgggugcaguuaucgaccuuucagguaaagaagggggagagaccuaucuuggcggcgacgagcgcggcgaagguaaaaacggcauucaauuagcaaagaaaaccucuuuagaaaaaggcucaacaauuaauguaucagguaaagaaaaaggcgggcgcgcuauuguauggggcgauauugcguuaauugacggcaauauuaaugcucaagguagcgauaucgcuaaaacugguggcuuuguggagacgucggggcauuacuuauccauugacagcaaugcaauuguuaaaacaaaagaguggcuacuggacccugauaauguaacaauugaagcuccuagccuuucucgcgcugacacugacauuaguucagaauuuccaaucggugacgguacugaaaacagcccuaaaaaaaacgccgacaaaacaauacugaccaaugaaaccauuucaaauuuucugcaaaaugccaaagugaugaacauaacggcaaagagaaaacuuaccguuaauagcucaaucaguaucggaaguagaucccacuuaauucuccauagugaaggucagggggacggaggcguucagauugauggagauauuacuucugaaggcggaaauuuaaccauuaacuccggcggauggguugacguucauaaaaauauuacgcuugguacggguuuuuuaaauauuaccgcuggggguucuguagcuuuugagaaagggggcaauaacgcacguaacgcaacagaugcucaaauuaccgcacaagggacgauaaccgucaauaaagaugauaaacaauuuagauucaauaauguaucuauuaacgggacgggcgaggguuuaaaguuuauugcaaaucaaaauaauuucacucauaaauuugauggcgaaauuaacauaucuggaauaguaacaauuaaccaaaccacgaaaaaagaugcuaaauacuggcaugcaucaaaagacucuuacuggaauguuucuucucuuacuuugaaugacgaugcaaaauuuaccuuuauaaaauucguugauagcggcucaaauucccaagauuugaggucagcacguagacguuuugcaggcguacauuuuaacggcaccggaggcaaaacaaacuucaacaucggagcuaacgcaaaagccuuauuuaaauuaaaaccaaacgccgcuacagacccaaaaaaagaauuaccuauuacuuuuaacgccaacauuacagcuaccgguagcagugauagcucugugauguuugacauacacgccaaucuuaccucuagagcugccagcauaaacauggauucaauuaacauuaccggcgggcuugacuuuuccauaacaucccauaaucgcaauaguaaugcuuuugaaaucaaaaaagacuuaacuauaaaugcaacuaauucaaaauuuagucuuaaacaaacgaaagauuuauuugaaaaucaauacacaggagacgccauuaauucaacucguaaucuaacuauucuuggcggcaaugucacucugggcggggagaauucaagcaguaacauuacaggaaauaucaccaucgcugccgaagcaaaugucacauuacaagcuuacgccgacaauagcaucaaaggucauaagaaaaaaacucuaacucuugguaauguguccaccucuggaaacuuaagcuuaacuggcucaaagguggagguaaaaggcgaucuugccguuuuaaacggugccacuuuuaaaggagaaaccaaugacagccuaaacauuaccggcaccuuuaccaacaacgguaccgccgacauuaauauaaaacgaggggugguaaacauccaaggugauauuaccaauaaaggugguuuaaacauuaccacuaaugcccaaaaaaaucaaaaaaccauuauuaacggaaauauaaccaacaaaaaaggcaacuuaaauauuacgaauaaugguaaugauacugaaauccaaauuggcggcaauaucucacaaaaagaaggcaaucucacaauuucuucugacaaaguaaauaucaccaagcagauaacaauuaaagcaggcguugaugagaaggauucuaguucaagcacagcaagugaugccaaucuaaccauuaaaaccaaagaguuaaaauugguagaagaccuaaacauuucaggcuuuaauaaagcagaaauuacagcuaaagaugguagugauuuaacuauugguaacaccaauagugcugaugguacuaaugccaaaaaaguaaccuuuaaccagguuaaagauucaaaaaucucugcuaacgaucacaaugugacacuaaauagcaaaguggaaacaucugguaauacugacaacacuggagacggcaguggcaauaaugccggcuuaacuauugccgcgaaaaauguagaaguaaaaaacaacauuacuucuaacaaaacaguaaauaucaccgcgucagaaaaacuuaccaccaaagcggaugcaaccauuaaugcaaccacugguaacguagaagugacagccaaaacaggugauauuaaaggugaagucaaauccacuuccgguaauguaaauauuacagcaaacggcgacacgcuuaauguaaguaauguuucaggcaaugcuguuaccaucacugcagauaagggcaaauuaaccacccaagaaagcucuacgauuagcggaacugagaguguaaccaccucaagccaaucaggcgauauuggcggugcaauuucugguaacacgguaagcguuaaagcaaccaaugauuuaauuaccaaagcaaauucaaaaauugaagcaaaaacaggcgaggcuaauguaacaagugcaacagguauaauuggcggcacaaucucuggcaauacaguaaauguuacagcaaauacugguaguuuaacuauuaaaggcggugcaaaaguugaugcgacaaauggagcugcaaccuuaacugcagaaucaggcaaauuaaccacucaagcaggcucuacuauuaccucaaacaauggccagacaacucuuacagccaaggauggcaguaucgcaggaaguauugaugccgcuaaugugacauuaaauaccacaggcacuuuaacuaccguaguggguucaagcauuaacgcaaacgaaggcaccuuaguuauuaacgcacaagacgccacacuaaauggugaugcaucaggcgaccguacagaagugaaugcagucaacgcaagcggcucugguagcguaacugcgguaaccucaagcagcguaaacaucacuggagauuuaagcacaauaaacggauuaaacaucauuucgaaaaaugguaaaaacacuguaguguuaaaaggugcugaaauugaugugaaauacauucagccagguguagcaagcgcagaagaaguaauugaagcgaagcgugcccuugagaagguaaaagauuuaucugaugaagaaagagaaacgcuagccaaacuugguguaagugccguacguuuuguugagccaaauaaugccauuacgguuaauacgcagaacgaguuuacaaccagaccaucaagucaagugacaauuucugaagguaaggcguguuucucaaguggugauggcgcagcaguauguaccaauguugcugacgauggacagcaguag

SEQ ID NO: 90 - RNA, 5385 nt, Haemophilus influenzae
augcuaaauaaaaaauucaaacucaauuuuauugcgcuuacugucgccuacgcauuaaccccuuauacagaagccgcguuagugagaaacgauguggauuaucaaauauuucgugauuuugcagagaauaaagggaaguuuucuguuggugcaacaaauguggaagugagagauaacaaaaauaauaauuugggcagugcgcuaccuaaagauauuccgaugauugauuuuagugcuguggauguagauaagagaauugccacguugguaaauccacaauauguuguggggguaaaacacguugguaacggugugggugaacuacauuuugggaauuuaaaugggaauuggaacccuaaauuuggaaauucuauucagcaucgagauguaucuugggaggaaaacagauauuacacuguugaaaaaaauaauuuuucuucugaauuaaacggaaaaacacaaaauaacgaaaaagauaaacaguauacaucuaauaaaaaagauguuccuuccgaauuauauggacaagccuuaguuaaagaacagcaaaaccaaaaacgccgugaagauuauuauaugccacgccuugauaaauuuguuaccgaaguugcaccgauagaggcuucaacuacaaguagugaugcuggaacauauaaugaucagaauaaauauccugcuuuugugagauuaggaaguggcucucaauuuaucuauaaaaaaggaagccauuaugaauuaauuuuagaagagaaaaacgagaaaaaggaaauaauacacagaugggauguagguggagauaaucuuaaauuaguugguaacgcuuauaccuaugguauugcaggcacgccuuauaaagugaaucauacagacgauggacucauuggguuuggugauucaaccgaagaucauaacgauccaaaagagauauuaucucguaaaccgcuuacuaauuaugcugucuugggagauaguggcucaccguuauuuguuuaugauaaaucuaaagagaaauggcuuuuccuuggugcuuaugauuuuuggggaggguauaagaaaaaaucuuggcaagaauggaauauuuauaaaccacaauuugcugaaaauauucucaaaaaagacagugcugguuuacugaaagguaauacgcaauauaacuggacaagcaaagguaauacaagcuugauaucuggaacaucugaaucauuaaguguugauuuaguggauaauaaaaaucucaaucaugggaaaaauguuacguuugaaggaaguggaaaccuuaccuuaaauaauaauaucgaucaaggugcaggcggauuguucuuugaaggugauuaugaaguuaaaggcacuucagaaaauacgacuuggaaaggggcugguauaucuguugcugaaggaaaaacaguaaaguggaaagugcauaacccucaauuugaucguuuagcuaaaauugguaaaggaaagcuaauuguugaaggaagaggagauaauaaagguucgcuaaaagugggcgauggcacaguuguuuuaaaacaacaaacaacuacaggacaacacgcuuuugcuucuguaggaauuguaaguggucgcucaacuguuguacuuaaugaugauaaccaaguagauccaaauucaauuuacuuugguuuuagaggcggucgauuagaugccaauggcaauaaucucacuuuugaacauauccguaauauugaugauggugcaagacuaguuaaucauaauaugacuaaugccucaaauauaacgauuacuggagcaggauuaauuacaaauccaagucaggucacuauauauacaccagcaauaaccgccgaugaugauaauuauuacuaugugccaucuauuccaagaggaaaagaucuuuacuuuaguaauaccuguuauaaauacuaugcguuaaagcaagguggaagcccuacugcagaaaugccuugcuauagcucugaaaaaucagaugcuaauugggaauucaugggcgacaaucaaaaugaugcacaaaagaaagccaugguguacaucaauaacagacguaugaauggcuuuaaugguuauuuuggcgaggaagcaacuaaggcagaucaaaacgguaaauuaaauguuaccuuuaguggcaaaagugaucaaaaucgcuuuuuauugacuggcggaacaaaccuuaacggugaauuaaaaguugaaaaagguacauuauuccuuucuggcagaccaacaccacacgcaagagauauugcaaauauuucuucaacagaaaaagauaaacacuuugcugaaaauaaugaaguggugguagaagacgacuggauuaaccgcacuuuuaaagcaacaaauauuaauguaaccaauaacgcaacccuuuauucaggucgcaauguugaaaguauuacuucaaauaucacagccucuaauaaagcaaaagugcauaucggcuauaaagcaggcgauaccguuuguguacguucugacuauacgggcuaugugacuugccauaaugacacuuuaucuacaaaagcacucaauaguuuuaauccaacuaauuugcgcgguaauguaaauuuaaccgaaagugcaaacuuuaccuuaggcaaagcuaacuuauuuggcacaauuaacagcacggaaaauagccaaguuaauuuaaaagaaaacagccauugguauuuaacaggaaauagugauguucaucaauuagaucuagcaaaugggcauauucauuuaaauaaugucucagaugcaacaaaagaaacaaaauaccacacgcucaauauuagcaauuuaucagguaaugguucuuucuauuauuggguugauuuuaccaaaaaucagggcgacaaaguuguuguaaccaaaucagccaaagguaccuuuacauuacaaguggcaaauaaaacaggcgagccaaaucauaaugaacuuacgcuuuuugaugcgucaaaugcaacugaaagaaguggcuuaaauguuucacuggcuaauggcaaaguugaucgaggugcuuggagcuauacuuugaaagaaaauaguggacguuauuacuugcauaacccagaaguagaaagaagaaaucaaacugucgauacgccaaguauugcaacagcuaauaauaugcaagcugauguaccuagcguaucaaauaaccaugaagaaaccgcucguguugaagcaccaauuccacugccagcaccaccugcaccugcuacuggaucagcaauggcaaacgagcaaccagaaacucguccugcagaaaccguucaaccuacaauggaagauacaaauacuacucacccaucgggaucugaaccacaagcugauacaacacaggcugaugauccaaauucagaaaguguuccaucagagacaauugaaaaaguggcugaaaauaguccgcaagaaagugaaacaguagcgaaaaacgagcaaaaagcaaccgagacaacagcucaaaaugaugaaguugcaaaagaagcuaaaccaacuguggaagcuaacacucaaacaaaugaacucgcccaaaauggaagcgaaaccgaggaaacucaagaggcugaaacugcucgucagucugaaauaaauucuacugaagaaacaguaguagaagaugauccuacaaucagugaaccaaagagcagaccuagaagaaguauuagcucgucuucuaauaauauaaaucuagcaggaacagaagauacugcuaaaguagaaacagagaaaacucaagaagccccucaaguggcuuuucaagcgucuccaaaacaagaagaaccagaaauggcuaaacagcaagaacagccuaaaacuguucaaucgcaagcacagccagaaacuaccacacagcaagcagagccugcucgugaaaauguuucgacuguuaauaauguaaaagaagcucaaccgcaagcaaaaccuacaacuguugcugcgaaagagacaacugcaucaaacagugaacagaaagaaacugcucaaccuguagcuaauccaaaaacagcugaaaauaaagcagaaaauccucaaucaacagaaacgacagaugaaaauauucaucagccugaagcccacacugcuguugcuucaacagaaguuguuacaccugaaaaugcuacaacaccuauuaaaccuguugagaauaaaacuacagaagcugaacaaccugugacagaaaccacaacaguaagcacugaaaacccuguagugaaaaauccagagaauacaacaccugcuacaacucaaucuacgguuaauucagaggcaguucaaucagaaacagcgacaacagaagcuguuguuagccagucuaaagugacuucugcugaagaaacaacaguagcuucuacucaagaaacaacaguggauaauucaggaucuacuccacaaccacgaagcagaagaacucguagaucagcucagaauaguuacgagccaguggaacuacauacugaaaaugcggagaauccgcaaucgggaaaugauguggcuacacaacuaguauugcgcgaucucacaaguacaaacaccaaugcgguaauuucugaugccauggcaaaagcacaauuuguugcauuaaauguagggaaagccguuucucaacauauuagccaguuagaaaugaauaacgaggggcaauauaacguuuggguaucuaauacuucaaugaaagaaaauuauuccucaagucaauaucgucauuuuaguucuaaaagugcgcaaacucaacuugguugggaucaaacaaucucaagcaauguucaguuaggugguguguuuacuuauguucgcaauaguaacaacuuugauaaggcaagcaguaaaaauaccuuagcacaagcaaaucucuauucuaaauauuauauggauaaucauugguauuuagcgguggauuuaggcuaugguaauuuccaaagcaacuuacaaacuaaucauaaugcgaaauuugcucgccauacugcacaauuugguuuaaccgcaggcaaagcauuuaaucuuggcaauuuugcuguuaaaccaacuguagggguucguuauaguuacuuaucaaacgcuaauuuugcauuagcuaaagaucgcauuaaaguaaauccaauaucugucaaaacagccuuugcucaaguugauuuaaguuauacuuaucacuuaggcgaguuuuccauuacgccaauuuugucugcucgauaugaugcaaaucaaggcagcggcaaaauuaauguagauagauaugauuuugcuuacaauguggaaaaccaacagcaauauaacgcaggacuuaaauugaaauaucauaaugugaaauuaagucuaauuggcggauuaacaaaagcaaaacaagcggaaaaacaaaaaacugcagaagugaaacuaaguuuuaguuuuuaa

SEQ ID NO: 91 - protein, 1040 aa, Haemophilus influenzae
ALVRNDVDYQIFRDFAENKGKFSVGATNVEVRDNKNNNLGSALPKDIPMIDFSAVDVDKRIATLVNPQYVVGVKHVGNGVGELHFGNLNGNWNPKFGNSIQHRDVSWEENRYYTVEKNNFSSELNGKTQNNEKDKQYTSNKKDVPSELYGQALVKEQQNQKRREDYYMPRLDKFVTEVAPIEASTTSSDAGTYNDQNKYPAFVRLGSGSQFIYKKGSHYELILEEKNEKKEIIHRWDVGGDNLKLVGNAYTYGIAGTPYKVNHTDDGLIGFGDSTEDHNDPKEILSRKPLTNYAVLGDSGSPLFVYDKSKEKWLFLGAYDFWGGYKKKSWQEWNIYKPQFAENILKKDSAGLLKGNTQYNWTSKGNTSLISGTSESLSVDLVDNKNLNHGKNVTFEGSGNLTLNNNIDQGAGGLFFEGDYEVKGTSENTTWKGAGISVAEGKTVKWKVHNPQFDRLAKIGKGKLIVEGRGDNKGSLKVGDGTVVLKQQTTTGQHAFASVGIVSGRSTVVLNDDNQVDPNSIYFGFRGGRLDANGNNLTFEHIRNIDDGARLVNHNMTNASNITITGAGLITNPSQVTIYTPAITADDDNYYYVPSIPRGKDLYFSNTCYKYYALKQGGSPTAEMPCYSSEKSDANWEFMGDNQNDAQKKAMVYINNRRMNGFNGYFGEEATKADQNGKLNVTFSGKSDQNRFLLTGGTNLNGELKVEKGTLFLSGRPTPHARDIANISSTEKDKHFAENNEVVVEDDWINRTFKATNINVTNNATLYSGRNVESITSNITASNKAKVHIGYKAGDTVCVRSDYTGYVTCHNDTLSTKALNSFNPTNLRGNVNLTESANFTLGKANLFGTINSTENSQVNLKENSHWYLTGNSDVHQLDLANGHIHLNNVSDATKETKYHTLNISNLSGNGSFYYWVDFTKNQGDKVVVTKSAKGTFTLQVANKTGEPNHNELTLFDASNATERSGLNVSLANGKVDRGAWSYTLKENSGRYYLHNPEVERRNQTVDTPSIATANNMQADVPSVSNNHEETARVEAPIPLP

SEQ ID NO: 92 - protein, 729 aa, Haemophilus influenzae
APPAPATGSAMANEQPETRPAETVQPTMEDTNTTHPSGSEPQADTTQADDPNSESVPSETIEKVAENSPQESETVAKNEQKATETTAQNDEVAKEAKPTVEANTQTNELAQNGSETEETQEAETARQSEINSTEETVVEDDPTISEPKSRPRRSISSSSNNINLAGTEDTAKVETEKTQEAPQVAFQASPKQEEPEMAKQQEQPKTVQSQAQPETTTQQAEPARENVSTVNNVKEAQPQAKPTTVAAKETTASNSEQKETAQPVANPKTAENKAENPQSTETTDENIHQPEAHTAVASTEVVTPENATTPIKPVENKTTEAEQPVTETTTVSTENPVVKNPENTTPATTQSTVNSEAVQSETATTEAVVSQSKVTSAEETTVASTQETTVDNSGSTPQPRSRRTRRSAQNSYEPVELHTENAENPQSGNDVATQLVLRDLTSTNTNAVISDAMAKAQFVALNVGKAVSQHISQLEMNNEGQYNVWVSNTSMKENYSSSQYRHFSSKSAQTQLGWDQTISSNVQLGGVFTYVRNSNNFDKASSKNTLAQANLYSKYYMDNHWYLAVDLGYGNFQSNLQTNHNAKFARHTAQFGLTAGKAFNLGNFAVKPTVGVRYSYLSNANFALAKDRIKVNPISVKTAFAQVDLSYTYHLGEFSITPILSARYDANQGSGKINVDRYDFAYNVENQQQYNAGLKLKYHNVKLSLIGGLTKAKQAEKQKTAEVKLSFSF

SEQ ID NO: 93 - protein, 375 aa, Haemophilus influenzae
HTVEIMGGGEGEGDVIGAVIVDTGTAAQEWGRIQQQKKGQADKQKRPEPVVEEKPPEPNQEEIKHQQEVQRQEEIKHQQEVQRQEELKRQQEQQRQQEIKKQQEQARQEALEKQKQAEEARAKQAAEAAKLKADAEAKRLAAAAKQAEEEAKAKAAEIAAQKAKQEAETKAKLEAEAKAKAAAEAKAKAEAEAKAKAAAEAKAKAEAEAKAKAAAEAKAKADAEAKAKAAAEAKAAAEAKRKADQASLDDFFNGGDVGGGSASKGGNTNKGGTQGSGAALGSGDGGKVGDQYAGVIKKEIQRRFLKDPNFAGKVCRIKIQLGRDGTILGYQKISGSDDICSAALSAVARTKKVPAAPSDEIYEKYKSPIIDFDIR

SEQ ID NO: 94 - protein, 900 aa, Haemophilus influenzae
STQGLPQEGLPQEDTVVFGNVTIDKTTADKMTITQSSPTTQINWKSFDIGQNKEVEFKQPSPNAVAYNRVTGGNASQIQGKLTANGKVYLANPNGVIITKGAEINVAGLLATTKDLEQISENGNGNGNSYQFTRKTKDGQELKEGKVLNEGKIKAKDFVVLNGDEVINKEEINVTNGKVYLSSGDNFTFTLLPDSGISVALEDNAVRGIVKNEGIIKAEDITLNAKGRKEALDSLVMNNGVLEATKVSNKNGKVVLSADEIKLDDKSNIKGDMVSFVADVTSNKELKDNIKITSQTGSKVTSPKIDFKGKSVNINGNFGRDDSKAHYNDEHKTLKTEVNIDVPDDENIRIANIEDEDKDSTGSFIQTDALSSLLANNGKVTLKGNNVNISGRIHIDSFRGSDSLLKLTNKGHIKINNADIHSKGRLFFITSLQNEEDFKSNITITDSKINLGNGAMGLGRSVNESDYDNKYQKTEGSQRKKFNVKMSNVEFNQVDDVILAGGFEKVNLDKIVATGQTNFYIDGGVSRNRTNGEPWKYEYGVLDLDKRTQLSELDQGRRRWGYYYDLELDMNRAYLYRFDLFAAKNTRRSTIKGTEINISNSNINLKNGFVHLLAEKIKLDNSKIDITFDKDNSQDTLAQTNRLGMNGKVSMINSHIKIVGDEKSGISPTGTYATMFLIGELIGEKSSIFVKSHQGYTFKTDGDTKIAGKNSKEDLKITAINTGGRAAEEVLINGALGSADNDANIANMAFTIGDSANTKTTIENADITALAPNGGTAYLSSKDVEIEVKPNSDFTFFELPREKNLNQTKINGASTKLSERGFARLYDKINGVRASNLSAEQLNVTDSSEKIINTNLVSSLDVEKLVSVAVCDAGKGCEEQQFGDKGNNTKVSVGELEAEQ

SEQ ID NO: 95 - protein, 610 aa, Haemophilus influenzae
GGGSFDVDDVSNPSSSKPRYQDDTSSSRTKSNLEKLSIPSLGGGMKLVAQNLSGNKEPSFLNENGYISYFSSPSTIEDDVKNVKTENKIHTNPIGLEPNRALQDPNLQKYVYSGLYYIENWKDFSKLATEKKAYSGHYGYAFYYGNKTATDLPVSGVATYKGTWDFITATKYGQNYSLFSNARGQAYFRRSATRGDIDLENNSKNGDIGLISEFSADFGTKKLTGQLSYTKRKTDIQQYEKEKLYDIDAHIYSNRFRGKVTPTKSTSDEHPFTSEGTLEGGFYGPNAEELGGKFLARDKRVFGVFSAKETPETEKEKLSKETLIDGKLITFSTKTADATTSTTASTTADVKTDEKNFTTKDISSFGEADYLLIDNYPVPLFPEGDTDDFVTSKHHDIGNKTYKVEACCKNLSYVKFGMYYEDKEKKNTNQTGQYHQFLLGLRTPSSQIPVTGNVKYLGSWFGYIGDDKTSYSTTGNKQQDKNAPAEFDVNFDNKTLTGKLKRADSQNTVFNIEATFKNGSNAFEGKATANVVIDPKNTQATSKVNFTTTVNGAFYGPHATELGGYFTYNGNNPTATNSESSSTVPSPPNSPNARAAVVFGAKRQVEKTNK

SEQ ID NO: 96 - protein, 1367 aa, Haemophilus influenzae
GHTYFGIDYQYYRDFAENKGKFAVGAKDIEVYNKKGELVGKSMTKAPMIDFASVARSGYAALVGDQYIVSVAHNGGYTNADFGAEGQNPDQHRFTYQIVKRNNYKPDQKHHYNGDYHMPRLHKFVTESSPFDMTGSMKGQTYADKEKYPMRVRIGSGDQYWRNEQDKGTQVAEAYGYLIAGNTHMQTGAGNGDVSLSGDVRKGNDYGPMPTAGSFGDSGSPMFIYDATEKKWLINGVLRTGNPSLGRENTFQLVRKNYFDEIFSANLKTTIFDFRTPKNQQNQHYTFHSKNDGSGTITSGAGKIYKVKLANYNLSMKGTEPVYTNGGVNNYSPALSHGENLYFIDYGKGVLTFSNNIDQGAGGLYFEGDFIVSPTHNETWKGAGISISDESTVIWKVNGVENDRLSKIGKGTLHIQAKGKNLGSISVGDGKVILEQQEDDQHKKQAFSEIGLVSGRGTVQLNDDKQFDTDKFYFGFRGGRLDLNGHSLTFKRIQNTDEGAMIVNHNTTEVANVTITGNESIVLPNGNNINKLNYSKEIAYNGWFGETDENKHNGRLNLIYKPTTEDRTLLLSGGTNLKGDITQEGGTLVFSGRPTPHAYNHLNRPNELGRPQGEVVIDDDWINRTFKAENFQIKGGSAVVSRNVSSIEGNWTISNNANATFGVVPNQQNTICTRSDWTGLTTCKTENLTDTKVINSIPTTQINGSINLTDNATANVKGLAKLNGNVTLTNHSQFTLSNNATQIGNIRLSDNSTATVDNANLNGNVHLTNSAQFYLKNSHLSHQIQGDKGTTVTLENATWTMPSDTTLQNLTLNNSTVTLNSAYSASPNNTPRRRRRSLETETTPTSAEHRFNTLTVNGKLSGQGTFQFTSSLFGYKSDKLKLSNDAEGDYILSVRNTGKEPETLEQLTLVESKDNQPLSDKLTFTLENDHVDAGALRYVLVKNNGEFRLHNPIKEQELHNDLVRAEQAEQTLEAKQVEPTAETQTSKAKVRLRRAVLPDTLPAQSLLNALEAKQAEPNAKTQKSKAKTKKARSKRALRAVFSDPPPDQSQLDVLKAALEVINAQPQVGKERQAQEKQGKQKDLISRYSNSALSELSATVNSMLSVQDELDRLFVDQAQSAVWTNIAQDKRRYDSDAFRAYQQKTNLRQIGVQKALDNGRIGAVFSHSRSDNTFDEQVKNHATLTMMSGFAQYQWGDLQFGVNVGAGISASKMAEEQSRKIHRKAINYGVNASYQFRLGQLGIQPYLGVNRYFIERENYQSEDVKVQTPSLAFNRYNAGIRVDYTFTPTDNISVKPYFFVNYVDVSNANVQTTVNSTMLQQSFGRYWQKEVGLKAEILHFQLSAFISKSQGSQLGKQQNVGVKLGYRW

SEQ ID NO: 97 - protein, 975 aa, Haemophilus influenzae
GHTYFGIDYQYYRDFAENKGKFAVGAKDIEVYNKKGELVGKSMTKAPMIDFASVARSGYAALVGDQYIVSVAHNGGYTNADFGAEGQNPDQHRFTYQIVKRNNYKPDQKHHYNGDYHMPRLHKFVTESSPFDMTGSMKGQTYADKEKYPMRVRIGSGDQYWRNEQDKGTQVAEAYGYLIAGNTHMQTGAGNGDVSLSGDVRKGNDYGPMPTAGSFGDSGSPMFIYDATEKKWLINGVLRTGNPSLGRENTFQLVRKNYFDEIFSANLKTTIFDFRTPKNQQNQHYTFHSKNDGSGTITSGAGKIYKVKLANYNLSMKGTEPVYTNGGVNNYSPALSHGENLYFIDYGKGVLTFSNNIDQGAGGLYFEGDFIVSPTHNETWKGAGISISDESTVIWKVNGVENDRLSKIGKGTLHIQAKGKNLGSISVGDGKVILEQQEDDQHKKQAFSEIGLVSGRGTVQLNDDKQFDTDKFYFGFRGGRLDLNGHSLTFKRIQNTDEGAMIVNHNTTEVANVTITGNESIVLPNGNNINKLNYSKEIAYNGWFGETDENKHNGRLNLIYKPTTEDRTLLLSGGTNLKGDITQEGGTLVFSGRPTPHAYNHLNRPNELGRPQGEVVIDDDWINRTFKAENFQIKGGSAVVSRNVSSIEGNWTISNNANATFGVVPNQQNTICTRSDWTGLTTCKTENLTDTKVINSIPTTQINGSINLTDNATANVKGLAKLNGNVTLTNHSQFTLSNNATQIGNIRLSDNSTATVDNANLNGNVHLTNSAQFYLKNSHLSHQIQGDKGTTVTLENATWTMPSDTTLQNLTLNNSTVTLNSAYSASPNNTPRRRRRSLETETTPTSAEHRFNTLTVNGKLSGQGTFQFTSSLFGYKSDKLKLSNDAEGDYILSVRNTGKEPETLEQLTLVESKDNQPLSDKLTFTLENDHVDAGALRYVLVKNNGEFRLHNPIKEQELHNDLVRAEQAEQTLEAK

SEQ ID NO: 98 - protein, 392 aa, Haemophilus influenzae
QVEPTAETQTSKAKVRLRRAVLPDTLPAQSLLNALEAKQAEPNAKTQKSKAKTKKARSKRALRAVFSDPPPDQSQLDVLKAALEVINAQPQVGKERQAQEKQGKQKDLISRYSNSALSELSATVNSMLSVQDELDRLFVDQAQSAVWTNIAQDKRRYDSDAFRAYQQKTNLRQIGVQKALDNGRIGAVFSHSRSDNTFDEQVKNHATLTMMSGFAQYQWGDLQFGVNVGAGISASKMAEEQSRKIHRKAINYGVNASYQFRLGQLGIQPYLGVNRYFIERENYQSEDVKVQTPSLAFNRYNAGIRVDYTFTPTDNISVKPYFFVNYVDVSNANVQTTVNSTMLQQSFGRYWQKEVGLKAEILHFQLSAFISKSQGSQLGKQQNVGVKLGYRW

SEQ ID NO: 99 - protein, 1480 aa, Haemophilus influenzae
PQSVLASGLQGMSVVHGTATMQVDGNKTTIRNSVNAIINWKQFNIDQNEMVQFLQENNNSAVFNRVTSDQISQLKGILDSNGQVFLINPNGITIGKEAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAATIRNKGKLSADSVSKDKSGNIILSAKEGEAEISGVISAQNQQAKGGKLMITGDKVTLKTGAVIDLSGKEGGETYLGGDERGEGKNGIQLAKKTSLEKGSTINVSGKEKGGRAIVWGDIALIDGNINAQGSDIAKTGGFVETSGHYLSIDSNAIVKTKEWLLDPDNVTIEAPSLSRADTDISSEFPIGDGTENSPKKNADKTILTNETISNFLQNAKVMNITAKRKLTVNSSISIGSRSHLILHSEGQGDGGVQIDGDITSEGGNLTINSGGWVDVHKNITLGTGFLNITAGGSVAFEKGGNNARNATDAQITAQGTITVNKDDKQFRFNNVSINGTGEGLKFIANQNNFTHKFDGEINISGIVTINQTTKKDAKYWHASKDSYWNVSSLTLNDDAKFTFIKFVDSGSNSQDLRSARRRFAGVHFNGTGGKTNFNIGANAKALFKLKPNAATDPKKELPITFNANITATGSSDSSVMFDIHANLTSRAASINMDSINITGGLDFSITSHNRNSNAFEIKKDLTINATNSKFSLKQTKDLFENQYTGDAINSTRNLTILGGNVTLGGENSSSNITGNITIAAEANVTLQAYADNSIKGHKKKTLTLGNVSTSGNLSLTGSKVEVKGDLAVLNGATFKGETNDSLNITGTFTNNGTADINIKRGVVNIQGDITNKGGLNITTNAQKNQKTIINGNITNKKGNLNITNNGNDTEIQIGGNISQKEGNLTISSDKVNITKQITIKAGVDEKDSSSSTASDANLTIKTKELKLVEDLNISGFNKAEITAKDGSDLTIGNTNSADGTNAKKVTFNQVKDSKISANDHNVTLNSKVETSGNTDNTGDGSGNNAGLTIAAKNVEVKNNITSNKTVNITASEKLTTKADATINATTGNVEVTAKTGDIKGEVKSTSGNVNITANGDTLNVSNVSGNAVTITADKGKLTTQESSTISGTESVTTSSQSGDIGGAISGNTVSVKATNDLITKANSKIEAKTGEANVTSATGIIGGTISGNTVNVTANTGSLTIKGGAKVDATNGAATLTAESGKLTTQAGSTITSNNGQTTLTAKDGSIAGSIDAANVTLNTTGTLTTVVGSSINANEGTLVINAQDATLNGDASGDRTEVNAVNASGSGSVTAVTSSSVNITGDLSTINGLNIISKNGKNTVVLKGAEIDVKYIQPGVASAEEVIEAKRALEKVKDLSDEERETLAKLGVSAVRFVEPNNAITVNTQNEFTTRPSSQVTISEGKACFSSGDGAAVCTNVADDGQQ

SEQ ID NO: 100 - protein, 673 aa, Haemophilus influenzae
PQSVLASGLQGMSVVHGTATMQVDGNKTTIRNSVNAIINWKQFNIDQNEMVQFLQENNNSAVFNRVTSDQISQLKGILDSNGQVFLINPNGITIGKEAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAATIRNKGKLSADSVSKDKSGNIILSAKEGEAEISGVISAQNQQAKGGKLMITGDKVTLKTGAVIDLSGKEGGETYLGGDERGEGKNGIQLAKKTSLEKGSTINVSGKEKGGRAIVWGDIALIDGNINAQGSDIAKTGGFVETSGHYLSIDSNAIVKTKEWLLDPDNVTIEAPSLSRADTDISSEFPIGDGTENSPKKNADKTILTNETISNFLQNAKVMNITAKRKLTVNSSISIGSRSHLILHSEGQGDGGVQIDGDITSEGGNLTINSGGWVDVHKNITLGTGFLNITAGGSVAFEKGGNNARNATDAQITAQGTITVNKDDKQFRFNNVSINGTGEGLKFIANQNNFTHKFDGEINISGIVTINQTTKKDAKYWHASKDSYWNVSSLTLNDDAKFTFIKFVDSGSNSQDLRSARRRFAGVHFNGTGGKTNFNIGANAKALFKLKPNAATDPKKEL

SEQ ID NO: 101 - protein, 807 aa, Haemophilus influenzae
PITFNANITATGSSDSSVMFDIHANLTSRAASINMDSINITGGLDFSITSHNRNSNAFEIKKDLTINATNSKFSLKQTKDLFENQYTGDAINSTRNLTILGGNVTLGGENSSSNITGNITIAAEANVTLQAYADNSIKGHKKKTLTLGNVSTSGNLSLTGSKVEVKGDLAVLNGATFKGETNDSLNITGTFTNNGTADINIKRGVVNIQGDITNKGGLNITTNAQKNQKTIINGNITNKKGNLNITNNGNDTEIQIGGNISQKEGNLTISSDKVNITKQITIKAGVDEKDSSSSTASDANLTIKTKELKLVEDLNISGFNKAEITAKDGSDLTIGNTNSADGTNAKKVTFNQVKDSKISANDHNVTLNSKVETSGNTDNTGDGSGNNAGLTIAAKNVEVKNNITSNKTVNITASEKLTTKADATINATTGNVEVTAKTGDIKGEVKSTSGNVNITANGDTLNVSNVSGNAVTITADKGKLTTQESSTISGTESVTTSSQSGDIGGAISGNTVSVKATNDLITKANSKIEAKTGEANVTSATGIIGGTISGNTVNVTANTGSLTIKGGAKVDATNGAATLTAESGKLTTQAGSTITSNNGQTTLTAKDGSIAGSIDAANVTLNTTGTLTTVVGSSINANEGTLVINAQDATLNGDASGDRTEVNAVNASGSGSVTAVTSSSVNITGDLSTINGLNIISKNGKNTVVLKGAEIDVKYIQPGVASAEEVIEAKRALEKVKDLSDEERETLAKLGVSAVRFVEPNNAITVNTQNEFTTRPSSQVTISEGKACFSSGDGAAVCTNVADDGQQ

SEQ ID NO: 102 - protein, 1430 aa, Haemophilus influenzae
PQSVLASGLQGMSVVHGTATMQVDGNKTTIRNSVNAIINWKQFNIDQNEMVQFLQESSNSAVFNRVTSDQISQLKGILDSNGQVFLINPNGITIGKDAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAANIRNQGKLSADSVSKDKSGNIVLSAKEGEAEIGGVISAQNQQAKGGKLMITGDKVTLKTGAVIDLSGKEGGETYLGGDERGEGKNGIQLAKKTSLEKGSTINVSGKEKGGRAIVWGDIALIDGNINAQGKDIAKTGGFVETSGHYLSIGNDAAVEAKEWLLDPDNVTISNGNDDQSQLKDDRGDSPNKILADNKHTVNNKTLSTALAKGIGVNISAKKKVNVTADINVHNGTLTLHSEQGGVEINGDITSEQNGNLTIKAGSWVDVHKNITIGMGFLNITAGGSVAFEKAGGDKGRAASDAKIVAQGVITAGSGQDFRFNNVSLNGTGRGLKFITAKGNKGNFSAKFDGVLNISGNISINHTANNQLSYFHRQGYTYWNLTQLNVDSDSSFSLTSIKDAIKVGGYDNAKDKKNTGGIGFTRDTIFNVKQGARVDISYTLPISPVKNSRIAAVNFDGNITVKGGGVVNLKFNALSNNYKTPGVNISSRFINVTEGSQLNITGSMPSTTLFNVANDLIINATNSFVSIKEIEGTDTHLDTGLKVNGNVTIKGGNVTLGSNKAKTKFDKNVTVEKGANLTLASANFGNHKGALTVAGNINTQGKLVATGDTIDVSGDFTVGNDATFNGNTNNNLNITGNFTNNGTSIIDVKKGAAKLGNITNEGSLNITTHANTNQKTIITGNITNKKGDLNIRDNKNNAEIQIGGNISQKEGNLTISSDKVNITKQITIKAGVNGENSDSGTENNANLTIKTKTLELTNNLNISGFHKAEITAKDNSDLIIGKASSDSGNAGAQKVIFDKVKDSKISAGNHNVTLNSEVETSNGNSNAAGDSNGNNAGLTISAKDVAVNNNITSHKTINISATTGNVTTKEGTTINATTGGVEVTAKTGDIKGGIESKSGGVTLTATGDTLAVGNISGNTVSVTANSGTLTTKADSTIKGTGSVTTLSQSGDIGGTISGKTVSVTATTDSLTVKGGAKINATEGTATLTASSGKLTTEASSSITSAKGQVDLSARDGNIGGSINAANVTLNTTGTLTTVKGSSINANSGTLVINAEDAKLDGTASGDRTVVNATNASGSGSVTAVTSSSVNITGDLSTINGLNIISKNGKNTVVLKGAEIDVKYIQPGVASAEEVIEAKRALEKVKDLSDEERETLAKLGVSAVRFVEPNNAITVNTQNEFTTRPSSQVTISEGKACFSSGDGAAVCTNVADDGQQ

SEQ ID NO: 103 - protein, 673 aa, Haemophilus influenzae
PQSVLASGLQGMSVVHGTATMQVDGNKTTIRNSVNAIINWKQFNIDQNEMVQFLQESSNSAVFNRVTSDQISQLKGILDSNGQVFLINPNGITIGKDAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAANIRNQGKLSADSVSKDKSGNIVLSAKEGEAEIGGVISAQNQQAKGGKLMITGDKVTLKTGAVIDLSGKEGGETYLGGDERGEGKNGIQLAKKTSLEKGSTINVSGKEKGGRAIVWGDIALIDGNINAQGKDIAKTGGFVETSGHYLSIGNDAAVEAKEWLLDPDNVTISNGNDDQSQLKDDRGDSPNKILADNKHTVNNKTLSTALAKGIGVNISAKKKVNVTADINVHNGTLTLHSEQGGVEINGDITSEQNGNLTIKAGSWVDVHKNITIGMGFLNITAGGSVAFEKAGGDKGRAASDAKIVAQGVITAGSGQDFRFNNVSLNGTGRGLKFITAKGNKGNFSAKFDGVLNISGNISINHTANNQLSYFHRQGYTYWNLTQLNVDSDSSFSLTSIKDAIKVGGYDNAKDKKNTGGIGFTRDTIFNVKQGARVDISYTLPISPVKNSRIAAVNFDGN

SEQ ID NO: 104 - protein, 757 aa, Haemophilus influenzae
ITVKGGGVVNLKFNALSNNYKTPGVNISSRFINVTEGSQLNITGSMPSTTLFNVANDLIINATNSFVSIKEIEGTDTHLDTGLKVNGNVTIKGGNVTLGSNKAKTKFDKNVTVEKGANLTLASANFGNHKGALTVAGNINTQGKLVATGDTIDVSGDFTVGNDATFNGNTNNNLNITGNFTNNGTSIIDVKKGAAKLGNITNEGSLNITTHANTNQKTIITGNITNKKGDLNIRDNKNNAEIQIGGNISQKEGNLTISSDKVNITKQITIKAGVNGENSDSGTENNANLTIKTKTLELTNNLNISGFHKAEITAKDNSDLIIGKASSDSGNAGAQKVIFDKVKDSKISAGNHNVTLNSEVETSNGNSNAAGDSNGNNAGLTISAKDVAVNNNITSHKTINISATTGNVTTKEGTTINATTGGVEVTAKTGDIKGGIESKSGGVTLTATGDTLAVGNISGNTVSVTANSGTLTTKADSTIKGTGSVTTLSQSGDIGGTISGKTVSVTATTDSLTVKGGAKINATEGTATLTASSGKLTTEASSSITSAKGQVDLSARDGNIGGSINAANVTLNTTGTLTTVKGSSINANSGTLVINAEDAKLDGTASGDRTVVNATNASGSGSVTAVTSSSVNITGDLSTINGLNIISKNGKNTVVLKGAEIDVKYIQPGVASAEEVIEAKRALEKVKDLSDEERETLAKLGVSAVRFVEPNNAITVNTQNEFTTRPSSQVTISEGKACFSSGDGAAVCTNVADDGQQ

SEQ ID NO: 105 - protein, 235 aa, Haemophilus influenzae
NEPSDSANSAQGDISTSISMELLQGMVLEEPAPEPEDVQKEPEPEPENVQKEPEPEKQEIVEDPTIKPEPKKIKEPEKEKPKPKEKPKNKPKKEVKPQKKPINKELPKGDENIDSSANVNDKASTTSAANSNAQVAGSGTDTSEIAAYRSAIRREIESHKRYPTRAKIMRKQGKVSVSFNVGADGSLSGAKVTKSSGDESLDKAALDAINVSRSVGTRPAGFPSSLSVQISFTLQ

SEQ ID NO: 106 - protein, 169 aa, Haemophilus influenzae
CFDKQEAKQKVEDTKQTVASVASETKDAAANTMTEVKEKAQQLSTDVKNKVAEKVEDAKEVIKSATETASEKATEIKEAVSEKASEMKEAASEKASEMKEAASEKASEMKEAASEKASEMKEAASEKASEMKEAASEKVGEMKEKATEMKEAVSEKATQAVDAVKEATK

SEQ ID NO: 107 - protein, 639 aa, Haemophilus influenzae
SNSNPFGFNKADSILSDMVFYQIGGGVGYMAPPSRGTIPAAEFGIGWKANLMCGNFDIKTSIKNQLNGLTEGFKDLYSNVIESATGAVASLPAMVIQRANPQLYDILTNGLYQGKIDFNSLKTSCEEMSKKLADATLNGRWSQSSDMESFKDITSTEPDATRAKKRLEESQGKEGKEWVGGKKRGGQGQEPIKIVEDVAKAGYNMQNKRNVLESGRISGSSCSGLLCQTWDNPKDMTDWLTNVIGEKQLTTCKTDCGTASSSRAGVGLTPEIEKENIKTVTQLQKALNMSTPSVEVLAELSSTTVPVTRGLIESLREDPDAQVLGQRLASEIAVAKTMEKMLLARRAVLTGMREPNVANNKDAQEELEKILTLIDREIGQIRMEIDLQKSLTGNSAVAILQNKELREYNTGTGNVTRDSVDKRISDLANGTNSNAAELADADNINIPRNNVTLEVPQMSNSLSYTGTGGTSGNSRGNTSYSSVPAIKGTALDQATGLLKKFEGFSDKAYWDVNAYRTGYGSDTITKADGTIVKVTKDTVVSRADAERDLARRTQEFANRARNNVSSSTWDKLPPNAQAALTSYAYNYGSLTKDVIAAAQASAQSGDMNALANAVRNRQNNNNGVNAKRRNQEADYILGK

SEQ ID NO: 108 - protein, 174 aa, Haemophilus influenzae
CSEEQVQRDVYQSLDDCLADWKKIELCEADKNTESTQKTETTPQQGLGLNIRDNGNAESAVKNPAENNVQANQSENNAESTAKAESTDPSLGAAIAGGVIGYMAARAISSFLGPSYHPGNRAVTTPTGQVVQPQTNRSVGKPMLVKGNAGSMNSKPVSRGGFSSPNNTHRSSGG

SEQ ID NO: 109 - protein, 187 aa, Haemophilus influenzae
DDAQKPADHVTYEPQLDNQRDPNQYCAKCHKFDKIDKNQTLDRSGGELHFGKFHGAHLDKKNPNNGKAITCVSCHGNVSENHRRGAKDVMRFEGDIFGNKKPMYSVQEQNQVCFACHQPDKLREKLWAHDVHAMKLPCASCHTLHPKEDAMKGIQPKQRVKLCVDCHGKQQKRKAEQDKLTKQKDKQ

SEQ ID NO: 110 - protein, 71 aa, Haemophilus influenzae
ESKSSNTDNTATPCVGDKCVKTKAAEGKCGEGKCGADKAKSAEGKCGEGKCGASKPKAAEGKCGEGKCGSK

SEQ ID NO: 111 - protein, 386 aa, Haemophilus influenzae
SNSPAPISDADGNLSPSVVQSVNGSNVGGAWQPEIQKNSLPTMGNMVTPQPNFQPINQQPAMPTKPAQPAFQPSPKTVVSAPTVQTKTVTKTIADCVDGQHINIPRNPNTNAPDYSKISKGSYKGNTYKVNKGDTMFLIAYLAGIDVKELAALNNLSEPYNLSLGQVLKISNCGTKTVTTTVSVKQPAVTASTAMPAKPAVTYTPGANGTQIGSDGTIIGPIKSEAGISSSVSVATSSTQVTSSVNNANSTPINANVVAPIASNVAWQWPTSGNIIQGFSSTDGGNKGIDISGSRGQAVKAAAAGRIVYAGNALRGYGNLIIIKHNDDFLSAYAHNDKILVADQQEVKAGQDIAKMGSSGTNTVKLHFEIRYKGKSVDPVRYLPKR

SEQ ID NO: 112 - protein, 889 aa, Haemophilus influenzae
ETQSIKDTKEAISSEVDTQSTEDSELETISVTAEKVRDRKDNEVTGLGKIIKTSESISREQVLNIRDLTRYDPGISVVEQGRGASSGYSIRGMDRNRVALLVDGLPQTQSYVVQSPLVARSGYSGTGAINEIEYENVKAVEISKGGSSSEYGNGALAGSVTFQSKSAADILEGDKSWGIQTKNAYSSKNKGFTHSLAVAGKQGGFEGLAIYTQRNSIETQVHKDALKGVQSYNRLIAKEDGSNAYFVMEDECPKDYNSCIPSAKPPAKLSSQRETVSVSDYTGANRIKPNPMKYESQSWFLRGGYHFSEQHYIGGIFEFTQQKFDIRDMTFPAYLRSTEKPDLENSSFYPKQDYGAYQRIEDGRGVKYASGLYFDEHHRKQRVGIEYIYENKNKAGIIDKAVLSANQQNIILDSYMQHTHCSLYPNPSKNCRPTLDKPYSYYHSDRNVYKEKHNMLQLNLEKKIQQNWLTHQIVFNLGFDDFTSALQHKDYLTRRVTATAKSISEKPGETPRRNGFKLQPYLYPKPNASFVGRDHCNYQGSSSNYSDCKVRLIKGKNYYFAARNNMALGKYVDLGLGIRYDVSRTKANESTISVGKFKNFSWNTGIVIKPTEWLDLSYRLSTGFRNPSFAEMYGWRYGGKNDEVYVGKFKPETSRNQEFGLALKGDFGNIEISHFSNAYRNLIAFAEELKNGKGKGNYGYHNAQNAKLVGVNITAQLDFNGLWKRIPYGWYATFAYNRVKVKDQKINAGLASVSSYLFDAIQPSRYIIGLGYDHPSNTWGINTMFTQSKAKSQNELLGQRSLGNTSRGVKSTRKLTRAWHILDVSGYYMVNRSILFRLGVYNLLNYRYVTWEAVRQTAQGAVNQHQNVGNYTRYAASGRNYTLTLEMKF

SEQ ID NO: 113 - protein, 438 aa, Haemophilus influenzae
SGSSSTNTAETKKEDTLDISFKDLSPEELRAMGIEGDTSKDTVRTLIGKVKEHNKRFDEVILQNERLVKENEQLKNQANNTDYQIQQAVQAETGALINEINALKDQVVQAGNQVVGNGNAQANPNAPLPINGATTGPQVETRGGINWVNPSDMPTTDRQGNALQGIGVNGAGIGFPKTFDNNVNDIRKSNDGLQSSTGNNAIFNTATPFFTIAANSTLTNSVAMTALVGRVPIDNKVTEPYPFKLLIGRDNLIANGIELPDIEGAIVSGTASGDWTLSCVRGDVKSLTFVFSDGRIVSTDNSDDKIGWLSDPHGVPCIPGERRTNAPEYLTTNFFLTGASAAAQGLSSSQSTTVVDGGAVIGAVTGNNGKYILGQALGGGLRETADWFRQRYGQMFDAVYVPPGKEVAVHIEKQIDIDYDRMSRKVKYGQASRQSNLD

SEQ ID NO: 114 - protein, 483 aa, Haemophilus influenzae
ELVYKPLEQPVEPAKPDLKIESVNEKFAEKYPNQYNSWRSTANGDGENIIYADEENPRLIVLWGGYAFAKEYNAPRGHFYAVTDVRNILRTGAPKTANDGPQAMACWTCKGPDVPRLIAEWGEKDYFNAKWAKGGPEIVNSIGCADCHDTTSKDFAEGKPALRIARPHVLRALDALEKATAEKDKAEGRPHNNLSFNTAARTEKRAEICANCHVEYYFAGDIKQVTFPWDNGQTADDIEKYYDDIGFTDWTHSLSKAPMLKAQHPDFEIWSLGMHGKNGVTCVDCHMPKVQGADGKVYTDHQIQNPFDAFDHTCANCHDQSKEKLRDIVTSRKKEVKDVMGRLEDQVVKSHFEAKAAWDAGATKEEMEAALMDIRHAQWRWDYTAASHGGHMHAPEVVLRVLASGLDKVADARTKLAVILTKHGVKTPVQIPDISTADKAWKVMGIDIEKERKAKEEFLKTVVPQWEQQAREKGLLVDPPAQK

SEQ ID NO: 115 - protein, 448 aa, Haemophilus influenzae; identical to SEQ ID NO: 12

SEQ ID NO: 116 - protein, 888 aa, Haemophilus influenzae
EETLGQIDVVEKVISNDKKPFTEAKAKSTRENVFKETQTIDQVIRSIPGAFTQQDKGSGVVSVNIRGENGLGRVNTMVDGVTQTFYSTALDSGQSGGSSQFGAAIDPNFIAGVDVNKSNFSGASGINALAGSANFRTLGVNDVITDDKPFGIILKGMTGSNATKSNFMTMAAGRKWLDNGGYVGVVYGYSQREVSQDYRIGGGERLASLGQDILAKEKEAYFRNAGYVLNDAGQWTPDLSKNAWSCNEDPPRLAENIENVKCTHYSFDPRKNDRKEILKKLKAGTKPENIDELQNGKDGIKETDKSFERNKDQYSVAPIEPGSLQSRSRSHLLKFEYGDDHQNLGAQIRTLDNKIGSRKIENRNYQVNYNFNNNNYLDLNLMAAHNIGKTIYPKGGFFAGWRVADKLITKNVANIVDINNSHTFLLPKEIDLKTTLGFNYFTNEYSKNRFPEELSLFYNDDSHDQGNYSNLGRFRGNRNLLPQRSVILQPSGKQKFKTVYFDTALSKGIYHLNYSVNFTHYAFNGEYVGYENTANQINEPILHKSGHKKAFNHSATLSAELSDYFMPFFTYSRTHRMPNIQEMFFSQVSNAGVNTALKPEQSDTYQLGFNTYKKGLFTQDDVLGIKLVGYRSFIKNYIHNVYGVWWRDGVVPTWADSTGFRFTIAHQNYQPIVKKSGAELELNYDMGRFFANVSYAYQRTNQPTNYADASSRPNNASKEDILKQGYGLSRVSMLPKDYGRLEIGTRWFDQKLTLGMAARYYGKSKRATIEEEYINGSHFELKTSGKRTYYVVKKTEEIKKQPIILDLHVSYEPIKDLIIKAEVQNLLDKRYVDPLDAGNDAASQRYYSSLNDSICSKSQDCEDGGKDKTVLYNFARGRTYILSLNYKF

SEQ ID NO: 117 - protein, 935 aa, Haemophilus influenzae; identical to SEQ ID NO: 25

SEQ ID NO: 118 - protein, 436 aa, Haemophilus influenzae
KDKSEENPIFSTSDSGEYHELNTSPNKNSTALQPDEDATSYDDELQAKDDEVDEVKLSSDDLGTLPQHAQDALNGLLDAADQAIRITDQFSYTVTEGDTLKDVLVLSGLDDSSVQPLIKLDPELAHLKAGQQFYWILNKNDNLEYLNWLVSEKEERIYERLEDGKFKRQVIEKKSIWRKEVLKGEIQNSLNSSLREQGLDTRQISQLSNALQWQVSLRKLKKGTQFAILVSREYLGDKLTGQGNVEALRISSGGKNYYAVQAANGRYYNQQGETLGKGFARYPLQRQARVSSPFNPNRRHPVTGRVRPHKGVDFSVSQGTPVIAPADGTVEKVAYQAGGAGRYVMLRHGREYQTVYMHLSKSLVKAGQTVKKGERIALSGNTGISTGPHLHYEFRINGRAVNPLTVKLPGTSSGMTSAERKQFLVRVREAEKMLKP

SEQ ID NO: 119 - protein, 138 aa, Haemophilus influenzae
ACDDKPKMTEQTKTTDQAKMAAEQPDQKPVLINYKQIALDKSQQAIDTAKVAQDKSVQANEAVKVAMQKQQEAEIALSQGGEEAEKLSKQKMDESNAAAELALKLSEESKAASALSIQLSKEAEDAANKANQQAQAEK

SEQ ID NO: 120 - protein, 964 aa, Haemophilus influenzae
AQPTNQPTNQPTNQPTNQNGNVSEQLEQINVSGSTENSDTKTPPKIAETVKTAKTLEREQANNIKDIVKYETGVTVVEAGRFGQSGFAIRGVDENRVAINIDGLRQAETLSSQGFKELFEGYGNFNNTRNGAEIETLKEVNITKGANSIKSGSGSLGGSVIYKTKDARDYLLNKDYYVSYKKGYATENNQSFNTLTLAGRYKKFDALVVTTSRNGHELENYDYKNANSLTQGKKREKADPYKIEQDSTLLKLSFNPTENHRFTLAADLYEHRSRGQDLSYTLKYQKTDPNLLEVDSRHTNDKTKRRNISFSYENFSQTPFWDTLKITYSDQRIKTRARTDDYCDAGVRYCEGTANPAGLKLTDGKITRRDGSELQFEKKDKNIDNNIYDFDKFIDTDDRVIEGKLGLRRSSGTWYDCSIFDCKDKTKMKIFETEHPYGYGTTGTWKKDFELEIKKLNDKNFARVKDANNKTYSILPSSPGYLERLWQERDLDTNTQQLNLDLTKDFKTWRVEHNLQYGSSYNTTMKRMVNRAGNDASDVQWWAEPTLGYSLLYDKPHTCKTAYGGWKANLCPRVDPKFSFLLPIKTKEKSVYLFDNVVITDYLSFDLGYRYDNIHYQPKYKHGVTPKLPDDIVKGLFIPLPSGKNNNDDPEVKKNVQQNIDYIAKQNKKYKAHSYSFVSTIDPTSFLRLQLKYSKGFRAPTSDEMYFTFKHPDFTILPNTNLKPEIAKTKEIAFTLHNDDWGFISTSLFKTNYKNFIDLIFKGEKDFKLVSGGSTLPFSLYQNINRDSAVVKGIEINSKVFLGKMAKFMDGFNLSYKYTYQKGRMDGNIPMNAIQPKTMVYGLGYDHPSQKFGFNFYTTHVASKNPEDTYDIYAKDKNQTNTSIKWRSKSYTILDLIGYVQPIKNLTIRAGVYNLTNRKYITWDSARSIRSFGTSNVIDQKTGQGINRFYAPGRNYKMSVQFEF

SEQ ID NO: 121 - protein, 274 aa, Haemophilus influenzae
KPSSDTVESFTQSNSNEVPVQFQSLDQSQPLETTILDNPPAQNQMAVEQANQSEFAPKAEEAANNTTAQNPLVENAPMQQNVVQSPSQMPNEMAAASVMPMQPAQAEQPKATVPVQPMKKAVEPQVAHKDTVKKEVKVAENAQAPSKATEQNVAKTAGNAPIVEAKPVQVKKEKKVQIVDAKPVSKSAASRLSAKTLTVPKGVSLMQVFRDNQLNISDVNAMSKAAGAGNVLSSFKSGDKVTVSVNNQGRVNEMRLSNGARFVRQSDGSYQYKK

SEQ ID NO: 122 - protein, 844 aa, Haemophilus influenzae; identical to SEQ ID NO: 21

SEQ ID NO: 123 - protein, 225 aa, Haemophilus influenzae; identical to SEQ ID NO: 6

SEQ ID NO: 124 - protein, 666 aa, Haemophilus influenzae
VQPTLKPSDISIPAISEESQLATKRATTRLTQSHYRKIKLDDDFSEKIFDRYIKNLDFSHNTFLQSDIDELRQKYGTKLDEQLSQGDLSAAFDIYDVMMKRRYERYTYALSLLDKEPDLNGQDQIEIDREKAAAPQTEADANKLWDARVKNDIINLKLKDKKWSEIKAKLTKRYNLAIRRLTQTKADDIVQIYLNAFAREIDPHTSYLSPRTAKSFNESINLSLEGIGTTLQSEDDEISIKSLVPGAPAERSKKLHPGDKIIGVGQATGDIEDVVGWRLEDLVEKIKGKKGTKVRLEIEPAKGGKSRIITLVRDKVRIEDQAAKLTFEKVSGKNIAVIKIPSFYIGLTEDVKKLLVKLENQKAEALIVDLRENGGGALTEAVALSGLFIADGPVVQVRDAYQRIRVHEDDDATQQYKGLLFVMINRYSASASEIFAAAMQDYRRGIIIGQNTFGKGTVQQSRSLNFIYDLDQSPLGVLQYTIQKFYRVNGGSTQLKGVAADINFPEIIDAKEYGEDKEDNALAWDKIPSASYTEVGNIDYIDNAVNILNEKHLARIAKDPEFVALNEELKVRNERRDRKFLSLNYKMRKAENDKDDARRLKDLNERFKREGKKALKDIDDLPKDYEAPDFFLKEAEKIAADFVIFNSDQKINQANGLSEAKTESKK

SEQ ID NO: 125 - protein, 781 aa, Haemophilus influenzae
APFVAKDIRVDGVQGDLKQQIRASLPVRAGQRVTDNDVANIVRSLFVSGRFDDVKAHQEGDVLVVSVVAKSIISDVKIKGNSIIPTEALKQNLDANGFKVGDVLIREKLNEFAKSVKEHYASVGRYNATVEPIVNTLPNNRAEILIQINEDDKAKLASLTFKGNESVSSSTLQEQMELQPDSWWKLWGNKFEGAQFEKDLQSIRDYYLNNGYAKAQITKTDVQLNDEKTKVNVTIDVNEGLQYDLRSARIIGNLGGMSAELEPLLSALHLNDTFRRSDIADVENAIKAKLGERGYGSATVNSVPDFDDANKTLAITLVVDAGRRLTVRQLRFEGNTVSADSTLRQEMRQQEGTWYNSQLVELGKIRLDRTGFFETVENRIDPINGSNDEVDVVYKVKERNTGSINFGIGYGTESGISYQASVKQDNFLGTGAAVSIAGTKNDYGTSVNLGYTEPYFTKDGVSLGGNVFFENYDNSKSDTSSNYKRTTYGSNVTLGFPVNENNSYYVGLGHTYNKISNFALEYNRNLYIQSMKFKGNGIKTNDFDFSFGWNYNSLNRGYFPTKGVKASLGGRVTIPGSDNKYYKLSADVQGFYPLDRDHLWVVSAKASAGYANGFGNKRLPFYQTYTAGGIGSLRGFAYGSIGPNAIYAAQNGNSNSNSNGTFNKISSDVIGGNAITTASAELIVPTPFVSDKSQNTVRTSLFVDAASVWNTKWKSDKSGLDNNVLKSLPDYGKSSRIRASTGVGFQWQSPIGPLVFSYAKPIKKYENDDVEQFQFSIGGSF

SEQ ID NO: 126 - protein, 379 aa, Haemophilus influenzae; identical to SEQ ID NO: 8

SEQ ID NO: 127 - protein, 688 aa, Haemophilus influenzae
QSVELDSINVIATRDPSRFTYTPQKQSKDSLLSKQATSVAAALEDIPNVDVRGGSRSIAQKPNIRGLSDNRVVQVIDGVRQNFDLAHRGSYFLPMSLIQEIEVIKGPSSSLWGSGALGGVVAMRTPNALDLLKNNDKFGVKIRQGYQTANNLSEKDVSVFAANDKFDVLISGFYNNADNLRIGKGNKLNNTAYKQFGGLAKFGWQINDANRVELSHRETRFKQTAPGNNEAKNELTNEQIIEKINEYHNPLNNFPPKAKPSLEEFYSGVRARLGGVSYLSDQQIPDQSTVFNYYLTPDNPYLNTHIALYNNKTIEKEQRKVSGVKDQTKLTTRGINLRNSSELSHISFVYGVDYMRDKISTERGTNDKDAKFRAEPYNANSNTTGVYLIAHIPLFGEKLLLSPSVRYDHYDTSSKTVKYKDNHLSPATKLTWKVTNWLDFTAKYNEAFRAPSMQERFVSGAHFGTSTRVGDIINSFVANPNLRPETAKNKEITANLHFDSLFKQGDKFKIEATYFRNDVKDLINLKRLDDPNANGALSRTNSQYQNIANARLSGIELQAQYQTERLTLFTNYGSTKGRDKDSGEALSNIAASKIGVGADYALVKDKFTVGATITHYAAQHRVPKDHAVTYPSYILTDLRATYAPLKGEWKNLRLDFALENLFDRKYQPAFSLMEGTGRNAKISAVYSF